(19) United States
(12) Patent Application Publication
(10) Pub. No.: US 2018/0273577 A1
(43) Pub. Date: Sep. 27, 2018

(54) MODULATORS OF KRAS EXPRESSION

(71) Applicant: Ionis Pharmaceuticals, Inc., Carlsbad, CA (US)

(72) Inventors: Alexey REVENKO, San Diego, CA (US); Susan M. FREIER, San Marcos, CA (US); Robert A. MACLEOD, San Diego, CA (US)

(21) Appl. No.: 15/762,616

(22) PCT Filed: Sep. 23, 2016
(86) PCT No.: PCT/US16/53334

§ 371 Date: Mar. 23, 2018

Related U.S. Application Data
(60) Provisional application No. 62/232,120, filed on Sep. 24, 2015.

(51) Int. Cl. C07H 21/04; C12N 15/113; C12Q 1/68; A61K 31/7088

(52) U.S. Cl. CPC C07H 21/04; C12N 15/113; C12Q 1/68; A61K 31/7088; C12N 2310/315; C12N 2310/11; C12N 2310/3525; C12N 2310/3341; C12N 2310/341; C12N 2310/346; C12N 2320/51; C12N 2310/321; C12N 2310/3231

(57) ABSTRACT

The present embodiments provide methods, compounds, and compositions for inhibiting KRAS expression, which can be useful for treating, preventing, or ameliorating a disease associated with KRAS.

### SEQUENCE LISTING

The present application is being filed along with a Sequence Listing in electronic format. The Sequence Listing is provided as a file entitled BIOL0276WOSEQ_ST25.txt created Aug. 30, 2016, which is 567 kb in size. The information in the electronic format of the sequence listing is incorporated herein by reference in its entirety.

### FIELD

The present embodiments provide methods, compounds, and compositions for inhibiting KRAS expression, which can be useful for treating, preventing, or ameliorating a disease associated with KRAS.

### BACKGROUND

Kirsten Rat Sarcoma Viral Oncogene Homologue (KRAS) is one of three RAS protein family members (N, H and K-RAS) that are small membrane bound intracellular GTPase proteins. KRAS cycles between an inactive guanosine diphosphate (GDP)-bound state and an active guanosine triphosphate (GTP)-bound state. The process of exchanging the bound nucleotide is facilitated by guanine nucleotide exchange factors (GEFs) and GTPase activating proteins (GAPs). GEFs promote release of GDP from KRAS in exchange for GTP, resulting in active GTP-bound KRAS. GAPs promote hydrolysis of GTP to GDP, resulting in inactive GDP-bound KRAS. Active GTP-bound KRAS interacts with numerous effector proteins to stimulate signaling pathways regulating various cellular processes including proliferation and survival. Activating mutations render KRAS resistant to GAP-catalyzed hydrolysis of GTP and therefore lock the protein in an activated state.

KRAS is the most commonly mutated oncogene in human cancer. Approximately 30% of all human cancers have activating KRAS mutations with the highest incidence in colon, lung and pancreatic tumors, where KRAS mutation is also associated with poor prognosis.

### SUMMARY

Despite the prevalent role of KRAS in several types of cancer, KRAS is considered an “undruggable” target and no inhibitors directly targeting KRAS have yet entered clinical development. The present embodiments provided herein are directed to potent and tolerable compounds and compositions for inhibiting KRAS expression, which can be useful for treating, preventing, ameliorating, or slowing progression of cancer.

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

It is understood that the sequence set forth in each SEQ ID NO in the examples contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase. Compounds described by ISIS number (ISIS #) indicate a combination of nucleobase sequence, chemical modification, and motif.

Unless otherwise indicated, the following terms have the following meanings:

“2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (e.g., uracil).

“2′-O-methoxyethyl” (also 2′-MOE and 2′-O(CH₂)₂—OCH₃) refers to an O-methoxy-ethyl modification at the 2′ position of a sugar ring, e.g. a furanose ring. A 2′-O-methoxyethyl modified sugar is a modified sugar.

“2′-MOE nucleoside” (also 2′-O-methoxyethyl nucleoside) means a nucleoside comprising a 2′-MOE modified sugar moiety.

“2′-substituted nucleoside” or “2-modified nucleoside” means a nucleoside comprising a 2′-substituted or 2′-modified sugar moiety. As used herein, “2′-substituted” or “2-modified” in reference to a sugar moiety means a sugar moiety comprising a 2′-substituent group other than H or OH. “3′ target site” refers to the nucleotide of a target nucleic acid which is complementary to the 3′-most nucleotide of a particular compound.

“5′ target site” refers to the nucleotide of a target nucleic acid which is complementary to the 5′-most nucleotide of a particular compound.

“5-methylcytosine” means a cytosine with a methyl group attached to the 5 position.

“About” means within ±10% of a value. For example, if it is stated, “the compounds affected at least about 70% inhibition of KRAS”, it is implied that KRAS levels are inhibited within a range of 60% and 80%.

“Administration” or “administering” refers to routes of introducing a compound or composition provided herein to an individual to perform its intended function. An example of a route of administration that can be used includes, but is not limited to parenteral administration, such as subcutaneous, intravenous, or intramuscular injection or infusion.

“Administered concomitantly” or “co-administration” means administration of two or more compounds in any manner in which the pharmacological effects of both are manifest in the patient. Concomitant administration does not require that both compounds be administered in a single pharmaceutical composition, in the same dosage form, by the same route of administration, or at the same time. The effects of both compounds need not manifest themselves at the same time. The effects need only be overlapping for a period of time and need not be coextensive. Concomitant administration or co-administration encompasses administration in parallel or sequentially.

“Amelioration” refers to a lessening of at least one indicator, sign, or symptom of an associated disease, disorder, or condition. In certain embodiments, amelioration includes a delay or slowing in the progression of one or more indicators of a condition or disease. The severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound to the target.

“Antisense compound” means a compound comprising an antisense oligonucleotide and optionally one or more additional features, such as a conjugate group or terminal group. Examples of antisense compounds include single-stranded and double-stranded compounds, such as, antisense oligonucleotides, ribozymes, siRNAs, shRNAs, ssRNAs, and occupancy-based compounds.

“Antisense inhibition” means reduction of target nucleic acid levels in the presence of an antisense compound complementary to a target nucleic acid compared to target nucleic acid levels in the absence of the antisense compound.

“Antisense mechanisms” are all those mechanisms involving hybridization of a compound with target nucleic acid, wherein the outcome or effect of the hybridization is either target degradation or target occupancy with concomitant stalling of the cellular machinery involving, for example, transcription or splicing.

“Antisense oligonucleotide” means an oligonucleotide having a nucleobase sequence that is complementary to a target nucleic acid or region or segment thereof. In certain embodiments, an antisense oligonucleotide is specifically hybridizable to a target nucleic acid or region or segment thereof.

“Bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety.

In certain embodiments, the bicyclic sugar moiety does not comprise a furanosyl moiety.

“Branching group” means a group of atoms having at least 3 positions that are capable of forming covalent linkages to at least 3 groups. In certain embodiments, a branching group provides a plurality of reactive sites for connecting tethered ligands to an oligonucleotide via a conjugate linker and/or a cleavable moiety.

“Cell-targeting moiety” means a conjugate group or portion of a conjugate group that is capable of binding to a particular cell type or particular cell types.

“cEt” or “constrained ethyl” means a bicyclic furanosyl sugar moiety comprising a bridge connecting the 4′-carbon and the 2′-carbon, wherein the bridge has the formula: 4′-CH(CH₃)—O-2′.

“Chemical modification” in a compound describes the substitutions or changes through chemical reaction, of any of the units 3n the compound, “Modified nucleoside” means a nucleoside having, independently, a modified sugar moiety and/or modified nucleobase. “Modified oligonucleotide” means an oligonucleotide comprising at least one modified internucleoside linkage, a modified sugar, and/or a modified nucleobase.

“Chemically distinct region” refers to a region of an antisense compound that is in some way chemically different than another region of the same antisense compound. For example, a region having 2′-O-methoxyethyl nucleotides is chemically distinct from a region having nucleotides without 2′-O-methoxyethyl modifications.

“Chimeric antisense compounds” means antisense compounds that have at least 2 chemically distinct regions, each position having a plurality of subunits.

“Cleavable bond” means any chemical bond capable of being split. In certain embodiments, a cleavable bond is selected from among: an amide, a polyamide, an ester, an ether, one or both esters of a phosphodiester, a phosphate ester, a carbamate, a di-sulfide, or a peptide.

“Cleavable moiety” means a bond or group of atoms that is cleaved under physiological conditions, for example, inside a cell, an animal, or a human.

“Constrained ethyl nucleoside” (also cEt nucleoside) means a nucleoside comprising a bicyclic sugar moiety comprising a 4′-CH(CH₃)—O-2′ bridge.

“Complementary” in reference to an oligonucleotide means the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described herein, are limited to adenine (A) and thymine (T), adenine (A) and uracil (U), cytosine (C) and guanine (G), and 5-methyl cytosine (mC) and guanine (G) unless otherwise specified. Complementary oligonucleotides and/or nucleic acids need not have nucleobase complementarity at each nucleoside and may include one or more nucleobase mismatches. By contrast, “fully complementary” or “100% complementary” in reference to oligonucleotides means that such oligonucleotides have nucleobase matches at each nucleoside without any nucleobase mismatches.

“Conjugate group” means a group of atoms that is attached to a parent compound, e.g., an oligonucleotide.

“Conjugate linker” means a group of atoms that connects a conjugate group to a parent compound, e.g., an oligonucleotide.

“Contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other.

“Designing” or “Designed to” refer to the process of designing an oligomeric compound that specifically hybridizes with a selected nucleic acid molecule.

“Differently modified” mean chemical modifications or chemical substituents that are different from one another, including absence of modifications. Thus, for example, a MOE nucleoside and an unmodified DNA nucleoside are “differently modified,” even though the DNA nucleoside is unmodified. Likewise, DNA and RNA are “differently modified,” even though both are naturally-occurring unmodified nucleosides. Nucleosides that are the same but for comprising different nucleobases are not differently modified. For example, a nucleoside comprising a 2′-OMe modified sugar and an unmodified adenine nucleobase and a nucleoside comprising a 2′-OMe modified sugar and an unmodified thymine nucleobase are not differently modified.

“Dose” means a specified quantity of a pharmaceutical agent provided in a single administration, or in a specified time period. In certain embodiments, a dose may be administered in two or more boluses, tablets, or injections. For example, in certain embodiments, where subcutaneous administration is desired, the desired dose may require a volume not easily accommodated by a single injection. In such embodiments, two or more injections may be used to achieve the desired dose. In certain embodiments, a dose may be administered in two or more injections to minimize injection site reaction in an individual. In other embodiments, the pharmaceutical agent is administered by infusion over an extended period of time or continuously. Doses may be stated as the amount of pharmaceutical agent per hour, day, week or month.

“Dosing regimen” is a combination of doses designed to achieve one or more desired effects.

“Double-stranded antisense compound” means an antisense compound comprising two oligomeric compounds that are complementary to each other and form a duplex, and wherein one of the two said oligomeric compounds comprises an antisense oligonucleotide.

“Effective amount” means the amount of compound sufficient to effectuate a desired physiological outcome in an individual in need of the agent. The effective amount may vary among individuals depending on the health and physical condition of the individual to be treated, the taxonomic group of the individuals to be treated, the formulation of the composition, assessment of the individual's medical condition, and other relevant factors.

“Efficacy” means the ability to produce a desired effect.

“Expression” includes all the functions by which a gene's coded information is converted into structures present and operating in a cell. Such structures include, but are not limited to the products of transcription and translation.

“Fully modified” in reference to an oligonucleotide means a modified oligonucleotide in which each nucleoside is modified. “Uniformly modified” in reference to an oligonucleotide means a fully modified oligonucleotide in which at least one modification of each nucleoside is the same. For example, the nucleosides of a uniformly modified oligonucleotide can each have a 2′-MOE modification but different nucleobase modifications, and the internucleoside linkages may be different.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

“Hybridization” means the annealing of complementary oligonucleotides and/or nucleic acid molecules. In certain embodiments, complementary nucleic acid molecules include, but are not limited to, an antisense compound and a nucleic acid target. In certain embodiments, complementary nucleic acid molecules include, but are not limited to, an antisense oligonucleotide and a nucleic acid target.

“Immediately adjacent” means there are no intervening elements between the immediately adjacent elements of the same kind (e.g. no intervening nucleobases between the immediately adjacent nucleobases).

“Individual” means a human or non-human animal selected for treatment or therapy.

“Inhibiting the expression or activity” refers to a reduction or blockade of the expression or activity relative to the expression of activity in an untreated or control sample and does not necessarily indicate a total elimination of expression or activity.

“Internucleoside linkage” means a group or bond that forms a covalent linkage between adjacent nucleosides in an oligonucleotide. As used herein “modified internucleoside linkage” means any internucleoside linkage other than a naturally occurring, phosphate internucleoside linkage.

“KRAS” means any nucleic acid or protein of KRAS. “KRAS nucleic acid” means any nucleic acid encoding KRAS. For example, in certain embodiments, a KRAS nucleic acid includes a DNA sequence encoding KRAS, an RNA sequence transcribed from DNA encoding KRAS (including genomic DNA comprising introns and exons), including a non-protein encoding (i.e. non-coding) RNA sequence, and an mRNA sequence encoding KRAS. “KRAS mRNA” means an mRNA encoding a KRAS protein. “KRAS,” “K-ras,” “kras,” “k-ras,” “Ki-ras,” and “ki-ras” can be used interchangably without capitalizaiton or italicization of their spelling referring to nucleic acid or protein in a mutually exclusive manner unless specifically indicated to the contrary.

“KRAS specific inhibitor” refers to any agent capable of specifically inhibiting KRAS RNA and/or KRAS protein expression or activity at the molecular level. For example, KRAS specific inhibitors include nucleic acids (including antisense compounds), peptides, antibodies, small molecules, and other agents capable of inhibiting the expression of KRAS RNA and/or KRAS protein.

“Lengthened antisense oligonucleotides” are those that have one or more additional nucleosides relative to an antisense oligonucleotide disclosed herein, e.g. a parent oligonucleotide.

“Linearly modified sugar” or “linearly modified sugar moiety” means a modified sugar moiety that comprises an acyclic or non-bridging modification. Such linear modifications are distinct from bicyclic sugar modifications.

“Linked nucleosides” means adjacent nucleosides linked together by an internucleoside linkage.

“Mismatch” or “non-complementary” means a nucleobase of a first oligonucleotide that is not complementary to the corresponding nucleobase of a second oligonucleotide or target nucleic acid when the first and second oligonucleotides are aligned. For example, nucleobases including but not limited to a universal nucleobase, inosine, and hypoxanthine, are capable of hybridizing with at least one nucleobase but are still mismatched or non-complementary with respect to nucleobase to which it hybridized. As another example, a nucleobase of a first oligonucleotide that is not capable of hybridizing to the corresponding nucleobase of a second oligonucleotide or target nucleic acid when the first and second oligonucleotides are aligned is a mismatch or non-complementary nucleobase.

“Modulating” refers to changing or adjusting a feature in a cell, tissue, organ or organism. For example, modulating KRAS RNA can mean to increase or decrease the level of KRAS RNA and/or KRAS protein in a cell, tissue, organ or organism. A “modulator” effects the change in the cell, tissue, organ or organism. For example, a KRAS antisense compound can be a modulator that decreases the amount of KRAS RNA and/or KRAS protein in a cell, tissue, organ or organism.

“Monomer” refers to a single unit of an oligomer. Monomers include, but are not limited to, nucleosides and nucleotides.

“Motif” means the pattern of unmodified and/or modified sugar moieties, nucleobases, and/or internucleoside linkages, in an oligonucleotide.

“Natural” or “naturally occurring” means found in nature.

“Nucleic acid” refers to molecules composed of monomeric nucleotides. A nucleic acid includes, but is not limited to, ribonucleic acids (RNA), deoxyribonucleic acids (DNA), single-stranded nucleic acids, and double-stranded nucleic acids.

“Nucleobase” means a heterocyclic moiety capable of pairing with a base of another nucleic acid.

“Nucleobase sequence” means the order of contiguous nucleobases independent of any sugar, linkage, and/or nucleobase modification.

“Nucleoside” means a compound comprising a nucleobase and a sugar moiety. The nucleobase and sugar moiety are each, independently, unmodified or modified.

“Oligomeric compound” means a compound comprising a single oligonucleotide and optionally one or more additional features, such as a conjugate group or terminal group.

“Oligonucleotide” means a polymer of linked nucleosides each of which can be modified or unmodified, independent one from another.

“Parent oligonucleotide” means an oligonucleotide whose sequence is used as the basis of design for more oligonucleotides of similar sequence but with different lengths, motifs, and/or chemistries. The newly designed oligonucleotides may have the same or overlapping sequence as the parent oligonucleotide.

“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration.

“Pharmaceutically acceptable carrier or diluent” means any substance suitable for use in administering to an animal. For example, a pharmaceutically acceptable carrier can be a sterile aqueous solution, such as PBS or water-for-injection. As used herein “pharmaceutically acceptable salts” means physiologically and pharmaceutically acceptable salts of compounds, such as oligomeric compounds, i.e., salts that retain the desired biological activity of the parent compound and do not impart undesired toxicological effects thereto.

“Pharmaceutical agent” means a compound that provides a therapeutic benefit when administered to an individual.

“Pharmaceutical composition” means a mixture of substances suitable for administering to an individual. For example, a pharmaceutical composition may comprise one or more compounds or salt thereof and a sterile aqueous solution.

“Phosphorothioate linkage” means a modified internucleoside linkage between nucleosides where the phosphodiester bond is modified by replacing one of the non-bridging oxygen atoms with a sulfur atom.

“Phosphorus moiety” means a group of atoms comprising a phosphorus atom. In certain embodiments, a phosphorus moiety comprises a mono-, di-, or tri-phosphate, or phosphorothioate.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an oligomeric compound.

“Prodrug” means a form of a compound which, when administered to an individual, is metabolized to another form. In certain embodiments, the metabolized form is the active, or more active, form of the compound (e.g., drug).

“Prophylactically effective amount” refers to an amount of a pharmaceutical agent that provides a prophylactic or preventative benefit to an animal.

“Region” is defined as a portion of the target nucleic acid having at least one identifiable structure, function, or characteristic.

“RNAi compound” means a compound that acts, at least in part, through RISC or Ago2, but not through RNase H, to modulate a target nucleic acid and/or protein encoded by a target nucleic acid. RNAi compounds include, but are not limited to double-stranded siRNA, single-stranded RNA (ssRNA), and microRNA, including microRNA mimics.

“Segments” are defined as smaller or sub-portions of regions within a nucleic acid.

“Side effects” means physiological disease and/or conditions attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality.

“Single-stranded” in reference to a compound means the compound has only one oligonucleotide. “Self-complementary” means an oligonucleotide that at least partially hybridizes to itself. A compound consisting of one oligonucleotide, wherein the oligonucleotide of the compound is self-complementary, is a single-stranded compound. A single-stranded antisense compound may be capable of binding to a complementary compound to form a duplex.

“Sites,” as used herein, are defined as unique nucleobase positions within a target nucleic acid.

“Specifically hybridizable” refers to an antisense compound having a sufficient degree of complementarity between an antisense oligonucleotide and a target nucleic acid to induce a desired effect, while exhibiting minimal or no effects on non-target nucleic acids. In certain embodiments, specific hybridization occurs under physiological conditions.

“Specifically inhibit” a target nucleic acid means to reduce or block expression of the target nucleic acid while exhibiting fewer, minimal, or no effects on non-target nucleic acids reduction and does not necessarily indicate a total elimination of the target nucleic acid's expression.

“Sugar moiety” means a group of atoms that can link a nucleobase to another group, such as an internucleoside linkage, conjugate group, or terminal group. In certain embodiments, a sugar moiety is attached to a nucleobase to form a nucleoside. As used herein, “unmodified sugar moiety” or “unmodified sugar” means a 2′-OH(H) furanosyl moiety, as found in RNA, or a 2′-H(H) moiety, as found in DNA. Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl moiety comprising a non-hydrogen substituent in place of at least one hydrogen of an unmodified sugar moiety, or a sugar surrogate. In certain embodiments, a modified sugar moiety is a 2′-substituted sugar moiety. Such modified sugar moieties include bicyclic sugars and linearly modified sugars.

“Sugar surrogate” means a modified sugar moiety having other than a furanosyl moiety that can link a nucleobase to another group, such as an internucleoside linkage, conjugate group, or terminal group. Modified nucleosides comprising sugar surrogates can be incorporated into one or more positions within an oligonucleotide. In certain embodiments, such oligonucleotides are capable of hybridizing to complementary oligomeric compounds or nucleic acids.

“Target gene” refers to a gene encoding a target.

“Target nucleic acid,” “target RNA,” “target RNA transcript” and “nucleic acid target” all mean a nucleic acid capable of being targeted by antisense compounds.

“Target region” means a portion of a target nucleic acid to which one or more antisense compounds is targeted.

“Target segment” means the sequence of nucleotides of a target nucleic acid to which an antisense compound is targeted. “5′ target site” refers to the 5′-most nucleotide of a target segment. “3′ target site” refers to the 3′-most nucleotide of a target segment.

“Terminal group” means a chemical group or group of atoms that is covalently linked to a terminus of an oligonucleotide.

“Therapeutically effective amount” means an amount of a compound, pharmaceutical agent, or composition that provides a therapeutic benefit to an individual.

“Treat” refers to administering a compound or pharmaceutical composition to an animal in order to effect an alteration or improvement of a disease, disorder, or condition in the animal.

### Certain Embodiments

Certain embodiments provide methods, compounds and compositions for inhibiting KRAS expression.

Certain embodiments provide compounds targeted to a KRAS nucleic acid. In certain embodiments, the KRAS nucleic acid has the sequence set forth in GENBANK Accession No. NM_004985.4 (herein incorporated by reference, disclosed herein as SEQ ID NO: 1); GENBANK Accession No. NT_009714.17_TRUNC_18116000_18166000_COMP (herein incorporated by reference, disclosed herein as SEQ ID NO: 2), or GENBANK Accession No. NM_033360.3 (herein incorporated by reference, disclosed herein as SEQ ID NO: 3). In certain embodiments, the compound is a single-stranded oligonucleotide. In certain embodiments, the compound is double-stranded.

Certain embodiments provide a compound comprising a modified oligonucleotide consisting of 8 to 80 linked nucleosides and having a nucleobase sequence comprising at least 8 contiguous nucleobases of any of the nucleobase sequences of SEQ ID NOs: 13-2190. In certain embodiments, the compound is a single-stranded oligonucleotide. In certain embodiments, the compound is double-stranded. In certain embodiments, the modified oligonucleotide consists of 10 to 30 linked nucleosides.

Certain embodiments provide a compound comprising a modified oligonucleotide consisting of 9 to 80 linked nucleosides and having a nucleobase sequence comprising at least 9 contiguous nucleobases of any of the nucleobase sequences of SEQ ID NOs: 13-2190. In certain embodiments, the compound is a single-stranded oligonucleotide. In certain embodiments, the compound is double-stranded. In certain embodiments, the modified oligonucleotide consists of 10 to 30 linked nucleosides.

Certain embodiments provide a compound comprising a modified oligonucleotide consisting of 10 to 80 linked nucleosides and having a nucleobase sequence comprising at least 10 contiguous nucleobases of any of the nucleobase sequences of SEQ ID NOs: 13-2190. In certain embodiments, the compound is a single-stranded oligonucleotide. In certain embodiments, the compound is double-stranded. In certain embodiments, the modified oligonucleotide consists of 10 to 30 linked nucleosides.

Certain embodiments provide a compound comprising a modified oligonucleotide consisting of 11 to 80 linked nucleosides and having a nucleobase sequence comprising at least 11 contiguous nucleobases of any of the nucleobase sequences of SEQ ID NOs: 13-2190. In certain embodiments, the compound is a single-stranded oligonucleotide. In certain embodiments, the compound is double-stranded. In certain embodiments, the modified oligonucleotide consists of 11 to 30 linked nucleosides.

Certain embodiments provide a compound comprising a modified oligonucleotide consisting of 12 to 80 linked nucleosides and having a nucleobase sequence comprising at least 12 contiguous nucleobases of any of the nucleobase sequences of SEQ ID NOs: 13-2190. In certain embodiments, the compound is a single-stranded oligonucleotide. In certain embodiments, the compound is double-stranded. In certain embodiments, the modified oligonucleotide consists of 12 to 30 linked nucleosides.

Certain embodiments provide a compound comprising a modified oligonucleotide consisting of 16 to 80 linked nucleosides and having a nucleobase sequence comprising the nucleobase sequence of any one of SEQ ID NOs: 13-2190. In certain embodiments, the compound is a single-stranded oligonucleotide. In certain embodiments, the compound is double-stranded. In certain embodiments, the modified oligonucleotide consists of 16 to 30 linked nucleosides.

Certain embodiments provide a compound comprising a modified oligonucleotide consisting of the nucleobase sequence of any one of SEQ ID NOs: 13-2190. In certain embodiments, the compound is a single-stranded oligonucleotide. In certain embodiments, the compound is double-stranded.

In certain embodiments, a compound comprises or consists of a modified oligonucleotide consisting of 8 to 80 linked nucleosides having at least an 8, 9, 10, 11, 12, 13, 14, 15, or 16 contiguous nucleobase portion complementary to an equal length portion within nucleotides 463-478, 877-892, 1129-1144, 1313-1328, 1447-1462, 1686-1701, 1690-1705, 1778-1793, 1915-1930, 1919-1934, 1920-1935, 2114-2129, 2115-2130, 2461-2476, 2462-2477, 2463-2478, 4035-4050 of SEQ ID NO: 1. In certain embodiments, the modified oligonucleotide consists of 10 to 30 linked nucleosides.

In certain embodiments, a compound comprises or consists of a modified oligonucleotide consisting of 8 to 80 linked nucleosides complementary within nucleotides 463-478, 877-892, 1129-1144, 1313-1328, 1447-1462, 1686-1701, 1690-1705, 1778-1793, 1915-1930, 1919-1934, 1920-1935, 2114-2129, 2115-2130, 2461-2476, 2462-2477, 2463-2478, 4035-4050 of SEQ ID NO: 1. In certain embodiments, the modified oligonucleotide consists of 10 to 30 linked nucleosides.

In certain embodiments, a compound comprises or consists of a modified oligonucleotide consisting of 8 to 80 linked nucleosides having a nucleobase sequence comprising at least an 8, 9, 10, 11, 12, 13, 14, 15, or 16 contiguous nucleobase portion of any one of SEQ ID NOs: 239, 272, 569, 607, 615, 621, 640, 655, 678, 715, 790, 804, 854, 1028, 2130, 2136, 2142, 2154, and 2158. In certain embodiments, the modified oligonucleotide consists of 10 to 30 linked nucleosides.

In certain embodiments, a compound comprises or consists of a modified oligonucleotide consisting of 8 to 80 linked nucleosides having a nucleobase sequence comprising any one of SEQ ID NOs: 239, 272, 569, 607, 615, 621, 640, 655, 678, 715, 790, 804, 854, 1028, 2130, 2136, 2142, 2154, and 2158. In certain embodiments, the modified oligonucleotide consists of 10 to 30 linked nucleosides.

In certain embodiments, a compound comprises or consists of a modified oligonucleotide having a nucleobase sequence consisting of any one of SEQ ID NOs: 239, 272, 569, 607, 615, 621, 640, 655, 678, 715, 790, 804, 854, 1028, 2130, 2136, 2142, 2154, and 2158.

In certain embodiments, a compound comprises or consists of ISIS #651530, 651987, 695785, 695823, 651555, 651587, 695980, 695995, 696018, 696044, 716600, 746275, 716655, 716772, 740179, 740191, 740201, 740223, or 740233. Out of over 2,000 antisense oligonucleotides that were screened as described in the Examples section below, ISIS #651530, 651987, 695785, 695823, 651555, 651587, 695980, 695995, 696018, 696044, 716600, 746275, 716655, 716772, 740179, 740191, 740201, 740223, and 740233 emerged as the top lead compounds in terms of potency and/or tolerability.

In certain embodiments, any of the foregoing oligonucleotides comprises at least one modified internucleoside linkage, at least one modified sugar, and/or at least one modified nucleobase.

In certain embodiments, any of the foregoing oligonucleotides comprises at least one modified sugar. In certain embodiments, at least one modified sugar comprises a 2′-O-methoxyethyl group. In certain embodiments, at least one modified sugar is a bicyclic sugar, such as a 4′-CH(CH₃)—O-2′ group, a 4′-CH₂-O-2′ group, or a 4′-(CH₂)₂-O-2′group.

In certain embodiments, the modified oligonucleotide comprises at least one modified internucleoside linkage, such as a phosphorothioate internucleoside linkage.

In certain embodiments, any of the foregoing oligonucleotides comprises at least one modified nucleobase, such as 5-methylcytosine.

In certain embodiments, any of the foregoing oligonucleotides comprises:
a gap segment consisting of linked deoxynucleosides;
a 5′ wing segment consisting of linked nucleosides; and
a 3′ wing segment consisting of linked nucleosides;

wherein the gap segment is positioned between the 5′ wing segment and the 3′ wing segment and wherein each nucleoside of each wing segment comprises a modified sugar. In certain embodiments, the oligonucleotide consists of 16 to 80 linked nucleosides having a nucleobase sequence comprising the sequence recited in any one of SEQ ID NOs: 13-2190. In certain embodiments, the oligonucleotide consists of 16 to 80 linked nucleosides having a nucleobase sequence comprising the sequence recited in any one of SEQ ID NOs: 239, 272, 569, 607, 615, 621, 640, 655, 678, 715, 790, 804, 854, 1028, 2130, 2136, 2142, 2154, and 2158. In certain embodiments, the oligonucleotide consists of 16 to 30 linked nucleosides having a nucleobase sequence comprising the sequence recited in any one of SEQ ID NOs: 239, 272, 569, 607, 615, 621, 640, 655, 678, 715, 790, 804, 854, 1028, 2130, 2136, 2142, 2154, and 2158. In certain embodiments, the oligonucleotide consists of 16 linked nucleosides having a nucleobase sequence consisting of the sequence recited in any one of SEQ ID NOs: 239, 272, 569, 607, 615, 621, 640, 655, 678, 715, 790, 804, 854, 1028, 2130, 2136, 2142, 2154, and 2158.

In certain embodiments, a compound comprises or consists of a modified oligonucleotide consisting of 16-80 linked nucleobases having a nucleobase sequence comprising or consisting of the sequence recited in any one of SEQ ID NOs: 239, 272, 569, 607, 615, 621, 640, 655, 678, 715, 790, and 854, wherein the modified oligonucleotide comprises a gap segment consisting of ten linked deoxynucleosides;

a 5′ wing segment consisting of three linked nucleosides; and

a 3′ wing segment consisting of three linked nucleosides;

wherein the gap segment is positioned between the 5′ wing segment and the 3′ wing segment, wherein each nucleoside of each wing segment comprises a constrained ethyl (cEt) nucleoside; wherein each internucleoside linkage is a phosphorothioate linkage and wherein each cytosine is a 5-methylcytosine. In certain embodiments, the modified oligonucleotide consists of 16-30 linked nucleosides. In certain embodiments, the modified oligonucleotide consists of 16 linked nucleosides.

In certain embodiments, a compound comprises or consists of a modified oligonucleotide consisting of 16-80 linked nucleobases having a nucleobase sequence comprising or consisting of the sequence recited in SEQ ID NO: 2130, wherein the modified oligonucleotide comprises

a gap segment consisting of nine linked deoxynucleosides;

a 5′ wing segment consisting of one linked nucleoside; and

a 3′ wing segment consisting of six linked nucleosides;

wherein the gap segment is positioned between the 5′ wing segment and the 3′ wing segment; wherein the 5′ wing segment comprises a cEt nucleoside; wherein the 3′ wing segment comprises a cEt nucleoside, a 2′-O-methoxyethyl nucleoside, a cEt nucleoside, a 2′-O-methoxyethyl nucleoside, a cEt nucleoside, and 2′-O-methoxyethyl nucleoside in the 5′ to 3′ direction; wherein each internucleoside linkage is a phosphorothioate linkage; and wherein each cytosine is a 5-methylcytosine. In certain embodiments, the modified oligonucleotide consists of 16-30 linked nucleosides. In certain embodiments, the modified oligonucleotide consists of 16 linked nucleosides.

In certain embodiments, a compound comprises or consists of a modified oligonucleotide consisting of 16-80 linked nucleobases having a nucleobase sequence comprising or consisting of the sequence recited in any one of SEQ ID NOs: 804, 1028, and 2136, wherein the modified oligonucleotide comprises

a gap segment consisting of ten linked deoxynucleosides;

a 5′ wing segment consisting of two linked nucleosides; and

a 3′ wing segment consisting of four linked nucleosides;

wherein the gap segment is positioned between the 5′ wing segment and the 3′ wing segment; wherein the 5′ wing segment comprises a cEt nucleoside and a cEt nucleoside in the 5′ to 3′ direction; wherein the 3′ wing segment comprises a cEt nucleoside, a 2′-O-methoxyethyl nucleoside, a cEt nucleoside, and a 2′-O-methoxyethyl nucleoside in the 5′ to 3′ direction; wherein each internucleoside linkage is a phosphorothioate linkage; and wherein each cytosine is a 5-methylcytosine. In certain embodiments, the modified oligonucleotide consists of 16-30 linked nucleosides. In certain embodiments, the modified oligonucleotide consists of 16 linked nucleosides.

In certain embodiments, a compound comprises or consists of a modified oligonucleotide consisting of 16-80 linked nucleobases having a nucleobase sequence comprising or consisting of the sequence recited in SEQ ID NO: 2142, wherein the modified oligonucleotide comprises

a gap segment consisting of eight linked deoxynucleosides;

a 5′ wing segment consisting of two linked nucleosides; and

a 3′ wing segment consisting of six linked nucleosides;

wherein the gap segment is positioned between the 5′ wing segment and the 3′ wing segment; wherein the 5′ wing segment comprises a cEt nucleoside and a cEt nucleoside in the 5′ to 3′ direction; wherein the 3′ wing segment comprises a cEt nucleoside, a 2′-O-methoxyethyl nucleoside, a cEt nucleoside, a 2′-O-methoxyethyl nucleoside, a cEt nucleoside, and a cEt nucleoside in the 5′ to 3′ direction; wherein each internucleoside linkage is a phosphorothioate linkage; and wherein each cytosine is a 5-methylcytosine. In certain embodiments, the modified oligonucleotide consists of 16-30 linked nucleosides. In certain embodiments, the modified oligonucleotide consists of 16 linked nucleosides.

In certain embodiments, a compound comprises or consists of a modified oligonucleotide consisting of 16-80 linked nucleobases having a nucleobase sequence comprising or consisting of the sequence recited in SEQ ID NO: 2154, wherein the modified oligonucleotide comprises

a gap segment consisting of nine linked deoxynucleosides;

a 5′ wing segment consisting of two linked nucleosides; and

a 3′ wing segment consisting of five linked nucleosides;

wherein the gap segment is positioned between the 5′ wing segment and the 3′ wing segment; wherein the 5′ wing segment comprises a cEt nucleoside and a cEt nucleoside in the 5′ to 3′ direction; wherein the 3′ wing segment comprises a cEt nucleoside, a 2′-O-methoxyethyl nucleoside, a cEt nucleoside, a 2′-O-methoxyethyl nucleoside, and a cEt nucleoside in the 5′ to 3′ direction; wherein each internucleoside linkage is a phosphorothioate linkage; and wherein each cytosine is a 5-methylcytosine. In certain embodiments, the modified oligonucleotide consists of 16-30 linked nucleosides. In certain embodiments, the modified oligonucleotide consists of 16 linked nucleosides.

In certain embodiments, a compound comprises or consists of a modified oligonucleotide consisting of 16-80 linked nucleobases having a nucleobase sequence comprising or consisting of the sequence recited in SEQ ID NO: 2158, wherein the modified oligonucleotide comprises

a gap segment consisting of eight linked deoxynucleosides;

a 5′ wing segment consisting of three linked nucleosides; and

a 3′ wing segment consisting of five linked nucleosides;

wherein the gap segment is positioned between the 5′ wing segment and the 3′ wing segment; wherein the 5′ wing segment comprises a cEt nucleoside, a cEt nucleoside, and a cEt nucleoside in the 5′ to 3′ direction; wherein the 3′ wing segment comprises a cEt nucleoside, a deoxynucleoside, a cEt nucleoside, a deoxynucleoside, and a cEt nucleoside in the 5′ to 3′ direction; wherein each internucleoside linkage is a phosphorothioate linkage; and wherein each cytosine is a 5-methylcytosine. In certain embodiments, the modified oligonucleotide consists of 16-30 linked nucleosides. In certain embodiments, the modified oligonucleotide consists of 16 linked nucleosides.

In certain embodiments, a compound comprises or consists of ISIS 651987, or a salt thereof, which has the following chemical structure:

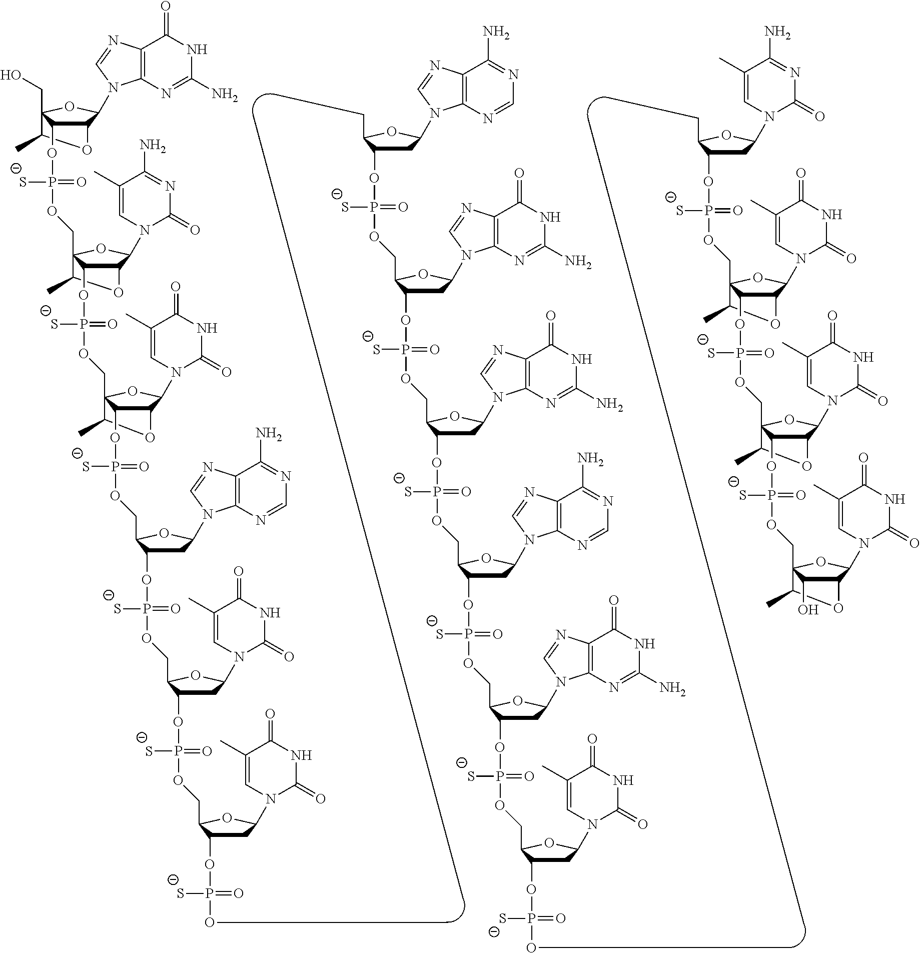

In certain embodiments, a compound comprises or consists of ISIS 696018, or a salt thereof, which has the following chemical structure:

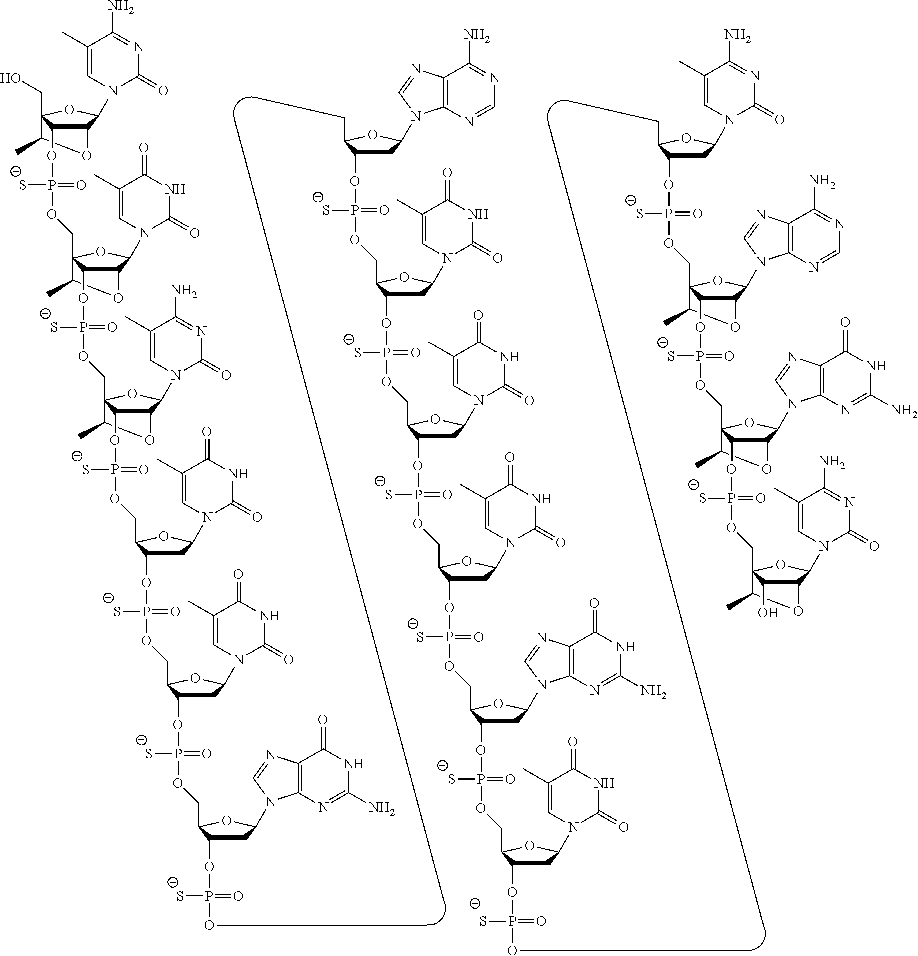

In certain embodiments, a compound comprises or consists of ISIS 696044, or a salt thereof, which has the following chemical structure:

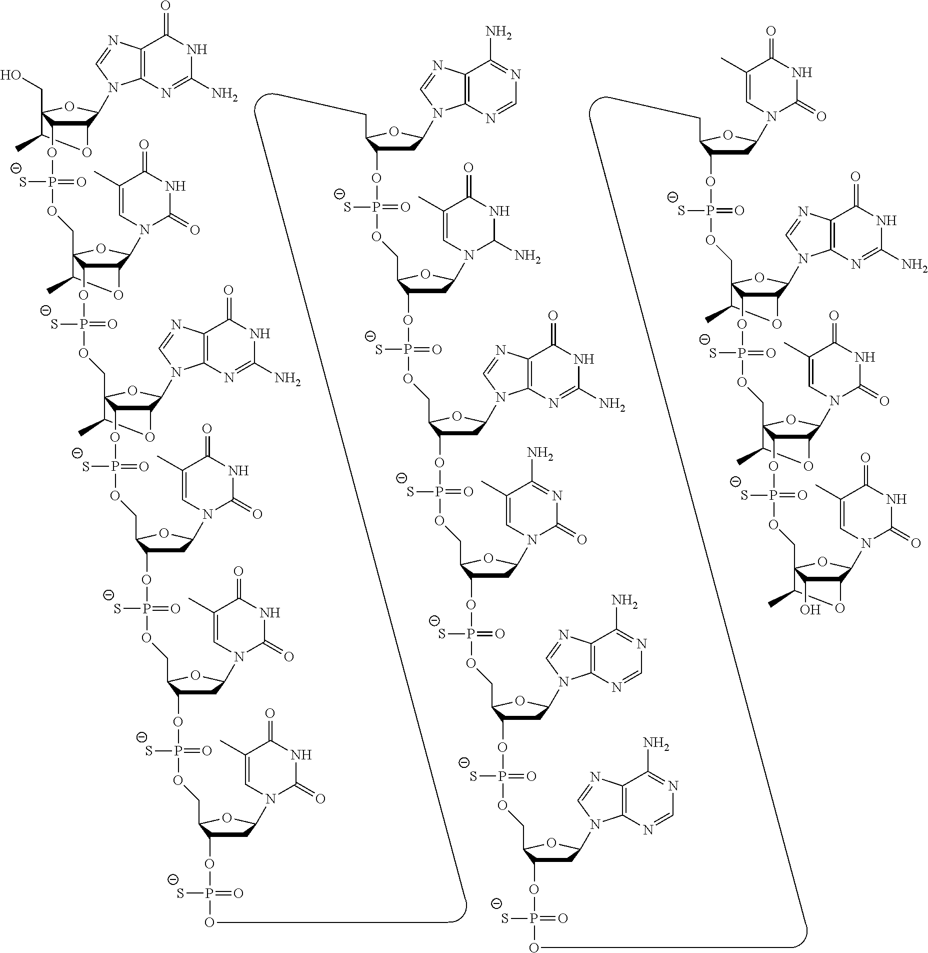

In certain embodiments, a compound comprises or consists of ISIS 716600, or a salt thereof, which has the following chemical structure:

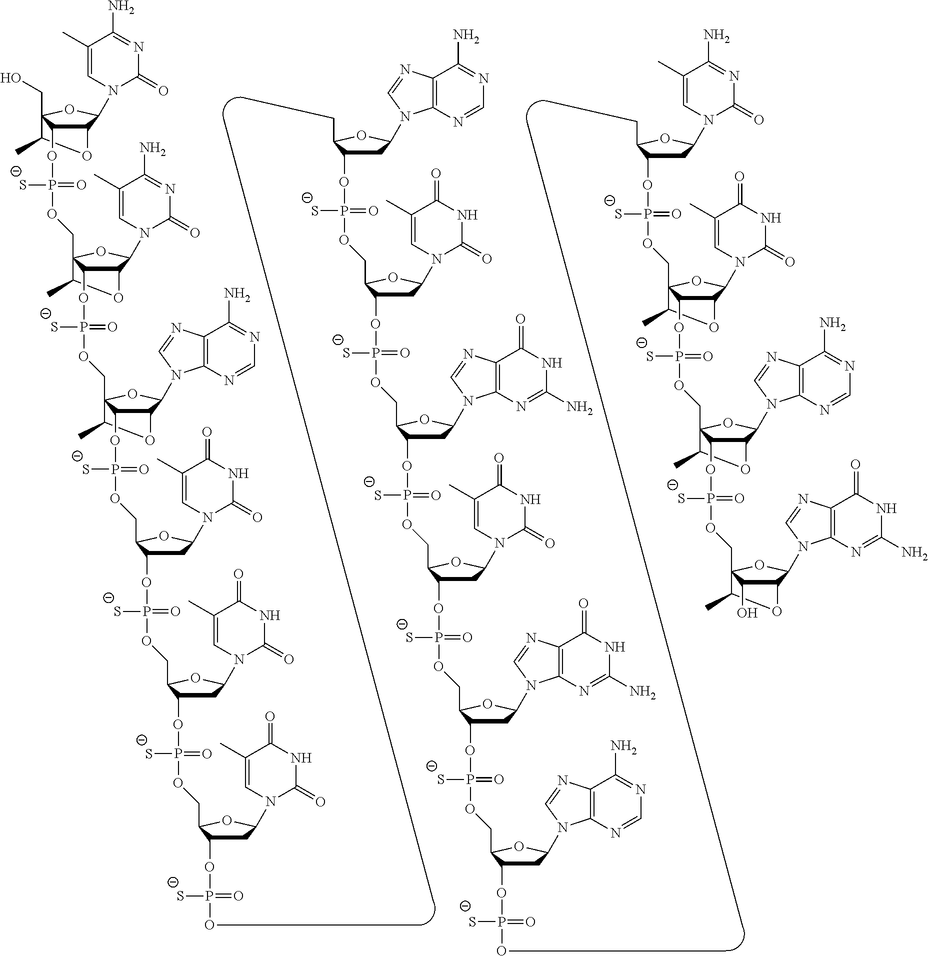

In certain embodiments, a compound comprises or consists of ISIS 716655, or a salt thereof, which has the following chemical structure:

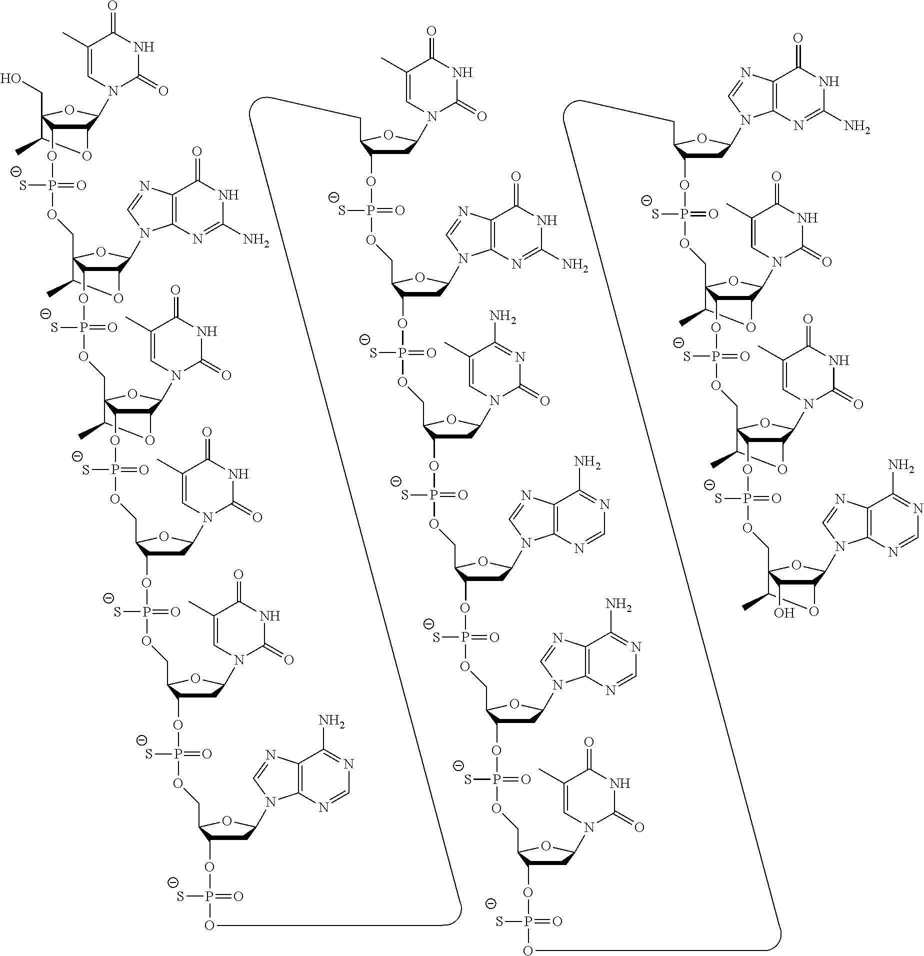

In certain embodiments, a compound comprises or consists of ISIS 740233, or a salt thereof, which has the following chemical structure:

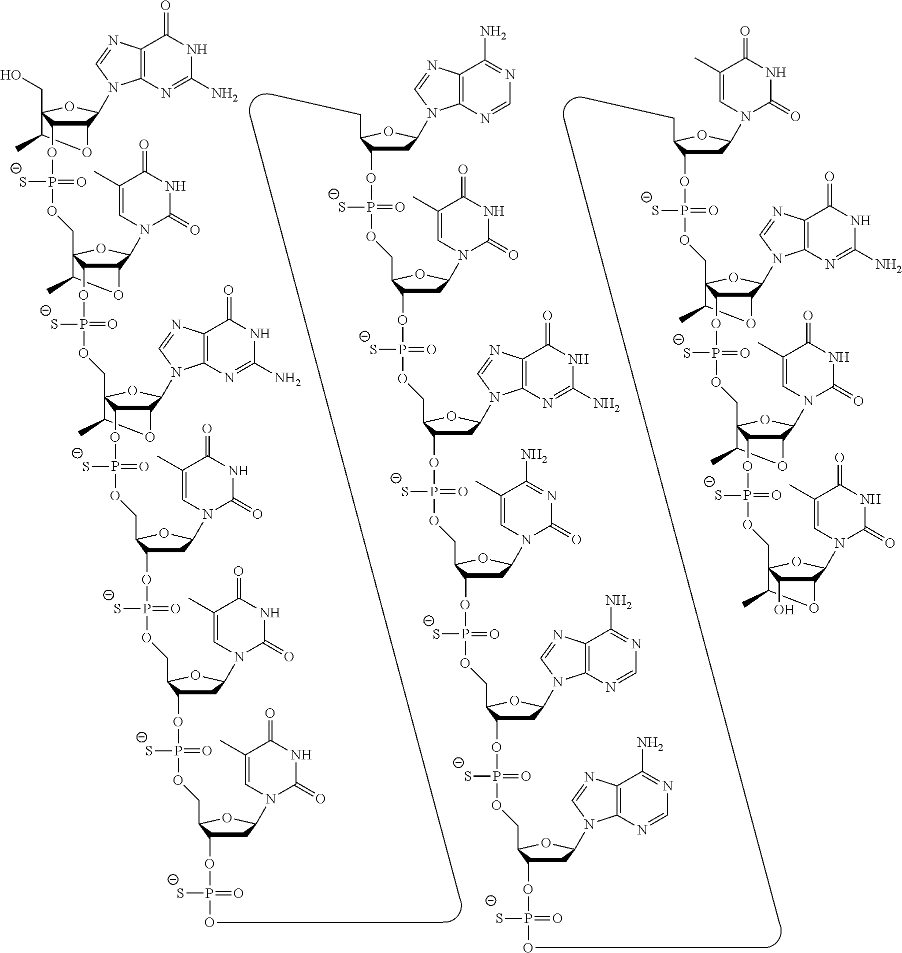

In certain embodiments, a compound comprises or consists of ISIS 746275, or a salt thereof, which has the following chemical structure:

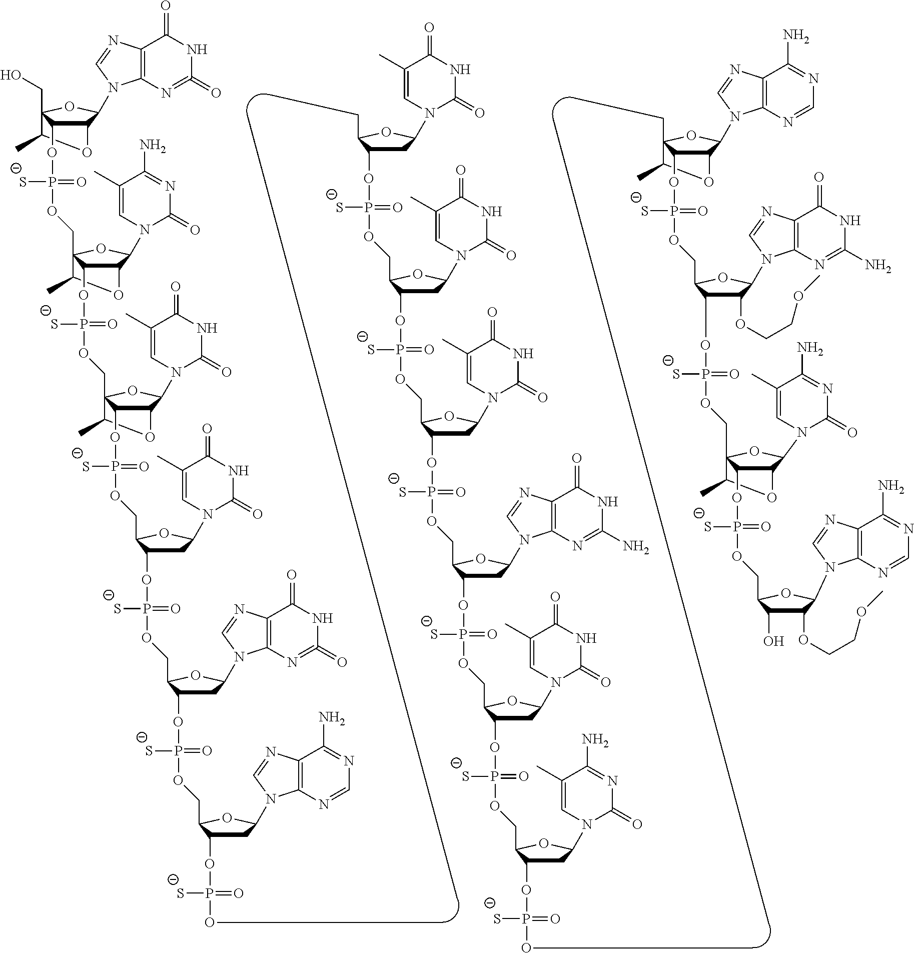

In any of the foregoing embodiments, the compound or oligonucleotide can be at least 85%, at least 90%, at least 95%, at least 98%, at least 99%, or 100% complementary to a nucleic acid encoding KRAS.

In any of the foregoing embodiments, the compound can be a single-stranded oligonucleotide. In certain embodiments, the compound comprises deoxyribonucleotides. In certain embodiments, the compound is double-stranded. In certain embodiments, the compound is double-stranded and comprises ribonucleotides.

In any of the foregoing embodiments, the oligonucleotide can consist of 8 to 80, 16 to 80, 10 to 30, 12 to 50, 13 to 30, 13 to 50, 14 to 30, 14 to 50, 15 to 30, 15 to 50, 16 to 30, or 16 to 50 linked nucleosides.

In certain embodiments, a compound comprises a modified oligonucleotide described herein and a conjugate group. In certain embodiments, the conjugate group is linked to the modified oligonucleotide at the 5′ end of the modified oligonucleotide. In certain embodiments, the conjugate group is linked to the modified oligonucleotide at the 3′ end of the modified oligonucleotide. In certain embodiments, the conjugate group comprises at least one N-Acetylgalactosamine (GalNAc), at least two N-Acetylgalactosamines (GalNAcs), or at least three N-Acetylgalactosamines (GalNAcs).

In certain embodiments, compounds or compositions provided herein comprise a salt of the modified oligonucleotide. In certain embodiments, the salt is a sodium salt. In certain embodiments, the salt is a potassium salt.

In certain embodiments, the compounds or compositions as described herein are active by virtue of having at least one of an in vitro IC₅₀ of less than 250 nM, less than 200 nM, less than 150 nM, less than 100 nM, less than 90 nM, less than 80 nM, less than 70 nM, less than 65 nM, less than 60 nM, less than 55 nM, less than 50 nM, less than 45 nM, less than 40 nM, less than 35 nM, less than 30 nM, less than 25 nM, or less than 20 nM.

In certain embodiments, the compounds or compositions as described herein are highly tolerable as demonstrated by having at least one of an increase an alanine transaminase (ALT) or aspartate transaminase (AST) value of no more than 4 fold, 3 fold, or 2 fold over control treated animals or an increase in liver, spleen, or kidney weight of no more than 30%, 20%, 15%, 12%, 10%, 5%, or 2% compared to control treated animals. In certain embodiments, the compounds or compositions as described herein are highly tolerable as demonstrated by having no increase of ALT or AST over control treated animals. In certain embodiments, the compounds or compositions as described herein are highly tolerable as demonstrated by having no increase in liver, spleen, or kidney weight over control treated animals.

### Certain Indications

Certain embodiments provided herein relate to methods of inhibiting KRAS expression by administration of a KRAS specific inhibitor, such as a compound targeted to KRAS, which can be useful for treating, preventing, or ameliorating cancer in an individual. Examples of types of cancer include but are not limited to lung cancer (e.g. non-small cell lung carcinoma (NSCLC) and small-cell lung carcinoma (SCLC)), gastrointestinal cancer (e.g. large intestinal cancer, small intestinal cancer, and stomach cancer), colon cancer, colorectal cancer, bladder cancer, liver cancer, esophageal cancer, pancreatic cancer, biliary tract cancer, breast cancer, ovarian cancer, endometrial cancer, cervical cancer, prostate cancer, hematopoetic cancer (e.g. leukemia, myeloid leukemia, and lymphoma), brain cancer (e.g. glioblastoma), malignant peripheral nerve sheath tumor (MPNST), neurofibromatosis type 1 (NF1) mutant MPNST, or neurofibroma. In certain embodiments, the cancer has cancer cells expressing mutant KRAS.

In certain embodiments, a method of treating, preventing, or ameliorating cancer comprises administering to the individual a KRAS specific inhibitor, thereby treating, preventing, or ameliorating cancer. In certain embodiments, the cancer is lung cancer (e.g. non-small cell lung carcinoma (NSCLC) and small-cell lung carcinoma (SCLC)), gastrointestinal cancer (e.g. large intestinal cancer, small intestinal cancer, and stomach cancer), colon cancer, colorectal cancer, bladder cancer, liver cancer, esophageal cancer, pancreatic cancer, biliary tract cancer, breast cancer, ovarian cancer, endometrial cancer, cervical cancer, prostate cancer, hematopoetic cancer (e.g. leukemia, myeloid leukemia, and lymphoma), brain cancer (e.g. glioblastoma), malignant peripheral nerve sheath tumor (MPNST), neurofibromatosis type 1 (NF1) mutant MPNST, or neurofibroma. In certain embodiments, the cancer has cancer cells expressing mutant KRAS. In certain embodiments, the KRAS specific inhibitor is a compound targeted to KRAS, such as an antisense oligonucleotide targeted to KRAS. In certain embodiments, the KRAS specific inhibitor is a compound comprising a modified oligonucleotide consisting of 8 to 80 linked nucleosides and having a nucleobase sequence comprising at least 8 contiguous nucleobases of any of the nucleobase sequences of SEQ ID NOs: 13-2190. In certain embodiments, the KRAS specific inhibitor is a compound comprising a modified oligonucleotide consisting of 16 to 80 linked nucleosides and having a nucleobase sequence comprising the nucleobase sequence of any one of SEQ ID NOs: 13-2190. In certain embodiments, the KRAS specific inhibitor is a compound comprising a modified oligonucleotide consisting of 16 linked nucleosides and having a nucleobase sequence consisting of the nucleobase sequence of any one of SEQ ID NOs: 13-2190. In certain embodiments, the KRAS specific inhibitor is a compound comprising a modified oligonucleotide consisting of 16 to 80 linked nucleosides having a nucleobase sequence comprising any one of SEQ ID NOs: 239, 272, 569, 607, 615, 621, 640, 655, 678, 715, 790, 804, 854, 1028, 2130, 2136, 2142, 2154, and 2158. In certain embodiments, the KRAS specific inhibitor is a compound comprising a modified oligonucleotide consisting of 16 linked nucleosides having a nucleobase sequence consisting of any one of SEQ ID NOs: 239, 272, 569, 607, 615, 621, 640, 655, 678, 715, 790, 804, 854, 1028, 2130, 2136, 2142, 2154, and 2158. In certain embodiments, the KRAS specific inhibitor is ISIS #651530, 651987, 695785, 695823, 651555, 651587, 695980, 695995, 696018, 696044, 716600, 746275, 716655, 716772, 740179, 740191, 740201, 740223, or 740233. In certain embodiments, the KRAS specific inhibitor is ISIS #651987. In certain embodiments, the KRAS specific inhibitor is ISIS #746275. In any of the foregoing embodiments, the compound can be a single-stranded oligonucleotide. In any of the foregoing embodiments, the modified oligonucleotide can consist of 10 to 30 linked nucleosides. In certain embodiments, the compound is administered to the individual parenterally. In certain embodiments, administering the compound reduces the number of cancer cells in an individual, reduces the size of a tumor in an individual, reduces or inhibits growth or proliferation of a tumor in an individual, prevents metastasis or reduces the extent of metastasis, and/or extends the survival of an individual having cancer, including but not limited to progression free survival (PFS) or overall survival.

In certain embodiments, a method of inhibiting expression of KRAS in an individual having, or at risk of having, cancer comprises administering a KRAS specific inhibitor to the individual, thereby inhibiting expression of KRAS in the individual. In certain embodiments, the cancer expresses mutant KRAS. In certain embodiments, administering the inhibitor inhibits expression of KRAS in a tumor, such as a tumor in the lung, gastrointestinal system, bladder, liver, esophagus, pancreas, biliary tract, breast, ovary, endometrium, cervix, prostate, or brain. In certain embodiments, administering the KRAS specific inhibitor inhibits expression of mutant KRAS. In certain embodiments, administering the KRAS specific inhibitor selectively inhibits expression of mutant KRAS relative to wildtype KRAS. In certain embodiments, the KRAS specific inhibitor is a compound comprising a modified oligonucleotide consisting of 8 to 80 linked nucleosides and having a nucleobase sequence comprising at least 8 contiguous nucleobases of any of the nucleobase sequences of SEQ ID NOs: 13-2190. In certain embodiments, the KRAS specific inhibitor is a compound comprising a modified oligonucleotide consisting of 16 to 80 linked nucleosides and having a nucleobase sequence comprising the nucleobase sequence of any one of SEQ ID NOs: 13-2190. In certain embodiments, the KRAS specific inhibitor is a compound comprising a modified oligonucleotide consisting of 16 linked nucleosides and having a nucleobase sequence consisting of the nucleobase sequence of any one of SEQ ID NOs: 13-2190. In certain embodiments, the KRAS specific inhibitor is a compound comprising a modified oligonucleotide consisting of 16 to 80 linked nucleosides having a nucleobase sequence comprising any one of SEQ ID NOs: 239, 272, 569, 607, 615, 621, 640, 655, 678, 715, 790, 804, 854, 1028, 2130, 2136, 2142, 2154, and 2158. In certain embodiments, the KRAS specific inhibitor is a compound comprising a modified oligonucleotide consisting of 16 linked nucleosides having a nucleobase sequence consisting of any one of SEQ ID NOs: 239, 272, 569, 607, 615, 621, 640, 655, 678, 715, 790, 804, 854, 1028, 2130, 2136, 2142, 2154, and 2158. In certain embodiments, the KRAS specific inhibitor is ISIS #651530, 651987, 695785, 695823, 651555, 651587, 695980, 695995, 696018, 696044, 716600, 746275, 716655, 716772, 740179, 740191, 740201, 740223, or 740233. In certain embodiments, the KRAS specific inhibitor is ISIS #651987. In certain embodiments, the KRAS specific inhibitor is ISIS #746275. In any of the foregoing embodiments, the compound can be a single-stranded oligonucleotide. In any of the foregoing embodiments, the modified oligonucleotide can consist of 10 to 30 linked nucleosides.

In certain embodiments, a method of inhibiting expression of KRAS in a cell comprises contacting the cell with a KRAS specific inhibitor, thereby inhibiting expression of KRAS in the cell. In certain embodiments, the cell is a cancer cell. In certain embodiments, the cell is in the lung, gastrointestinal system, bladder, liver, esophagus, pancreas, biliary tract, breast, ovary, endometrium, cervix, prostate, or brain. In certain embodiments, the cell is in the lung, gastrointestinal system, bladder, liver, esophagus, pancreas, biliary tract, breast, ovary, endometrium, cervix, prostate, or brain of an individual who has, or is at risk of having cancer. In certain embodiments, the cancer cell expresses mutant KRAS and contacting the cancer cell with the KRAS specific inhibitor inhibits expression of mutant KRAS in the cancer cell. In certain embodiments, contacting the cancer cell with the KRAS specific inhibitor selectively inhibits expression of mutant KRAS. In certain embodiments, the KRAS specific inhibitor is a compound comprising a modified oligonucleotide consisting of 8 to 80 linked nucleosides and having a nucleobase sequence comprising at least 8 contiguous nucleobases of any of the nucleobase sequences of SEQ ID NOs: 13-2190. In certain embodiments, the KRAS specific inhibitor is a compound comprising a modified oligonucleotide consisting of 16 to 80 linked nucleosides and having a nucleobase sequence comprising the nucleobase sequence of any one of SEQ ID NOs: 13-2190. In certain embodiments, the KRAS specific inhibitor is a compound comprising a modified oligonucleotide consisting of 16 linked nucleosides and having a nucleobase sequence consisting of the nucleobase sequence of any one of SEQ ID NOs: 13-2190. In certain embodiments, the KRAS specific inhibitor is a compound comprising a modified oligonucleotide consisting of 16 to 80 linked nucleosides having a nucleobase sequence comprising any one of SEQ ID NOs: 239, 272, 569, 607, 615, 621, 640, 655, 678, 715, 790, 804, 854, 1028, 2130, 2136, 2142, 2154, and 2158. In certain embodiments, the KRAS specific inhibitor is a compound comprising a modified oligonucleotide consisting of 16 linked nucleosides having a nucleobase sequence consisting of any one of SEQ ID NOs: 239, 272, 569, 607, 615, 621, 640, 655, 678, 715, 790, 804, 854, 1028, 2130, 2136, 2142, 2154, and 2158. In certain embodiments, the KRAS specific inhibitor is ISIS #651530, 651987, 695785, 695823, 651555, 651587, 695980, 695995, 696018, 696044, 716600, 746275, 716655, 716772, 740179, 740191, 740201, 740223, or 740233. In certain embodiments, the KRAS specific inhibitor is ISIS #651987. In certain embodiments, the KRAS specific inhibitor is ISIS #746275. In any of the foregoing embodiments, the compound can be a single-stranded oligonucleotide. In any of the foregoing embodiments, the modified oligonucleotide can consist of 10 to 30 linked nucleosides.

In certain embodiments, a method of reducing the number of cancer cells in an individual, reducing the size of a tumor in an individual, reducing or inhibiting growth or proliferation of a tumor in an individual, preventing metastasis or reducing the extent of metastasis, and/or extending the survival (including but not limited to progression free survival (PFS) or overall survival) of an individual having cancer comprises administering a KRAS specific inhibitor to the individual. In certain embodiments, the inhibitor is a compound targeted to KRAS. In certain embodiments, the inhibitor is a compound targeted to mutant KRAS. In certain embodiments, the inhibitor is a compound selectively targeted to mutant KRAS. In certain embodiments, the cancer cells or tumor expresses mutant KRAS. In certain embodiments, administering the KRAS specific inhibitor to the individual selectively reduces the number of mutant KRAS expressing cancer cells, selectively reduces the size of a mutant KRAS expressing tumor, selectively reduces or inhibits growth or proliferation of a mutant KRAS expressing tumor, selectively prevents metastasis or reduces the extent of metastasis of a mutant KRAS expressing tumor, and/or selectively extends the survival of an individual having a mutant KRAS expressing cancer relative to cells, tumors, and cancer expressing wildtype KRAS. In certain embodiments, the KRAS specific inhibitor is a compound comprising a modified oligonucleotide consisting of 8 to 80 linked nucleosides and having a nucleobase sequence comprising at least 8 contiguous nucleobases of any of the nucleobase sequences of SEQ ID NOs: 13-2190. In certain embodiments, the KRAS specific inhibitor is a compound comprising a modified oligonucleotide consisting of 16 to 80 linked nucleosides and having a nucleobase sequence comprising the nucleobase sequence of any one of SEQ ID NOs: 13-2190. In certain embodiments, the KRAS specific inhibitor is a compound comprising a modified oligonucleotide consisting of 16 linked nucleosides and having a nucleobase sequence consisting of the nucleobase sequence of any one of SEQ ID NOs: 13-2190. In certain embodiments, the KRAS specific inhibitor is a compound comprising a modified oligonucleotide consisting of 16 to 80 linked nucleosides having a nucleobase sequence comprising any one of SEQ ID NOs: 239, 272, 569, 607, 615, 621, 640, 655, 678, 715, 790, 804, 854, 1028, 2130, 2136, 2142, 2154, and 2158. In certain embodiments, the KRAS specific inhibitor is a compound comprising a modified oligonucleotide consisting of 16 linked nucleosides having a nucleobase sequence consisting of any one of SEQ ID NOs: 239, 272, 569, 607, 615, 621, 640, 655, 678, 715, 790, 804, 854, 1028, 2130, 2136, 2142, 2154, and 2158. In certain embodiments, the KRAS specific inhibitor is ISIS #651530, 651987, 695785, 695823, 651555, 651587, 695980, 695995, 696018, 696044, 716600, 746275, 716655, 716772, 740179, 740191, 740201, 740223, or 740233. In certain embodiments, the KRAS specific inhibitor is ISIS #651987. In certain embodiments, the KRAS specific inhibitor is ISIS #746275. In any of the foregoing embodiments, the compound can be a single-stranded oligonucleotide. In any of the foregoing embodiments, the modified oligonucleotide can consist of 10 to 30 linked nucleosides. In certain embodiments, the compound is administered to the individual parenterally.

Certain embodiments are drawn to a KRAS specific inhibitor for use in treating cancer. In certain embodiments, the cancer is lung cancer (e.g. non-small cell lung carcinoma (NSCLC) and small-cell lung carcinoma (SCLC)), gastrointestinal cancer (e.g. large intestinal cancer, small intestinal cancer, and stomach cancer), colon cancer, colorectal cancer, bladder cancer, liver cancer, esophageal cancer, pancreatic cancer, biliary tract cancer, breast cancer, ovarian cancer, endometrial cancer, cervical cancer, prostate cancer, hematopoetic cancer (e.g. leukemia, myeloid leukemia, and lymphoma), brain cancer (e.g. glioblastoma), malignant peripheral nerve sheath tumor (MPNST), neurofibromatosis type 1 (NF1) mutant MPNST, or neurofibroma. In certain embodiments, the cancer expresses mutant KRAS. In certain embodiments, the inhibitor is a compound targeted to KRAS. In certain embodiments, the inhibitor is a compound targeted to mutant KRAS. In certain embodiments, the inhibitor is a compound selectively targeted to mutant KRAS. In certain embodiments, the KRAS specific inhibitor is a compound comprising a modified oligonucleotide consisting of 8 to 80 linked nucleosides and having a nucleobase sequence comprising at least 8 contiguous nucleobases of any of the nucleobase sequences of SEQ ID NOs: 13-2190. In certain embodiments, the KRAS specific inhibitor is a compound comprising a modified oligonucleotide consisting of 16 to 80 linked nucleosides and having a nucleobase sequence comprising the nucleobase sequence of any one of SEQ ID NOs: 13-2190. In certain embodiments, the KRAS specific inhibitor is a compound comprising a modified oligonucleotide consisting of 16 linked nucleosides and having a nucleobase sequence consisting of the nucleobase sequence of any one of SEQ ID NOs: 13-2190. In certain embodiments, the KRAS specific inhibitor is a compound comprising a modified oligonucleotide consisting of 16 to 80 linked nucleosides having a nucleobase sequence comprising any one of SEQ ID NOs: 239, 272, 569, 607, 615, 621, 640, 655, 678, 715, 790, 804, 854, 1028, 2130, 2136, 2142, 2154, and 2158. In certain embodiments, the KRAS specific inhibitor is a compound comprising a modified oligonucleotide consisting of 16 linked nucleosides having a nucleobase sequence consisting of any one of SEQ ID NOs: 239, 272, 569, 607, 615, 621, 640, 655, 678, 715, 790, 804, 854, 1028, 2130, 2136, 2142, 2154, and 2158. In certain embodiments, the KRAS specific inhibitor is ISIS #651530, 651987, 695785, 695823, 651555, 651587, 695980, 695995, 696018, 696044, 716600, 746275, 716655, 716772, 740179, 740191, 740201, 740223, or 740233. In certain embodiments, the KRAS specific inhibitor is ISIS #651987. In certain embodiments, the KRAS specific inhibitor is ISIS #746275. In any of the foregoing embodiments, the compound can be a single-stranded oligonucleotide. In any of the foregoing embodiments, the modified oligonucleotide can consist of 10 to 30 linked nucleosides. In certain embodiments, the compound is administered to the individual parenterally.

Certain embodiments are drawn to a KRAS specific inhibitor for use in reducing the number of cancer cells in an individual, reducing the size of a tumor in an individual, reducing or inhibiting growth or proliferation of a tumor in an individual, preventing metastasis or reducing the extent of metastasis, and/or extending the survival (including but not limited to progression free survival (PFS) or overall survival) of an individual having or at risk of having cancer. In certain embodiments, the cancer cells or tumor express mutant KRAS. In certain embodiments, the inhibitor is a compound targeted to KRAS. In certain embodiments, the inhibitor is a compound targeted to mutant KRAS. In certain embodiments, the inhibitor is a compound selectively targeted to mutant KRAS for use in selectively reducing the number of cancer cells in an individual, selectively reducing the size of a tumor in an individual, selectively reducing or inhibiting growth or proliferation of a tumor in an individual, selectively preventing metastasis or reducing the extent of metastasis, and/or selectively extending the survival (including but not limited to progression free survival (PFS) or overall survival) of an individual having or at risk of having cancer expressing mutant KRAS. In certain embodiments, the KRAS specific inhibitor is a compound comprising a modified oligonucleotide consisting of 8 to 80 linked nucleosides and having a nucleobase sequence comprising at least 8 contiguous nucleobases of any of the nucleobase sequences of SEQ ID NOs: 13-2190. In certain embodiments, the KRAS specific inhibitor is a compound comprising a modified oligonucleotide consisting of 16 to 80 linked nucleosides and having a nucleobase sequence comprising the nucleobase sequence of any one of SEQ ID NOs: 13-2190. In certain embodiments, the KRAS specific inhibitor is a compound comprising a modified oligonucleotide consisting of 16 linked nucleosides and having a nucleobase sequence consisting of the nucleobase sequence of any one of SEQ ID NOs: 13-2190. In certain embodiments, the KRAS specific inhibitor is a compound comprising a modified oligonucleotide consisting of 16 to 80 linked nucleosides having a nucleobase sequence comprising any one of SEQ ID NOs: 239, 272, 569, 607, 615, 621, 640, 655, 678, 715, 790, 804, 854, 1028, 2130, 2136, 2142, 2154, and 2158. In certain embodiments, the KRAS specific inhibitor is a compound comprising a modified oligonucleotide consisting of 16 linked nucleosides having a nucleobase sequence consisting of any one of SEQ ID NOs: 239, 272, 569, 607, 615, 621, 640, 655, 678, 715, 790, 804, 854, 1028, 2130, 2136, 2142, 2154, and 2158. In certain embodiments, the KRAS specific inhibitor is ISIS #651530, 651987, 695785, 695823, 651555, 651587, 695980, 695995, 696018, 696044, 716600, 746275, 716655, 716772, 740179, 740191, 740201, 740223, or 740233. In certain embodiments, the KRAS specific inhibitor is ISIS #651987. In certain embodiments, the KRAS specific inhibitor is ISIS #746275. In any of the foregoing embodiments, the compound can be a single-stranded oligonucleotide. In any of the foregoing embodiments, the modified oligonucleotide can consist of 10 to 30 linked nucleosides. In certain embodiments, the compound is administered to the individual parenterally.

Certain embodiments are drawn to use of a KRAS specific inhibitor for the manufacture of a medicament for treating cancer. Certain embodiments are drawn to use of a KRAS specific inhibitor for the preparation of a medicament for treating cancer. In certain embodiments, the cancer expresses mutant KRAS. In certain embodiments, the cancer is lung cancer (e.g. non-small cell lung carcinoma (NSCLC) and small-cell lung carcinoma (SCLC)), gastrointestinal cancer (e.g. large intestinal cancer, small intestinal cancer, and stomach cancer), colon cancer, colorectal cancer, bladder cancer, liver cancer, esophageal cancer, pancreatic cancer, biliary tract cancer, breast cancer, ovarian cancer, endometrial cancer, cervical cancer, prostate cancer, hematopoetic cancer (e.g. leukemia, myeloid leukemia, and lymphoma), brain cancer (e.g. glioblastoma), malignant peripheral nerve sheath tumor (MPNST), neurofibromatosis type 1 (NF1) mutant MPNST, or neurofibroma. In certain embodiments, the inhibitor is a compound targeted to KRAS. In certain embodiments, the inhibitor is a compound targeted to mutant KRAS. In certain embodiments, the inhibitor is a compound selectively targeted to mutant KRAS. In certain embodiments, the KRAS specific inhibitor is a compound comprising a modified oligonucleotide consisting of 8 to 80 linked nucleosides and having a nucleobase sequence comprising at least 8 contiguous nucleobases of any of the nucleobase sequences of SEQ ID NOs: 13-2190. In certain embodiments, the KRAS specific inhibitor is a compound comprising a modified oligonucleotide consisting of 16 to 80 linked nucleosides and having a nucleobase sequence comprising the nucleobase sequence of any one of SEQ ID NOs: 13-2190. In certain embodiments, the KRAS specific inhibitor is a compound comprising a modified oligonucleotide consisting of 16 linked nucleosides and having a nucleobase sequence consisting of the nucleobase sequence of any one of SEQ ID NOs: 13-2190. In certain embodiments, the KRAS specific inhibitor is a compound comprising a modified oligonucleotide consisting of 16 to 80 linked nucleosides having a nucleobase sequence comprising any one of SEQ ID NOs: 239, 272, 569, 607, 615, 621, 640, 655, 678, 715, 790, 804, 854, 1028, 2130, 2136, 2142, 2154, and 2158. In certain embodiments, the KRAS specific inhibitor is a compound comprising a modified oligonucleotide consisting of 16 linked nucleosides having a nucleobase sequence consisting of any one of SEQ ID NOs: 239, 272, 569, 607, 615, 621, 640, 655, 678, 715, 790, 804, 854, 1028, 2130, 2136, 2142, 2154, and 2158. In certain embodiments, the KRAS specific inhibitor is ISIS #651530, 651987, 695785, 695823, 651555, 651587, 695980, 695995, 696018, 696044, 716600, 746275, 716655, 716772, 740179, 740191, 740201, 740223, or 740233. In certain embodiments, the KRAS specific inhibitor is ISIS #651987. In certain embodiments, the KRAS specific inhibitor is ISIS #746275. In any of the foregoing embodiments, the compound can be a single-stranded oligonucleotide. In any of the foregoing embodiments, the modified oligonucleotide can consist of 10 to 30 linked nucleosides. In certain embodiments, the compound is administered to the individual parenterally.

Certain embodiments are drawn to use of a KRAS specific inhibitor for the manufacture or preparation of a medicament for use in reducing the number of cancer cells in an individual, reducing the size of a tumor in an individual, reducing or inhibiting growth or proliferation of a tumor in an individual, preventing metastasis or reducing the extent of metastasis, and/or extending the survival (including but not limited to progression free survival (PFS) or overall survival) in an individual having or at risk of having cancer. In certain embodiments, the cancer cells or tumor expresses mutant KRAS. In certain embodiments, the inhibitor is a compound targeted to KRAS. In certain embodiments, the inhibitor is a compound targeted to KRAS. In certain embodiments, the inhibitor is a compound targeted to mutant KRAS. In certain embodiments, the inhibitor is a compound selectively targeted to mutant KRAS for the manufacture or preparation of a medicament for use in selectively reducing the number of cancer cells in an individual, selectively reducing the size of a tumor in an individual, selectively reducing or inhibiting growth or proliferation of a tumor in an individual, selectively preventing metastasis or reducing the extent of metastasis, and/or selectively extending the survival (including but not limited to progression free survival (PFS) or overall survival) of an individual having or at risk of having cancer expressing mutant KRAS. In certain embodiments, the KRAS specific inhibitor is a compound comprising a modified oligonucleotide consisting of 8 to 80 linked nucleosides and having a nucleobase sequence comprising at least 8 contiguous nucleobases of any of the nucleobase sequences of SEQ ID NOs: 13-2190. In certain embodiments, the KRAS specific inhibitor is a compound comprising a modified oligonucleotide consisting of 16 to 80 linked nucleosides and having a nucleobase sequence comprising the nucleobase sequence of any one of SEQ ID NOs: 13-2190. In certain embodiments, the KRAS specific inhibitor is a compound comprising a modified oligonucleotide consisting of 16 linked nucleosides and having a nucleobase sequence consisting of the nucleobase sequence of any one of SEQ ID NOs: 13-2190. In certain embodiments, the KRAS specific inhibitor is a compound comprising a modified oligonucleotide consisting of 16 to 80 linked nucleosides having a nucleobase sequence comprising any one of SEQ ID NOs: 239, 272, 569, 607, 615, 621, 640, 655, 678, 715, 790, 804, 854, 1028, 2130, 2136, 2142, 2154, and 2158. In certain embodiments, the KRAS specific inhibitor is a compound comprising a modified oligonucleotide consisting of 16 linked nucleosides having a nucleobase sequence consisting of any one of SEQ ID NOs: 239, 272, 569, 607, 615, 621, 640, 655, 678, 715, 790, 804, 854, 1028, 2130, 2136, 2142, 2154, and 2158. In certain embodiments, the KRAS specific inhibitor is ISIS #651530, 651987, 695785, 695823, 651555, 651587, 695980, 695995, 696018, 696044, 716600, 746275, 716655, 716772, 740179, 740191, 740201, 740223, or 740233. In certain embodiments, the KRAS specific inhibitor is ISIS #651987. In certain embodiments, the KRAS specific inhibitor is ISIS #746275. In any of the foregoing embodiments, the compound can be a single-stranded oligonucleotide. In any of the foregoing embodiments, the modified oligonucleotide can consist of 10 to 30 linked nucleosides. In certain embodiments, the compound is administered to the individual parenterally.

In any of the foregoing methods or uses, the KRAS specific inhibitor can be a compound targeted to KRAS, a compound targeted to mutant KRAS, or a compound selectively targeted to mutant KRAS. In certain embodiments, the compound is an antisense oligonucleotide, for example an antisense oligonucleotide consisting of 8 to 80 linked nucleosides, 10 to 30 linked nucleosides, 12 to 30 linked nucleosides, or 16 linked nucleosides. In certain embodiments, the antisense oligonucleotide is at least 80%, 85%, 90%, 95% or 100% complementary to any of the nucleobase sequences recited in SEQ ID NOs: 1-3. In certain embodiments, the antisense oligonucleotide comprises at least one modified internucleoside linkage, at least one modified sugar and/or at least one modified nucleobase. In certain embodiments, the modified internucleoside linkage is a phosphorothioate internucleoside linkage, the modified sugar is a bicyclic sugar or a 2′-O-methoxyethyl, and the modified nucleobase is a 5-methylcytosine. In certain embodiments, the modified oligonucleotide comprises a gap segment consisting of linked deoxynucleosides; a 5′ wing segment consisting of linked nucleosides; and a 3′ wing segment consisting of linked nucleosides, wherein the gap segment is positioned immediately adjacent to and between the 5′ wing segment and the 3′ wing segment and wherein each nucleoside of each wing segment comprises a modified sugar.

In any of the foregoing embodiments, the antisense oligonucleotide consists of 12 to 30, 15 to 30, 15 to 25, 15 to 24, 16 to 24, 17 to 24, 18 to 24, 19 to 24, 20 to 24, 19 to 22, 20 to 22, 16 to 20, or 17 or 20 linked nucleosides. In certain aspects, the antisense oligonucleotide is at least 80%, 85%, 90%, 95% or 100% complementary to any of the nucleobase sequences recited in SEQ ID NOs: 1-3. In certain aspects, the antisense oligonucleotide comprises at least one modified internucleoside linkage, at least one modified sugar and/or at least one modified nucleobase. In certain aspects, the modified internucleoside linkage is a phosphorothioate internucleoside linkage, the modified sugar is a bicyclic sugar or a 2′-O-methoxyethyl, and the modified nucleobase is a 5-methylcytosine. In certain aspects, the modified oligonucleotide comprises a gap segment consisting of linked 2′-deoxynucleosides; a 5′ wing segment consisting of linked nucleosides; and a 3′ wing segment consisting of linked nucleosides, wherein the gap segment is positioned immediately adjacent to and between the 5′ wing segment and the 3′ wing segment and wherein each nucleoside of each wing segment comprises a modified sugar.

In any of the foregoing methods or uses, the KRAS specific inhibitor can be a compound comprising or consisting of a modified oligonucleotide consisting of 16 to 30 linked nucleosides having a nucleobase sequence comprising any one of SEQ ID NOs: 13-2190, wherein the modified oligonucleotide comprises:
a gap segment consisting of linked deoxynucleosides;
a 5′ wing segment consisting of linked nucleosides; and
a 3′ wing segment consisting of linked nucleosides;

wherein the gap segment is positioned between the 5′ wing segment and the 3′ wing segment and wherein each nucleoside of each wing segment comprises a modified sugar.

In any of the foregoing methods or uses, the KRAS specific inhibitor can be a compound comprising or consisting of a modified oligonucleotide having a nucleobase sequence comprising or consisting of the sequence recited in any one of SEQ ID NOs: 239, 272, 569, 607, 615, 621, 640, 655, 678, 715, 790, and 854, wherein the modified oligonucleotide comprises

a gap segment consisting of ten linked deoxynucleosides;

a 5′ wing segment consisting of three linked nucleosides; and

a 3′ wing segment consisting of three linked nucleosides;

wherein the gap segment is positioned between the 5′ wing segment and the 3′ wing segment, wherein each nucleoside of each wing segment comprises a constrained ethyl (cEt) nucleoside; wherein each internucleoside linkage is a phosphorothioate linkage and wherein each cytosine is a 5-methylcytosine. In certain embodiments, the modified oligonucleotide consists of 16-80 linked nucleosides. In certain embodiments, the modified oligonucleotide consists of 16-30 linked nucleosides. In certain embodiments, the modified oligonucleotide consists of 16 linked nucleosides.

In any of the foregoing methods or uses, the KRAS specific inhibitor can be a compound comprising or consisting of a modified oligonucleotide having a nucleobase sequence comprising or consisting of the sequence recited in SEQ ID NO: 2130, wherein the modified oligonucleotide comprises

a gap segment consisting of nine linked deoxynucleosides;

a 5′ wing segment consisting of one linked nucleoside; and

a 3′ wing segment consisting of six linked nucleosides;

wherein the gap segment is positioned between the 5′ wing segment and the 3′ wing segment;

wherein the 5′ wing segment comprises a cEt nucleoside; wherein the 3′ wing segment comprises a cEt nucleoside, a 2′-O-methoxyethyl nucleoside, a cEt nucleoside, a 2′-O-methoxyethyl nucleoside, a cEt nucleoside, and 2′-O-methoxyethyl nucleoside in the 5′ to 3′ direction; wherein each internucleoside linkage is a phosphorothioate linkage; and wherein each cytosine is a 5-methylcytosine. In certain embodiments, the modified oligonucleotide consists of 16-80 linked nucleosides. In certain embodiments, the modified oligonucleotide consists of 16-30 linked nucleosides. In certain embodiments, the modified oligonucleotide consists of 16 linked nucleosides.

In any of the foregoing methods or uses, the KRAS specific inhibitor can be a compound comprising or consisting of a modified oligonucleotide having a nucleobase sequence comprising or consisting of the sequence recited in any one of SEQ ID NOs: 804, 1028, and 2136, wherein the modified oligonucleotide comprises

a gap segment consisting of ten linked deoxynucleosides;

a 5′ wing segment consisting of two linked nucleosides; and

a 3′ wing segment consisting of four linked nucleosides;

wherein the gap segment is positioned between the 5′ wing segment and the 3′ wing segment;

wherein the 5′ wing segment comprises a cEt nucleoside and a cEt nucleoside in the 5′ to 3′ direction; wherein the 3′ wing segment comprises a cEt nucleoside, a 2′-O-methoxyethyl nucleoside, a cEt nucleoside, and a 2′-O-methoxyethyl nucleoside in the 5′ to 3′ direction; wherein each internucleoside linkage is a phosphorothioate linkage; and wherein each cytosine is a 5-methylcytosine. In certain embodiments, the modified oligonucleotide consists of 16-80 linked nucleosides. In certain embodiments, the modified oligonucleotide consists of 16-30 linked nucleosides. In certain embodiments, the modified oligonucleotide consists of 16 linked nucleosides.

In any of the foregoing methods or uses, the KRAS specific inhibitor can be a compound comprising or consisting of a modified oligonucleotide having a nucleobase sequence comprising or consisting of the sequence recited in SEQ ID NO: 2142, wherein the modified oligonucleotide comprises

a gap segment consisting of eight linked deoxynucleosides;

a 5′ wing segment consisting of two linked nucleosides; and

a 3′ wing segment consisting of six linked nucleosides;

wherein the gap segment is positioned between the 5′ wing segment and the 3′ wing segment; wherein the 5′ wing segment comprises a cEt nucleoside and a cEt nucleoside in the 5′ to 3′ direction; wherein the 3′ wing segment comprises a cEt nucleoside, a 2′-O-methoxyethyl nucleoside, a cEt nucleoside, a 2′-O-methoxyethyl nucleoside, a cEt nucleoside, and a cEt nucleoside in the 5′ to 3′ direction; wherein each internucleoside linkage is a phosphorothioate linkage; and wherein each cytosine is a 5-methylcytosine. In certain embodiments, the modified oligonucleotide consists of 16-80 linked nucleosides. In certain embodiments, the modified oligonucleotide consists of 16-30 linked nucleosides. In certain embodiments, the modified oligonucleotide consists of 16 linked nucleosides.

In any of the foregoing methods or uses, the KRAS specific inhibitor can be a compound comprising or consisting of a modified oligonucleotide having a nucleobase sequence comprising or consisting of the sequence recited in SEQ ID NO: 2154, wherein the modified oligonucleotide comprises

a gap segment consisting of nine linked deoxynucleosides;

a 5′ wing segment consisting of two linked nucleosides; and

a 3′ wing segment consisting of five linked nucleosides;

wherein the gap segment is positioned between the 5′ wing segment and the 3′ wing segment; wherein the 5′ wing segment comprises a cEt nucleoside and a cEt nucleoside in the 5′ to 3′ direction; wherein the 3′ wing segment comprises a cEt nucleoside, a 2′-O-methoxyethyl nucleoside, a cEt nucleoside, a 2′-O-methoxyethyl nucleoside, and a cEt nucleoside in the 5′ to 3′ direction; wherein each internucleoside linkage is a phosphorothioate linkage; and wherein each cytosine is a 5-methylcytosine. In certain embodiments, the modified oligonucleotide consists of 16-80 linked nucleosides. In certain embodiments, the modified oligonucleotide consists of 16-30 linked nucleosides. In certain embodiments, the modified oligonucleotide consists of 16 linked nucleosides.

In any of the foregoing methods or uses, the KRAS specific inhibitor can be a compound comprising or consisting of a modified oligonucleotide having a nucleobase sequence comprising or consisting of the sequence recited in SEQ ID NO: 2158, wherein the modified oligonucleotide comprises

a gap segment consisting of eight linked deoxynucleosides;

a 5′ wing segment consisting of three linked nucleosides; and

a 3′ wing segment consisting of five linked nucleosides;

wherein the gap segment is positioned between the 5′ wing segment and the 3′ wing segment; wherein the 5′ wing segment comprises a cEt nucleoside, a cEt nucleoside, and a cEt nucleoside in the 5′ to 3′ direction; wherein the 3′ wing segment comprises a cEt nucleoside, a deoxynucleoside, a cEt nucleoside, a deoxynucleoside, and a cEt nucleoside in the 5′ to 3′ direction; wherein each internucleoside linkage is a phosphorothioate linkage; and wherein each cytosine is a 5-methylcytosine. In certain embodiments, the modified oligonucleotide consists of 16-80 linked nucleosides. In certain embodiments, the modified oligonucleotide consists of 16-30 linked nucleosides. In certain embodiments, the modified oligonucleotide consists of 16 linked nucleosides.

In any of the foregoing methods or uses, the KRAS specific inhibitor can be administered parenterally. For example, in certain embodiments the KRAS specific inhibitor can be administered through injection or infusion. Parenteral administration includes subcutaneous administration, intravenous administration, intramuscular administration, intraarterial administration, intraperitoneal administration, or intracranial administration, e.g. intrathecal or intracerebroventricular administration.

### Antisense Compounds

Antisense compounds are provided in certain embodiments. In certain embodiments, antisense compounds comprise at least one oligonucleotide. In certain embodiments, antisense compounds consist of an oligonucleotide. In certain embodiments, antisense compounds consist of an oligonucleotide attached to one or more conjugate groups. In certain embodiments, antisense compounds consist of an oligonucleotide attached to one or more conjugate groups via one or more conjugate linkers and/or a cleavable moiety. In certain embodiments, the oligonucleotide of an antisense compound is modified. In certain embodiments, the oligonucleotide of an antisense compound may have any nucleobase sequence. In certain embodiments, the oligonucleotide of an antisense compound is an antisense oligonucleotide having a nucleobase sequence that is complementary to a target nucleic acid. In certain embodiments, antisense oligonucleotides are complementary to a messenger RNA (mRNA).

In certain embodiments, an antisense compound has a nucleobase sequence that, when written in the 5′ to 3′ direction, comprises the reverse complement of the target segment of a target nucleic acid to which it is targeted.

In certain embodiments, an antisense compound is 10 to 30 subunits in length. In certain embodiments, an antisense compound is 12 to 30 subunits in length. In certain embodiments, an antisense compound is 12 to 22 subunits in length. In certain embodiments, an antisense compound is 14 to 30 subunits in length. In certain embodiments, an antisense compound is 14 to 20 subunits in length. In certain embodiments, an antisense compound is 15 to 30 subunits in length. In certain embodiments, an antisense compound is 15 to 20 subunits in length. In certain embodiments, an antisense compound is 16 to 30 subunits in length. In certain embodiments, an antisense compound is 16 to 20 subunits in length. In certain embodiments, an antisense compound is 17 to 30 subunits in length. In certain embodiments, an antisense compound is 17 to 20 subunits in length. In certain embodiments, an antisense compound is 18 to 30 subunits in length. In certain embodiments, an antisense compound is 18 to 21 subunits in length. In certain embodiments, an antisense compound is 18 to 20 subunits in length. In certain embodiments, an antisense compound is 20 to 30 subunits in length. In other words, such antisense compounds are from 12 to 30 linked subunits, 14 to 30 linked subunits, 14 to 20 subunits, 15 to 30 subunits, 15 to 20 subunits, 16 to 30 subunits, 16 to 20 subunits, 17 to 30 subunits, 17 to 20 subunits, 18 to 30 subunits, 18 to 20 subunits, 18 to 21 subunits, 20 to 30 subunits, or 12 to 22 linked subunits, respectively. In certain embodiments, an antisense compound is 14 subunits in length. In certain embodiments, an antisense compound is 16 subunits in length. In certain embodiments, an antisense compound is 17 subunits in length. In certain embodiments, an antisense compound is 18 subunits in length. In certain embodiments, an antisense compound is 19 subunits in length. In certain embodiments, an antisense compound is 20 subunits in length. In other embodiments, the antisense compound is 8 to 80, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked subunits. In certain such embodiments, the antisense compounds are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments the antisense compound is an antisense oligonucleotide, and the linked subunits are nucleotides, nucleosides, or nucleobases.

In certain embodiments, the antisense compound or oligomeric compound may further comprise additional features or elements, such as a conjugate group, that are attached to the oligonucleotide. In embodiments where a conjugate group comprises a nucleoside (i.e. a nucleoside that links the conjugate group to the oligonucleotide), the nucleoside of the conjugate group is not counted in the length of the oligonucleotide.

In certain embodiments antisense compounds may be shortened or truncated. For example, a single subunit may be deleted from the 5′ end (5′ truncation), or alternatively from the 3′ end (3′ truncation). A shortened or truncated antisense compound targeted to an KRAS nucleic acid may have two subunits deleted from the 5′ end, or alternatively may have two subunits deleted from the 3′ end, of the antisense compound. Alternatively, the deleted nucleosides may be dispersed throughout the antisense compound.

When a single additional subunit is present in a lengthened antisense compound, the additional subunit may be located at the 5′ or 3′ end of the antisense compound. When two or more additional subunits are present, the added subunits may be adjacent to each other, for example, in an antisense compound having two subunits added to the 5′ end (5′ addition), or alternatively to the 3′ end (3′ addition), of the antisense compound. Alternatively, the added subunits may be dispersed throughout the antisense compound, for example, in an antisense compound having one subunit added to the 5′ end and one subunit added to the 3′ end.

It is possible to increase or decrease the length of an antisense compound, such as an antisense oligonucleotide, and/or introduce mismatch bases without eliminating activity (Woolf et al. (Proc. Natl. Acad. Sci. USA 89:7305-7309, 1992; Gautschi et al. J. Natl. Cancer Inst. 93:463-471, March 2001; Maher and Dolnick Nuc. Acid. Res. 16:3341-3358, 1988). However, seemingly small changes in oligonucleotide sequence, chemistry and motif can make large differences in one or more of the many properties required for clinical development (Seth et al. J. Med. Chem. 2009, 52, 10; Egli et al. J. Am. Chem. Soc. 2011, 133, 16642).

In certain embodiments, antisense compounds are single-stranded, consisting of one oligomeric compound. The oligonucleotide of such single-stranded antisense compounds is an antisense oligonucleotide. In certain embodiments, the antisense oligonucleotide of a single-stranded antisense compound is modified. In certain embodiments, the oligonucleotide of a single-stranded antisense compound or oligomeric compound comprises a self-complementary nucleobase sequence. In certain embodiments, antisense compounds are double-stranded, comprising two oligomeric compounds that form a duplex. In certain such embodiments, one oligomeric compound of a double-stranded antisense compound comprises one or more conjugate groups. In certain embodiments, each oligomeric compound of a double-stranded antisense compound comprises one or more conjugate groups. In certain embodiments, each oligonucleotide of a double-stranded antisense compound is a modified oligonucleotide. In certain embodiments, one oligonucleotide of a double-stranded antisense compound is a modified oligonucleotide. In certain embodiments, one oligonucleotide of a double-stranded antisense compound is an antisense oligonucleotide. In certain such embodiments, the antisense oligonucleotide is a modified oligonucleotide. Examples of single-stranded and double-stranded antisense compounds include but are not limited to antisense oligonucleotides, siRNAs, microRNA targeting oligonucleotides, and single-stranded RNAi compounds, such as small hairpin RNAs (shRNAs), single-stranded siRNAs (ssRNAs), and microRNA mimics.

In certain embodiments, antisense compounds are interfering RNA compounds (RNAi), which include double-stranded RNA compounds (also referred to as short-interfering RNA or siRNA) and single-stranded RNAi compounds (or ssRNA). Such compounds work at least in part through the RISC pathway to degrade and/or sequester a target nucleic acid (thus, include microRNA/microRNA-mimic compounds). As used herein, the term siRNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics.

In certain embodiments, a double-stranded compound can comprise any of the oligonucleotide sequences targeted to KRAS described herein. In certain embodiments, a double-stranded compound comprises a first strand comprising at least an 8, 9, 10, 11, 12, 13, 14, 15, 16, 17, 18, 19, or 20 contiguous nucleobase portion of any one of SEQ ID NOs: 13-2190 and a second strand. In certain embodiments, a double-stranded compound comprises a first strand comprising the nucleobase sequence of any one of SEQ ID NOs: 13-2190 and a second strand. In certain embodiments, the double-stranded compound comprises ribonucleotides in which the first strand has uracil (U) in place of thymine (T) in any one of SEQ ID NOs: 13-2190. In certain embodiments, a double-stranded compound comprises (i) a first strand comprising a nucleobase sequence complementary to the site on KRAS to which any of SEQ ID NOs: 13-2190 is targeted, and (ii) a second strand. In certain embodiments, the double-stranded compound comprises one or more modified nucleotides in which the 2′ position in the sugar contains a halogen (such as fluorine group; 2′-F) or contains an alkoxy group (such as a methoxy group; 2′-OMe). In certain embodiments, the double-stranded compound comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the dsRNA compound. In certain embodiments, the double-stranded compound comprises one or more linkages between adjacent nucleotides other than a naturally-occurring phosphodiester linkage. Examples of such linkages include phosphoramide, phosphorothioate, and phosphorodithioate linkages. The double-stranded compounds may also be chemically modified nucleic acid molecules as taught in U.S. Pat. No. 6,673,661. In other embodiments, the dsRNA contains one or two capped strands, as disclosed, for example, by WO 00/63364, filed Apr. 19, 2000. In certain embodiments, the first strand of the double-stranded compound is an siRNA guide strand and the second strand of the double-stranded compound is an siRNA passenger strand. In certain embodiments, the second strand of the double-stranded compound is complementary to the first strand. In certain embodiments, each strand of the double-stranded compound consists of 16, 17, 18, 19, 20, 21, 22, or 23 linked nucleosides. In certain embodiments, the first or second strand of the double-stranded compound can comprise a conjugate group.

In certain embodiments, a single-stranded RNAi (ssRNAi) compound can comprise any of the oligonucleotide sequences targeted to KRAS described herein. In certain embodiments, an ssRNAi compound comprises at least an 8, 9, 10, 11, 12, 13, 14, 15, 16, 17, 18, 19, or 20 contiguous nucleobase portion of any one of SEQ ID NOs: 13-2190. In certain embodiments, an ssRNAi compound comprises the nucleobase sequence of any one of SEQ ID NOs: 13-2190. In certain embodiments, the ssRNAi compound comprises ribonucleotides in which uracil (U) is in place of thymine (T) in any one of SEQ ID NOs: 13-2190. In certain embodiments, an ssRNAi compound comprises a nucleobase sequence complementary to the site on KRAS to which any of SEQ ID NOs: 13-2190 is targeted. In certain embodiments, an ssRNAi compound comprises one or more modified nucleotides in which the 2′ position in the sugar contains a halogen (such as fluorine group; 2′-F) or contains an alkoxy group (such as a methoxy group; 2′-OMe). In certain embodiments, an ssRNAi compound comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the ssRNAi compound. In certain embodiments, the ssRNAi compound comprises one or more linkages between adjacent nucleotides other than a naturally-occurring phosphodiester linkage. Examples of such linkages include phosphoramide, phosphorothioate, and phosphorodithioate linkages. The ssRNAi compounds may also be chemically modified nucleic acid molecules as taught in U.S. Pat. No. 6,673,661. In other embodiments, the ssRNAi contains a capped strand, as disclosed, for example, by WO 00/63364, filed Apr. 19, 2000. In certain embodiments, the ssRNAi compound consists of 16, 17, 18, 19, 20, 21, 22, or 23 linked nucleosides. In certain embodiments, the ssRNAi compound can comprise a conjugate group.

In certain embodiments, antisense compounds comprise modified oligonucleotides. Certain modified oligonucleotides have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L) such as for amino acids etc. Included in the modified oligonucleotides provided herein are all such possible isomers, including their racemic and optically pure forms, unless specified otherwise. Likewise, all cis- and trans-isomers and tautomeric forms are also included.

### Certain Antisense Compound Mechanisms

In certain embodiments, antisense compounds are capable of hybridizing to a target nucleic acid, resulting in at least one antisense activity. In certain embodiments, antisense compounds specifically affect one or more target nucleic acid. Such specific antisense compounds comprises a nucleobase sequence that hybridizes to one or more target nucleic acid, resulting in one or more desired antisense activity and does not hybridize to one or more non-target nucleic acid or does not hybridize to one or more non-target nucleic acid in such a way that results in an undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, the invention provides antisense compounds that are sufficiently “DNA-like” to elicit RNase H activity. Further, in certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. In certain embodiments, antisense compounds that are loaded into RISC are RNAi compounds.

In certain embodiments, hybridization of an antisense compound to a target nucleic acid does not result in recruitment of a protein that cleaves that target nucleic acid. In certain such embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain such embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

In certain embodiments, modified oligonucleotides having a gapmer sugar motif described herein have desirable properties compared to non-gapmer oligonucleotides or to gapmers having other sugar motifs. In certain circumstances, it is desirable to identify motifs resulting in a favorable combination of potent antisense activity and relatively low toxicity. In certain embodiments, compounds of the present invention have a favorable therapeutic index (measure of activity divided by measure of toxicity).

### Target Nucleic Acids, Target Regions and Nucleotide Sequences

In certain embodiments, antisense compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: an mRNA and a pre-mRNA, including intronic, exonic and untranslated regions. In certain embodiments, the target RNA is an mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron.

Nucleotide sequences that encode KRAS include, without limitation, GENBANK Accession No. NM_004985.4 (incorporated by reference, disclosed herein as SEQ ID NO: 1); GENBANK Accession No. NT_009714.17_TRUNC_18116000_18166000_COMP (incorporated by reference, disclosed herein as SEQ ID NO: 2), and GENBANK Accession No. NM_033360.3 (incorporated by reference, disclosed herein as SEQ ID NO: 3).

### Hybridization

In some embodiments, hybridization occurs between an antisense compound disclosed herein and a KRAS nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Hybridization conditions are sequence-dependent and are determined by the nature and composition of the nucleic acid molecules to be hybridized.

Methods of determining whether a sequence is specifically hybridizable to a target nucleic acid are well known in the art. In certain embodiments, the antisense compounds provided herein are specifically hybridizable with a KRAS nucleic acid.

### Complementarity

An oligonucleotide is said to be complementary to another nucleic acid when the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described herein, are limited to adenine (A) and thymine (T), adenine (A) and uracil (U), cytosine (C) and guanine (G), and 5-methyl cytosine (mC) and guanine (G) unless otherwise specified. Complementary oligonucleotides and/or nucleic acids need not have nucleobase complementarity at each nucleoside and may include one or more nucleobase mismatches. An oligonucleotide is fully complementary or 100% complementary when such oligonucleotides have nucleobase matches at each nucleoside without any nucleobase mismatches.

Non-complementary nucleobases between an antisense compound and a KRAS nucleic acid may be tolerated provided that the antisense compound remains able to specifically hybridize to a target nucleic acid. Moreover, an antisense compound may hybridize over one or more segments of a KRAS nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a KRAS nucleic acid, a target region, target segment, or specified portion thereof. Percent complementarity of an antisense compound with a target nucleic acid can be determined using routine methods.

For example, an antisense compound in which 18 of 20 nucleobases of the antisense compound are complementary to a target region, and would therefore specifically hybridize, would represent 90 percent complementarity. In this example, the remaining non-complementary nucleobases may be clustered or interspersed with complementary nucleobases and need not be contiguous to each other or to complementary nucleobases. As such, an antisense compound which is 18 nucleobases in length having four non-complementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e. 100% complementary) to a target nucleic acid, or specified portion thereof. For example, an antisense compound may be fully complementary to a KRAS nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

In certain embodiments, antisense compounds comprise one or more mismatched nucleobases relative to the target nucleic acid. In certain such embodiments, antisense activity against the target is reduced by such mismatch, but activity against a non-target is reduced by a greater amount. Thus, in certain such embodiments selectivity of the antisense compound is improved. In certain embodiments, the mismatch is specifically positioned within an oligonucleotide having a gapmer motif. In certain such embodiments, the mismatch is at position 1, 2, 3, 4, 5, 6, 7, or 8 from the 5′-end of the gap region. In certain such embodiments, the mismatch is at position 9, 8, 7, 6, 5, 4, 3, 2, 1 from the 3′-end of the gap region. In certain such embodiments, the mismatch is at position 1, 2, 3, or 4 from the 5′-end of the wing region. In certain such embodiments, the mismatch is at position 4, 3, 2, or 1 from the 3′-end of the wing region.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e. linked) or non-contiguous. In one embodiment, a non-complementary nucleobase is located in the wing segment of a gapmer antisense oligonucleotide.

In certain embodiments, antisense compounds that are, or are up to 11, 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a KRAS nucleic acid, or specified portion thereof.

In certain embodiments, antisense compounds that are, or are up to 11, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a KRAS nucleic acid, or specified portion thereof.

The antisense compounds provided also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 9 nucleobase portion of a target segment. In certain embodiments, the antisense compounds are complementary to at least a 10 nucleobase portion of a target segment. In certain embodiments, the antisense compounds are complementary to at least an 11 nucleobase portion of a target segment. In certain embodiments, the antisense compounds are complementary to at least a 12 nucleobase portion of a target segment. In certain embodiments, the antisense compounds are complementary to at least a 13 nucleobase portion of a target segment. In certain embodiments, the antisense compounds are complementary to at least a 14 nucleobase portion of a target segment. In certain embodiments, the antisense compounds are complementary to at least a 15 nucleobase portion of a target segment. In certain embodiments, the antisense compounds are complementary to at least a 16 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

### Identity

The antisense compounds provided herein may also have a defined percent identity to a particular nucleotide sequence, SEQ ID NO, or compound represented by a specific Isis number, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases may be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are, or are at least, 70%, 75%, 80%, 85%, 90%, 91%, 92%, 93%, 94%, 95%, 96%, 97%, 98%, 99% or 100% identical to one or more of the antisense compounds or SEQ ID NOs, or a portion thereof, disclosed herein.

In certain embodiments, a portion of the antisense compound is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

In certain embodiments, a portion of the antisense oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

### Modifications

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH2-N(CH3)-O—CH2-), thiodiester (—O—C(═O)—S—), thionocarbamate (—O—C(═O)(NH)—S—); siloxane (—O—SiH2-O—); and N,N′-dimethylhydrazine (—CH2-N(CH3)-N(CH3)-). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Representative chiral internucleoside linkages include but are not limited to alkylphosphonates and phosphorothioates. Methods of preparation of phosphorous-containing and non-phosphorous-containing internucleoside linkages are well known to those skilled in the art.

Neutral internucleoside linkages include, without limitation, phosphotriesters, methylphosphonates, MMI (3′-CH2-N(CH3)-O-5′), amide-3 (3′-CH2-C(═O)—N(H)-5′), amide-4 (3′-CH2-N(H)—C(═O)-5′), formacetal (3′-O—CH2-O-5′), methoxypropyl, and thioformacetal (3′-S—CH2-O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH2 component parts.

In certain embodiments, antisense compounds targeted to a KRAS nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

In certain embodiments, oligonucleotides comprise modified internucleoside linkages arranged along the oligonucleotide or region thereof in a defined pattern or modified internucleoside linkage motif. In certain embodiments, internucleoside linkages are arranged in a gapped motif. In such embodiments, the internucleoside linkages in each of two wing regions are different from the internucleoside linkages in the gap region. In certain embodiments the internucleoside linkages in the wings are phosphodiester and the internucleoside linkages in the gap are phosphorothioate. The nucleoside motif is independently selected, so such oligonucleotides having a gapped internucleoside linkage motif may or may not have a gapped nucleoside motif and if it does have a gapped nucleoside motif, the wing and gap lengths may or may not be the same.

In certain embodiments, oligonucleotides comprise a region having an alternating internucleoside linkage motif. In certain embodiments, oligonucleotides of the present invention comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least one block of at least 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

In certain embodiments, oligonucleotides comprise one or more methylphosponate linkages. In certain embodiments, oligonucleotides having a gapmer nucleoside motif comprise a linkage motif comprising all phosphorothioate linkages except for one or two methylphosponate linkages. In certain embodiments, one methylphosponate linkage is in the central gap of an oligonucleotide having a gapmer nucleoside motif.

In certain embodiments, it is desirable to arrange the number of phosphorothioate internucleoside linkages and phosphodiester internucleoside linkages to maintain nuclease resistance. In certain embodiments, it is desirable to arrange the number and position of phosphorothioate internucleoside linkages and the number and position of phosphodiester internucleoside linkages to maintain nuclease resistance. In certain embodiments, the number of phosphorothioate internucleoside linkages may be decreased and the number of phosphodiester internucleoside linkages may be increased. In certain embodiments, the number of phosphorothioate internucleoside linkages may be decreased and the number of phosphodiester internucleoside linkages may be increased while still maintaining nuclease resistance. In certain embodiments it is desirable to decrease the number of phosphorothioate internucleoside linkages while retaining nuclease resistance. In certain embodiments it is desirable to increase the number of phosphodiester internucleoside linkages while retaining nuclease resistance.

### Modified Sugar Moieties

Antisense compounds can optionally contain one or more nucleosides wherein the sugar group has been modified. Such sugar modified nucleosides may impart enhanced nuclease stability, increased binding affinity, or some other beneficial biological property to the antisense compounds.

In certain embodiments, modified oligonucleotides comprise one or more modified nucleosides comprising a modified sugar moiety. Such modified oligonucleotides comprising one or more sugar-modified nucleosides may have desirable properties, such as enhanced nuclease stability or increased binding affinity with a target nucleic acid relative to oligonucleotides lacking such sugar-modified nucleosides. In certain embodiments, modified sugar moieties are linearly modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of substituted sugar moieties.

In certain embodiments, modified sugar moieties are linearly modified sugar moieties comprising a furanosyl ring with one or more acyclic substituent, including but not limited to substituents at the 2′ and/or 5′ positions. Examples of 2′-substituent groups suitable for linearly modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 5′-substituent groups suitable for linearly modified sugar moieties include but are not limited to: 5′-methyl (R or S), 5′-vinyl, and 5′-methoxy. In certain embodiments, linearly modified sugars comprise more than one non-bridging sugar substituent, for example, 2′-F-5′-methyl sugar moieties (see, e.g., PCT International Application WO 2008/101157, for additional 2′, 5′-bis substituted sugar moieties and nucleosides).

In certain embodiments, a 2′-substituted nucleoside or 2′-linearly modified nucleoside comprises a sugar moiety comprising a linear 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂₀N(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or 2′-linearly modified nucleoside comprises a sugar moiety comprising a linear 2′-substituent group selected from: F, OCF₃, OCH₃, OCH₂CH₂OCH₃, O(CH₂)₂SCH₃, O(CH₂)₂₀N(CH₃)₂, O(CH₂)₂O(CH₂)₂N(CH₃)₂, and OCH₂C(═O)—N(H)CH₃ (“NMA”).

In certain embodiments, a 2′-substituted nucleoside or 2′-linearly modified nucleoside comprises a sugar moiety comprising a linear 2′-substituent group selected from: F, OCH₃, and OCH₂CH₂OCH₃.

Nucleosides comprising modified sugar moieties, such as linearly modified sugar moieties, are referred to by the position(s) of the substitution(s) on the sugar moiety of the nucleoside. For example, nucleosides comprising 2′-substituted or 2-modified sugar moieties are referred to as 2′-substituted nucleosides or 2-modified nucleosides.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂-O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., U.S. Pat. No. 7,399,845), 4′-C(CH₃)(CH₃)—O-2′ and analogs thereof (see, e.g., WO2009/006478), 4′-CH₂—N(OCH₃)-2′ and analogs thereof (see, e.g., WO2008/150729), 4′-CH₂—O—N(CH₃)-2′ (see, e.g., US2004/0171570), 4′-CH₂—C(H)(CH₃)-2′ (see, e.g., Chattopadhyaya, et al., J. Org. Chem., 2009, 74, 118-134), 4′-CH₂—C(═CH₂)-2′ and analogs thereof (see, published PCT International Application WO 2008/154401), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-Jᵣ), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, Cᵣ-Cᵤ aminoalkyl, substituted C₁-C₁₂ aminoalkyl, or a protecting group.

Additional bicyclic sugar moieties are known in the art, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 20017, 129, 8362-8379; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; U.S. Pat. Nos. 7,053,207, 6,268,490, 6,770,748, 6,794,499, 7,034,133, 6,525,191, 6,670,461, and 7,399,845; WO 2004/106356, WO 1994/14226, WO 2005/021570, and WO 2007/134181; U.S. Patent Publication Nos. U52004/0171570, U52007/0287831, and U52008/0039618; U.S. patent Ser. Nos. 12/129,154, 60/989,574, 61/026,995, 61/026,998, 61/056,564, 61/086,231, 61/097,787, and 61/099,844; and PCT International Applications Nos. PCT/US2008/064591, PCT/US2008/066154, and PCT/US2008/068922.

In certain embodiments, bicyclic sugar moieties and nucleosides incorporating such bicyclic sugar moieties are further defined by isomeric configuration. For example, an LNA nucleoside (described above) may be in the α-L configuration or in the β-D configuration.

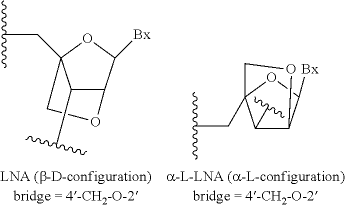

α-L-methyleneoxy (4′-CH₂—O-2′) or α-L-LNA bicyclic nucleosides have been incorporated into antisense oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars). (see, e.g., WO 2007/134181, wherein LNA nucleosides are further substituted with, for example, a 5′-methyl or a 5′-vinyl group, and see, e.g., U.S. Pat. Nos. 7,547,684; 7,750,131; 8,030,467; 8,268,980; 7,666, 854; and 8,088,746).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described above. For example, certain sugar surrogates comprise a 4′-sulfur atom and a substitution at the 2′-position (see, e.g., US2005/0130923) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see Leumann, C J. Bioorg. & Med. Chem. 2002, 10, 841-854), fluoro HNA:

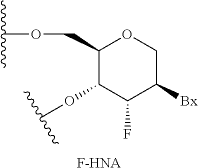

(“F-HNA”, see e.g., U.S. Pat. Nos. 8,088,904; 8,440,803; and 8,796,437, F-HNA can also be referred to as a F-THP or 3′-fluoro tetrahydropyran), and nucleosides comprising additional modified THP compounds having the formula:

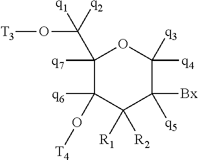

wherein, independently, for each of said modified THP nucleoside:

Bx is a nucleobase moiety;

T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group;

q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and

each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and U.S. Pat. Nos. 5,698,685; 5,166,315; 5,185,444; and 5,034,506). As used here, the term “morpholino” means a sugar surrogate having the following structure:

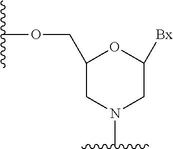

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieites. Examples of nucleosides and oligonucleotieds comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in WO2011/133876.

Many other bicyclic and tricyclic sugar and sugar surrogate ring systems are known in the art that can be used in modified nucleosides (see, e.g., Leumann, J. C, Bioorganic & Medicinal Chemistry, 2002, 10, 841-854).

### Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to antisense compounds.

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimi-idines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, 5-methylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., U52003/0158403, Manoharan et al., U52003/0175906; Dinh et al., U.S. Pat. No. 4,845,205; Spielvogel et al., U.S. Pat. No. 5,130,302; Rogers et al., U.S. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

In certain embodiments, antisense compounds targeted to a KRAS nucleic acid comprise one or more modified nucleobases. In certain embodiments, the modified nucleobase is 5-methylcytosine. In certain embodiments, each cytosine is a 5-methylcytosine.

### Certain Motifs

Oligonucleotides can have a motif, e.g. a pattern of unmodified and/or modified sugar moieties, nucleobases, and/or internucleoside linkages. In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which comprises two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, the wings of a gapmer comprise 2-5 nucleosides. In certain embodiments, the wings of a gapmer comprise 3-5 nucleosides. In certain embodiments, the nucleosides of a gapmer are all modified nucleosides.

In certain embodiments, the gap of a gapmer comprises 7-12 nucleosides. In certain embodiments, the gap of a gapmer comprises 7-10 nucleosides. In certain embodiments, the gap of a gapmer comprises 8-10 nucleosides. In certain embodiments, the gap of a gapmer comprises 10 nucleosides. In certain embodiment, each nucleoside of the gap of a gapmer is an unmodified 2′-deoxy nucleoside.

In certain embodiments, the gapmer is a deoxy gapmer. In such embodiments, the nucleosides on the gap side of each wing/gap junction are unmodified 2′-deoxy nucleosides and the nucleosides on the wing sides of each wing/gap junction are modified nucleosides. In certain such embodiments, each nucleoside of the gap is an unmodified 2′-deoxy nucleoside. In certain such embodiments, each nucleoside of each wing is a modified nucleoside.

In certain embodiments, modified oligonucleotides comprise or consist of a region having a fully modified sugar motif. In such embodiments, each nucleoside of the fully modified region of the modified oligonucleotide comprises a modified sugar moiety. In certain such embodiments, each nucleoside to the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, essentially each internucleoside linking group is a phosphate internucleoside linkage (P═O). In certain embodiments, each internucleoside linking group of a modified oligonucleotide is a phosphorothioate (P═S). In certain embodiments, each internucleoside linking group of a modified oligonucleotide is independently selected from a phosphorothioate and phosphate internucleoside linkage. In certain embodiments, the sugar motif of a modified oligonucleotide is a gapmer and the internucleoside linkages within the gap are all modified. In certain such embodiments, some or all of the internucleoside linkages in the wings are unmodified phosphate linkages. In certain embodiments, the terminal internucleoside linkages are modified.

### Certain Oligonucleotides

In certain embodiments, oligonucleotides are characterized by their motifs and overall lengths. In certain embodiments, such parameters are each independent of one another. Thus, unless otherwise indicated, each internucleoside linkage of an oligonucleotide having a gapmer motif may be modified or unmodified and may or may not follow the gapmer modification pattern of the sugar modifications. For example, the internucleoside linkages within the wing regions of a gapmer may be the same or different from one another and may be the same or different from the internucleoside linkages of the gap region. Likewise, such gapmer oligonucleotides may comprise one or more modified nucleobase independent of the gapmer pattern of the sugar modifications. Furthermore, unless otherwise indicated, each internucleoside linkage and each nucleobase of a fully modified oligonucleotide may be modified or unmodified. One of skill in the art will appreciate that such motifs may be combined to create a variety of oligonucleotides. Herein, if a description of an oligonucleotide is silent with respect to one or more parameter, such parameter is not limited. Thus, a modified oligonucleotide described only as having a gapmer motif without further description may have any length, internucleoside linkage motif, and nucleobase motif. Unless otherwise indicated, all modifications are independent of nucleobase sequence.

In certain embodiments, oligonucleotides have a nucleobase sequence that is complementary to a second oligonucleotide or a target nucleic acid. In certain such embodiments, a region of an oligonucleotide has a nucleobase sequence that is complementary to a second oligonucleotide or a target nucleic acid. In certain embodiments, the nucleobase sequence of a region or entire length of an oligonucleotide is at least 50%, at least 60%, at least 70%, at least 80%, at least 90%, at least 95%, or 100% complementary to the second oligonucleotide or target nucleic acid. In certain embodiments, antisense compounds comprise two oligomeric compounds, wherein the two oligonucleotides of the oligomeric compounds are at least 80%, at least 90%, or 100% complementary to each other. In certain embodiments, one or both oligonucleotides of a double-stranded antisense compound comprise two nucleosides that are not complementary to the other oligonucleotide.

### Certain Conjugate Groups and Terminal Groups

In certain embodiments, antisense compounds and oligomeric compounds comprise conjugate groups and/or terminal groups. In certain such embodiments, oligonucleotides are covalently attached to one or more conjugate group. In certain embodiments, conjugate groups modify one or more properties of the attached oligonucleotide, including but not limited to pharmacodynamics, pharmacokinetics, stability, binding, absorption, cellular distribution, cellular uptake, charge and clearance. In certain embodiments, conjugate groups impart a new property on the attached oligonucleotide, e.g., fluorophores or reporter groups that enable detection of the oligonucleotide. Conjugate groups and/or terminal groups may be added to oligonucleotides having any of the modifications or motifs described above. Thus, for example, an antisense compound or oligomeric compound comprising an oligonucleotide having a gapmer motif may also comprise a conjugate group.

Conjugate groups include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes. Certain conjugate groups have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; doi:10.1038/mtna.2014.72 and Nishina et al., Molecular Therapy, 2008, 16, 734-740), or a GalNAc cluster (e.g., WO2014/179620).

In certain embodiments, a conjugate group comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

Conjugate groups are attached directly or via an optional conjugate linker to a parent compound, such as an oligonucleotide. In certain embodiments, conjugate groups are directly attached to oligonucleotides. In certain embodiments, conjugate groups are indirectly attached to oligonucleotides via conjugate linkers. In certain embodiments, the conjugate linker comprises a chain structure, such as a hydrocarbyl chain, or an oligomer of repeating units such as ethylene glycol or amino acid units. In certain embodiments, conjugate groups comprise a cleavable moiety. In certain embodiments, conjugate groups are attached to oligonucleotides via a cleavable moiety. In certain embodiments, conjugate linkers comprise a cleavable moiety. In certain such embodiments, conjugate linkers are attached to oligonucleotides via a cleavable moiety. In certain embodiments, oligonucleotides comprise a cleavable moiety, wherein the cleavable moiety is a nucleoside is attached to a cleavable internucleoside linkage, such as a phosphate internucleoside linkage. In certain embodiments, a conjugate group comprises a nucleoside or oligonucleotide, wherein the nucleoside or oligonucleotide of the conjugate group is indirectly attached to a parent oligonucleotide.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, a cleavable moiety is a cleavable bond. In certain embodiments, a cleavable moiety comprises a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate linker or conjugate group.

In certain embodiments, a cleavable moiety is a nucleoside. In certain such embodiments, the unmodified or modified nucleoside comprises an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. In certain embodiments, a cleavable moiety is 2′-deoxy nucleoside that is attached to either the 3′ or 5′-terminal nucleoside of an oligonucleotide by a phosphate internucleoside linkage and covalently attached to the conjugate linker or conjugate group by a phosphate or phosphorothioate linkage. In certain such embodiments, the cleavable moiety is 2′-deoxyadenosine.

Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

In certain embodiments, a conjugate group is a cell-targeting moiety. In certain embodiments, a conjugate group, optional conjugate linker, and optional cleavable moiety have the general formula:

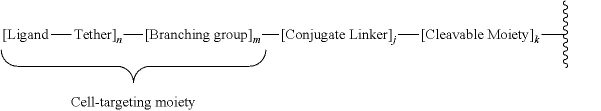

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amid, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate. In certain embodiments, each ligand is, independently selected from galactose, N-acetyl galactoseamine (GalNAc), mannose, glucose, glucoseamine and fucose. In certain embodiments, each ligand is N-acetyl galactoseamine (GalNAc). In certain embodiments, the cell-targeting moiety comprises 3 GalNAc ligands. In certain embodiments, the cell-targeting moiety comprises 2 GalNAc ligands. In certain embodiments, the cell-targeting moiety comprises 1 GalNAc ligand.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29, or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808, which are incorporated herein by reference in their entirety). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycoloyl-α-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, conjugate groups comprise a cell-targeting moiety having the formula:

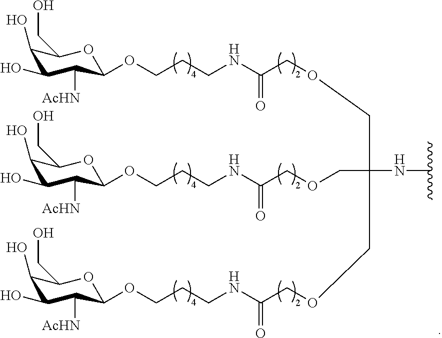

In certain embodiments, conjugate groups comprise a cell-targeting moiety having the formula:

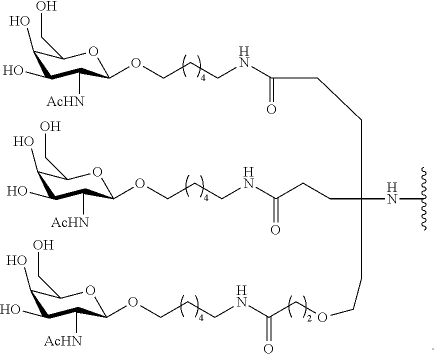

In certain embodiments, conjugate groups comprise a cell-targeting moiety having the formula:

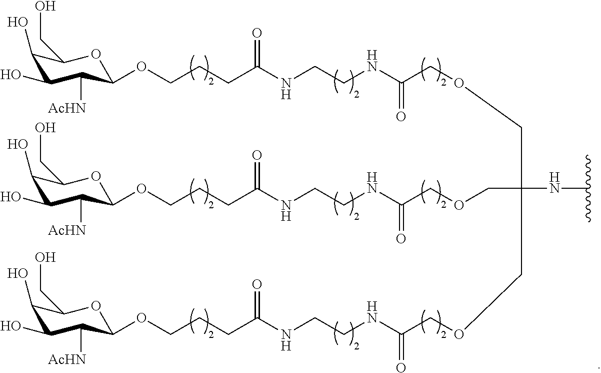

In certain embodiments, antisense compounds and oligomeric compounds comprise a conjugate group and conjugate linker described herein as “LICA-1”. LICA-1 has the formula:

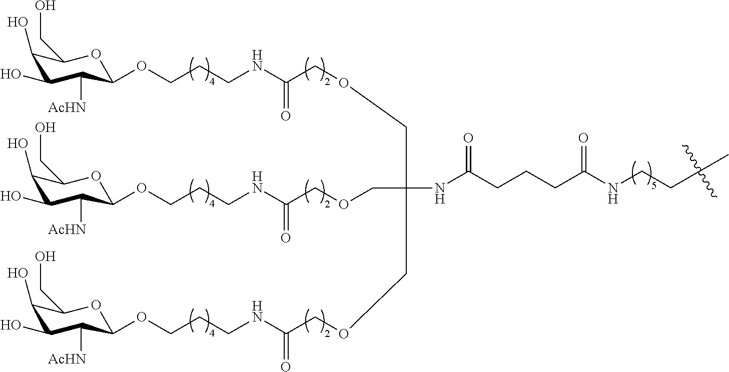

In certain embodiments, antisense compounds and oligomeric compounds comprising LICA-1 have the formula:

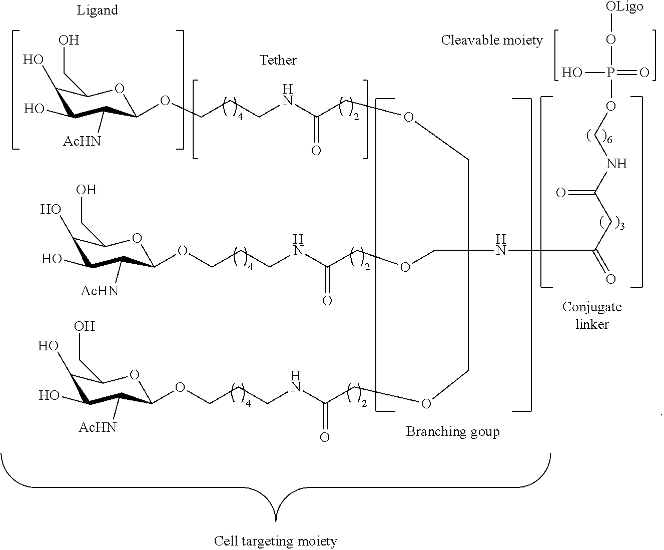

wherein oligo is an oligonucleotide.

Representative publications that teach the preparation of certain of the above noted conjugate groups, oligomeric compounds and antisense compounds comprising conjugate groups, tethers, conjugate linkers, branching groups, ligands, cleavable moieties as well as other modifications include without limitation, U.S. Pat. No. 5,994,517, U.S. Pat. No. 6,300,319, U.S. Pat. No. 6,660,720, U.S. Pat. No. 6,906,182, U.S. Pat. No. 7,262,177, U.S. Pat. No. 7,491,805, U.S. Pat. No. 8,106,022, U.S. Pat. No. 7,723,509, US 2006/0148740, US 2011/0123520, WO 2013/033230 and WO 2012/037254, Biessen et al., J. Med. Chem. 1995, 38, 1846-1852, Lee et al., Bioorganic & Medicinal Chemistry 2011, 19, 2494-2500, Rensen et al., J. Biol. Chem. 2001, 276, 37577-37584, Rensen et al., J. Med. Chem. 2004, 47, 5798-5808, Sliedregt et al., J. Med. Chem. 1999, 42, 609-618, and Valentijn et al., Tetrahedron, 1997, 53, 759-770, each of which is incorporated by reference herein in its entirety.

In certain embodiments, antisense compounds and oligomeric compounds comprise modified oligonucleotides comprising a gapmer or fully modified motif and a conjugate group comprising at least one, two, or three GalNAc ligands. In certain embodiments antisense compounds and oligomeric compounds comprise a conjugate group found in any of the following references: Lee, Carbohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications 052011/0097264; 052011/0097265; 052013/0004427; 052005/0164235; 052006/0148740; 052008/0281044; 052010/0240730; 052003/0119724; 052006/0183886; 052008/0206869; 052011/0269814; 052009/0286973; 052011/0207799; 052012/0136042; 052012/0165393; 052008/0281041; 052009/0203135; 052012/0035115; 052012/0095075; 052012/0101148; 052012/0128760; 052012/0157509; 052012/0230938; 052013/0109817; 052013/0121954; 052013/0178512; 052013/0236968; 052011/0123520; 052003/0077829; 052008/0108801; and U52009/0203132; each of which is incorporated by reference in its entirety.

### Compositions and Methods for Formulating Pharmaceutical Compositions

Compounds may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

In certain embodiments, the present invention provides pharmaceutical compositions comprising one or more compounds or a salt thereof. In certain such embodiments, the pharmaceutical composition comprises a suitable pharmaceutically acceptable diluent or carrier. In certain embodiments, a pharmaceutical composition comprises a sterile saline solution and one or more compounds. In certain embodiments, such pharmaceutical composition consists of a sterile saline solution and one or more compounds. In certain embodiments, the sterile saline is pharmaceutical grade saline. In certain embodiments, a pharmaceutical composition comprises one or more antisense compound and sterile water. In certain embodiments, a pharmaceutical composition consists of one compounds and sterile water. In certain embodiments, the sterile water is pharmaceutical grade water. In certain embodiments, a pharmaceutical composition comprises one or more compounds and phosphate-buffered saline (PBS). In certain embodiments, a pharmaceutical composition consists of one or more compounds and sterile PBS. In certain embodiments, the sterile PBS is pharmaceutical grade PBS. Compositions and methods for the formulation of pharmaceutical compositions are dependent upon a number of criteria, including, but not limited to, route of administration, extent of disease, or dose to be administered.

A compound targeted to KRAS nucleic acid can be utilized in pharmaceutical compositions by combining the compound with a suitable pharmaceutically acceptable diluent or carrier. In certain embodiments, a pharmaceutically acceptable diluent is water, such as sterile water suitable for injection. Accordingly, in one embodiment, employed in the methods described herein is a pharmaceutical composition comprising a compound targeted to KRAS nucleic acid and a pharmaceutically acceptable diluent. In certain embodiments, the pharmaceutically acceptable diluent is water. In certain embodiments, the compound is an antisense oligonucleotide provided herein.

Pharmaceutical compositions comprising compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of a compound which are cleaved by endogenous nucleases within the body, to form the active compound. In certain embodiments, the compounds or compositions further comprise a pharmaceutically acceptable carrier or diluent.

### EXAMPLES

The Examples below describe the screening process to identify lead compounds targeted to KRAS. Approximately 2,000 newly designed compounds were tested for their effect on human KRAS mRNA. New compounds were compared with a previously described compound, ISIS 6957, which was reported as one of the most potent antisense compounds in U.S. Pat. No. 6,784,290. Out of over 2,000 antisense oligonucleotides that were screened, ISIS #651530, 651987, 695785, 695823, 651555, 651587, 695980, 695995, 696018, 696044, 716600, 746275, 716655, 716772, 740179, 740191, 740201, 740223, and 740233 emerged as the top lead compounds.

### Non-Limiting Disclosure and Incorporation by Reference

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH for the natural 2′-H of DNA) or as an RNA having a modified base (thymine (methylated uracil) for natural uracil of RNA).

Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligonucleotide having the nucleobase sequence “ATCGATCG” encompasses any oligonucleotides having such nucleobase sequence, whether modified or unmodified, including, but not limited to, such compounds comprising RNA bases, such as those having sequence “AUCGAUCG” and those having some DNA bases and some RNA bases such as “AUCGATCG”.

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1: Antisense Inhibition of Human K-Ras in SKOV3 Cells by cEt Gapmers

Antisense oligonucleotides were designed targeting a K-Ras nucleic acid and were tested for their effects on K-Ras mRNA in vitro. The antisense oligonucleotides were tested in a series of experiments that had similar culture conditions. The results for each experiment are presented in separate tables shown below. Cultured SKOV3 cells at a density of 20,000 cells per well were transfected using electroporation with 2,500 nM antisense oligonucleotide. After a treatment period of approximately 24 hours, RNA was isolated from the cells and K-Ras mRNA levels were measured by quantitative real-time PCR. Human primer probe set RTS246 (forward sequence CCCAGGTGCGGGAGAGA, designated herein as SEQ ID NO: 4; reverse sequence GCTGTATCGTCAAGGCACTCTTG; designated herein as SEQ ID NO: 5; probe sequence CTTGTGGTAGTTGGAGCTGGTGGCGTAG, designated herein as SEQ ID NO: 6) was used to measure mRNA levels. K-Ras mRNA levels were adjusted according to total RNA content, as measured by RIBOGREEN®. Results are presented as percent inhibition of K-Ras, relative to untreated control cells. As used herein, a value of ‘0’ indicates that treatment with the antisense oligonucleotide did not inhibit mRNA levels.

The newly designed chimeric antisense oligonucleotides in the Tables below were designed as 3-10-3 cEt gapmers. The gapmers are 16 nucleosides in length, wherein the central gap segment comprises of ten 2′-deoxynucleosides and is flanked by wing segments on the 5′ direction and the 3′ direction comprising three nucleosides each. Each nucleoside in the 5′ wing segment and each nucleoside in the 3′ wing segment has a cEt sugar modification. The internucleoside linkages throughout each gapmer are phosphorothioate (P═S) linkages. All cytosine residues throughout each gapmer are 5-methylcytosines. “Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either a human K-Ras mRNA, designated herein as SEQ ID NO: 1 (GENBANK Accession No. NM_004985.4), the human K-Ras genomic sequence, designated herein as SEQ ID NO: 2 (the complement of GENBANK Accession No. NT_009714.17 truncated from nucleotides 18116000 to Ser. No. 18/166,000), or a human K-Ras mRNA sequence, designated herein as SEQ ID NO: 3 (GENBANK Accession No. NM_033360.3). ‘N/A’ indicates that the antisense oligonucleotide does not target that particular gene sequence with 100% complementarity.

TABLE 1
Inhibition of K-Ras mRNA by 3-10-3 cEt gapmers targeting SEQ ID NO: 1, 2,
and 3

| | SEQ ID | SEQ ID | SEQ ID | SEQ ID | SEQ ID | SEQ ID | | | |
|---|---|---|---|---|---|---|---|---|---|
| | NO: 1 | NO: 1 | NO: 2 | NO: 2 | NO: 3 | NO: 3 | | | SEQ |
| ISIS | Start | Stop | Start | Stop | Start | Stop | | % | ID |
| NO | Site | Site | Site | Site | Site | Site | Sequence | Inhibition | NO |
| 540729 | N/A | N/A | N/A | N/A | 754 | 769 | ACCCAGATTACATTAT | 0 | 13 |
| 540731 | 669 | 684 | 43058 | 43073 | 793 | 808 | TCGAATTTCTCGAACT | 41 | 14 |
| 540733 | 830 | 845 | 43219 | 43234 | 954 | 969 | GCTAAAACAAATGCTA | 54 | 15 |
| 540741 | 346 | 361 | 25573 | 25588 | 346 | 361 | CGAGAATATCCAAGAG | 48 | 16 |
| 540743 | 356 | 371 | 25583 | 25598 | 356 | 371 | CCTGCTGTGTCGAGAA | 66 | 17 |
| 540745 | 358 | 373 | 25585 | 25600 | 358 | 373 | GACCTGCTGTGTCGAG | 52 | 18 |
| 540747 | 466 | 481 | 25693 | 25708 | 466 | 481 | TATAATGGTGAATATC | 37 | 19 |
| 540749 | 476 | 491 | N/A | N/A | 476 | 491 | ATTTGTTCTCTATAAT | 21 | 20 |
| 540751 | 586 | 601 | 27273 | 27288 | 586 | 601 | AACTTCTTGCTAAGTC | 43 | 21 |
| 540753 | 658 | 673 | 43047 | 43062 | 782 | 797 | GAACTAATGTATAGAA | 44 | 22 |
| 540755 | 789 | 804 | 43178 | 43193 | 913 | 928 | TACCACTTGTACTAGT | 74 | 23 |
| 540757 | 868 | 883 | 43257 | 43272 | 992 | 1007 | CTAACAGTCTGCATGG | 63 | 24 |
| 540759 | 934 | 949 | 43323 | 43338 | 1058 | 1073 | AATACTGGCACTTAGA | 42 | 25 |
| 540761 | 1072 | 1087 | 43461 | 43476 | 1196 | 1211 | TGTTTCACACCAACAT | 61 | 26 |
| 540763 | 1228 | 1243 | 43617 | 43632 | 1352 | 1367 | TGCCTAGAAGAATCAT | 58 | 27 |
| 540765 | 1291 | 1306 | 43680 | 43695 | 1415 | 1430 | GACAAAACCTTTGTGA | 54 | 28 |
| 540767 | 1316 | 1331 | 43705 | 43720 | 1440 | 1455 | CCATGACTAATAGCAG | 88 | 29 |
| 540769 | 1473 | 1488 | 43862 | 43877 | 1597 | 1612 | ATACTGGGTCTGCCTT | 79 | 30 |
| 540771 | 1507 | 1522 | 43896 | 43911 | 1631 | 1646 | GCCCCAAAATGGTTGC | 55 | 31 |
| 540773 | 1526 | 1541 | 43915 | 43930 | 1650 | 1665 | TTAGTAGCATGTAAAT | 46 | 32 |
| 540775 | 1637 | 1652 | 44026 | 44041 | 1761 | 1776 | GAAAAGATTTAAAGTT | 0 | 33 |
| 540777 | 1709 | 1724 | 44098 | 44113 | 1833 | 1848 | GCTATAACTGGCCCAA | 83 | 34 |
| 540779 | 1898 | 1913 | 44287 | 44302 | 2022 | 2037 | ACCACAGAGTGAGATT | 79 | 35 |
| 540781 | 2102 | 2117 | 44491 | 44506 | 2226 | 2241 | GTTAATTTAACCAGTG | 80 | 36 |
| 540783 | 2223 | 2238 | 44612 | 44627 | 2347 | 2362 | TGCCATCTCACTTCAT | 57 | 37 |
| 540785 | 2318 | 2333 | 44707 | 44722 | 2442 | 2457 | TAGTAAGTGATGTCCT | 73 | 38 |
| 540787 | 2460 | 2475 | 44849 | 44864 | 2584 | 2599 | GTGTAACATAGGTTAA | 75 | 39 |
| 540789 | 2490 | 2505 | 44879 | 44894 | 2614 | 2629 | CAATTTTGCCCAAGAC | 42 | 40 |
| 540791 | 2542 | 2557 | 44931 | 44946 | 2666 | 2681 | GAAGAGTCCTAAAACG | 50 | 41 |
| 540793 | 2571 | 2586 | 44960 | 44975 | 2695 | 2710 | TAGGGAGGCAAGATGA | 56 | 42 |
| 540795 | 2599 | 2614 | 44988 | 45003 | 2723 | 2738 | TGCATCAAGTCATGGG | 83 | 43 |
| 540797 | 2694 | 2709 | 45083 | 45098 | 2818 | 2833 | TAGGGCATTTCTGATG | 38 | 44 |
| 540799 | 2794 | 2809 | 45183 | 45198 | 2918 | 2933 | GAGATGTTCAAAGCAT | 49 | 45 |
| 540801 | 2818 | 2833 | 45207 | 45222 | 2942 | 2957 | GTCGCTAATGGATTGG | 92 | 46 |
| 540803 | 2879 | 2894 | 45268 | 45283 | 3003 | 3018 | TAAATTCTCCTTCCAC | 49 | 47 |
| 540805 | 2957 | 2972 | 45346 | 45361 | 3081 | 3096 | ACAATGGAATGTATTA | 40 | 48 |
| 540807 | 3335 | 3350 | 45777 | 45792 | 3459 | 3474 | CGGTGACTGGCATCTG | 76 | 49 |
| 540809 | 3388 | 3403 | N/A | N/A | 3512 | 3527 | AGGACCGGGATTATGT | 70 | 50 |
| 540811 | 3428 | 3443 | 45817 | 45832 | 3552 | 3567 | GGCCTTAGTAAGATAT | 28 | 51 |
| 540813 | 3673 | 3688 | 46062 | 46077 | 3797 | 3812 | TGAATATCTGACATAC | 60 | 52 |
| 540815 | 3780 | 3795 | 46169 | 46184 | 3904 | 3919 | CTAGTTCAGGCACCTG | 65 | 53 |
| 540817 | 3871 | 3886 | 46260 | 46275 | 3995 | 4010 | CCTACCTAAACAGTGT | 21 | 54 |
| 540819 | 3896 | 3911 | 46285 | 46300 | 4020 | 4035 | CGAGGTACTGTGTAAG | 82 | 55 |
| 540821 | 3926 | 3941 | 46315 | 46330 | 4050 | 4065 | AGTATGGCCATTTCTT | 74 | 56 |
| 540823 | 3954 | 3969 | 46343 | 46358 | 4078 | 4093 | ATCCCCTCATAAGCAC | 61 | 57 |
| 540825 | 4107 | 4122 | 46496 | 46511 | 4231 | 4246 | AATAATTAGGTAACAT | 17 | 58 |
| 540827 | 4205 | 4220 | 46594 | 46609 | 4329 | 4344 | GTCTGCTATATTCTTC | 69 | 59 |
| 540829 | 4240 | 4255 | 46629 | 46644 | 4364 | 4379 | TACTTGGGAACATTCA | 63 | 60 |
| 540831 | 4276 | 4291 | 46665 | 46680 | 4400 | 4415 | TGCAGTGTGACTCAGT | 76 | 61 |
| 540833 | 4278 | 4293 | 46667 | 46682 | 4402 | 4417 | TATGCAGTGTGACTCA | 78 | 62 |
| 540835 | 4284 | 4299 | 46673 | 46688 | 4408 | 4423 | AATTCCTATGCAGTGT | 67 | 63 |
| 540839 | 4343 | 4358 | 46732 | 46747 | 4467 | 4482 | TAGGACAAAATTGTGC | 75 | 64 |
| 540842 | 4365 | 4380 | 46754 | 46769 | 4489 | 4504 | CACAAAGTTTCTATGT | 29 | 65 |
| 540844 | 4531 | 4546 | 46920 | 46935 | 4655 | 4670 | ATCATTACTTTTTGAC | 17 | 66 |
| 540846 | 4579 | 4594 | 46968 | 46983 | 4703 | 4718 | AAGGTAACTGCTGGGT | 86 | 67 |
| 540848 | 4642 | 4657 | 47031 | 47046 | 4766 | 4781 | CTCAATGCAGAATTCA | 75 | 68 |
| 540850 | 4872 | 4887 | 47261 | 47276 | 4996 | 5011 | ACCCAGTTAGCTCTGT | 51 | 69 |
| 540852 | 4910 | 4925 | 47299 | 47314 | 5034 | 5049 | AGACAGTGGAATTGGA | 63 | 70 |
| 540854 | 4964 | 4979 | 47353 | 47368 | 5088 | 5103 | AAGAAATTGGCACTCA | 64 | 71 |
| 540856 | 4966 | 4981 | 47355 | 47370 | 5090 | 5105 | GTAAGAAATTGGCACT | 66 | 72 |
| 540858 | 4998 | 5013 | 47387 | 47402 | 5122 | 5137 | AGGTAAACATGTTACA | 72 | 73 |
| 540860 | 5089 | 5104 | 47478 | 47493 | 5213 | 5228 | TCACACTGCATATGTC | 57 | 74 |
| 540862 | 5091 | 5106 | 47480 | 47495 | 5215 | 5230 | GATCACACTGCATATG | 31 | 75 |
| 540868 | N/A | N/A | 17921 | 17936 | N/A | N/A | GCCCTTACTTATATGC | 13 | 76 |
| 540870 | N/A | N/A | 20681 | 20696 | N/A | N/A | ATCTTGCCCACTGTTT | 15 | 77 |
| 540872 | N/A | N/A | 25497 | 25512 | N/A | N/A | AGTCTGGATTATTACA | 19 | 78 |
| 540874 | N/A | N/A | 25507 | 25522 | N/A | N/A | GGAGAAACACAGTCTG | 16 | 79 |
| 540876 | N/A | N/A | 25700 | 25715 | N/A | N/A | ACCCACCTATAATGGT | 13 | 80 |
| 540878 | N/A | N/A | 34485 | 34500 | N/A | N/A | GAAGCCAATAATTAAA | 27 | 81 |
| 540880 | N/A | N/A | 34495 | 34510 | N/A | N/A | GAGAGAATTGGAAGCC | 74 | 82 |
| 540882 | N/A | N/A | 35991 | 36006 | N/A | N/A | TTAAAGCTGGTATATT | 34 | 83 |
| 540884 | N/A | N/A | 37456 | 37471 | 716 | 731 | CAGCCAGGAGTCTTTT | 26 | 84 |
| 540886 | N/A | N/A | 43024 | 43039 | N/A | N/A | TCAACACCCTGAAATA | 16 | 85 |

TABLE 2
Inhibition of K-Ras mRNA by 3-10-3 cEt gapmers targeting SEQ ID NO: 1, 2,
and 3

| | SEQ ID | SEQ ID | SEQ ID | SEQ ID | SEQ ID | SEQ ID | | | |
|---|---|---|---|---|---|---|---|---|---|
| | NO: 1 | NO: 1 | NO: 2 | NO: 2 | NO: 3 | NO: 3 | | | SEQ |
| ISIS | Start | Stop | Start | Stop | Start | Stop | | % | ID |
| NO | Site | Site | Site | Site | Site | Site | Sequence | Inhibition | NO |
| 540728 | N/A | N/A | 37402 | 37417 | 662 | 677 | TCCCTCACCAATGTAT | 0 | 86 |
| 540730 | N/A | N/A | N/A | N/A | 759 | 774 | TCAACACCCAGATTAC | 7 | 87 |
| 540732 | 673 | 688 | 43062 | 43077 | 797 | 812 | GTTTTCGAATTTCTCG | 65 | 88 |
| 540734 | 1924 | 1939 | 44313 | 44328 | 2048 | 2063 | AATGCTCTTGATTTGT | 74 | 89 |
| 540742 | 351 | 366 | 25578 | 25593 | 351 | 366 | TGTGTCGAGAATATCC | 76 | 90 |
| 540744 | 357 | 372 | 25584 | 25599 | 357 | 372 | ACCTGCTGTGTCGAGA | 65 | 91 |
| 540746 | 359 | 374 | 25586 | 25601 | 359 | 374 | TGACCTGCTGTGTCGA | 18 | 92 |
| 540748 | 471 | 486 | N/A | N/A | 471 | 486 | TTCTCTATAATGGTGA | 55 | 93 |
| 540750 | 495 | 510 | 27182 | 27197 | 495 | 510 | AGAGTCCTTAACTCTT | 24 | 94 |
| 540752 | 601 | 616 | 27288 | 27303 | 601 | 616 | TAAAAGGAATTCCATA | 19 | 95 |
| 540754 | 777 | 792 | 43166 | 43181 | 901 | 916 | TAGTATGCCTTAAGAA | 55 | 96 |
| 540756 | 810 | 825 | 43199 | 43214 | 934 | 949 | TTTAGTGTAATGTACA | 72 | 97 |
| 540758 | 933 | 948 | 43322 | 43337 | 1057 | 1072 | ATACTGGCACTTAGAG | 40 | 98 |
| 540760 | 996 | 1011 | 43385 | 43400 | 1120 | 1135 | AAATCTTAGGTATTCA | 47 | 99 |
| 540762 | 1096 | 1111 | 43485 | 43500 | 1220 | 1235 | GATGATTCAAAAGCTT | 78 | 100 |
| 540764 | 1240 | 1255 | 43629 | 43644 | 1364 | 1379 | TATAGGACATGATGCC | 67 | 101 |
| 540766 | 1304 | 1319 | 43693 | 43708 | 1428 | 1443 | GCAGTGGAAAGGAGAC | 85 | 102 |
| 540768 | 1462 | 1477 | 43851 | 43866 | 1586 | 1601 | GCCTTAACAGGAAAAG | 58 | 103 |
| 540770 | 1488 | 1503 | 43877 | 43892 | 1612 | 1627 | AATAATCCCCATTTCA | 24 | 104 |
| 540772 | 1520 | 1535 | 43909 | 43924 | 1644 | 1659 | GCATGTAAATATAGCC | 61 | 105 |
| 540774 | 1606 | 1621 | 43995 | 44010 | 1730 | 1745 | AGTCTGACACAGGGAG | 87 | 106 |
| 540776 | 1680 | 1695 | 44069 | 44084 | 1804 | 1819 | GTCACAAGCAGAATTA | 70 | 107 |
| 540778 | 1841 | 1856 | 44230 | 44245 | 1965 | 1980 | TTTTGACTAACCAATG | 33 | 108 |
| 540780 | 1910 | 1925 | 44299 | 44314 | 2034 | 2049 | GTCAGCAGGACCACCA | 79 | 109 |
| 540782 | 2132 | 2147 | 44521 | 44536 | 2256 | 2271 | TGGATCAGACTTGAAA | 80 | 110 |
| 540784 | 2263 | 2278 | 44652 | 44667 | 2387 | 2402 | GTCACCTTCTTCCTAG | 61 | 111 |
| 540786 | 2441 | 2456 | 44830 | 44845 | 2565 | 2580 | TTTACAGATTGTGCTG | 60 | 112 |
| 540788 | 2485 | 2500 | 44874 | 44889 | 2609 | 2624 | TTGCCCAAGACTGGCA | 0 | 113 |
| 540790 | 2502 | 2517 | 44891 | 44906 | 2626 | 2641 | TCACCTCTTGCACAAT | 60 | 114 |
| 540792 | 2556 | 2571 | 44945 | 44960 | 2680 | 2695 | ACACTAATATGGAAGA | 55 | 115 |
| 540794 | 2583 | 2598 | 44972 | 44987 | 2707 | 2722 | GCATGTGGAAGGTAGG | 73 | 116 |
| 540796 | 2681 | 2696 | 45070 | 45085 | 2805 | 2820 | ATGTGACTCAGTGGGA | 83 | 117 |
| 540798 | 2738 | 2753 | 45127 | 45142 | 2862 | 2877 | TATGGTATCTGTCAGA | 80 | 118 |
| 540800 | 2806 | 2821 | 45195 | 45210 | 2930 | 2945 | TTGGGCAGCAAAGAGA | 39 | 119 |
| 540802 | 2848 | 2863 | 45237 | 45252 | 2972 | 2987 | CTATTCATACCAGGTT | 74 | 120 |
| 540804 | 2944 | 2959 | 45333 | 45348 | 3068 | 3083 | TTACTGTTACCAGGAG | 81 | 121 |
| 540806 | 2981 | 2996 | 45370 | 45385 | 3105 | 3120 | GCATGAAGATTTCTGG | 91 | 122 |
| 540808 | 3376 | 3391 | 45765 | 45780 | 3500 | 3515 | ATGTCTCTTGTTTGGG | 83 | 123 |
| 540810 | 3416 | 3431 | 45805 | 45820 | 3540 | 3555 | ATATTACAGACCACAC | 52 | 124 |
| 540812 | 3627 | 3642 | 46016 | 46031 | 3751 | 3766 | GAATCACAGTTATGCC | 78 | 125 |
| 540814 | 3688 | 3703 | 46077 | 46092 | 3812 | 3827 | ACATTTGGGTCAATAT | 46 | 126 |
| 540816 | 3834 | 3849 | 46223 | 46238 | 3958 | 3973 | ATAGCAATTCAGAAAT | 18 | 127 |
| 540818 | 3883 | 3898 | 46272 | 46287 | 4007 | 4022 | AAGTCTTAACACCCTA | 68 | 128 |
| 540820 | 3908 | 3923 | 46297 | 46312 | 4032 | 4047 | TCTGTGTAGAAACGAG | 76 | 129 |
| 540822 | 3942 | 3957 | 46331 | 46346 | 4066 | 4081 | GCACTGCAGTTCCTGA | 82 | 130 |
| 540824 | 4013 | 4028 | 46402 | 46417 | 4137 | 4152 | TAATTAACCACTACCT | 6 | 131 |
| 540826 | 4167 | 4182 | 46556 | 46571 | 4291 | 4306 | TCTATGTAATTTAGCT | 65 | 132 |
| 540828 | 4220 | 4235 | 46609 | 46624 | 4344 | 4359 | AATGATACAATATACG | 33 | 133 |
| 540830 | 4261 | 4276 | 46650 | 46665 | 4385 | 4400 | TTAAATAGAGCCTAGA | 28 | 134 |
| 540832 | 4277 | 4292 | 46666 | 46681 | 4401 | 4416 | ATGCAGTGTGACTCAG | 79 | 135 |
| 540834 | 4279 | 4294 | 46668 | 46683 | 4403 | 4418 | CTATGCAGTGTGACTC | 73 | 136 |
| 540836 | 4338 | 4353 | 46727 | 46742 | 4462 | 4477 | CAAAATTGTGCAATGG | 74 | 137 |
| 540840 | 4351 | 4366 | 46740 | 46755 | 4475 | 4490 | GTATATATTAGGACAA | 37 | 138 |
| 540843 | 4455 | 4470 | 46844 | 46859 | 4579 | 4594 | TACTGTTTGAAGAAAA | 9 | 139 |
| 540845 | 4546 | 4561 | 46935 | 46950 | 4670 | 4685 | CACAATTATCAAGAAA | 18 | 140 |
| 540847 | 4641 | 4656 | 47030 | 47045 | 4765 | 4780 | TCAATGCAGAATTCAT | 67 | 141 |
| 540849 | 4655 | 4670 | 47044 | 47059 | 4779 | 4794 | AGCTATTCAGTTTCTC | 30 | 142 |
| 540851 | 4887 | 4902 | 47276 | 47291 | 5011 | 5026 | GGATAAAACACTGTAA | 58 | 143 |
| 540853 | 4956 | 4971 | 47345 | 47360 | 5080 | 5095 | GGCACTCAAAGGAAAA | 60 | 144 |
| 540855 | 4965 | 4980 | 47354 | 47369 | 5089 | 5104 | TAAGAAATTGGCACTC | 48 | 145 |
| 540857 | 4993 | 5008 | 47382 | 47397 | 5117 | 5132 | AACATGTTACATTAAG | 43 | 146 |
| 540859 | 5006 | 5021 | 47395 | 47410 | 5130 | 5145 | TACATTCCAGGTAAAC | 51 | 147 |
| 540861 | 5090 | 5105 | 47479 | 47494 | 5214 | 5229 | ATCACACTGCATATGT | 50 | 148 |
| 540863 | 5132 | 5147 | 47521 | 47536 | 5256 | 5271 | ACATTCCTAGGTCAGC | 76 | 149 |
| 540867 | N/A | N/A | 9205 | 9220 | N/A | N/A | AAACTTCCTTTTACAT | 19 | 150 |
| 540869 | N/A | N/A | 17927 | 17942 | N/A | N/A | TACTGAGCCCTTACTT | 15 | 151 |
| 540871 | N/A | N/A | 25492 | 25507 | N/A | N/A | GGATTATTACAGTGCA | 16 | 152 |
| 540873 | N/A | N/A | 25502 | 25517 | N/A | N/A | AACACAGTCTGGATTA | 6 | 153 |
| 540875 | N/A | N/A | 25695 | 25710 | N/A | N/A | CCTATAATGGTGAATA | 32 | 154 |
| 540877 | N/A | N/A | 32700 | 32715 | N/A | N/A | GATAAATGTGAACTAG | 33 | 155 |
| 540879 | N/A | N/A | 34490 | 34505 | N/A | N/A | AATTGGAAGCCAATAA | 29 | 156 |
| 540881 | N/A | N/A | 34511 | 34526 | N/A | N/A | TGTTTCCAGCAATGCA | 59 | 157 |
| 540883 | N/A | N/A | 37365 | 37380 | N/A | N/A | GCATTGTAAAACACAA | 16 | 158 |
| 540885 | N/A | N/A | 37494 | 37509 | N/A | N/A | TACCAGATTACATTAT | 0 | 159 |

### Example 2: Antisense Inhibition of Human K-Ras in Hep3B Cells by cEt Gapmers

Antisense oligonucleotides were designed targeting a K-Ras nucleic acid and were tested for their effects on K-Ras mRNA in vitro. The antisense oligonucleotides were tested in a series of experiments that had similar culture conditions. The results for each experiment are presented in separate tables shown below. Cultured Hep3B cells at a density of 20,000 cells per well were transfected using electroporation with 2,000 nM antisense oligonucleotide. After a treatment period of approximately 24 hours, RNA was isolated from the cells and K-Ras mRNA levels were measured by quantitative real-time PCR. Human primer probe set RTS3496_MGB (forward sequence GACACAAAACAGGCTCAGGACTT, designated herein as SEQ ID NO: 7; reverse sequence TCTTGTCTTTGCTGATGTTTCAATAA, designated herein as SEQ ID NO: 8; probe sequence AAGAAGTTATGGAATTCC, designated herein as SEQ ID NO: 9) was used to measure mRNA levels. K-Ras mRNA levels were adjusted according to total RNA content, as measured by RIBOGREEN®. Results are presented as percent inhibition of K-Ras, relative to untreated control cells. As used herein, a value of ‘0’ indicates that treatment with the antisense oligonucleotide did not inhibit mRNA levels.

The newly designed chimeric antisense oligonucleotides in the Tables below were designed as 3-10-3 cEt gapmers. The gapmers are 16 nucleosides in length, wherein the central gap segment comprises of ten 2′-deoxynucleosides and is flanked by wing segments on the 5′ direction and the 3′ direction comprising three nucleosides each. Each nucleoside in the 5′ wing segment and each nucleoside in the 3′ wing segment has a cEt sugar modification. The internucleoside linkages throughout each gapmer are phosphorothioate (P═S) linkages. All cytosine residues throughout each gapmer are 5-methylcytosines. “Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either SEQ ID NO: 1 or SEQ ID NO: 2. ‘N/A’ indicates that the antisense oligonucleotide does not target that particular gene sequence with 100% complementarity.

TABLE 3
Inhibition of K-Ras mRNA by 3-10-3 cEt gapmers targeting
SEQ ID NO: 1 and 2

| | SEQ ID | SEQ ID | SEQ ID | SEQ ID | | | |
|---|---|---|---|---|---|---|---|
| | NO: 1 | NO: 1 | NO: 2 | NO: 2 | | | SEQ |
| ISIS | Start | Stop | Start | Stop | | % | ID |
| NO | Site | Site | Site | Site | Sequence | Inhibition | NO |
| 540806 | 2981 | 2996 | 45370 | 45385 | GCATGAAGATTTCTGG | 87 | 122 |
| 651467 | 148 | 163 | 2159 | 2174 | AGCCGCTGAGCCTCTG | 35 | 160 |
| 651470 | 228 | 243 | 7594 | 7609 | ACTCTTGCCTACGCCA | 73 | 161 |
| 651474 | 336 | 351 | 25563 | 25578 | CAAGAGACAGGTTTCT | 24 | 162 |
| 651475 | 348 | 363 | 25575 | 25590 | GTCGAGAATATCCAAG | 77 | 163 |
| 651476 | 354 | 369 | 25581 | 25596 | TGCTGTGTCGAGAATA | 58 | 164 |
| 651477 | 420 | 435 | 25647 | 25662 | TACACAAAGAAAGCCC | 76 | 165 |
| 651487 | 690 | 705 | 43079 | 43094 | GCTCATCTTTTCTTTA | 26 | 166 |
| 651490 | 786 | 801 | 43175 | 43190 | CACTTGTACTAGTATG | 63 | 167 |
| 651491 | 792 | 807 | 43181 | 43196 | AATTACCACTTGTACT | 21 | 168 |
| 651494 | 882 | 897 | 43271 | 43286 | ATTTAAGGTAAAAGCT | 4 | 169 |
| 651506 | 1093 | 1108 | 43482 | 43497 | GATTCAAAAGCTTCAT | 79 | 170 |
| 651507 | 1099 | 1114 | 43488 | 43503 | AGGGATGATTCAAAAG | 63 | 171 |
| 651512 | 1157 | 1172 | 43546 | 43561 | AAGGTCTCAACTGAAA | 39 | 172 |
| 651514 | 1175 | 1190 | 43564 | 43579 | TCAGTAAAAACCAATT | 24 | 173 |
| 651515 | 1184 | 1199 | 43573 | 43588 | CTCAATGTTTCAGTAA | 28 | 174 |
| 651524 | 1301 | 1316 | 43690 | 43705 | GTGGAAAGGAGACAAA | 48 | 175 |
| 651921 | 95 | 110 | 2106 | 2121 | TCCCAGTCCGAAATGG | 25 | 176 |
| 651922 | 159 | 174 | 2170 | 2185 | CCGCACCTGGGAGCCG | 32 | 177 |
| 651923 | 183 | 198 | 7549 | 7564 | AGTCATTTTCAGCAGG | 87 | 178 |
| 651924 | 195 | 210 | 7561 | 7576 | AAGTTTATATTCAGTC | 70 | 179 |
| 651925 | 204 | 219 | 7570 | 7585 | AACTACCACAAGTTTA | 43 | 180 |
| 651926 | 237 | 252 | 7603 | 7618 | CGTCAAGGCACTCTTG | 75 | 181 |
| 651927 | 252 | 267 | 7618 | 7633 | CTGAATTAGCTGTATC | 63 | 182 |
| 651928 | 312 | 327 | 25539 | 25554 | TACTACTTGCTTCCTG | 59 | 183 |
| 651929 | 322 | 337 | 25549 | 25564 | CTCCATCAATTACTAC | 48 | 184 |
| 651930 | 439 | 454 | 25666 | 25681 | TAGTATTATTTATGGC | 77 | 185 |
| 651931 | 448 | 463 | 25675 | 25690 | CAAATGATTTAGTATT | 8 | 186 |
| 651932 | 457 | 472 | 25684 | 25699 | GAATATCTTCAAATGA | 23 | 187 |
| 651933 | 485 | 500 | 27172 | 27187 | ACTCTTTTAATTTGTT | 27 | 188 |
| 651934 | 528 | 543 | 27215 | 27230 | TTTATTTCCTACTAGG | 45 | 189 |
| 651935 | 540 | 555 | 27227 | 27242 | AGGCAAATCACATTTA | 79 | 190 |
| 651936 | 551 | 566 | 27238 | 27253 | ACTGTTCTAGAAGGCA | 75 | 191 |
| 651938 | 648 | 663 | 43037 | 43052 | ATAGAAGGCATCATCA | 42 | 192 |
| 651939 | 679 | 694 | 43068 | 43083 | CTTTATGTTTTCGAAT | 5 | 193 |
| 651940 | 732 | 747 | 43121 | 43136 | ACACTTTGTCTTTGAC | 61 | 194 |
| 651941 | 741 | 756 | 43130 | 43145 | CATAATTACACACTTT | 32 | 195 |
| 651942 | 756 | 771 | 43145 | 43160 | TACAAATTGTATTTAC | 0 | 196 |
| 651943 | 771 | 786 | 43160 | 43175 | GCCTTAAGAAAAAAGT | 38 | 197 |
| 651944 | 816 | 831 | 43205 | 43220 | TAATAATTTAGTGTAA | 9 | 198 |
| 651945 | 835 | 850 | 43224 | 43239 | GTAATGCTAAAACAAA | 12 | 199 |
| 651946 | 844 | 859 | 43233 | 43248 | AAAAATTAGGTAATGC | 0 | 200 |
| 651947 | 860 | 875 | 43249 | 43264 | CTGCATGGAGCAGGAA | 31 | 201 |
| 651948 | 873 | 888 | 43262 | 43277 | AAAAGCTAACAGTCTG | 56 | 202 |
| 651949 | 907 | 922 | 43296 | 43311 | CTTCCACTGTCATTTT | 59 | 203 |
| 651950 | 927 | 942 | 43316 | 43331 | GCACTTAGAGGAAAAA | 44 | 204 |
| 651951 | 942 | 957 | 43331 | 43346 | ACTCTGGGAATACTGG | 82 | 205 |
| 651952 | 958 | 973 | 43347 | 43362 | TAGTTCAAAAACCAAA | 6 | 206 |
| 651953 | 967 | 982 | 43356 | 43371 | AGGCATTGCTAGTTCA | 83 | 207 |
| 651954 | 976 | 991 | 43365 | 43380 | CTTTTTCACAGGCATT | 75 | 208 |
| 651955 | 989 | 1004 | 43378 | 43393 | AGGTATTCAGTTTCTT | 79 | 209 |
| 651956 | 1001 | 1016 | 43390 | 43405 | GACAGAAATCTTAGGT | 51 | 210 |
| 651957 | 1010 | 1025 | N/A | N/A | AAACCCCAAGACAGAA | 16 | 211 |
| 651958 | 1019 | 1034 | N/A | N/A | ATGCACCAAAAACCCC | 69 | 212 |
| 651959 | 1028 | 1043 | 43417 | 43432 | ATCAACTGCATGCACC | 85 | 213 |
| 651960 | 1037 | 1052 | 43426 | 43441 | TAAGAAGTAATCAACT | 11 | 214 |
| 651961 | 1054 | 1069 | 43443 | 43458 | ACAATTGGTAAGAAAA | 4 | 215 |
| 651962 | 1063 | 1078 | 43452 | 43467 | CCAACATTCACAATTG | 53 | 216 |
| 651963 | 1078 | 1093 | 43467 | 43482 | TTAATTTGTTTCACAC | 34 | 217 |
| 651964 | 1110 | 1125 | 43499 | 43514 | AAACACAGAATAGGGA | 21 | 218 |
| 651965 | 1119 | 1134 | 43508 | 43523 | GACTAGATAAAACACA | 67 | 219 |
| 651966 | 1128 | 1143 | 43517 | 43532 | CATTTATGTGACTAGA | 79 | 220 |
| 651967 | 1138 | 1153 | 43527 | 43542 | GTAATTAATCCATTTA | 53 | 221 |
| 651968 | 1147 | 1162 | 43536 | 43551 | CTGAAATTAGTAATTA | 6 | 222 |
| 651969 | 1166 | 1181 | 43555 | 43570 | ACCAATTAGAAGGTCT | 38 | 223 |
| 651970 | 1195 | 1210 | 43584 | 43599 | ATTTGTGTTCCCTCAA | 68 | 224 |
| 651971 | 1204 | 1219 | 43593 | 43608 | AGCCCATAAATTTGTG | 42 | 225 |
| 651972 | 1213 | 1228 | 43602 | 43617 | TCATCAGGAAGCCCAT | 83 | 226 |
| 651973 | 1222 | 1237 | 43611 | 43626 | GAAGAATCATCATCAG | 78 | 227 |
| 651974 | 1233 | 1248 | 43622 | 43637 | CATGATGCCTAGAAGA | 41 | 228 |
| 651975 | 1245 | 1260 | 43634 | 43649 | CAAACTATAGGACATG | 56 | 229 |
| 651976 | 1254 | 1269 | 43643 | 43658 | CAGGGATGACAAACTA | 63 | 230 |
| 651977 | 1264 | 1279 | 43653 | 43668 | TTACATTCATCAGGGA | 9 | 231 |
| 651978 | 1273 | 1288 | 43662 | 43677 | AGTGTAACTTTACATT | 41 | 232 |
| 651979 | 1283 | 1298 | 43672 | 43687 | CTTTGTGAACAGTGTA | 79 | 233 |
| 651980 | 1296 | 1311 | 43685 | 43700 | AAGGAGACAAAACCTT | 3 | 234 |

TABLE 4
Inhibition of K-Ras mRNA by 3-10-3 cEt gapmers targeting
SEQ ID NO: 1 and 2

| | SEQ ID | SEQ ID | SEQ ID | SEQ ID | | | |
|---|---|---|---|---|---|---|---|
| | NO: 1 | NO: 1 | NO: 2 | NO: 2 | | | SEQ |
| ISIS | Start | Stop | Start | Stop | | % | ID |
| NO | Site | Site | Site | Site | Sequence | Inhibition | NO |
| 540734 | 1924 | 1939 | 44313 | 44328 | AATGCTCTTGATTTGT | 71 | 89 |
| 540767 | 1316 | 1331 | 43705 | 43720 | CCATGACTAATAGCAG | 77 | 29 |
| 540777 | 1709 | 1724 | 44098 | 44113 | GCTATAACTGGCCCAA | 71 | 34 |
| 540779 | 1898 | 1913 | 44287 | 44302 | ACCACAGAGTGAGATT | 73 | 35 |
| 540806 | 2981 | 2996 | 45370 | 45385 | GCATGAAGATTTCTGG | 81 | 122 |
| 651526 | 1305 | 1320 | 43694 | 43709 | AGCAGTGGAAAGGAGA | 61 | 235 |
| 651527 | 1307 | 1322 | 43696 | 43711 | ATAGCAGTGGAAAGGA | 61 | 236 |
| 651528 | 1309 | 1324 | 43698 | 43713 | TAATAGCAGTGGAAAG | 24 | 237 |
| 651529 | 1311 | 1326 | 43700 | 43715 | ACTAATAGCAGTGGAA | 44 | 238 |
| 651530 | 1313 | 1328 | 43702 | 43717 | TGACTAATAGCAGTGG | 83 | 239 |
| 651531 | 1315 | 1330 | 43704 | 43719 | CATGACTAATAGCAGT | 56 | 240 |
| 651533 | 1319 | 1334 | 43708 | 43723 | TGACCATGACTAATAG | 52 | 241 |
| 651534 | 1321 | 1336 | 43710 | 43725 | AGTGACCATGACTAAT | 68 | 242 |
| 651537 | 1398 | 1413 | 43787 | 43802 | CTTATAATAGTTTCCA | 65 | 243 |
| 651542 | 1470 | 1485 | 43859 | 43874 | CTGGGTCTGCCTTAAC | 59 | 244 |
| 651543 | 1476 | 1491 | 43865 | 43880 | TTCATACTGGGTCTGC | 78 | 245 |
| 651547 | 1601 | 1616 | 43990 | 44005 | GACACAGGGAGACTAC | 67 | 246 |
| 651548 | 1603 | 1618 | 43992 | 44007 | CTGACACAGGGAGACT | 71 | 247 |
| 651551 | 1609 | 1624 | 43998 | 44013 | AGCAGTCTGACACAGG | 80 | 248 |
| 651557 | 1704 | 1719 | 44093 | 44108 | AACTGGCCCAAATAAT | 29 | 249 |
| 651558 | 1706 | 1721 | 44095 | 44110 | ATAACTGGCCCAAATA | 35 | 250 |
| 651559 | 1708 | 1723 | 44097 | 44112 | CTATAACTGGCCCAAA | 23 | 251 |
| 651560 | 1710 | 1725 | 44099 | 44114 | AGCTATAACTGGCCCA | 73 | 252 |
| 651561 | 1712 | 1727 | 44101 | 44116 | TAAGCTATAACTGGCC | 70 | 253 |
| 651562 | 1714 | 1729 | 44103 | 44118 | AATAAGCTATAACTGG | 30 | 254 |
| 651571 | 1816 | 1831 | 44205 | 44220 | ACTGGATGACCGTGGG | 63 | 255 |
| 651578 | 1897 | 1912 | 44286 | 44301 | CCACAGAGTGAGATTG | 67 | 256 |
| 651579 | 1899 | 1914 | 44288 | 44303 | CACCACAGAGTGAGAT | 48 | 257 |
| 651580 | 1901 | 1916 | 44290 | 44305 | ACCACCACAGAGTGAG | 72 | 258 |
| 651581 | 1903 | 1918 | 44292 | 44307 | GGACCACCACAGAGTG | 62 | 259 |
| 651583 | 1907 | 1922 | 44296 | 44311 | AGCAGGACCACCACAG | 52 | 260 |
| 651586 | 1913 | 1928 | 44302 | 44317 | TTTGTCAGCAGGACCA | 86 | 261 |
| 651589 | 1921 | 1936 | 44310 | 44325 | GCTCTTGATTTGTCAG | 68 | 262 |
| 651591 | 1927 | 1942 | 44316 | 44331 | AGCAATGCTCTTGATT | 49 | 263 |
| 651592 | 1929 | 1944 | 44318 | 44333 | AAAGCAATGCTCTTGA | 53 | 264 |
| 651595 | 2020 | 2035 | 44409 | 44424 | ATGTCTTGGCACACCA | 81 | 265 |
| 651981 | 1340 | 1355 | 43729 | 43744 | AATATAATATTTTGGG | 10 | 266 |
| 651982 | 1387 | 1402 | 43776 | 43791 | TTCCATTGCCTTGTAA | 49 | 267 |
| 651983 | 1408 | 1423 | 43797 | 43812 | AGGAAATGGCCTTATA | 50 | 268 |
| 651984 | 1419 | 1434 | 43808 | 43823 | CTAATGTGAAAAGGAA | 16 | 269 |
| 651985 | 1429 | 1444 | 43818 | 43833 | AGTAATTTATCTAATG | 5 | 270 |
| 651986 | 1438 | 1453 | 43827 | 43842 | AGTCTTTATAGTAATT | 42 | 271 |
| 651987 | 1447 | 1462 | 43836 | 43851 | GCTATTAGGAGTCTTT | 82 | 272 |
| 651988 | 1456 | 1471 | 43845 | 43860 | ACAGGAAAAGCTATTA | 51 | 273 |
| 651989 | 1481 | 1496 | 43870 | 43885 | CCCATTTCATACTGGG | 2 | 274 |
| 651990 | 1493 | 1508 | 43882 | 43897 | GCTATAATAATCCCCA | 86 | 275 |
| 651991 | 1502 | 1517 | 43891 | 43906 | AAAATGGTTGCTATAA | 0 | 276 |
| 651992 | 1513 | 1528 | 43902 | 43917 | AATATAGCCCCAAAAT | 22 | 277 |
| 651993 | 1531 | 1546 | 43920 | 43935 | AAAATTTAGTAGCATG | 13 | 278 |
| 651994 | 1561 | 1576 | 43950 | 43965 | ATACTTGTTAAAATCT | 23 | 279 |
| 651995 | 1570 | 1585 | 43959 | 43974 | GAATTTTTTATACTTG | 13 | 280 |
| 651996 | 1579 | 1594 | 43968 | 43983 | TTCCTATGAGAATTTT | 62 | 281 |
| 651997 | 1588 | 1603 | 43977 | 43992 | TACATTTAATTCCTAT | 4 | 282 |
| 651998 | 1620 | 1635 | 44009 | 44024 | TACTATGAAAGAGCAG | 78 | 283 |
| 651999 | 1629 | 1644 | 44018 | 44033 | TTAAAGTTATACTATG | 10 | 284 |
| 652000 | 1643 | 1658 | 44032 | 44047 | GTTGAAGAAAAGATTT | 15 | 285 |
| 652001 | 1652 | 1667 | 44041 | 44056 | AAGACTCAAGTTGAAG | 72 | 286 |
| 652002 | 1661 | 1676 | 44050 | 44065 | CTATCTTCAAAGACTC | 87 | 287 |
| 652003 | 1672 | 1687 | 44061 | 44076 | CAGAATTAAAACTATC | 15 | 288 |
| 652004 | 1685 | 1700 | 44074 | 44089 | TTAATGTCACAAGCAG | 88 | 289 |
| 652005 | 1699 | 1714 | 44088 | 44103 | GCCCAAATAATCTTTT | 47 | 290 |
| 652006 | 1723 | 1738 | 44112 | 44127 | TCAACACCTAATAAGC | 46 | 291 |
| 652007 | 1736 | 1751 | 44125 | 44140 | AACCTTGGTCTCTTCA | 77 | 292 |
| 652008 | 1758 | 1773 | 44147 | 44162 | TTCACACAGGGCCTGG | 65 | 293 |
| 652009 | 1767 | 1782 | 44156 | 44171 | GCTCAAAGGTTCACAC | 71 | 294 |
| 652010 | 1776 | 1791 | 44165 | 44180 | TCTATGAAAGCTCAAA | 51 | 295 |
| 652011 | 1785 | 1800 | 44174 | 44189 | GTGAAACTCTCTATGA | 55 | 296 |
| 652012 | 1794 | 1809 | 44183 | 44198 | GTCCATGCTGTGAAAC | 31 | 297 |
| 652013 | 1825 | 1840 | 44214 | 44229 | CATGACAACACTGGAT | 56 | 298 |
| 652014 | 1834 | 1849 | 44223 | 44238 | TAACCAATGCATGACA | 67 | 299 |
| 652015 | 1846 | 1861 | 44235 | 44250 | CCCCATTTTGACTAAC | 38 | 300 |
| 652016 | 1862 | 1877 | 44251 | 44266 | AACTGCCCTAGTCCCT | 61 | 301 |
| 652017 | 1871 | 1886 | 44260 | 44275 | AGCTATCCAAACTGCC | 44 | 302 |
| 652018 | 1880 | 1895 | 44269 | 44284 | ATCTTGTTGAGCTATC | 75 | 303 |
| 652019 | 1918 | 1933 | 44307 | 44322 | CTTGATTTGTCAGCAG | 80 | 304 |
| 652020 | 1990 | 2005 | 44379 | 44394 | CAACTTTTGAGTTAAT | 14 | 305 |
| 652021 | 2001 | 2016 | 44390 | 44405 | CCCCAAAATCTCAACT | 20 | 306 |
| 652022 | 2010 | 2025 | 44399 | 44414 | ACACCACCACCCCAAA | 46 | 307 |

TABLE 5
Inhibition of K-Ras mRNA by 3-10-3 cEt gapmers targeting
SEQ ID NO: 1 and 2

| | SEQ ID | SEQ ID | SEQ ID | SEQ ID | | | |
|---|---|---|---|---|---|---|---|
| | NO: 1 | NO: 1 | NO: 2 | NO: 2 | | | SEQ |
| ISIS | Start | Stop | Start | Stop | | % | ID |
| NO | Site | Site | Site | Site | Sequence | Inhibition | NO |
| 540806 | 2981 | 2996 | 45370 | 45385 | GCATGAAGATTTCTGG | 83 | 122 |
| 651601 | 2099 | 2114 | 44488 | 44503 | AATTTAACCAGTGTTA | 40 | 308 |
| 651602 | 2105 | 2120 | 44494 | 44509 | AATGTTAATTTAACCA | 27 | 309 |
| 651604 | 2129 | 2144 | 44518 | 44533 | ATCAGACTTGAAAAGT | 58 | 310 |
| 651605 | 2135 | 2150 | 44524 | 44539 | ATATGGATCAGACTTG | 74 | 311 |
| 651623 | 2466 | 2481 | 44855 | 44870 | AAGATGGTGTAACATA | 68 | 312 |
| 651629 | 2600 | 2615 | 44989 | 45004 | CTGCATCAAGTCATGG | 73 | 313 |
| 651630 | 2602 | 2617 | 44991 | 45006 | AACTGCATCAAGTCAT | 64 | 314 |
| 651631 | 2604 | 2619 | 44993 | 45008 | AAAACTGCATCAAGTC | 72 | 315 |
| 651634 | 2634 | 2649 | 45023 | 45038 | AATCTTATGGTTAGGG | 85 | 316 |
| 651637 | 2676 | 2691 | 45065 | 45080 | ACTCAGTGGGAAAACT | 33 | 317 |
| 651638 | 2678 | 2693 | 45067 | 45082 | TGACTCAGTGGGAAAA | 59 | 318 |
| 651641 | 2684 | 2699 | 45073 | 45088 | CTGATGTGACTCAGTG | 76 | 319 |
| 651642 | 2686 | 2701 | 45075 | 45090 | TTCTGATGTGACTCAG | 65 | 320 |
| 651645 | 2733 | 2748 | 45122 | 45137 | TATCTGTCAGATTCTC | 83 | 321 |
| 651646 | 2735 | 2750 | 45124 | 45139 | GGTATCTGTCAGATTC | 89 | 322 |
| 651647 | 2737 | 2752 | 45126 | 45141 | ATGGTATCTGTCAGAT | 84 | 323 |
| 651649 | 2741 | 2756 | 45130 | 45145 | CTTTATGGTATCTGTC | 67 | 324 |
| 651650 | 2743 | 2758 | 45132 | 45147 | CCCTTTATGGTATCTG | 76 | 325 |
| 651659 | 2815 | 2830 | 45204 | 45219 | GCTAATGGATTGGGCA | 17 | 326 |
| 651660 | 2817 | 2832 | 45206 | 45221 | TCGCTAATGGATTGGG | 67 | 327 |
| 651662 | 2821 | 2836 | 45210 | 45225 | ACTGTCGCTAATGGAT | 43 | 328 |
| 652023 | 2047 | 2062 | 44436 | 44451 | TCACTTCATTGTTTAA | 70 | 329 |
| 652024 | 2056 | 2071 | 44445 | 44460 | AAAACTTTTTCACTTC | 62 | 330 |
| 652025 | 2065 | 2080 | 44454 | 44469 | AGAGATTGTAAAACTT | 21 | 331 |
| 652026 | 2074 | 2089 | 44463 | 44478 | GCCAAACCTAGAGATT | 42 | 332 |
| 652027 | 2083 | 2098 | 44472 | 44487 | AGAGAACTAGCCAAAC | 62 | 333 |
| 652028 | 2093 | 2108 | 44482 | 44497 | ACCAGTGTTAAGAGAA | 78 | 334 |
| 652029 | 2118 | 2133 | 44507 | 44522 | AAAGTGTTTATGCAAT | 49 | 335 |
| 652030 | 2146 | 2161 | 44535 | 44550 | AGCATTATTAAATATG | 14 | 336 |
| 652031 | 2176 | 2191 | 44565 | 44580 | TCAAAAGGATTGTTTT | 2 | 337 |
| 652032 | 2228 | 2243 | 44617 | 44632 | CACCATGCCATCTCAC | 47 | 338 |
| 652033 | 2237 | 2252 | 44626 | 44641 | CTTTCACCTCACCATG | 38 | 339 |
| 652034 | 2248 | 2263 | 44637 | 44652 | GTCCAGTGATACTTTC | 77 | 340 |
| 652035 | 2258 | 2273 | 44647 | 44662 | CTTCTTCCTAGTCCAG | 72 | 341 |
| 652036 | 2268 | 2283 | 44657 | 44672 | CCTAAGTCACCTTCTT | 47 | 342 |
| 652037 | 2277 | 2292 | 44666 | 44681 | TATCTAGAACCTAAGT | 8 | 343 |
| 652038 | 2286 | 2301 | 44675 | 44690 | AAAGACACCTATCTAG | 1 | 344 |
| 652039 | 2297 | 2312 | 44686 | 44701 | TCAGAGTCCTAAAAGA | 2 | 345 |
| 652040 | 2306 | 2321 | 44695 | 44710 | TCCTCAAAATCAGAGT | 42 | 346 |
| 652041 | 2323 | 2338 | 44712 | 44727 | ATGGATAGTAAGTGAT | 51 | 347 |
| 652042 | 2333 | 2348 | 44722 | 44737 | ACATGAAGAAATGGAT | 12 | 348 |
| 652043 | 2342 | 2357 | 44731 | 44746 | CTTCTTTTAACATGAA | 9 | 349 |
| 652044 | 2352 | 2367 | 44741 | 44756 | TTTGAGATGACTTCTT | 51 | 350 |
| 652045 | 2361 | 2376 | 44750 | 44765 | AACTAAGAGTTTGAGA | 17 | 351 |
| 652046 | 2385 | 2400 | 44774 | 44789 | AATTACATAGTTGTAA | 0 | 352 |
| 652047 | 2405 | 2420 | 44794 | 44809 | CCTTATGTAAATGGAA | 33 | 353 |
| 652048 | 2414 | 2429 | 44803 | 44818 | TAAGTGTATCCTTATG | 56 | 354 |
| 652049 | 2423 | 2438 | 44812 | 44827 | CTTGACAAATAAGTGT | 48 | 355 |
| 652050 | 2434 | 2449 | 44823 | 44838 | ATTGTGCTGAGCTTGA | 78 | 356 |
| 652051 | 2450 | 2465 | 44839 | 44854 | GGTTAAAAATTTACAG | 22 | 357 |
| 652052 | 2478 | 2493 | 44867 | 44882 | AGACTGGCACTGAAGA | 54 | 358 |
| 652053 | 2507 | 2522 | 44896 | 44911 | AAACTTCACCTCTTGC | 62 | 359 |
| 652054 | 2522 | 2537 | 44911 | 44926 | TGGATATTCAAATATA | 12 | 360 |
| 652055 | 2531 | 2546 | 44920 | 44935 | AAACGAGAATGGATAT | 28 | 361 |
| 652056 | 2547 | 2562 | 44936 | 44951 | TGGAAGAAGAGTCCTA | 14 | 362 |
| 652057 | 2561 | 2576 | 44950 | 44965 | AGATGACACTAATATG | 49 | 363 |
| 652058 | 2576 | 2591 | 44965 | 44980 | GAAGGTAGGGAGGCAA | 38 | 364 |
| 652059 | 2613 | 2628 | 45002 | 45017 | ACAAGTATTAAAACTG | 17 | 365 |
| 652060 | 2643 | 2658 | 45032 | 45047 | GCAGCAGTAAATCTTA | 60 | 366 |
| 652061 | 2652 | 2667 | 45041 | 45056 | ATATCCACAGCAGCAG | 70 | 367 |
| 652062 | 2662 | 2677 | 45051 | 45066 | CTTCATGGAGATATCC | 68 | 368 |
| 652063 | 2699 | 2714 | 45088 | 45103 | AGATGTAGGGCATTTC | 54 | 369 |
| 652064 | 2708 | 2723 | 45097 | 45112 | GAGGAAATAAGATGTA | 15 | 370 |
| 652065 | 2723 | 2738 | 45112 | 45127 | ATTCTCTTGAGCCCTG | 65 | 371 |
| 652066 | 2755 | 2770 | 45144 | 45159 | ATTAGGTCAAATCCCT | 72 | 372 |
| 652067 | 2764 | 2779 | 45153 | 45168 | AAATTAGTGATTAGGT | 32 | 373 |
| 652068 | 2773 | 2788 | 45162 | 45177 | ACCACCTGAAAATTAG | 24 | 374 |
| 652069 | 2785 | 2800 | 45174 | 45189 | AAAGCATCAGCCACCA | 46 | 375 |
| 652070 | 2799 | 2814 | 45188 | 45203 | GCAAAGAGATGTTCAA | 43 | 376 |
| 652071 | 2832 | 2847 | 45221 | 45236 | TGAAAAATCCTACTGT | 12 | 377 |
| 652072 | 2841 | 2856 | 45230 | 45245 | TACCAGGTTTGAAAAA | 23 | 378 |
| 652073 | 2864 | 2879 | 45253 | 45268 | CTGGATAGGGTTCTGT | 40 | 379 |
| 652074 | 2873 | 2888 | 45262 | 45277 | CTCCTTCCACTGGATA | 31 | 380 |
| 652075 | 2885 | 2900 | 45274 | 45289 | CTTTATTAAATTCTCC | 26 | 381 |
| 652076 | 2894 | 2909 | 45283 | 45298 | CAGCACTATCTTTATT | 33 | 382 |
| 652077 | 2906 | 2921 | 45295 | 45310 | AAGGAATTCTTTCAGC | 58 | 383 |
| 652078 | 2915 | 2930 | 45304 | 45319 | AGATTACCTAAGGAAT | 23 | 384 |

### Example 3: Antisense Inhibition of Human K-Ras in A431 Cells by cEt Gapmers

Antisense oligonucleotides were designed targeting a K-Ras nucleic acid and were tested for their effects on K-Ras mRNA in vitro. The antisense oligonucleotides were tested in a series of experiments that had similar culture conditions. The results for each experiment are presented in separate tables shown below. Cultured A431 cells at a density of 5,000 cells per well were treated with 1,000 nM antisense oligonucleotide by free uptake. After a treatment period of approximately 24 hours, RNA was isolated from the cells and K-Ras mRNA levels were measured by quantitative real-time PCR. Human primer probe set RTS3496_MGB was used to measure mRNA levels. K-Ras mRNA levels were adjusted according to total RNA content, as measured by RIBOGREEN®. Results are presented as percent inhibition of K-Ras, relative to untreated control cells. As used herein, a value of ‘0’ indicates that treatment with the antisense oligonucleotide did not inhibit mRNA levels.

The newly designed chimeric antisense oligonucleotides in the Tables below were designed as 3-10-3 cEt gapmers. The gapmers are 16 nucleosides in length, wherein the central gap segment comprises of ten 2′-deoxynucleosides and is flanked by wing segments on the 5′ direction and the 3′ direction comprising three nucleosides each. Each nucleoside in the 5′ wing segment and each nucleoside in the 3′ wing segment has a cEt sugar modification. The internucleoside linkages throughout each gapmer are phosphorothioate (P═S) linkages. All cytosine residues throughout each gapmer are 5-methylcytosines. “Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either SEQ ID NO: 1 or SEQ ID NO: 2. ‘N/A’ indicates that the antisense oligonucleotide does not target that particular gene sequence with 100% complementarity.

TABLE 6
Inhibition of K-Ras mRNA by 3-10-3 cEt gapmers targeting
SEQ ID NO: 1 and 2

| | SEQ ID | SEQ ID | SEQ ID | SEQ ID | | | |
|---|---|---|---|---|---|---|---|
| | NO: 1 | NO: 1 | NO: 2 | NO: 2 | | | SEQ |
| ISIS | Start | Stop | Start | Stop | | % | ID |
| NO | Site | Site | Site | Site | Sequence | Inhibition | NO |
| 540806 | 2981 | 2996 | 45370 | 45385 | GCATGAAGATTTCTGG | 65 | 122 |
| 540806 | 2981 | 2996 | 45370 | 45385 | GCATGAAGATTTCTGG | 63 | 122 |
| 651530 | 1313 | 1328 | 43702 | 43717 | TGACTAATAGCAGTGG | 69 | 239 |
| 651538 | 1421 | 1436 | 43810 | 43825 | ATCTAATGTGAAAAGG | 8 | 385 |
| 651539 | 1427 | 1442 | 43816 | 43831 | TAATTTATCTAATGTG | 0 | 386 |
| 651540 | 1443 | 1458 | 43832 | 43847 | TTAGGAGTCTTTATAG | 33 | 387 |
| 651541 | 1449 | 1464 | 43838 | 43853 | AAGCTATTAGGAGTCT | 60 | 388 |
| 651544 | 1494 | 1509 | 43883 | 43898 | TGCTATAATAATCCCC | 47 | 389 |
| 651545 | 1582 | 1597 | 43971 | 43986 | TAATTCCTATGAGAAT | 18 | 390 |
| 651552 | 1611 | 1626 | 44000 | 44015 | AGAGCAGTCTGACACA | 40 | 391 |
| 651987 | 1447 | 1462 | 43836 | 43851 | GCTATTAGGAGTCTTT | 72 | 272 |
| 651990 | 1493 | 1508 | 43882 | 43897 | GCTATAATAATCCCCA | 53 | 275 |
| 663529 | 1522 | 1537 | 43911 | 43926 | TAGCATGTAAATATAG | 24 | 392 |
| 695897 | 1249 | 1264 | 43638 | 43653 | ATGACAAACTATAGGA | 34 | 393 |
| 695898 | 1251 | 1266 | 43640 | 43655 | GGATGACAAACTATAG | 35 | 394 |
| 695899 | 1256 | 1271 | 43645 | 43660 | ATCAGGGATGACAAAC | 15 | 395 |
| 695900 | 1258 | 1273 | 43647 | 43662 | TCATCAGGGATGACAA | 17 | 396 |
| 695901 | 1260 | 1275 | 43649 | 43664 | ATTCATCAGGGATGAC | 20 | 397 |
| 695902 | 1262 | 1277 | 43651 | 43666 | ACATTCATCAGGGATG | 0 | 398 |
| 695903 | 1269 | 1284 | 43658 | 43673 | TAACTTTACATTCATC | 21 | 399 |
| 695904 | 1275 | 1290 | 43664 | 43679 | ACAGTGTAACTTTACA | 38 | 400 |
| 695905 | 1277 | 1292 | 43666 | 43681 | GAACAGTGTAACTTTA | 26 | 401 |
| 695906 | 1279 | 1294 | 43668 | 43683 | GTGAACAGTGTAACTT | 44 | 402 |
| 695907 | 1281 | 1296 | 43670 | 43685 | TTGTGAACAGTGTAAC | 44 | 403 |
| 695908 | 1285 | 1300 | 43674 | 43689 | ACCTTTGTGAACAGTG | 48 | 404 |
| 695909 | 1287 | 1302 | 43676 | 43691 | AAACCTTTGTGAACAG | 48 | 405 |
| 695910 | 1289 | 1304 | 43678 | 43693 | CAAAACCTTTGTGAAC | 3 | 406 |
| 695911 | 1293 | 1308 | 43682 | 43697 | GAGACAAAACCTTTGT | 10 | 407 |
| 695912 | 1312 | 1327 | 43701 | 43716 | GACTAATAGCAGTGGA | 67 | 408 |
| 695913 | 1314 | 1329 | 43703 | 43718 | ATGACTAATAGCAGTG | 35 | 409 |
| 695914 | 1323 | 1338 | 43712 | 43727 | AGAGTGACCATGACTA | 42 | 410 |
| 695915 | 1385 | 1400 | 43774 | 43789 | CCATTGCCTTGTAATT | 37 | 411 |
| 695916 | 1391 | 1406 | 43780 | 43795 | TAGTTTCCATTGCCTT | 44 | 412 |
| 695917 | 1393 | 1408 | 43782 | 43797 | AATAGTTTCCATTGCC | 64 | 413 |
| 695918 | 1395 | 1410 | 43784 | 43799 | ATAATAGTTTCCATTG | 14 | 414 |
| 695919 | 1400 | 1415 | 43789 | 43804 | GCCTTATAATAGTTTC | 44 | 415 |
| 695920 | 1403 | 1418 | 43792 | 43807 | ATGGCCTTATAATAGT | 25 | 416 |
| 695921 | 1405 | 1420 | 43794 | 43809 | AAATGGCCTTATAATA | 0 | 417 |
| 695922 | 1439 | 1454 | 43828 | 43843 | GAGTCTTTATAGTAAT | 32 | 418 |
| 695923 | 1440 | 1455 | 43829 | 43844 | GGAGTCTTTATAGTAA | 45 | 419 |
| 695924 | 1441 | 1456 | 43830 | 43845 | AGGAGTCTTTATAGTA | 69 | 420 |
| 695925 | 1442 | 1457 | 43831 | 43846 | TAGGAGTCTTTATAGT | 48 | 421 |
| 695926 | 1444 | 1459 | 43833 | 43848 | ATTAGGAGTCTTTATA | 37 | 422 |
| 695927 | 1445 | 1460 | 43834 | 43849 | TATTAGGAGTCTTTAT | 18 | 423 |
| 695928 | 1446 | 1461 | 43835 | 43850 | CTATTAGGAGTCTTTA | 30 | 424 |
| 695929 | 1448 | 1463 | 43837 | 43852 | AGCTATTAGGAGTCTT | 29 | 425 |
| 695930 | 1450 | 1465 | 43839 | 43854 | AAAGCTATTAGGAGTC | 70 | 426 |
| 695931 | 1451 | 1466 | 43840 | 43855 | AAAAGCTATTAGGAGT | 29 | 427 |
| 695932 | 1452 | 1467 | 43841 | 43856 | GAAAAGCTATTAGGAG | 32 | 428 |
| 695933 | 1453 | 1468 | 43842 | 43857 | GGAAAAGCTATTAGGA | 43 | 429 |
| 695934 | 1454 | 1469 | 43843 | 43858 | AGGAAAAGCTATTAGG | 48 | 430 |
| 695935 | 1455 | 1470 | 43844 | 43859 | CAGGAAAAGCTATTAG | 47 | 431 |
| 695936 | 1464 | 1479 | 43853 | 43868 | CTGCCTTAACAGGAAA | 43 | 432 |
| 695937 | 1478 | 1493 | 43867 | 43882 | ATTTCATACTGGGTCT | 46 | 433 |
| 695938 | 1483 | 1498 | 43872 | 43887 | TCCCCATTTCATACTG | 14 | 434 |
| 695939 | 1492 | 1507 | 43881 | 43896 | CTATAATAATCCCCAT | 23 | 435 |
| 695940 | 1495 | 1510 | 43884 | 43899 | TTGCTATAATAATCCC | 57 | 436 |
| 695941 | 1496 | 1511 | 43885 | 43900 | GTTGCTATAATAATCC | 29 | 437 |
| 695942 | 1497 | 1512 | 43886 | 43901 | GGTTGCTATAATAATC | 35 | 438 |
| 695943 | 1498 | 1513 | 43887 | 43902 | TGGTTGCTATAATAAT | 0 | 439 |
| 695944 | 1499 | 1514 | 43888 | 43903 | ATGGTTGCTATAATAA | 26 | 440 |
| 695945 | 1500 | 1515 | 43889 | 43904 | AATGGTTGCTATAATA | 12 | 441 |
| 695946 | 1501 | 1516 | 43890 | 43905 | AAATGGTTGCTATAAT | 5 | 442 |
| 695947 | 1504 | 1519 | 43893 | 43908 | CCAAAATGGTTGCTAT | 18 | 443 |
| 695948 | 1516 | 1531 | 43905 | 43920 | GTAAATATAGCCCCAA | 45 | 444 |
| 695949 | 1518 | 1533 | 43907 | 43922 | ATGTAAATATAGCCCC | 36 | 445 |
| 695950 | 1524 | 1539 | 43913 | 43928 | AGTAGCATGTAAATAT | 28 | 446 |
| 695951 | 1528 | 1543 | 43917 | 43932 | ATTTAGTAGCATGTAA | 17 | 447 |
| 695952 | 1584 | 1599 | 43973 | 43988 | TTTAATTCCTATGAGA | 20 | 448 |
| 695953 | 1591 | 1606 | 43980 | 43995 | GACTACATTTAATTCC | 0 | 449 |
| 695954 | 1594 | 1609 | 43983 | 43998 | GGAGACTACATTTAAT | 12 | 450 |
| 695955 | 1597 | 1612 | 43986 | 44001 | CAGGGAGACTACATTT | 22 | 451 |
| 695956 | 1610 | 1625 | 43999 | 44014 | GAGCAGTCTGACACAG | 41 | 452 |
| 695957 | 1613 | 1628 | 44002 | 44017 | AAAGAGCAGTCTGACA | 36 | 453 |
| 695958 | 1614 | 1629 | 44003 | 44018 | GAAAGAGCAGTCTGAC | 49 | 454 |
| 695959 | 1615 | 1630 | 44004 | 44019 | TGAAAGAGCAGTCTGA | 36 | 455 |
| 695960 | 1616 | 1631 | 44005 | 44020 | ATGAAAGAGCAGTCTG | 41 | 456 |
| 695961 | 1617 | 1632 | 44006 | 44021 | TATGAAAGAGCAGTCT | 23 | 457 |
| 695962 | 1618 | 1633 | 44007 | 44022 | CTATGAAAGAGCAGTC | 33 | 458 |

TABLE 7
Inhibition of K-Ras mRNA by 3-10-3 cEt gapmers targeting
SEQ ID NO: 1 and 2

| | SEQ ID | SEQ ID | SEQ ID | SEQ ID | | | |
|---|---|---|---|---|---|---|---|
| | NO: 1 | NO: 1 | NO: 2 | NO: 2 | | | SEQ |
| ISIS | Start | Stop | Start | Stop | | % | ID |
| NO | Site | Site | Site | Site | Sequence | Inhibition | NO |
| 540806 | 2981 | 2996 | 45370 | 45385 | GCATGAAGATTTCTGG | 58 | 122 |
| 540806 | 2981 | 2996 | 45370 | 45385 | GCATGAAGATTTCTGG | 63 | 122 |
| 651497 | 948 | 963 | 43337 | 43352 | ACCAAAACTCTGGGAA | 19 | 459 |
| 651498 | 959 | 974 | 43348 | 43363 | CTAGTTCAAAAACCAA | 0 | 460 |
| 651499 | 965 | 980 | 43354 | 43369 | GCATTGCTAGTTCAAA | 53 | 461 |
| 651501 | 1014 | 1029 | N/A | N/A | CCAAAAACCCCAAGAC | 33 | 462 |
| 651502 | 1026 | 1041 | 43415 | 43430 | CAACTGCATGCACCAA | 49 | 463 |
| 651503 | 1032 | 1047 | 43421 | 43436 | AGTAATCAACTGCATG | 35 | 464 |
| 651509 | 1124 | 1139 | 43513 | 43528 | TATGTGACTAGATAAA | 10 | 465 |
| 651510 | 1142 | 1157 | 43531 | 43546 | ATTAGTAATTAATCCA | 34 | 466 |
| 663502 | 1025 | 1040 | 43414 | 43429 | AACTGCATGCACCAAA | 30 | 467 |
| 695829 | 943 | 958 | 43332 | 43347 | AACTCTGGGAATACTG | 36 | 468 |
| 695830 | 944 | 959 | 43333 | 43348 | AAACTCTGGGAATACT | 6 | 469 |
| 695831 | 949 | 964 | 43338 | 43353 | AACCAAAACTCTGGGA | 5 | 470 |
| 695832 | 960 | 975 | 43349 | 43364 | GCTAGTTCAAAAACCA | 52 | 471 |
| 695833 | 962 | 977 | 43351 | 43366 | TTGCTAGTTCAAAAAC | 31 | 472 |
| 695834 | 963 | 978 | 43352 | 43367 | ATTGCTAGTTCAAAAA | 0 | 473 |
| 695835 | 964 | 979 | 43353 | 43368 | CATTGCTAGTTCAAAA | 32 | 474 |
| 695836 | 966 | 981 | 43355 | 43370 | GGCATTGCTAGTTCAA | 51 | 475 |
| 695837 | 970 | 985 | 43359 | 43374 | CACAGGCATTGCTAGT | 12 | 476 |
| 695838 | 971 | 986 | 43360 | 43375 | TCACAGGCATTGCTAG | 2 | 477 |
| 695839 | 972 | 987 | 43361 | 43376 | TTCACAGGCATTGCTA | 27 | 478 |
| 695840 | 994 | 1009 | 43383 | 43398 | ATCTTAGGTATTCAGT | 30 | 479 |
| 695841 | 999 | 1014 | 43388 | 43403 | CAGAAATCTTAGGTAT | 13 | 480 |
| 695842 | 1007 | 1022 | 43396 | 43411 | CCCCAAGACAGAAATC | 4 | 481 |
| 695843 | 1017 | 1032 | N/A | N/A | GCACCAAAAACCCCAA | 16 | 482 |
| 695844 | 1020 | 1035 | N/A | N/A | CATGCACCAAAAACCC | 29 | 483 |
| 695845 | 1023 | 1038 | N/A | N/A | CTGCATGCACCAAAAA | 12 | 484 |
| 695846 | 1024 | 1039 | 43413 | 43428 | ACTGCATGCACCAAAA | 14 | 485 |
| 695847 | 1027 | 1042 | 43416 | 43431 | TCAACTGCATGCACCA | 63 | 486 |
| 695848 | 1029 | 1044 | 43418 | 43433 | AATCAACTGCATGCAC | 14 | 487 |
| 695849 | 1030 | 1045 | 43419 | 43434 | TAATCAACTGCATGCA | 34 | 488 |
| 695850 | 1033 | 1048 | 43422 | 43437 | AAGTAATCAACTGCAT | 8 | 489 |
| 695851 | 1034 | 1049 | 43423 | 43438 | GAAGTAATCAACTGCA | 31 | 490 |
| 695852 | 1035 | 1050 | 43424 | 43439 | AGAAGTAATCAACTGC | 54 | 491 |
| 695853 | 1056 | 1071 | 43445 | 43460 | TCACAATTGGTAAGAA | 34 | 492 |
| 695854 | 1058 | 1073 | 43447 | 43462 | ATTCACAATTGGTAAG | 31 | 493 |
| 695855 | 1060 | 1075 | 43449 | 43464 | ACATTCACAATTGGTA | 13 | 494 |
| 695856 | 1065 | 1080 | 43454 | 43469 | CACCAACATTCACAAT | 32 | 495 |
| 695857 | 1068 | 1083 | 43457 | 43472 | TCACACCAACATTCAC | 20 | 496 |
| 695858 | 1070 | 1085 | 43459 | 43474 | TTTCACACCAACATTC | 10 | 497 |
| 695859 | 1074 | 1089 | 43463 | 43478 | TTTGTTTCACACCAAC | 36 | 498 |
| 695860 | 1076 | 1091 | 43465 | 43480 | AATTTGTTTCACACCA | 43 | 499 |
| 695861 | 1101 | 1116 | 43490 | 43505 | ATAGGGATGATTCAAA | 33 | 500 |
| 695862 | 1103 | 1118 | 43492 | 43507 | GAATAGGGATGATTCA | 6 | 501 |
| 695863 | 1105 | 1120 | 43494 | 43509 | CAGAATAGGGATGATT | 38 | 502 |
| 695864 | 1107 | 1122 | 43496 | 43511 | CACAGAATAGGGATGA | 29 | 503 |
| 695865 | 1121 | 1136 | 43510 | 43525 | GTGACTAGATAAAACA | 19 | 504 |
| 695866 | 1126 | 1141 | 43515 | 43530 | TTTATGTGACTAGATA | 26 | 505 |
| 695867 | 1130 | 1145 | 43519 | 43534 | TCCATTTATGTGACTA | 74 | 506 |
| 695868 | 1151 | 1166 | 43540 | 43555 | TCAACTGAAATTAGTA | 0 | 507 |
| 695869 | 1160 | 1175 | 43549 | 43564 | TAGAAGGTCTCAACTG | 28 | 508 |
| 695870 | 1162 | 1177 | 43551 | 43566 | ATTAGAAGGTCTCAAC | 0 | 509 |
| 695871 | 1164 | 1179 | 43553 | 43568 | CAATTAGAAGGTCTCA | 21 | 510 |
| 695872 | 1168 | 1183 | 43557 | 43572 | AAACCAATTAGAAGGT | 12 | 511 |
| 695873 | 1172 | 1187 | 43561 | 43576 | GTAAAAACCAATTAGA | 37 | 512 |
| 695874 | 1186 | 1201 | 43575 | 43590 | CCCTCAATGTTTCAGT | 10 | 513 |
| 695875 | 1188 | 1203 | 43577 | 43592 | TTCCCTCAATGTTTCA | 35 | 514 |
| 695876 | 1197 | 1212 | 43586 | 43601 | AAATTTGTGTTCCCTC | 39 | 515 |
| 695877 | 1199 | 1214 | 43588 | 43603 | ATAAATTTGTGTTCCC | 48 | 516 |
| 695878 | 1201 | 1216 | 43590 | 43605 | CCATAAATTTGTGTTC | 31 | 517 |
| 695879 | 1205 | 1220 | 43594 | 43609 | AAGCCCATAAATTTGT | 21 | 518 |
| 695880 | 1208 | 1223 | 43597 | 43612 | AGGAAGCCCATAAATT | 28 | 519 |
| 695881 | 1209 | 1224 | 43598 | 43613 | CAGGAAGCCCATAAAT | 37 | 520 |
| 695882 | 1210 | 1225 | 43599 | 43614 | TCAGGAAGCCCATAAA | 26 | 521 |
| 695883 | 1211 | 1226 | 43600 | 43615 | ATCAGGAAGCCCATAA | 55 | 522 |
| 695884 | 1212 | 1227 | 43601 | 43616 | CATCAGGAAGCCCATA | 48 | 523 |
| 695885 | 1214 | 1229 | 43603 | 43618 | ATCATCAGGAAGCCCA | 67 | 524 |
| 695886 | 1215 | 1230 | 43604 | 43619 | CATCATCAGGAAGCCC | 45 | 525 |
| 695887 | 1216 | 1231 | 43605 | 43620 | TCATCATCAGGAAGCC | 52 | 526 |
| 695888 | 1217 | 1232 | 43606 | 43621 | ATCATCATCAGGAAGC | 39 | 527 |
| 695889 | 1219 | 1234 | 43608 | 43623 | GAATCATCATCAGGAA | 50 | 528 |
| 695890 | 1220 | 1235 | 43609 | 43624 | AGAATCATCATCAGGA | 43 | 529 |
| 695891 | 1224 | 1239 | 43613 | 43628 | TAGAAGAATCATCATC | 27 | 530 |
| 695892 | 1226 | 1241 | 43615 | 43630 | CCTAGAAGAATCATCA | 40 | 531 |
| 695893 | 1235 | 1250 | 43624 | 43639 | GACATGATGCCTAGAA | 32 | 532 |
| 695894 | 1237 | 1252 | 43626 | 43641 | AGGACATGATGCCTAG | 4 | 533 |
| 695895 | 1242 | 1257 | 43631 | 43646 | ACTATAGGACATGATG | 27 | 534 |
| 695896 | 1247 | 1262 | 43636 | 43651 | GACAAACTATAGGACA | 14 | 535 |

TABLE 8
Inhibition of K-Ras mRNA by 3-10-3 cEt gapmers targeting
SEQ ID NO: 1 and 2

| | SEQ ID | SEQ ID | SEQ ID | SEQ ID | | | |
|---|---|---|---|---|---|---|---|
| | NO: 1 | NO: 1 | NO: 2 | NO: 2 | | | SEQ |
| ISIS | Start | Stop | Start | Stop | | % | ID |
| NO | Site | Site | Site | Site | Sequence | Inhibition | NO |
| 540806 | 2981 | 2996 | 45370 | 45385 | GCATGAAGATTTCTGG | 51 | 122 |
| 540806 | 2981 | 2996 | 45370 | 45385 | GCATGAAGATTTCTGG | 58 | 122 |
| 651468 | 201 | 216 | 7567 | 7582 | TACCACAAGTTTATAT | 0 | 536 |
| 651472 | 258 | 273 | 7624 | 7639 | ATGATTCTGAATTAGC | 39 | 537 |
| 651478 | 451 | 466 | 25678 | 25693 | CTTCAAATGATTTAGT | 0 | 538 |
| 651479 | 460 | 475 | 25687 | 25702 | GGTGAATATCTTCAAA | 18 | 539 |
| 651483 | 572 | 587 | 27259 | 27274 | TCCTGAGCCTGTTTTG | 97 | 540 |
| 651486 | 651 | 666 | 43040 | 43055 | TGTATAGAAGGCATCA | 0 | 541 |
| 651489 | 738 | 753 | 43127 | 43142 | AATTACACACTTTGTC | 0 | 542 |
| 651495 | 909 | 924 | 43298 | 43313 | AACTTCCACTGTCATT | 7 | 543 |
| 663453 | 184 | 199 | 7550 | 7565 | CAGTCATTTTCAGCAG | 35 | 544 |
| 663455 | 255 | 270 | 7621 | 7636 | ATTCTGAATTAGCTGT | 41 | 545 |
| 663469 | 544 | 559 | 27231 | 27246 | TAGAAGGCAAATCACA | 13 | 546 |
| 663470 | 547 | 562 | 27234 | 27249 | TTCTAGAAGGCAAATC | 7 | 547 |
| 663472 | 553 | 568 | 27240 | 27255 | CTACTGTTCTAGAAGG | 5 | 548 |
| 663479 | 676 | 691 | 43065 | 43080 | TATGTTTTCGAATTTC | 0 | 549 |
| 667550 | 224 | 239 | 7590 | 7605 | TTGCCTACGCCACCAG | 5 | 550 |
| 695767 | 48 | 63 | 2059 | 2074 | CACCTTCGCCGCCGCC | 4 | 551 |
| 695768 | 67 | 82 | 2078 | 2093 | GTACTGGCCGAGCCGC | 0 | 552 |
| 695769 | 91 | 106 | 2102 | 2117 | AGTCCGAAATGGCGGG | 0 | 553 |
| 695770 | 120 | 135 | 2131 | 2146 | CCTTCAGTGCCTGCGC | 23 | 554 |
| 695771 | 123 | 138 | 2134 | 2149 | CCGCCTTCAGTGCCTG | 0 | 555 |
| 695772 | 156 | 171 | 2167 | 2182 | CACCTGGGAGCCGCTG | 0 | 556 |
| 695773 | 167 | 182 | 2178 | 2193 | CCTCTCTCCCGCACCT | 11 | 557 |
| 695774 | 180 | 195 | 7546 | 7561 | CATTTTCAGCAGGCCT | 0 | 558 |
| 695775 | 182 | 197 | 7548 | 7563 | GTCATTTTCAGCAGGC | 22 | 559 |
| 695776 | 232 | 247 | 7598 | 7613 | AGGCACTCTTGCCTAC | 0 | 560 |
| 695777 | 314 | 329 | 25541 | 25556 | ATTACTACTTGCTTCC | 17 | 561 |
| 695778 | 316 | 331 | 25543 | 25558 | CAATTACTACTTGCTT | 0 | 562 |
| 695779 | 318 | 333 | 25545 | 25560 | ATCAATTACTACTTGC | 9 | 563 |
| 695780 | 320 | 335 | 25547 | 25562 | CCATCAATTACTACTT | 4 | 564 |
| 695781 | 339 | 354 | 25566 | 25581 | ATCCAAGAGACAGGTT | 15 | 565 |
| 695782 | 343 | 358 | 25570 | 25585 | GAATATCCAAGAGACA | 6 | 566 |
| 695783 | 363 | 378 | 25590 | 25605 | CTCTTGACCTGCTGTG | 15 | 567 |
| 695784 | 367 | 382 | 25594 | 25609 | ACTCCTCTTGACCTGC | 18 | 568 |
| 695785 | 463 | 478 | 25690 | 25705 | AATGGTGAATATCTTC | 63 | 569 |
| 695786 | 469 | 484 | N/A | N/A | CTCTATAATGGTGAAT | 10 | 570 |
| 695787 | 492 | 507 | 27179 | 27194 | GTCCTTAACTCTTTTA | 0 | 571 |
| 695788 | 498 | 513 | 27185 | 27200 | TTCAGAGTCCTTAACT | 0 | 572 |
| 695789 | 542 | 557 | 27229 | 27244 | GAAGGCAAATCACATT | 16 | 573 |
| 695790 | 555 | 570 | 27242 | 27257 | GTCTACTGTTCTAGAA | 2 | 574 |
| 695791 | 580 | 595 | 27267 | 27282 | TTGCTAAGTCCTGAGC | 86 | 575 |
| 695792 | 583 | 598 | 27270 | 27285 | TTCTTGCTAAGTCCTG | 96 | 576 |
| 695793 | 588 | 603 | 27275 | 27290 | ATAACTTCTTGCTAAG | 18 | 577 |
| 695794 | 590 | 605 | 27277 | 27292 | CCATAACTTCTTGCTA | 48 | 578 |
| 695795 | 597 | 612 | 27284 | 27299 | AGGAATTCCATAACTT | 31 | 579 |
| 695796 | 599 | 614 | 27286 | 27301 | AAAGGAATTCCATAAC | 4 | 580 |
| 695797 | 615 | 630 | 27302 | 27317 | TGCTGATGTTTCAATA | 6 | 581 |
| 695798 | 654 | 669 | 43043 | 43058 | TAATGTATAGAAGGCA | 7 | 582 |
| 695799 | 656 | 671 | 43045 | 43060 | ACTAATGTATAGAAGG | 0 | 583 |
| 695800 | 660 | 675 | 43049 | 43064 | TCGAACTAATGTATAG | 2 | 584 |
| 695801 | 662 | 677 | 43051 | 43066 | TCTCGAACTAATGTAT | 0 | 585 |
| 695802 | 665 | 680 | 43054 | 43069 | ATTTCTCGAACTAATG | 0 | 586 |
| 695803 | 667 | 682 | 43056 | 43071 | GAATTTCTCGAACTAA | 0 | 587 |
| 695804 | 671 | 686 | 43060 | 43075 | TTTCGAATTTCTCGAA | 0 | 588 |
| 695805 | 681 | 696 | 43070 | 43085 | TTCTTTATGTTTTCGA | 7 | 589 |
| 695806 | 734 | 749 | 43123 | 43138 | ACACACTTTGTCTTTG | 8 | 590 |
| 695807 | 779 | 794 | 43168 | 43183 | ACTAGTATGCCTTAAG | 7 | 591 |
| 695808 | 781 | 796 | 43170 | 43185 | GTACTAGTATGCCTTA | 0 | 592 |
| 695809 | 783 | 798 | 43172 | 43187 | TTGTACTAGTATGCCT | 51 | 593 |
| 695810 | 794 | 809 | 43183 | 43198 | AAAATTACCACTTGTA | 1 | 594 |
| 695811 | 799 | 814 | 43188 | 43203 | GTACAAAAATTACCAC | 0 | 595 |
| 695812 | 807 | 822 | 43196 | 43211 | AGTGTAATGTACAAAA | 12 | 596 |
| 695813 | 814 | 829 | 43203 | 43218 | ATAATTTAGTGTAATG | 0 | 597 |
| 695814 | 818 | 833 | 43207 | 43222 | GCTAATAATTTAGTGT | 0 | 598 |
| 695815 | 820 | 835 | 43209 | 43224 | ATGCTAATAATTTAGT | 39 | 599 |
| 695816 | 837 | 852 | 43226 | 43241 | AGGTAATGCTAAAACA | 17 | 600 |
| 695817 | 839 | 854 | 43228 | 43243 | TTAGGTAATGCTAAAA | 0 | 601 |
| 695818 | 841 | 856 | 43230 | 43245 | AATTAGGTAATGCTAA | 0 | 602 |
| 695819 | 862 | 877 | 43251 | 43266 | GTCTGCATGGAGCAGG | 39 | 603 |
| 695820 | 866 | 881 | 43255 | 43270 | AACAGTCTGCATGGAG | 4 | 604 |
| 695821 | 870 | 885 | 43259 | 43274 | AGCTAACAGTCTGCAT | 10 | 605 |
| 695822 | 875 | 890 | 43264 | 43279 | GTAAAAGCTAACAGTC | 0 | 606 |
| 695823 | 877 | 892 | 43266 | 43281 | AGGTAAAAGCTAACAG | 52 | 607 |
| 695824 | 879 | 894 | 43268 | 43283 | TAAGGTAAAAGCTAAC | 4 | 608 |
| 695825 | 884 | 899 | 43273 | 43288 | GCATTTAAGGTAAAAG | 21 | 609 |
| 695826 | 912 | 927 | 43301 | 43316 | AAAAACTTCCACTGTC | 17 | 610 |
| 695827 | 929 | 944 | 43318 | 43333 | TGGCACTTAGAGGAAA | 1 | 611 |
| 695828 | 935 | 950 | 43324 | 43339 | GAATACTGGCACTTAG | 22 | 612 |

TABLE 9
Inhibition of K-Ras mRNA by 3-10-3 cEt gapmers targeting
SEQ ID NO: 1 and 2

| | SEQ ID | SEQ ID | SEQ ID | SEQ ID | | | |
|---|---|---|---|---|---|---|---|
| | NO: 1 | NO: 1 | NO: 2 | NO: 2 | | | SEQ |
| ISIS | Start | Stop | Start | Stop | | % | ID |
| NO | Site | Site | Site | Site | Sequence | Inhibition | NO |
| 540806 | 2981 | 2996 | 45370 | 45385 | GCATGAAGATTTCTGG | 65 | 122 |
| 540806 | 2981 | 2996 | 45370 | 45385 | GCATGAAGATTTCTGG | 66 | 122 |
| 651553 | 1656 | 1671 | 44045 | 44060 | TTCAAAGACTCAAGTT | 27 | 613 |
| 651554 | 1662 | 1677 | 44051 | 44066 | ACTATCTTCAAAGACT | 33 | 614 |
| 651555 | 1686 | 1701 | 44075 | 44090 | TTTAATGTCACAAGCA | 68 | 615 |
| 651565 | 1740 | 1755 | 44129 | 44144 | TTGCAACCTTGGTCTC | 24 | 616 |
| 651568 | 1771 | 1786 | 44160 | 44175 | GAAAGCTCAAAGGTTC | 12 | 617 |
| 651572 | 1822 | 1837 | 44211 | 44226 | GACAACACTGGATGAC | 18 | 618 |
| 651573 | 1828 | 1843 | 44217 | 44232 | ATGCATGACAACACTG | 14 | 619 |
| 651585 | 1911 | 1926 | 44300 | 44315 | TGTCAGCAGGACCACC | 39 | 620 |
| 651587 | 1915 | 1930 | 44304 | 44319 | GATTTGTCAGCAGGAC | 63 | 621 |
| 651593 | 1992 | 2007 | 44381 | 44396 | CTCAACTTTTGAGTTA | 22 | 622 |
| 652004 | 1685 | 1700 | 44074 | 44089 | TTAATGTCACAAGCAG | 63 | 289 |
| 695963 | 1619 | 1634 | 44008 | 44023 | ACTATGAAAGAGCAGT | 2 | 623 |
| 695964 | 1622 | 1637 | 44011 | 44026 | TATACTATGAAAGAGC | 26 | 624 |
| 695965 | 1624 | 1639 | 44013 | 44028 | GTTATACTATGAAAGA | 48 | 625 |
| 695966 | 1633 | 1648 | 44022 | 44037 | AGATTTAAAGTTATAC | 6 | 626 |
| 695967 | 1650 | 1665 | 44039 | 44054 | GACTCAAGTTGAAGAA | 17 | 627 |
| 695968 | 1654 | 1669 | 44043 | 44058 | CAAAGACTCAAGTTGA | 20 | 628 |
| 695969 | 1655 | 1670 | 44044 | 44059 | TCAAAGACTCAAGTTG | 33 | 629 |
| 695970 | 1657 | 1672 | 44046 | 44061 | CTTCAAAGACTCAAGT | 26 | 630 |
| 695971 | 1658 | 1673 | 44047 | 44062 | TCTTCAAAGACTCAAG | 25 | 631 |
| 695972 | 1663 | 1678 | 44052 | 44067 | AACTATCTTCAAAGAC | 9 | 632 |
| 695973 | 1681 | 1696 | 44070 | 44085 | TGTCACAAGCAGAATT | 39 | 633 |
| 695974 | 1682 | 1697 | 44071 | 44086 | ATGTCACAAGCAGAAT | 25 | 634 |
| 695975 | 1683 | 1698 | 44072 | 44087 | AATGTCACAAGCAGAA | 53 | 635 |
| 695976 | 1684 | 1699 | 44073 | 44088 | TAATGTCACAAGCAGA | 61 | 636 |
| 695977 | 1687 | 1702 | 44076 | 44091 | TTTTAATGTCACAAGC | 58 | 637 |
| 695978 | 1688 | 1703 | 44077 | 44092 | CTTTTAATGTCACAAG | 2 | 638 |
| 695979 | 1689 | 1704 | 44078 | 44093 | TCTTTTAATGTCACAA | 38 | 639 |
| 695980 | 1690 | 1705 | 44079 | 44094 | ATCTTTTAATGTCACA | 64 | 640 |
| 695981 | 1691 | 1706 | 44080 | 44095 | AATCTTTTAATGTCAC | 57 | 641 |
| 695982 | 1692 | 1707 | 44081 | 44096 | TAATCTTTTAATGTCA | 0 | 642 |
| 695983 | 1693 | 1708 | 44082 | 44097 | ATAATCTTTTAATGTC | 6 | 643 |
| 695984 | 1716 | 1731 | 44105 | 44120 | CTAATAAGCTATAACT | 16 | 644 |
| 695985 | 1718 | 1733 | 44107 | 44122 | ACCTAATAAGCTATAA | 9 | 645 |
| 695986 | 1720 | 1735 | 44109 | 44124 | ACACCTAATAAGCTAT | 1 | 646 |
| 695987 | 1725 | 1740 | 44114 | 44129 | CTTCAACACCTAATAA | 4 | 647 |
| 695988 | 1727 | 1742 | 44116 | 44131 | CTCTTCAACACCTAAT | 27 | 648 |
| 695989 | 1729 | 1744 | 44118 | 44133 | GTCTCTTCAACACCTA | 52 | 649 |
| 695990 | 1744 | 1759 | 44133 | 44148 | GGCCTTGCAACCTTGG | 36 | 650 |
| 695991 | 1763 | 1778 | 44152 | 44167 | AAAGGTTCACACAGGG | 43 | 651 |
| 695992 | 1765 | 1780 | 44154 | 44169 | TCAAAGGTTCACACAG | 30 | 652 |
| 695993 | 1769 | 1784 | 44158 | 44173 | AAGCTCAAAGGTTCAC | 48 | 653 |
| 695994 | 1773 | 1788 | 44162 | 44177 | ATGAAAGCTCAAAGGT | 18 | 654 |
| 695995 | 1778 | 1793 | 44167 | 44182 | TCTCTATGAAAGCTCA | 60 | 655 |
| 695996 | 1780 | 1795 | 44169 | 44184 | ACTCTCTATGAAAGCT | 30 | 656 |
| 695997 | 1782 | 1797 | 44171 | 44186 | AAACTCTCTATGAAAG | 20 | 657 |
| 695998 | 1790 | 1805 | 44179 | 44194 | ATGCTGTGAAACTCTC | 64 | 658 |
| 695999 | 1792 | 1807 | 44181 | 44196 | CCATGCTGTGAAACTC | 35 | 659 |
| 696000 | 1798 | 1813 | 44187 | 44202 | CACAGTCCATGCTGTG | 17 | 660 |
| 696001 | 1800 | 1815 | 44189 | 44204 | GACACAGTCCATGCTG | 19 | 661 |
| 696002 | 1818 | 1833 | 44207 | 44222 | ACACTGGATGACCGTG | 6 | 662 |
| 696003 | 1820 | 1835 | 44209 | 44224 | CAACACTGGATGACCG | 35 | 663 |
| 696004 | 1830 | 1845 | 44219 | 44234 | CAATGCATGACAACAC | 31 | 664 |
| 696005 | 1832 | 1847 | 44221 | 44236 | ACCAATGCATGACAAC | 38 | 665 |
| 696006 | 1836 | 1851 | 44225 | 44240 | ACTAACCAATGCATGA | 25 | 666 |
| 696007 | 1838 | 1853 | 44227 | 44242 | TGACTAACCAATGCAT | 0 | 667 |
| 696008 | 1843 | 1858 | 44232 | 44247 | CATTTTGACTAACCAA | 7 | 668 |
| 696009 | 1865 | 1880 | 44254 | 44269 | CCAAACTGCCCTAGTC | 18 | 669 |
| 696010 | 1867 | 1882 | 44256 | 44271 | ATCCAAACTGCCCTAG | 19 | 670 |
| 696011 | 1875 | 1890 | 44264 | 44279 | GTTGAGCTATCCAAAC | 10 | 671 |
| 696012 | 1878 | 1893 | 44267 | 44282 | CTTGTTGAGCTATCCA | 74 | 672 |
| 696013 | 1882 | 1897 | 44271 | 44286 | GTATCTTGTTGAGCTA | 73 | 673 |
| 696014 | 1884 | 1899 | 44273 | 44288 | TTGTATCTTGTTGAGC | 52 | 674 |
| 696015 | 1912 | 1927 | 44301 | 44316 | TTGTCAGCAGGACCAC | 39 | 675 |
| 696016 | 1914 | 1929 | 44303 | 44318 | ATTTGTCAGCAGGACC | 56 | 676 |
| 696017 | 1917 | 1932 | 44306 | 44321 | TTGATTTGTCAGCAGG | 74 | 677 |
| 696018 | 1920 | 1935 | 44309 | 44324 | CTCTTGATTTGTCAGC | 67 | 678 |
| 696019 | 1994 | 2009 | 44383 | 44398 | ATCTCAACTTTTGAGT | 17 | 679 |
| 696020 | 2005 | 2020 | 44394 | 44409 | ACCACCCCAAAATCTC | 23 | 680 |
| 696021 | 2021 | 2036 | 44410 | 44425 | AATGTCTTGGCACACC | 46 | 681 |
| 696022 | 2022 | 2037 | 44411 | 44426 | TAATGTCTTGGCACAC | 49 | 682 |
| 696023 | 2023 | 2038 | 44412 | 44427 | TTAATGTCTTGGCACA | 23 | 683 |
| 696024 | 2024 | 2039 | 44413 | 44428 | ATTAATGTCTTGGCAC | 35 | 684 |
| 696025 | 2025 | 2040 | 44414 | 44429 | AATTAATGTCTTGGCA | 25 | 685 |
| 696026 | 2026 | 2041 | 44415 | 44430 | AAATTAATGTCTTGGC | 64 | 686 |
| 696027 | 2067 | 2082 | 44456 | 44471 | CTAGAGATTGTAAAAC | 11 | 687 |
| 696028 | 2069 | 2084 | 44458 | 44473 | ACCTAGAGATTGTAAA | 4 | 688 |

TABLE 10
Inhibition of K-Ras mRNA by 3-10-3 cEt gapmers targeting
SEQ ID NO: 1 and 2

| | SEQ ID | SEQ ID | SEQ ID | SEQ ID | | | |
|---|---|---|---|---|---|---|---|
| | NO: 1 | NO: 1 | NO: 2 | NO: 2 | | | SEQ |
| ISIS | Start | Stop | Start | Stop | | % | ID |
| NO | Site | Site | Site | Site | Sequence | Inhibition | NO |
| 540806 | 2981 | 2996 | 45370 | 45385 | GCATGAAGATTTCTGG | 61 | 122 |
| 540806 | 2981 | 2996 | 45370 | 45385 | GCATGAAGATTTCTGG | 69 | 122 |
| 651597 | 2076 | 2091 | 44465 | 44480 | TAGCCAAACCTAGAGA | 7 | 689 |
| 651599 | 2088 | 2103 | 44477 | 44492 | TGTTAAGAGAACTAGC | 0 | 690 |
| 651600 | 2095 | 2110 | 44484 | 44499 | TAACCAGTGTTAAGAG | 37 | 691 |
| 651603 | 2120 | 2135 | 44509 | 44524 | GAAAAGTGTTTATGCA | 75 | 692 |
| 651610 | 2253 | 2268 | 44642 | 44657 | TCCTAGTCCAGTGATA | 12 | 693 |
| 651613 | 2281 | 2296 | 44670 | 44685 | CACCTATCTAGAACCT | 14 | 694 |
| 651616 | 2302 | 2317 | 44691 | 44706 | CAAAATCAGAGTCCTA | 44 | 695 |
| 651620 | 2418 | 2433 | 44807 | 44822 | CAAATAAGTGTATCCT | 45 | 696 |
| 651621 | 2424 | 2439 | 44813 | 44828 | GCTTGACAAATAAGTG | 24 | 697 |
| 651622 | 2452 | 2467 | 44841 | 44856 | TAGGTTAAAAATTTAC | 14 | 698 |
| 663561 | 2440 | 2455 | 44829 | 44844 | TTACAGATTGTGCTGA | 29 | 699 |
| 696029 | 2071 | 2086 | 44460 | 44475 | AAACCTAGAGATTGTA | 26 | 700 |
| 696030 | 2078 | 2093 | 44467 | 44482 | ACTAGCCAAACCTAGA | 14 | 701 |
| 696031 | 2080 | 2095 | 44469 | 44484 | GAACTAGCCAAACCTA | 17 | 702 |
| 696032 | 2084 | 2099 | 44473 | 44488 | AAGAGAACTAGCCAAA | 33 | 703 |
| 696033 | 2085 | 2100 | 44474 | 44489 | TAAGAGAACTAGCCAA | 21 | 704 |
| 696034 | 2086 | 2101 | 44475 | 44490 | TTAAGAGAACTAGCCA | 26 | 705 |
| 696035 | 2087 | 2102 | 44476 | 44491 | GTTAAGAGAACTAGCC | 22 | 706 |
| 696036 | 2089 | 2104 | 44478 | 44493 | GTGTTAAGAGAACTAG | 39 | 707 |
| 696037 | 2090 | 2105 | 44479 | 44494 | AGTGTTAAGAGAACTA | 0 | 708 |
| 696038 | 2091 | 2106 | 44480 | 44495 | CAGTGTTAAGAGAACT | 17 | 709 |
| 696039 | 2092 | 2107 | 44481 | 44496 | CCAGTGTTAAGAGAAC | 47 | 710 |
| 696040 | 2096 | 2111 | 44485 | 44500 | TTAACCAGTGTTAAGA | 19 | 711 |
| 696041 | 2097 | 2112 | 44486 | 44501 | TTTAACCAGTGTTAAG | 15 | 712 |
| 696042 | 2107 | 2122 | 44496 | 44511 | GCAATGTTAATTTAAC | 0 | 713 |
| 696043 | 2113 | 2128 | 44502 | 44517 | GTTTATGCAATGTTAA | 60 | 714 |
| 696044 | 2115 | 2130 | 44504 | 44519 | GTGTTTATGCAATGTT | 83 | 715 |
| 696045 | 2127 | 2142 | 44516 | 44531 | CAGACTTGAAAAGTGT | 0 | 716 |
| 696046 | 2137 | 2152 | 44526 | 44541 | AAATATGGATCAGACT | 11 | 717 |
| 696047 | 2139 | 2154 | 44528 | 44543 | TTAAATATGGATCAGA | 32 | 718 |
| 696048 | 2141 | 2156 | 44530 | 44545 | TATTAAATATGGATCA | 28 | 719 |
| 696049 | 2178 | 2193 | 44567 | 44582 | TATCAAAAGGATTGTT | 23 | 720 |
| 696050 | 2180 | 2195 | 44569 | 44584 | TTTATCAAAAGGATTG | 9 | 721 |
| 696051 | 2232 | 2247 | 44621 | 44636 | ACCTCACCATGCCATC | 41 | 722 |
| 696052 | 2239 | 2254 | 44628 | 44643 | TACTTTCACCTCACCA | 22 | 723 |
| 696053 | 2241 | 2256 | 44630 | 44645 | GATACTTTCACCTCAC | 36 | 724 |
| 696054 | 2246 | 2261 | 44635 | 44650 | CCAGTGATACTTTCAC | 35 | 725 |
| 696055 | 2249 | 2264 | 44638 | 44653 | AGTCCAGTGATACTTT | 15 | 726 |
| 696056 | 2250 | 2265 | 44639 | 44654 | TAGTCCAGTGATACTT | 22 | 727 |
| 696057 | 2251 | 2266 | 44640 | 44655 | CTAGTCCAGTGATACT | 20 | 728 |
| 696058 | 2254 | 2269 | 44643 | 44658 | TTCCTAGTCCAGTGAT | 12 | 729 |
| 696059 | 2261 | 2276 | 44650 | 44665 | CACCTTCTTCCTAGTC | 30 | 730 |
| 696060 | 2270 | 2285 | 44659 | 44674 | AACCTAAGTCACCTTC | 16 | 731 |
| 696061 | 2272 | 2287 | 44661 | 44676 | AGAACCTAAGTCACCT | 32 | 732 |
| 696062 | 2274 | 2289 | 44663 | 44678 | CTAGAACCTAAGTCAC | 24 | 733 |
| 696063 | 2279 | 2294 | 44668 | 44683 | CCTATCTAGAACCTAA | 21 | 734 |
| 696064 | 2284 | 2299 | 44673 | 44688 | AGACACCTATCTAGAA | 12 | 735 |
| 696065 | 2288 | 2303 | 44677 | 44692 | TAAAAGACACCTATCT | 36 | 736 |
| 696066 | 2290 | 2305 | 44679 | 44694 | CCTAAAAGACACCTAT | 16 | 737 |
| 696067 | 2292 | 2307 | 44681 | 44696 | GTCCTAAAAGACACCT | 18 | 738 |
| 696068 | 2295 | 2310 | 44684 | 44699 | AGAGTCCTAAAAGACA | 21 | 739 |
| 696069 | 2304 | 2319 | 44693 | 44708 | CTCAAAATCAGAGTCC | 38 | 740 |
| 696070 | 2308 | 2323 | 44697 | 44712 | TGTCCTCAAAATCAGA | 29 | 741 |
| 696071 | 2315 | 2330 | 44704 | 44719 | TAAGTGATGTCCTCAA | 38 | 742 |
| 696072 | 2320 | 2335 | 44709 | 44724 | GATAGTAAGTGATGTC | 31 | 743 |
| 696073 | 2325 | 2340 | 44714 | 44729 | AAATGGATAGTAAGTG | 14 | 744 |
| 696074 | 2329 | 2344 | 44718 | 44733 | GAAGAAATGGATAGTA | 52 | 745 |
| 696075 | 2344 | 2359 | 44733 | 44748 | GACTTCTTTTAACATG | 44 | 746 |
| 696076 | 2347 | 2362 | 44736 | 44751 | GATGACTTCTTTTAAC | 20 | 747 |
| 696077 | 2354 | 2369 | 44743 | 44758 | AGTTTGAGATGACTTC | 35 | 748 |
| 696078 | 2356 | 2371 | 44745 | 44760 | AGAGTTTGAGATGACT | 23 | 749 |
| 696079 | 2359 | 2374 | 44748 | 44763 | CTAAGAGTTTGAGATG | 41 | 750 |
| 696080 | 2387 | 2402 | 44776 | 44791 | TAAATTACATAGTTGT | 12 | 751 |
| 696081 | 2407 | 2422 | 44796 | 44811 | ATCCTTATGTAAATGG | 2 | 752 |
| 696082 | 2409 | 2424 | 44798 | 44813 | GTATCCTTATGTAAAT | 27 | 753 |
| 696083 | 2411 | 2426 | 44800 | 44815 | GTGTATCCTTATGTAA | 50 | 754 |
| 696084 | 2416 | 2431 | 44805 | 44820 | AATAAGTGTATCCTTA | 12 | 755 |
| 696085 | 2420 | 2435 | 44809 | 44824 | GACAAATAAGTGTATC | 52 | 756 |
| 696086 | 2425 | 2440 | 44814 | 44829 | AGCTTGACAAATAAGT | 31 | 757 |
| 696087 | 2426 | 2441 | 44815 | 44830 | GAGCTTGACAAATAAG | 21 | 758 |
| 696088 | 2429 | 2444 | 44818 | 44833 | GCTGAGCTTGACAAAT | 22 | 759 |
| 696089 | 2430 | 2445 | 44819 | 44834 | TGCTGAGCTTGACAAA | 27 | 760 |
| 696090 | 2435 | 2450 | 44824 | 44839 | GATTGTGCTGAGCTTG | 68 | 761 |
| 696091 | 2436 | 2451 | 44825 | 44840 | AGATTGTGCTGAGCTT | 59 | 762 |
| 696092 | 2438 | 2453 | 44827 | 44842 | ACAGATTGTGCTGAGC | 48 | 763 |
| 696093 | 2439 | 2454 | 44828 | 44843 | TACAGATTGTGCTGAG | 42 | 764 |
| 696094 | 2443 | 2458 | 44832 | 44847 | AATTTACAGATTGTGC | 22 | 765 |

### Example 4: Antisense Inhibition of Human K-Ras in A431 Cells by cEt Gapmers

Antisense oligonucleotides were designed targeting a K-Ras nucleic acid and were tested for their effects on K-Ras mRNA in vitro. Cultured A431 cells at a density of 5,000 cells per well were with 1,000 nM antisense oligonucleotide by free uptake. After a treatment period of approximately 24 hours, RNA was isolated from the cells and K-Ras mRNA levels were measured by quantitative real-time PCR. Human primer probe set RTS132 (forward sequence CAAGTAGTAATTGATGGAGAAACCTGTCT, designated herein as SEQ ID NO: 10; reverse sequence CTGGTCCCTCATTGCACTGTAC; designated herein as SEQ ID NO: 11; probe sequence TGGATATTCTCGACACAGCAGGTCAAGAGG, designated herein as SEQ ID NO: 12) was used to measure mRNA levels. K-Ras mRNA levels were adjusted according to total RNA content, as measured by RIBOGREEN®. Results are presented as percent inhibition of K-Ras, relative to untreated control cells. As used herein, a value of ‘0’ indicates that treatment with the antisense oligonucleotide did not inhibit mRNA levels.

The newly designed chimeric antisense oligonucleotides in the Table below were designed as 3-10-3 cEt gapmers. The gapmers are 16 nucleosides in length, wherein the central gap segment comprises of ten 2′-deoxynucleosides and is flanked by wing segments on the 5′ direction and the 3′ direction comprising three nucleosides each. Each nucleoside in the 5′ wing segment and each nucleoside in the 3′ wing segment has a cEt sugar modification. The internucleoside linkages throughout each gapmer are phosphorothioate (P═S) linkages. All cytosine residues throughout each gapmer are 5-methylcytosines. “Start site” indicates the 5′-most nucleoside to which the gapmer is targeted in the human gene sequence. “Stop site” indicates the 3′-most nucleoside to which the gapmer is targeted human gene sequence. Each gapmer listed in the Table below is targeted to either SEQ ID NO: 1 or SEQ ID NO: 2. ‘N/A’ indicates that the antisense oligonucleotide does not target that particular gene sequence with 100% complementarity.

TABLE 11
Inhibition of K-Ras mRNA by 3-10-3 cEt gapmers targeting
SEQ ID NO: 1 and 2

| | SEQ ID | SEQ ID | SEQ ID | SEQ ID | | | |
|---|---|---|---|---|---|---|---|
| | NO: 1 | NO: 1 | NO: 2 | NO: 2 | | | SEQ |
| ISIS | Start | Stop | Start | Stop | | % | ID |
| NO | Site | Site | Site | Site | Sequence | Inhibition | NO |
| 540806 | 2981 | 2996 | 45370 | 45385 | GCATGAAGATTTCTGG | 50 | 122 |
| 540806 | 2981 | 2996 | 45370 | 45385 | GCATGAAGATTTCTGG | 53 | 122 |
| 651468 | 201 | 216 | 7567 | 7582 | TACCACAAGTTTATAT | 11 | 536 |
| 651472 | 258 | 273 | 7624 | 7639 | ATGATTCTGAATTAGC | 13 | 537 |
| 651478 | 451 | 466 | 25678 | 25693 | CTTCAAATGATTTAGT | 24 | 538 |
| 651479 | 460 | 475 | 25687 | 25702 | GGTGAATATCTTCAAA | 47 | 539 |
| 651483 | 572 | 587 | 27259 | 27274 | TCCTGAGCCTGTTTTG | 44 | 540 |
| 651486 | 651 | 666 | 43040 | 43055 | TGTATAGAAGGCATCA | 0 | 541 |
| 651489 | 738 | 753 | 43127 | 43142 | AATTACACACTTTGTC | 0 | 542 |
| 651495 | 909 | 924 | 43298 | 43313 | AACTTCCACTGTCATT | 14 | 543 |
| 663453 | 184 | 199 | 7550 | 7565 | CAGTCATTTTCAGCAG | 0 | 544 |
| 663455 | 255 | 270 | 7621 | 7636 | ATTCTGAATTAGCTGT | 31 | 545 |
| 663469 | 544 | 559 | 27231 | 27246 | TAGAAGGCAAATCACA | 18 | 546 |
| 663470 | 547 | 562 | 27234 | 27249 | TTCTAGAAGGCAAATC | 3 | 547 |
| 663472 | 553 | 568 | 27240 | 27255 | CTACTGTTCTAGAAGG | 16 | 548 |
| 663479 | 676 | 691 | 43065 | 43080 | TATGTTTTCGAATTTC | 0 | 549 |
| 667550 | 224 | 239 | 7590 | 7605 | TTGCCTACGCCACCAG | 0 | 550 |
| 695767 | 48 | 63 | 2059 | 2074 | CACCTTCGCCGCCGCC | 0 | 551 |
| 695768 | 67 | 82 | 2078 | 2093 | GTACTGGCCGAGCCGC | 5 | 552 |
| 695769 | 91 | 106 | 2102 | 2117 | AGTCCGAAATGGCGGG | 0 | 553 |
| 695770 | 120 | 135 | 2131 | 2146 | CCTTCAGTGCCTGCGC | 45 | 554 |
| 695771 | 123 | 138 | 2134 | 2149 | CCGCCTTCAGTGCCTG | 0 | 555 |
| 695772 | 156 | 171 | 2167 | 2182 | CACCTGGGAGCCGCTG | 0 | 556 |
| 695773 | 167 | 182 | 2178 | 2193 | CCTCTCTCCCGCACCT | 0 | 557 |
| 695774 | 180 | 195 | 7546 | 7561 | CATTTTCAGCAGGCCT | 0 | 558 |
| 695775 | 182 | 197 | 7548 | 7563 | GTCATTTTCAGCAGGC | 0 | 559 |
| 695776 | 232 | 247 | 7598 | 7613 | AGGCACTCTTGCCTAC | 0 | 560 |
| 695777 | 314 | 329 | 25541 | 25556 | ATTACTACTTGCTTCC | 0 | 561 |
| 695778 | 316 | 331 | 25543 | 25558 | CAATTACTACTTGCTT | 5 | 562 |
| 695779 | 318 | 333 | 25545 | 25560 | ATCAATTACTACTTGC | 13 | 563 |
| 695780 | 320 | 335 | 25547 | 25562 | CCATCAATTACTACTT | 17 | 564 |
| 695781 | 339 | 354 | 25566 | 25581 | ATCCAAGAGACAGGTT | 70 | 565 |
| 695782 | 343 | 358 | 25570 | 25585 | GAATATCCAAGAGACA | 52 | 566 |
| 695783 | 363 | 378 | 25590 | 25605 | CTCTTGACCTGCTGTG | 80 | 567 |
| 695784 | 367 | 382 | 25594 | 25609 | ACTCCTCTTGACCTGC | 96 | 568 |
| 695785 | 463 | 478 | 25690 | 25705 | AATGGTGAATATCTTC | 54 | 569 |
| 695786 | 469 | 484 | N/A | N/A | CTCTATAATGGTGAAT | 3 | 570 |
| 695787 | 492 | 507 | 27179 | 27194 | GTCCTTAACTCTTTTA | 6 | 571 |
| 695788 | 498 | 513 | 27185 | 27200 | TTCAGAGTCCTTAACT | 0 | 572 |
| 695789 | 542 | 557 | 27229 | 27244 | GAAGGCAAATCACATT | 18 | 573 |
| 695790 | 555 | 570 | 27242 | 27257 | GTCTACTGTTCTAGAA | 0 | 574 |
| 695791 | 580 | 595 | 27267 | 27282 | TTGCTAAGTCCTGAGC | 0 | 575 |
| 695792 | 583 | 598 | 27270 | 27285 | TTCTTGCTAAGTCCTG | 46 | 576 |
| 695793 | 588 | 603 | 27275 | 27290 | ATAACTTCTTGCTAAG | 20 | 577 |
| 695794 | 590 | 605 | 27277 | 27292 | CCATAACTTCTTGCTA | 0 | 578 |
| 695795 | 597 | 612 | 27284 | 27299 | AGGAATTCCATAACTT | 6 | 579 |
| 695796 | 599 | 614 | 27286 | 27301 | AAAGGAATTCCATAAC | 1 | 580 |
| 695797 | 615 | 630 | 27302 | 27317 | TGCTGATGTTTCAATA | 0 | 581 |
| 695798 | 654 | 669 | 43043 | 43058 | TAATGTATAGAAGGCA | 0 | 582 |
| 695799 | 656 | 671 | 43045 | 43060 | ACTAATGTATAGAAGG | 10 | 583 |
| 695800 | 660 | 675 | 43049 | 43064 | TCGAACTAATGTATAG | 0 | 584 |
| 695801 | 662 | 677 | 43051 | 43066 | TCTCGAACTAATGTAT | 0 | 585 |
| 695802 | 665 | 680 | 43054 | 43069 | ATTTCTCGAACTAATG | 0 | 586 |
| 695803 | 667 | 682 | 43056 | 43071 | GAATTTCTCGAACTAA | 4 | 587 |
| 695804 | 671 | 686 | 43060 | 43075 | TTTCGAATTTCTCGAA | 0 | 588 |
| 695805 | 681 | 696 | 43070 | 43085 | TTCTTTATGTTTTCGA | 0 | 589 |
| 695806 | 734 | 749 | 43123 | 43138 | ACACACTTTGTCTTTG | 8 | 590 |
| 695807 | 779 | 794 | 43168 | 43183 | ACTAGTATGCCTTAAG | 3 | 591 |
| 695808 | 781 | 796 | 43170 | 43185 | GTACTAGTATGCCTTA | 0 | 592 |
| 695809 | 783 | 798 | 43172 | 43187 | TTGTACTAGTATGCCT | 43 | 593 |
| 695810 | 794 | 809 | 43183 | 43198 | AAAATTACCACTTGTA | 2 | 594 |
| 695811 | 799 | 814 | 43188 | 43203 | GTACAAAAATTACCAC | 0 | 595 |
| 695812 | 807 | 822 | 43196 | 43211 | AGTGTAATGTACAAAA | 0 | 596 |
| 695813 | 814 | 829 | 43203 | 43218 | ATAATTTAGTGTAATG | 0 | 597 |
| 695814 | 818 | 833 | 43207 | 43222 | GCTAATAATTTAGTGT | 0 | 598 |
| 695815 | 820 | 835 | 43209 | 43224 | ATGCTAATAATTTAGT | 0 | 599 |
| 695816 | 837 | 852 | 43226 | 43241 | AGGTAATGCTAAAACA | 12 | 600 |
| 695817 | 839 | 854 | 43228 | 43243 | TTAGGTAATGCTAAAA | 0 | 601 |
| 695818 | 841 | 856 | 43230 | 43245 | AATTAGGTAATGCTAA | 0 | 602 |
| 695819 | 862 | 877 | 43251 | 43266 | GTCTGCATGGAGCAGG | 2 | 603 |
| 695820 | 866 | 881 | 43255 | 43270 | AACAGTCTGCATGGAG | 9 | 604 |
| 695821 | 870 | 885 | 43259 | 43274 | AGCTAACAGTCTGCAT | 0 | 605 |
| 695822 | 875 | 890 | 43264 | 43279 | GTAAAAGCTAACAGTC | 45 | 606 |
| 695823 | 877 | 892 | 43266 | 43281 | AGGTAAAAGCTAACAG | 52 | 607 |
| 695824 | 879 | 894 | 43268 | 43283 | TAAGGTAAAAGCTAAC | 0 | 608 |
| 695825 | 884 | 899 | 43273 | 43288 | GCATTTAAGGTAAAAG | 10 | 609 |
| 695826 | 912 | 927 | 43301 | 43316 | AAAAACTTCCACTGTC | 14 | 610 |
| 695827 | 929 | 944 | 43318 | 43333 | TGGCACTTAGAGGAAA | 19 | 611 |
| 695828 | 935 | 950 | 43324 | 43339 | GAATACTGGCACTTAG | 13 | 612 |

### Example 5: Antisense Inhibition of Human K-Ras in A431 Cells by cEt Gapmers

Antisense oligonucleotides were designed targeting a K-Ras nucleic acid and were tested for their effects on K-Ras mRNA in vitro. The antisense oligonucleotides were tested in a series of experiments that had similar culture conditions. The results for each experiment are presented in separate tables shown below. Cultured A431cells at a density of 5,000 cells per well were treated with 2,000 nM antisense oligonucleotide by free uptake. After a treatment period of approximately 24 hours, RNA was isolated from the cells and K-Ras mRNA levels were measured by quantitative real-time PCR. Human primer probe set RTS3496_MGB was used to measure mRNA levels. K-Ras mRNA levels were adjusted according to total RNA content, as measured by RIBOGREEN®. Results are presented as percent inhibition of K-Ras, relative to untreated control cells. As used herein, a value of ‘0’ indicates that treatment with the antisense oligonucleotide did not inhibit mRNA levels.

The newly designed chimeric antisense oligonucleotides in the Tables below were designed as 3-10-3 cEt gapmers. The gapmers are 16 nucleosides in length, wherein the central gap segment comprises of ten 2′-deoxynucleosides and is flanked by wing segments on the 5′ direction and the 3′ direction comprising three nucleosides each. Each nucleoside in the 5′ wing segment and each nucleoside in the 3′ wing segment has a cEt sugar modification. The internucleoside linkages throughout each gapmer are phosphorothioate (P═S) linkages. All cytosine residues throughout each gapmer are 5-methylcytosines. “Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either SEQ ID NO: 1 or SEQ ID NO: 2. ‘N/A’ indicates that the antisense oligonucleotide does not target that particular gene sequence with 100% complementarity.

TABLE 12
Inhibition of K-Ras mRNA by 3-10-3 cEt gapmers
targeting SEQ ID NO: 1 and 2

| | SEQ ID | SEQ ID | SEQ ID | SEQ ID | | | |
|---|---|---|---|---|---|---|---|
| | NO: 1 | NO: 1 | NO: 2 | NO: 2 | | | SEQ |
| ISIS | Start | Stop | Start | Stop | | % | ID |
| NO | Site | Site | Site | Site | Sequence | Inhibition | NO |
| 540733 | 830 | 845 | 43219 | 43234 | GCTAAAACAAATGCTA | 33 | 15 |
| 540747 | 466 | 481 | 25693 | 25708 | TATAATGGTGAATATC | 7 | 19 |
| 540806 | 2981 | 2996 | 45370 | 45385 | GCATGAAGATTTCTGG | 62 | 122 |
| 651479 | 460 | 475 | 25687 | 25702 | GGTGAATATCTTCAAA | 28 | 539 |
| 651490 | 786 | 801 | 43175 | 43190 | CACTTGTACTAGTATG | 36 | 167 |
| 651499 | 965 | 980 | 43354 | 43369 | GCATTGCTAGTTCAAA | 46 | 461 |
| 651502 | 1026 | 1041 | 43415 | 43430 | CAACTGCATGCACCAA | 31 | 463 |
| 651529 | 1311 | 1326 | 43700 | 43715 | ACTAATAGCAGTGGAA | 27 | 238 |
| 651530 | 1313 | 1328 | 43702 | 43717 | TGACTAATAGCAGTGG | 58 | 239 |
| 651540 | 1443 | 1458 | 43832 | 43847 | TTAGGAGTCTTTATAG | 30 | 387 |
| 651541 | 1449 | 1464 | 43838 | 43853 | AAGCTATTAGGAGTCT | 65 | 388 |
| 651953 | 967 | 982 | 43356 | 43371 | AGGCATTGCTAGTTCA | 55 | 207 |
| 651959 | 1028 | 1043 | 43417 | 43432 | ATCAACTGCATGCACC | 42 | 213 |
| 651966 | 1128 | 1143 | 43517 | 43532 | CATTTATGTGACTAGA | 56 | 220 |
| 651967 | 1138 | 1153 | 43527 | 43542 | GTAATTAATCCATTTA | 15 | 221 |
| 651987 | 1447 | 1462 | 43836 | 43851 | GCTATTAGGAGTCTTT | 76 | 272 |
| 663467 | 461 | 476 | 25688 | 25703 | TGGTGAATATCTTCAA | 9 | 766 |
| 663485 | 819 | 834 | 43208 | 43223 | TGCTAATAATTTAGTG | 6 | 767 |
| 663486 | 823 | 838 | 43212 | 43227 | CAAATGCTAATAATTT | 0 | 768 |
| 695785 | 463 | 478 | 25690 | 25705 | AATGGTGAATATCTTC | 50 | 569 |
| 695808 | 781 | 796 | 43170 | 43185 | GTACTAGTATGCCTTA | 50 | 592 |
| 695809 | 783 | 798 | 43172 | 43187 | TTGTACTAGTATGCCT | 55 | 593 |
| 695814 | 818 | 833 | 43207 | 43222 | GCTAATAATTTAGTGT | 18 | 598 |
| 695815 | 820 | 835 | 43209 | 43224 | ATGCTAATAATTTAGT | 11 | 599 |
| 695822 | 875 | 890 | 43264 | 43279 | GTAAAAGCTAACAGTC | 21 | 606 |
| 695823 | 877 | 892 | 43266 | 43281 | AGGTAAAAGCTAACAG | 41 | 607 |
| 695824 | 879 | 894 | 43268 | 43283 | TAAGGTAAAAGCTAAC | 20 | 608 |
| 695835 | 964 | 979 | 43353 | 43368 | CATTGCTAGTTCAAAA | 9 | 474 |
| 695836 | 966 | 981 | 43355 | 43370 | GGCATTGCTAGTTCAA | 43 | 475 |
| 695847 | 1027 | 1042 | 43416 | 43431 | TCAACTGCATGCACCA | 52 | 486 |
| 695867 | 1130 | 1145 | 43519 | 43534 | TCCATTTATGTGACTA | 68 | 506 |
| 695912 | 1312 | 1327 | 43701 | 43716 | GACTAATAGCAGTGGA | 62 | 408 |
| 695913 | 1314 | 1329 | 43703 | 43718 | ATGACTAATAGCAGTG | 49 | 409 |
| 695916 | 1391 | 1406 | 43780 | 43795 | TAGTTTCCATTGCCTT | 16 | 412 |
| 695917 | 1393 | 1408 | 43782 | 43797 | AATAGTTTCCATTGCC | 56 | 413 |
| 695918 | 1395 | 1410 | 43784 | 43799 | ATAATAGTTTCCATTG | 8 | 414 |
| 695923 | 1440 | 1455 | 43829 | 43844 | GGAGTCTTTATAGTAA | 38 | 419 |
| 695924 | 1441 | 1456 | 43830 | 43845 | AGGAGTCTTTATAGTA | 66 | 420 |
| 695925 | 1442 | 1457 | 43831 | 43846 | TAGGAGTCTTTATAGT | 41 | 421 |
| 695926 | 1444 | 1459 | 43833 | 43848 | ATTAGGAGTCTTTATA | 20 | 422 |
| 695927 | 1445 | 1460 | 43834 | 43849 | TATTAGGAGTCTTTAT | 13 | 423 |
| 695928 | 1446 | 1461 | 43835 | 43850 | CTATTAGGAGTCTTTA | 34 | 424 |
| 695929 | 1448 | 1463 | 43837 | 43852 | AGCTATTAGGAGTCTT | 35 | 425 |
| 695930 | 1450 | 1465 | 43839 | 43854 | AAAGCTATTAGGAGTC | 65 | 426 |
| 695931 | 1451 | 1466 | 43840 | 43855 | AAAAGCTATTAGGAGT | 25 | 427 |
| 696286 | 3841 | 3856 | 46230 | 46245 | GTTTCACATAGCAATT | 29 | 769 |
| 696287 | 3844 | 3859 | 46233 | 46248 | GTAGTTTCACATAGCA | 46 | 770 |
| 696288 | 3846 | 3861 | 46235 | 46250 | CTGTAGTTTCACATAG | 25 | 771 |
| 716582 | 459 | 474 | 25686 | 25701 | GTGAATATCTTCAAAT | 0 | 772 |
| 716583 | 462 | 477 | 25689 | 25704 | ATGGTGAATATCTTCA | 64 | 773 |
| 716584 | 464 | 479 | 25691 | 25706 | TAATGGTGAATATCTT | 11 | 774 |
| 716585 | 465 | 480 | 25692 | 25707 | ATAATGGTGAATATCT | 41 | 775 |
| 716586 | 780 | 795 | 43169 | 43184 | TACTAGTATGCCTTAA | 16 | 776 |
| 716587 | 782 | 797 | 43171 | 43186 | TGTACTAGTATGCCTT | 66 | 777 |
| 716588 | 784 | 799 | 43173 | 43188 | CTTGTACTAGTATGCC | 59 | 778 |
| 716589 | 785 | 800 | 43174 | 43189 | ACTTGTACTAGTATGC | 51 | 779 |
| 716590 | 821 | 836 | 43210 | 43225 | AATGCTAATAATTTAG | 0 | 780 |
| 716591 | 822 | 837 | 43211 | 43226 | AAATGCTAATAATTTA | 13 | 781 |
| 716592 | 824 | 839 | 43213 | 43228 | ACAAATGCTAATAATT | 1 | 782 |
| 716593 | 825 | 840 | 43214 | 43229 | AACAAATGCTAATAAT | 0 | 783 |
| 716594 | 826 | 841 | 43215 | 43230 | AAACAAATGCTAATAA | 9 | 784 |
| 716595 | 827 | 842 | 43216 | 43231 | AAAACAAATGCTAATA | 12 | 785 |
| 716596 | 828 | 843 | 43217 | 43232 | TAAAACAAATGCTAAT | 5 | 786 |
| 716597 | 829 | 844 | 43218 | 43233 | CTAAAACAAATGCTAA | 15 | 787 |
| 716598 | 876 | 891 | 43265 | 43280 | GGTAAAAGCTAACAGT | 49 | 788 |
| 716599 | 878 | 893 | 43267 | 43282 | AAGGTAAAAGCTAACA | 21 | 789 |
| 716600 | 1129 | 1144 | 43518 | 43533 | CCATTTATGTGACTAG | 75 | 790 |
| 716601 | 1131 | 1146 | 43520 | 43535 | ATCCATTTATGTGACT | 38 | 791 |
| 716602 | 1132 | 1147 | 43521 | 43536 | AATCCATTTATGTGAC | 26 | 792 |
| 716603 | 1133 | 1148 | 43522 | 43537 | TAATCCATTTATGTGA | 31 | 793 |
| 716604 | 1134 | 1149 | 43523 | 43538 | TTAATCCATTTATGTG | 34 | 794 |
| 716605 | 1135 | 1150 | 43524 | 43539 | ATTAATCCATTTATGT | 3 | 795 |
| 716606 | 1136 | 1151 | 43525 | 43540 | AATTAATCCATTTATG | 16 | 796 |
| 716607 | 1137 | 1152 | 43526 | 43541 | TAATTAATCCATTTAT | 0 | 797 |
| 716608 | 1392 | 1407 | 43781 | 43796 | ATAGTTTCCATTGCCT | 65 | 798 |
| 716609 | 1394 | 1409 | 43783 | 43798 | TAATAGTTTCCATTGC | 53 | 799 |
| 716610 | 3842 | 3857 | 46231 | 46246 | AGTTTCACATAGCAAT | 38 | 800 |
| 716611 | 3843 | 3858 | 46232 | 46247 | TAGTTTCACATAGCAA | 51 | 801 |
| 716612 | 3845 | 3860 | 46234 | 46249 | TGTAGTTTCACATAGC | 59 | 802 |

TABLE 13
Inhibition of K-Ras mRNA by 3-10-3 cEt gapmers
targeting SEQ ID NO: 1 and 2

| | SEQ ID | SEQ ID | SEQ ID | SEQ ID | | | |
|---|---|---|---|---|---|---|---|
| | NO: 1 | NO: 1 | NO: 2 | NO: 2 | | | SEQ |
| ISIS | Start | Stop | Start | Stop | | % | ID |
| NO | Site | Site | Site | Site | Sequence | Inhibition | NO |
| 540770 | 1488 | 1503 | 43877 | 43892 | AATAATCCCCATTTCA | 16 | 104 |
| 540772 | 1520 | 1535 | 43909 | 43924 | GCATGTAAATATAGCC | 11 | 105 |
| 540806 | 2981 | 2996 | 45370 | 45385 | GCATGAAGATTTCTGG | 60 | 122 |
| 651544 | 1494 | 1509 | 43883 | 43898 | TGCTATAATAATCCCC | 43 | 389 |
| 651555 | 1686 | 1701 | 44075 | 44090 | TTTAATGTCACAAGCA | 57 | 615 |
| 651569 | 1789 | 1804 | 44178 | 44193 | TGCTGTGAAACTCTCT | 27 | 803 |
| 651587 | 1915 | 1930 | 44304 | 44319 | GATTTGTCAGCAGGAC | 47 | 621 |
| 651588 | 1919 | 1934 | 44308 | 44323 | TCTTGATTTGTCAGCA | 62 | 804 |
| 651589 | 1921 | 1936 | 44310 | 44325 | GCTCTTGATTTGTCAG | 23 | 262 |
| 651990 | 1493 | 1508 | 43882 | 43897 | GCTATAATAATCCCCA | 56 | 275 |
| 652004 | 1685 | 1700 | 44074 | 44089 | TTAATGTCACAAGCAG | 52 | 289 |
| 652010 | 1776 | 1791 | 44165 | 44180 | TCTATGAAAGCTCAAA | 18 | 295 |
| 652011 | 1785 | 1800 | 44174 | 44189 | GTGAAACTCTCTATGA | 28 | 296 |
| 652018 | 1880 | 1895 | 44269 | 44284 | ATCTTGTTGAGCTATC | 40 | 303 |
| 652019 | 1918 | 1933 | 44307 | 44322 | CTTGATTTGTCAGCAG | 44 | 304 |
| 652023 | 2047 | 2062 | 44436 | 44451 | TCACTTCATTGTTTAA | 32 | 329 |
| 695867 | 1130 | 1145 | 43519 | 43534 | TCCATTTATGTGACTA | 63 | 506 |
| 695939 | 1492 | 1507 | 43881 | 43896 | CTATAATAATCCCCAT | 6 | 435 |
| 695940 | 1495 | 1510 | 43884 | 43899 | TTGCTATAATAATCCC | 39 | 436 |
| 695948 | 1516 | 1531 | 43905 | 43920 | GTAAATATAGCCCCAA | 29 | 444 |
| 695949 | 1518 | 1533 | 43907 | 43922 | ATGTAAATATAGCCCC | 28 | 445 |
| 695975 | 1683 | 1698 | 44072 | 44087 | AATGTCACAAGCAGAA | 49 | 635 |
| 695976 | 1684 | 1699 | 44073 | 44088 | TAATGTCACAAGCAGA | 52 | 636 |
| 695977 | 1687 | 1702 | 44076 | 44091 | TTTTAATGTCACAAGC | 52 | 637 |
| 695978 | 1688 | 1703 | 44077 | 44092 | CTTTTAATGTCACAAG | 9 | 638 |
| 695979 | 1689 | 1704 | 44078 | 44093 | TCTTTTAATGTCACAA | 46 | 639 |
| 695980 | 1690 | 1705 | 44079 | 44094 | ATCTTTTAATGTCACA | 58 | 640 |
| 695981 | 1691 | 1706 | 44080 | 44095 | AATCTTTTAATGTCAC | 44 | 641 |
| 695982 | 1692 | 1707 | 44081 | 44096 | TAATCTTTTAATGTCA | 7 | 642 |
| 695995 | 1778 | 1793 | 44167 | 44182 | TCTCTATGAAAGCTCA | 50 | 655 |
| 695996 | 1780 | 1795 | 44169 | 44184 | ACTCTCTATGAAAGCT | 29 | 656 |
| 695998 | 1790 | 1805 | 44179 | 44194 | ATGCTGTGAAACTCTC | 58 | 658 |
| 695999 | 1792 | 1807 | 44181 | 44196 | CCATGCTGTGAAACTC | 26 | 659 |
| 696011 | 1875 | 1890 | 44264 | 44279 | GTTGAGCTATCCAAAC | 2 | 671 |
| 696012 | 1878 | 1893 | 44267 | 44282 | CTTGTTGAGCTATCCA | 63 | 672 |
| 696013 | 1882 | 1897 | 44271 | 44286 | GTATCTTGTTGAGCTA | 56 | 673 |
| 696014 | 1884 | 1899 | 44273 | 44288 | TTGTATCTTGTTGAGC | 50 | 674 |
| 696016 | 1914 | 1929 | 44303 | 44318 | ATTTGTCAGCAGGACC | 35 | 676 |
| 696017 | 1917 | 1932 | 44306 | 44321 | TTGATTTGTCAGCAGG | 65 | 677 |
| 696018 | 1920 | 1935 | 44309 | 44324 | CTCTTGATTTGTCAGC | 53 | 678 |
| 696025 | 2025 | 2040 | 44414 | 44429 | AATTAATGTCTTGGCA | 12 | 685 |
| 696026 | 2026 | 2041 | 44415 | 44430 | AAATTAATGTCTTGGC | 47 | 686 |
| 696816 | 1519 | 1534 | 43908 | 43923 | CATGTAAATATAGCCC | 5 | 805 |
| 716613 | 1489 | 1504 | 43878 | 43893 | TAATAATCCCCATTTC | 9 | 806 |
| 716614 | 1490 | 1505 | 43879 | 43894 | ATAATAATCCCCATTT | 4 | 807 |
| 716615 | 1491 | 1506 | 43880 | 43895 | TATAATAATCCCCATT | 7 | 808 |
| 716616 | 1517 | 1532 | 43906 | 43921 | TGTAAATATAGCCCCA | 34 | 809 |
| 716617 | 1777 | 1792 | 44166 | 44181 | CTCTATGAAAGCTCAA | 39 | 810 |
| 716618 | 1779 | 1794 | 44168 | 44183 | CTCTCTATGAAAGCTC | 29 | 811 |
| 716619 | 1786 | 1801 | 44175 | 44190 | TGTGAAACTCTCTATG | 4 | 812 |
| 716620 | 1787 | 1802 | 44176 | 44191 | CTGTGAAACTCTCTAT | 29 | 813 |
| 716621 | 1788 | 1803 | 44177 | 44192 | GCTGTGAAACTCTCTA | 42 | 814 |
| 716622 | 1791 | 1806 | 44180 | 44195 | CATGCTGTGAAACTCT | 8 | 815 |
| 716623 | 1876 | 1891 | 44265 | 44280 | TGTTGAGCTATCCAAA | 37 | 816 |
| 716624 | 1877 | 1892 | 44266 | 44281 | TTGTTGAGCTATCCAA | 3 | 817 |
| 716625 | 1879 | 1894 | 44268 | 44283 | TCTTGTTGAGCTATCC | 56 | 818 |
| 716626 | 1881 | 1896 | 44270 | 44285 | TATCTTGTTGAGCTAT | 33 | 819 |
| 716627 | 1883 | 1898 | 44272 | 44287 | TGTATCTTGTTGAGCT | 31 | 820 |
| 716628 | 1916 | 1931 | 44305 | 44320 | TGATTTGTCAGCAGGA | 59 | 821 |
| 716629 | 2027 | 2042 | 44416 | 44431 | AAAATTAATGTCTTGG | 12 | 822 |
| 716630 | 2028 | 2043 | 44417 | 44432 | AAAAATTAATGTCTTG | 20 | 823 |
| 716631 | 2029 | 2044 | 44418 | 44433 | AAAAAATTAATGTCTT | 9 | 824 |
| 716632 | 2030 | 2045 | 44419 | 44434 | AAAAAAATTAATGTCT | 7 | 825 |
| 716633 | 2031 | 2046 | 44420 | 44435 | AAAAAAAATTAATGTC | 14 | 826 |
| 716634 | 2032 | 2047 | 44421 | 44436 | AAAAAAAAATTAATGT | 16 | 827 |
| 716635 | 2033 | 2048 | 44422 | 44437 | AAAAAAAAAATTAATG | 6 | 828 |
| 716636 | 2034 | 2049 | 44423 | 44438 | TAAAAAAAAAATTAAT | 1 | 829 |
| 716637 | 2035 | 2050 | 44424 | 44439 | TTAAAAAAAAAATTAA | 0 | 830 |
| 716638 | 2036 | 2051 | 44425 | 44440 | TTTAAAAAAAAAATTA | 3 | 831 |
| 716639 | 2037 | 2052 | 44426 | 44441 | GTTTAAAAAAAAAATT | 2 | 832 |
| 716640 | 2038 | 2053 | 44427 | 44442 | TGTTTAAAAAAAAAAT | 2 | 833 |
| 716641 | 2039 | 2054 | 44428 | 44443 | TTGTTTAAAAAAAAAA | 0 | 834 |
| 716642 | 2040 | 2055 | 44429 | 44444 | ATTGTTTAAAAAAAAA | 0 | 835 |
| 716643 | 2041 | 2056 | 44430 | 44445 | CATTGTTTAAAAAAAA | 0 | 836 |
| 716644 | 2042 | 2057 | 44431 | 44446 | TCATTGTTTAAAAAAA | 0 | 837 |
| 716645 | 2043 | 2058 | 44432 | 44447 | TTCATTGTTTAAAAAA | 6 | 838 |
| 716646 | 2044 | 2059 | 44433 | 44448 | CTTCATTGTTTAAAAA | 11 | 839 |
| 716647 | 2045 | 2060 | 44434 | 44449 | ACTTCATTGTTTAAAA | 0 | 840 |
| 716648 | 2046 | 2061 | 44435 | 44450 | CACTTCATTGTTTAAA | 12 | 841 |

### Example 6: Antisense Inhibition of Human K-Ras in A431 Cells

Antisense oligonucleotides were designed targeting a K-Ras nucleic acid and were tested for their effects on K-Ras mRNA in vitro. The antisense oligonucleotides were tested in a series of experiments that had similar culture conditions. The results for each experiment are presented in separate tables shown below. Cultured A431cells at a density of 5,000 cells per well were treated with 2,000 nM antisense oligonucleotide by free uptake. After a treatment period of approximately 24 hours, RNA was isolated from the cells and K-Ras mRNA levels were measured by quantitative real-time PCR. Human primer probe set RTS3496_MGB was used to measure mRNA levels. K-Ras mRNA levels were adjusted according to total RNA content, as measured by RIBOGREEN®. Results are presented as percent inhibition of K-Ras, relative to untreated control cells. As used herein, a value of ‘0’ indicates that treatment with the antisense oligonucleotide did not inhibit mRNA levels.

The newly designed chimeric antisense oligonucleotides in the Tables below were designed as 3-10-3 cEt gapmers or deoxy, MOE, and (S)-cEt gapmers. The 3-10-3 cEt gapmers are 16 nucleosides in length, wherein the central gap segment comprises of ten 2′-deoxynucleosides and is flanked by wing segments on the 5′ direction and the 3′ direction comprising three nucleosides each. The deoxy, MOE and (S)-cEt oligonucleotides are 16 nucleosides in length wherein the nucleoside have either a MOE sugar modification, an (S)-cEt sugar modification, or a deoxy modification. The ‘Chemistry’ column describes the sugar modifications of each oligonucleotide. ‘k’ indicates an (S)-cEt sugar modification; ‘d’ indicates deoxyribose; the number after ‘d’ indicates the number of deoxynucleosides; and ‘e’ indicates a MOE modification. The internucleoside linkages throughout each gapmer are phosphorothioate (P═S) linkages. All cytosine residues throughout each gapmer are 5-methylcytosines. “Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either SEQ ID NO: 1 or SEQ ID NO: 2. ‘N/A’ indicates that the antisense oligonucleotide does not target that particular gene sequence with 100% complementarity.

TABLE 14
Inhibition of K-Ras mRNA by gapmers
targeting SEQ ID NO: 1 and 2

| | SEQ ID | SEQ ID | SEQ ID | SEQ ID | | | | |
|---|---|---|---|---|---|---|---|---|
| | NO: 1 | NO: 1 | NO: 2 | NO: 2 | | | | SEQ |
| ISIS | Start | Stop | Start | Stop | | | % | ID |
| NO | Site | Site | Site | Site | Sequence | Chemistry | Inhibition | NO |
| 540806 | 2981 | 2996 | 45370 | 45385 | GCATGAAGATTTCTGG | kkk-d10-kkk | 62 | 122 |
| 651600 | 2095 | 2110 | 44484 | 44499 | TAACCAGTGTTAAGAG | kkk-d10-kkk | 40 | 691 |
| 651603 | 2120 | 2135 | 44509 | 44524 | GAAAAGTGTTTATGCA | kkk-d10-kkk | 60 | 692 |
| 651633 | 2620 | 2635 | 45009 | 45024 | GGGAATTACAAGTATT | kkk-d10-kkk | 28 | 842 |
| 651634 | 2634 | 2649 | 45023 | 45038 | AATCTTATGGTTAGGG | kkk-d10-kkk | 45 | 316 |
| 651770 | 4332 | 4347 | 46721 | 46736 | TGTGCAATGGTGACAA | kkk-d10-kkk | 28 | 843 |
| 652019 | 1918 | 1933 | 44307 | 44322 | CTTGATTTGTCAGCAG | kkk-d10-kkk | 51 | 304 |
| 652028 | 2093 | 2108 | 44482 | 44497 | ACCAGTGTTAAGAGAA | kkk-d10-kkk | 60 | 334 |
| 652029 | 2118 | 2133 | 44507 | 44522 | AAAGTGTTTATGCAAT | kkk-d10-kkk | 29 | 335 |
| 695867 | 1130 | 1145 | 43519 | 43534 | TCCATTTATGTGACTA | kkk-d10-kkk | 70 | 506 |
| 696039 | 2092 | 2107 | 44481 | 44496 | CCAGTGTTAAGAGAAC | kkk-d10-kkk | 33 | 710 |
| 696042 | 2107 | 2122 | 44496 | 44511 | GCAATGTTAATTTAAC | kkk-d10-kkk | 17 | 713 |
| 696043 | 2113 | 2128 | 44502 | 44517 | GTTTATGCAATGTTAA | kkk-d10-kkk | 55 | 714 |
| 696044 | 2115 | 2130 | 44504 | 44519 | GTGTTTATGCAATGTT | kkk-d10-kkk | 79 | 715 |
| 696045 | 2127 | 2142 | 44516 | 44531 | CAGACTTGAAAAGTGT | kkk-d10-kkk | 24 | 716 |
| 696121 | 2635 | 2650 | 45024 | 45039 | AAATCTTATGGTTAGG | kkk-d10-kkk | 34 | 844 |
| 696357 | 4331 | 4346 | 46720 | 46735 | GTGCAATGGTGACAAC | kkk-d10-kkk | 38 | 845 |
| 696358 | 4334 | 4349 | 46723 | 46738 | ATTGTGCAATGGTGAC | kkk-d10-kkk | 57 | 846 |
| 696359 | 4336 | 4351 | 46725 | 46740 | AAATTGTGCAATGGTG | kkk-d10-kkk | 48 | 847 |
| 716649 | 2094 | 2109 | 44483 | 44498 | AACCAGTGTTAAGAGA | kkk-d10-kkk | 35 | 848 |
| 716650 | 2108 | 2123 | 44497 | 44512 | TGCAATGTTAATTTAA | kkk-d10-kkk | 13 | 849 |
| 716651 | 2109 | 2124 | 44498 | 44513 | ATGCAATGTTAATTTA | kkk-d10-kkk | 9 | 850 |
| 716652 | 2110 | 2125 | 44499 | 44514 | TATGCAATGTTAATTT | kkk-d10-kkk | 8 | 851 |
| 716653 | 2111 | 2126 | 44500 | 44515 | TTATGCAATGTTAATT | kkk-d10-kkk | 3 | 852 |
| 716654 | 2112 | 2127 | 44501 | 44516 | TTTATGCAATGTTAAT | kkk-d10-kkk | 34 | 853 |
| 716655 | 2114 | 2129 | 44503 | 44518 | TGTTTATGCAATGTTA | kkk-d10-kkk | 58 | 854 |
| 716656 | 2116 | 2131 | 44505 | 44520 | AGTGTTTATGCAATGT | kkk-d10-kkk | 69 | 855 |
| 716657 | 2117 | 2132 | 44506 | 44521 | AAGTGTTTATGCAATG | kkk-d10-kkk | 45 | 856 |
| 716658 | 2119 | 2134 | 44508 | 44523 | AAAAGTGTTTATGCAA | kkk-d10-kkk | 32 | 857 |
| 716659 | 2121 | 2136 | 44510 | 44525 | TGAAAAGTGTTTATGC | kkk-d10-kkk | 40 | 858 |
| 716660 | 2122 | 2137 | 44511 | 44526 | TTGAAAAGTGTTTATG | kkk-d10-kkk | 26 | 859 |
| 716661 | 2123 | 2138 | 44512 | 44527 | CTTGAAAAGTGTTTAT | kkk-d10-kkk | 0 | 860 |
| 716662 | 2124 | 2139 | 44513 | 44528 | ACTTGAAAAGTGTTTA | kkk-d10-kkk | 16 | 861 |
| 716663 | 2125 | 2140 | 44514 | 44529 | GACTTGAAAAGTGTTT | kkk-d10-kkk | 14 | 862 |
| 716664 | 2126 | 2141 | 44515 | 44530 | AGACTTGAAAAGTGTT | kkk-d10-kkk | 28 | 863 |
| 716665 | 2624 | 2639 | 45013 | 45028 | TTAGGGGAATTACAAG | kkk-d10-kkk | 23 | 864 |
| 716666 | 2626 | 2641 | 45015 | 45030 | GGTTAGGGGAATTACA | kkk-d10-kkk | 26 | 865 |
| 716667 | 2628 | 2643 | 45017 | 45032 | ATGGTTAGGGGAATTA | kkk-d10-kkk | 33 | 866 |
| 716668 | 2630 | 2645 | 45019 | 45034 | TTATGGTTAGGGGAAT | kkk-d10-kkk | 21 | 867 |
| 716669 | 2632 | 2647 | 45021 | 45036 | TCTTATGGTTAGGGGA | kkk-d10-kkk | 8 | 868 |
| 716670 | 2633 | 2648 | 45022 | 45037 | ATCTTATGGTTAGGGG | kkk-d10-kkk | 20 | 869 |
| 716671 | 4333 | 4348 | 46722 | 46737 | TTGTGCAATGGTGACA | kkk-d10-kkk | 29 | 870 |
| 716672 | 4335 | 4350 | 46724 | 46739 | AATTGTGCAATGGTGA | kkk-d10-kkk | 46 | 871 |
| 716719 | 1918 | 1933 | 44307 | 44322 | CTTGATTTGTCAGCAG | kkk-d9-kkke | 46 | 872 |
| 716720 | 2093 | 2108 | 44482 | 44497 | ACCAGTGTTAAGAGAA | kkk-d9-kkke | 44 | 873 |
| 716724 | 1918 | 1933 | 44307 | 44322 | CTTGATTTGTCAGCAG | kkk-d8-kekek | 41 | 874 |
| 716725 | 2093 | 2108 | 44482 | 44497 | ACCAGTGTTAAGAGAA | kkk-d8-kekek | 51 | 875 |
| 716729 | 1918 | 1933 | 44307 | 44322 | CTTGATTTGTCAGCAG | kkk-d9-keke | 46 | 876 |
| 716730 | 2093 | 2108 | 44482 | 44497 | ACCAGTGTTAAGAGAA | kkk-d9-keke | 40 | 877 |
| 716734 | 1918 | 1933 | 44307 | 44322 | CTTGATTTGTCAGCAG | kk-d10-keke | 54 | 878 |
| 716735 | 2093 | 2108 | 44482 | 44497 | ACCAGTGTTAAGAGAA | kk-d10-keke | 42 | 879 |
| 716739 | 1918 | 1933 | 44307 | 44322 | CTTGATTTGTCAGCAG | kk-d9-kekek | 49 | 880 |
| 716740 | 2093 | 2108 | 44482 | 44497 | ACCAGTGTTAAGAGAA | kk-d9-kekek | 49 | 881 |
| 716744 | 1918 | 1933 | 44307 | 44322 | CTTGATTTGTCAGCAG | k-d10-kekek | 45 | 882 |
| 716745 | 2093 | 2108 | 44482 | 44497 | ACCAGTGTTAAGAGAA | k-d10-kekek | 44 | 883 |
| 716749 | 1918 | 1933 | 44307 | 44322 | CTTGATTTGTCAGCAG | k-d9-kekeke | 44 | 884 |
| 716750 | 2093 | 2108 | 44482 | 44497 | ACCAGTGTTAAGAGAA | k-d9-kekeke | 50 | 885 |
| 716754 | 1918 | 1933 | 44307 | 44322 | CTTGATTTGTCAGCAG | kk-d8-kekekk | 33 | 886 |
| 716755 | 2093 | 2108 | 44482 | 44497 | ACCAGTGTTAAGAGAA | kk-d8-kekekk | 52 | 887 |
| 716759 | 1918 | 1933 | 44307 | 44322 | CTTGATTTGTCAGCAG | kkk-d8-kdkdk | 33 | 888 |
| 716760 | 2093 | 2108 | 44482 | 44497 | ACCAGTGTTAAGAGAA | kkk-d8-kdkdk | 50 | 889 |
| 716764 | 1917 | 1932 | 44306 | 44321 | TTGATTTGTCAGCAGG | kkk-d9-kkke | 55 | 890 |
| 716765 | 2092 | 2107 | 44481 | 44496 | CCAGTGTTAAGAGAAC | kkk-d9-kkke | 55 | 891 |
| 716769 | 1917 | 1932 | 44306 | 44321 | TTGATTTGTCAGCAGG | kk-d10-keke | 66 | 892 |
| 716770 | 2092 | 2107 | 44481 | 44496 | CCAGTGTTAAGAGAAC | kk-d10-keke | 42 | 893 |
| 716774 | 1917 | 1932 | 44306 | 44321 | TTGATTTGTCAGCAGG | kk-d9-kekek | 23 | 894 |
| 716775 | 2092 | 2107 | 44481 | 44496 | CCAGTGTTAAGAGAAC | kk-d9-kekek | 43 | 895 |
| 716779 | 1917 | 1932 | 44306 | 44321 | TTGATTTGTCAGCAGG | kk-d8-kekekk | 29 | 896 |
| 716780 | 2092 | 2107 | 44481 | 44496 | CCAGTGTTAAGAGAAC | kk-d8-kekekk | 53 | 897 |
| 716784 | 1917 | 1932 | 44306 | 44321 | TTGATTTGTCAGCAGG | kk-d9-kdkdk | 53 | 898 |
| 716785 | 2092 | 2107 | 44481 | 44496 | CCAGTGTTAAGAGAAC | kk-d9-kdkdk | 38 | 899 |
| 716789 | 1917 | 1932 | 44306 | 44321 | TTGATTTGTCAGCAGG | kkk-d8-kekek | 37 | 900 |
| 716790 | 2092 | 2107 | 44481 | 44496 | CCAGTGTTAAGAGAAC | kkk-d8-kekek | 37 | 901 |
| 716794 | 1916 | 1931 | 44305 | 44320 | TGATTTGTCAGCAGGA | k-d10-kekek | 47 | 902 |
| 716795 | 2091 | 2106 | 44480 | 44495 | CAGTGTTAAGAGAACT | k-d10-kekek | 23 | 903 |
| 716799 | 1916 | 1931 | 44305 | 44320 | TGATTTGTCAGCAGGA | k-d9-kekeke | 32 | 904 |
| 716800 | 2091 | 2106 | 44480 | 44495 | CAGTGTTAAGAGAACT | k-d9-kekeke | 40 | 905 |
| 716804 | 1916 | 1931 | 44305 | 44320 | TGATTTGTCAGCAGGA | kk-d8-kekekk | 32 | 906 |
| 716805 | 2091 | 2106 | 44480 | 44495 | CAGTGTTAAGAGAACT | kk-d8-kekekk | 35 | 907 |

TABLE 15
Inhibition of K-Ras mRNA by gapmers
targeting SEQ ID NO: 1 and 2

| | SEQ ID | SEQ ID | SEQ ID | SEQ ID | | | | |
|---|---|---|---|---|---|---|---|---|
| | NO: 1 | NO: 1 | NO: 2 | NO: 2 | | | | SEQ |
| ISIS | Start | Stop | Start | Stop | | | % | ID |
| NO | Site | Site | Site | Site | Sequence | Chemistry | Inhibition | NO |
| 540787 | 2460 | 2475 | 44849 | 44864 | GTGTAACATAGGTTAA | kkk-d10-kkk | 30 | 39 |
| 540797 | 2694 | 2709 | 45083 | 45098 | TAGGGCATTTCTGATG | kkk-d10-kkk | 20 | 44 |
| 540802 | 2848 | 2863 | 45237 | 45252 | CTATTCATACCAGGTT | kkk-d10-kkk | 33 | 120 |
| 540806 | 2981 | 2996 | 45370 | 45385 | GCATGAAGATTTCTGG | kkk-d10-kkk | 60 | 122 |
| 651609 | 2247 | 2262 | 44636 | 44651 | TCCAGTGATACTTTCA | kkk-d10-kkk | 51 | 908 |
| 651623 | 2466 | 2481 | 44855 | 44870 | AAGATGGTGTAACATA | kkk-d10-kkk | 30 | 312 |
| 651626 | 2527 | 2542 | 44916 | 44931 | GAGAATGGATATTCAA | kkk-d10-kkk | 8 | 909 |
| 651635 | 2654 | 2669 | 45043 | 45058 | AGATATCCACAGCAGC | kkk-d10-kkk | 58 | 910 |
| 651642 | 2686 | 2701 | 45075 | 45090 | TTCTGATGTGACTCAG | kkk-d10-kkk | 26 | 320 |
| 651653 | 2762 | 2777 | 45151 | 45166 | ATTAGTGATTAGGTCA | kkk-d10-kkk | 55 | 911 |
| 651666 | 2855 | 2870 | 45244 | 45259 | GTTCTGTCTATTCATA | kkk-d10-kkk | 31 | 912 |
| 652034 | 2248 | 2263 | 44637 | 44652 | GTCCAGTGATACTTTC | kkk-d10-kkk | 44 | 340 |
| 652050 | 2434 | 2449 | 44823 | 44838 | ATTGTGCTGAGCTTGA | kkk-d10-kkk | 51 | 356 |
| 652055 | 2531 | 2546 | 44920 | 44935 | AAACGAGAATGGATAT | kkk-d10-kkk | 12 | 361 |
| 652061 | 2652 | 2667 | 45041 | 45056 | ATATCCACAGCAGCAG | kkk-d10-kkk | 39 | 367 |
| 652067 | 2764 | 2779 | 45153 | 45168 | AAATTAGTGATTAGGT | kkk-d10-kkk | 5 | 373 |
| 663560 | 2437 | 2452 | 44826 | 44841 | CAGATTGTGCTGAGCT | kkk-d10-kkk | 29 | 913 |
| 695867 | 1130 | 1145 | 43519 | 43534 | TCCATTTATGTGACTA | kkk-d10-kkk | 57 | 506 |
| 695912 | 1312 | 1327 | 43701 | 43716 | GACTAATAGCAGTGGA | kkk-d10-kkk | 52 | 408 |
| 696044 | 2115 | 2130 | 44504 | 44519 | GTGTTTATGCAATGTT | kkk-d10-kkk | 72 | 715 |
| 696054 | 2246 | 2261 | 44635 | 44650 | CCAGTGATACTTTCAC | kkk-d10-kkk | 15 | 725 |
| 696055 | 2249 | 2264 | 44638 | 44653 | AGTCCAGTGATACTTT | kkk-d10-kkk | 8 | 726 |
| 696090 | 2435 | 2450 | 44824 | 44839 | GATTGTGCTGAGCTTG | kkk-d10-kkk | 63 | 761 |
| 696091 | 2436 | 2451 | 44825 | 44840 | AGATTGTGCTGAGCTT | kkk-d10-kkk | 39 | 762 |
| 696092 | 2438 | 2453 | 44827 | 44842 | ACAGATTGTGCTGAGC | kkk-d10-kkk | 46 | 763 |
| 696096 | 2463 | 2478 | 44852 | 44867 | ATGGTGTAACATAGGT | kkk-d10-kkk | 59 | 914 |
| 696108 | 2529 | 2544 | 44918 | 44933 | ACGAGAATGGATATTC | kkk-d10-kkk | 29 | 915 |
| 696116 | 2563 | 2578 | 44952 | 44967 | CAAGATGACACTAATA | kkk-d10-kkk | 18 | 916 |
| 696117 | 2565 | 2580 | 44954 | 44969 | GGCAAGATGACACTAA | kkk-d10-kkk | 30 | 917 |
| 696118 | 2567 | 2582 | 44956 | 44971 | GAGGCAAGATGACACT | kkk-d10-kkk | 0 | 918 |
| 696132 | 2656 | 2671 | 45045 | 45060 | GGAGATATCCACAGCA | kkk-d10-kkk | 0 | 919 |
| 696137 | 2688 | 2703 | 45077 | 45092 | ATTTCTGATGTGACTC | kkk-d10-kkk | 49 | 920 |
| 696138 | 2692 | 2707 | 45081 | 45096 | GGGCATTTCTGATGTG | kkk-d10-kkk | 7 | 921 |
| 696139 | 2697 | 2712 | 45086 | 45101 | ATGTAGGGCATTTCTG | kkk-d10-kkk | 9 | 922 |
| 696151 | 2759 | 2774 | 45148 | 45163 | AGTGATTAGGTCAAAT | kkk-d10-kkk | 23 | 923 |
| 696152 | 2761 | 2776 | 45150 | 45165 | TTAGTGATTAGGTCAA | kkk-d10-kkk | 59 | 924 |
| 696167 | 2850 | 2865 | 45239 | 45254 | GTCTATTCATACCAGG | kkk-d10-kkk | 60 | 925 |
| 716673 | 2461 | 2476 | 44850 | 44865 | GGTGTAACATAGGTTA | kkk-d10-kkk | 55 | 926 |
| 716674 | 2462 | 2477 | 44851 | 44866 | TGGTGTAACATAGGTT | kkk-d10-kkk | 58 | 927 |
| 716675 | 2464 | 2479 | 44853 | 44868 | GATGGTGTAACATAGG | kkk-d10-kkk | 60 | 928 |
| 716676 | 2465 | 2480 | 44854 | 44869 | AGATGGTGTAACATAG | kkk-d10-kkk | 39 | 929 |
| 716677 | 2528 | 2543 | 44917 | 44932 | CGAGAATGGATATTCA | kkk-d10-kkk | 14 | 930 |
| 716678 | 2530 | 2545 | 44919 | 44934 | AACGAGAATGGATATT | kkk-d10-kkk | 4 | 931 |
| 716679 | 2564 | 2579 | 44953 | 44968 | GCAAGATGACACTAAT | kkk-d10-kkk | 30 | 932 |
| 716680 | 2566 | 2581 | 44955 | 44970 | AGGCAAGATGACACTA | kkk-d10-kkk | 21 | 933 |
| 716681 | 2653 | 2668 | 45042 | 45057 | GATATCCACAGCAGCA | kkk-d10-kkk | 10 | 934 |
| 716682 | 2655 | 2670 | 45044 | 45059 | GAGATATCCACAGCAG | kkk-d10-kkk | 49 | 935 |
| 716683 | 2687 | 2702 | 45076 | 45091 | TTTCTGATGTGACTCA | kkk-d10-kkk | 57 | 936 |
| 716684 | 2689 | 2704 | 45078 | 45093 | CATTTCTGATGTGACT | kkk-d10-kkk | 32 | 937 |
| 716685 | 2690 | 2705 | 45079 | 45094 | GCATTTCTGATGTGAC | kkk-d10-kkk | 24 | 938 |
| 716686 | 2691 | 2706 | 45080 | 45095 | GGCATTTCTGATGTGA | kkk-d10-kkk | 8 | 939 |
| 716687 | 2695 | 2710 | 45084 | 45099 | GTAGGGCATTTCTGAT | kkk-d10-kkk | 14 | 940 |
| 716688 | 2696 | 2711 | 45085 | 45100 | TGTAGGGCATTTCTGA | kkk-d10-kkk | 0 | 941 |
| 716689 | 2698 | 2713 | 45087 | 45102 | GATGTAGGGCATTTCT | kkk-d10-kkk | 0 | 942 |
| 716690 | 2760 | 2775 | 45149 | 45164 | TAGTGATTAGGTCAAA | kkk-d10-kkk | 21 | 943 |
| 716691 | 2763 | 2778 | 45152 | 45167 | AATTAGTGATTAGGTC | kkk-d10-kkk | 32 | 944 |
| 716692 | 2849 | 2864 | 45238 | 45253 | TCTATTCATACCAGGT | kkk-d10-kkk | 26 | 945 |
| 716693 | 2851 | 2866 | 45240 | 45255 | TGTCTATTCATACCAG | kkk-d10-kkk | 16 | 946 |
| 716694 | 2852 | 2867 | 45241 | 45256 | CTGTCTATTCATACCA | kkk-d10-kkk | 34 | 947 |
| 716695 | 2853 | 2868 | 45242 | 45257 | TCTGTCTATTCATACC | kkk-d10-kkk | 12 | 948 |
| 716696 | 2854 | 2869 | 45243 | 45258 | TTCTGTCTATTCATAC | kkk-d10-kkk | 10 | 949 |
| 716721 | 2248 | 2263 | 44637 | 44652 | GTCCAGTGATACTTTC | kkk-d9-kkke | 48 | 950 |
| 716726 | 2248 | 2263 | 44637 | 44652 | GTCCAGTGATACTTTC | kkk-d8-kekek | 21 | 951 |
| 716731 | 2248 | 2263 | 44637 | 44652 | GTCCAGTGATACTTTC | kkk-d9-keke | 33 | 952 |
| 716736 | 2248 | 2263 | 44637 | 44652 | GTCCAGTGATACTTTC | kk-d10-keke | 39 | 953 |
| 716741 | 2248 | 2263 | 44637 | 44652 | GTCCAGTGATACTTTC | kk-d9-kekek | 43 | 954 |
| 716746 | 2248 | 2263 | 44637 | 44652 | GTCCAGTGATACTTTC | k-d10-kekek | 36 | 955 |
| 716751 | 2248 | 2263 | 44637 | 44652 | GTCCAGTGATACTTTC | k-d9-kekeke | 20 | 956 |
| 716756 | 2248 | 2263 | 44637 | 44652 | GTCCAGTGATACTTTC | kk-d8-kekekk | 22 | 957 |
| 716761 | 2248 | 2263 | 44637 | 44652 | GTCCAGTGATACTTTC | kkk-d8-kdkdk | 30 | 958 |
| 716766 | 2247 | 2262 | 44636 | 44651 | TCCAGTGATACTTTCA | kkk-d9-kkke | 47 | 959 |
| 716771 | 2247 | 2262 | 44636 | 44651 | TCCAGTGATACTTTCA | kk-d10-keke | 39 | 960 |
| 716776 | 2247 | 2262 | 44636 | 44651 | TCCAGTGATACTTTCA | kk-d9-kekek | 37 | 961 |
| 716781 | 2247 | 2262 | 44636 | 44651 | TCCAGTGATACTTTCA | kk-d8-kekekk | 24 | 962 |
| 716786 | 2247 | 2262 | 44636 | 44651 | TCCAGTGATACTTTCA | kk-d9-kdkdk | 35 | 963 |
| 716791 | 2247 | 2262 | 44636 | 44651 | TCCAGTGATACTTTCA | kkk-d8-kekek | 48 | 964 |
| 716796 | 2246 | 2261 | 44635 | 44650 | CCAGTGATACTTTCAC | k-d10-kekek | 0 | 965 |
| 716801 | 2246 | 2261 | 44635 | 44650 | CCAGTGATACTTTCAC | k-d9-kekeke | 16 | 966 |
| 716806 | 2246 | 2261 | 44635 | 44650 | CCAGTGATACTTTCAC | kk-d8-kekekk | 17 | 967 |

TABLE 16
Inhibition of K-Ras mRNA by gapmers
targeting SEQ ID NO: 1 and 2

| | SEQ ID | SEQ ID | SEQ ID | SEQ ID | | | | |
|---|---|---|---|---|---|---|---|---|
| | NO: 1 | NO: 1 | NO: 2 | NO: 2 | | | | SEQ |
| ISIS | Start | Stop | Start | Stop | | | % | ID |
| NO | Site | Site | Site | Site | Sequence | Chemistry | Inhibition | NO |
| 540806 | 2981 | 2996 | 45370 | 45385 | GCATGAAGATTTCTGG | kkk-d10-kkk | 66 | 122 |
| 540831 | 4276 | 4291 | 46665 | 46680 | TGCAGTGTGACTCAGT | kkk-d10-kkk | 55 | 61 |
| 651720 | 3715 | 3730 | 46104 | 46119 | CTTATGCAGAGAAAAC | kkk-d10-kkk | 8 | 968 |
| 651721 | 3721 | 3736 | 46110 | 46125 | TAATTACTTATGCAGA | kkk-d10-kkk | 17 | 969 |
| 651759 | 4273 | 4288 | 46662 | 46677 | AGTGTGACTCAGTTAA | kkk-d10-kkk | 53 | 970 |
| 651760 | 4274 | 4289 | 46663 | 46678 | CAGTGTGACTCAGTTA | kkk-d10-kkk | 53 | 971 |
| 651761 | 4275 | 4290 | 46664 | 46679 | GCAGTGTGACTCAGTT | kkk-d10-kkk | 48 | 972 |
| 651795 | 4620 | 4635 | 47009 | 47024 | CCAGTATTAACACAGA | kkk-d10-kkk | 37 | 973 |
| 652112 | 3722 | 3737 | 46111 | 46126 | TTAATTACTTATGCAG | kkk-d10-kkk | 32 | 974 |
| 652132 | 4036 | 4051 | 46425 | 46440 | ACCATTCAAAGTTCAC | kkk-d10-kkk | 34 | 975 |
| 652157 | 4524 | 4539 | 46913 | 46928 | CTTTTTGACAAATGGA | kkk-d10-kkk | 22 | 976 |
| 695867 | 1130 | 1145 | 43519 | 43534 | TCCATTTATGTGACTA | kkk-d10-kkk | 63 | 506 |
| 696271 | 3719 | 3734 | 46108 | 46123 | ATTACTTATGCAGAGA | kkk-d10-kkk | 60 | 977 |
| 696316 | 4029 | 4044 | 46418 | 46433 | AAAGTTCACATAAAGG | kkk-d10-kkk | 38 | 978 |
| 696317 | 4035 | 4050 | 46424 | 46439 | CCATTCAAAGTTCACA | kkk-d10-kkk | 42 | 979 |
| 696318 | 4037 | 4052 | 46426 | 46441 | AACCATTCAAAGTTCA | kkk-d10-kkk | 35 | 980 |
| 696377 | 4525 | 4540 | 46914 | 46929 | ACTTTTTGACAAATGG | kkk-d10-kkk | 46 | 981 |
| 696378 | 4530 | 4545 | 46919 | 46934 | TCATTACTTTTTGACA | kkk-d10-kkk | 4 | 982 |
| 696385 | 4617 | 4632 | 47006 | 47021 | GTATTAACACAGAAGT | kkk-d10-kkk | 0 | 983 |
| 696386 | 4622 | 4637 | 47011 | 47026 | ATCCAGTATTAACACA | kkk-d10-kkk | 0 | 984 |
| 696556 | N/A | N/A | 10213 | 10228 | CTGAATTAGTCTCCAT | kkk-d10-kkk | 48 | 985 |
| 716697 | 3716 | 3731 | 46105 | 46120 | ACTTATGCAGAGAAAA | kkk-d10-kkk | 37 | 986 |
| 716698 | 3717 | 3732 | 46106 | 46121 | TACTTATGCAGAGAAA | kkk-d10-kkk | 0 | 987 |
| 716699 | 3718 | 3733 | 46107 | 46122 | TTACTTATGCAGAGAA | kkk-d10-kkk | 46 | 988 |
| 716700 | 3720 | 3735 | 46109 | 46124 | AATTACTTATGCAGAG | kkk-d10-kkk | 44 | 989 |
| 716701 | 4030 | 4045 | 46419 | 46434 | CAAAGTTCACATAAAG | kkk-d10-kkk | 21 | 990 |
| 716702 | 4031 | 4046 | 46420 | 46435 | TCAAAGTTCACATAAA | kkk-d10-kkk | 19 | 991 |
| 716703 | 4032 | 4047 | 46421 | 46436 | TTCAAAGTTCACATAA | kkk-d10-kkk | 7 | 992 |
| 716704 | 4033 | 4048 | 46422 | 46437 | ATTCAAAGTTCACATA | kkk-d10-kkk | 0 | 993 |
| 716705 | 4034 | 4049 | 46423 | 46438 | CATTCAAAGTTCACAT | kkk-d10-kkk | 2 | 994 |
| 716706 | 4526 | 4541 | 46915 | 46930 | TACTTTTTGACAAATG | kkk-d10-kkk | 37 | 995 |
| 716707 | 4527 | 4542 | 46916 | 46931 | TTACTTTTTGACAAAT | kkk-d10-kkk | 0 | 996 |
| 716708 | 4528 | 4543 | 46917 | 46932 | ATTACTTTTTGACAAA | kkk-d10-kkk | 0 | 997 |
| 716709 | 4529 | 4544 | 46918 | 46933 | CATTACTTTTTGACAA | kkk-d10-kkk | 8 | 998 |
| 716710 | 4618 | 4633 | 47007 | 47022 | AGTATTAACACAGAAG | kkk-d10-kkk | 27 | 999 |
| 716711 | 4619 | 4634 | 47008 | 47023 | CAGTATTAACACAGAA | kkk-d10-kkk | 7 | 1000 |
| 716712 | 4621 | 4636 | 47010 | 47025 | TCCAGTATTAACACAG | kkk-d10-kkk | 34 | 1001 |
| 716713 | N/A | N/A | 10204 | 10219 | TCTCCATTAGTAAATA | kkk-d10-kkk | 13 | 1002 |
| 716714 | N/A | N/A | 10209 | 10224 | ATTAGTCTCCATTAGT | kkk-d10-kkk | 29 | 1003 |
| 716715 | N/A | N/A | 10212 | 10227 | TGAATTAGTCTCCATT | kkk-d10-kkk | 17 | 1004 |
| 716716 | N/A | N/A | 10214 | 10229 | TCTGAATTAGTCTCCA | kkk-d10-kkk | 62 | 1005 |
| 716717 | N/A | N/A | 10217 | 10232 | AAATCTGAATTAGTCT | kkk-d10-kkk | 25 | 1006 |
| 716718 | N/A | N/A | 10222 | 10237 | CTTACAAATCTGAATT | kkk-d10-kkk | 7 | 1007 |
| 716722 | 4036 | 4051 | 46425 | 46440 | ACCATTCAAAGTTCAC | kkk-d9-kkke | 42 | 1008 |
| 716723 | 4274 | 4289 | 46663 | 46678 | CAGTGTGACTCAGTTA | kkk-d9-kkke | 42 | 1009 |
| 716727 | 4036 | 4051 | 46425 | 46440 | ACCATTCAAAGTTCAC | kkk-d8-kekek | 26 | 1010 |
| 716728 | 4274 | 4289 | 46663 | 46678 | CAGTGTGACTCAGTTA | kkk-d8-kekek | 63 | 1011 |
| 716732 | 4036 | 4051 | 46425 | 46440 | ACCATTCAAAGTTCAC | kkk-d9-keke | 30 | 1012 |
| 716733 | 4274 | 4289 | 46663 | 46678 | CAGTGTGACTCAGTTA | kkk-d9-keke | 55 | 1013 |
| 716737 | 4036 | 4051 | 46425 | 46440 | ACCATTCAAAGTTCAC | kk-d10-keke | 41 | 1014 |
| 716738 | 4274 | 4289 | 46663 | 46678 | CAGTGTGACTCAGTTA | kk-d10-keke | 51 | 1015 |
| 716742 | 4036 | 4051 | 46425 | 46440 | ACCATTCAAAGTTCAC | kk-d9-kekek | 36 | 1016 |
| 716743 | 4274 | 4289 | 46663 | 46678 | CAGTGTGACTCAGTTA | kk-d9-kekek | 56 | 1017 |
| 716747 | 4036 | 4051 | 46425 | 46440 | ACCATTCAAAGTTCAC | k-d10-kekek | 18 | 1018 |
| 716748 | 4274 | 4289 | 46663 | 46678 | CAGTGTGACTCAGTTA | k-d10-kekek | 32 | 1019 |
| 716752 | 4036 | 4051 | 46425 | 46440 | ACCATTCAAAGTTCAC | k-d9-kekeke | 15 | 1020 |
| 716753 | 4274 | 4289 | 46663 | 46678 | CAGTGTGACTCAGTTA | k-d9-kekeke | 54 | 1021 |
| 716757 | 4036 | 4051 | 46425 | 46440 | ACCATTCAAAGTTCAC | kk-d8-kekekk | 7 | 1022 |
| 716758 | 4274 | 4289 | 46663 | 46678 | CAGTGTGACTCAGTTA | kk-d8-kekekk | 63 | 1023 |
| 716762 | 4036 | 4051 | 46425 | 46440 | ACCATTCAAAGTTCAC | kkk-d8-kdkdk | 12 | 1024 |
| 716763 | 4274 | 4289 | 46663 | 46678 | CAGTGTGACTCAGTTA | kkk-d8-kdkdk | 60 | 1025 |
| 716767 | 4035 | 4050 | 46424 | 46439 | CCATTCAAAGTTCACA | kkk-d9-kkke | 41 | 1026 |
| 716768 | 4273 | 4288 | 46662 | 46677 | AGTGTGACTCAGTTAA | kkk-d9-kkke | 15 | 1027 |
| 716772 | 4035 | 4050 | 46424 | 46439 | CCATTCAAAGTTCACA | kk-d10-keke | 63 | 1028 |
| 716773 | 4273 | 4288 | 46662 | 46677 | AGTGTGACTCAGTTAA | kk-d10-keke | 41 | 1029 |
| 716777 | 4035 | 4050 | 46424 | 46439 | CCATTCAAAGTTCACA | kk-d9-kekek | 36 | 1030 |
| 716778 | 4273 | 4288 | 46662 | 46677 | AGTGTGACTCAGTTAA | kk-d9-kekek | 48 | 1031 |
| 716782 | 4035 | 4050 | 46424 | 46439 | CCATTCAAAGTTCACA | kk-d8-kekekk | 57 | 1032 |
| 716783 | 4273 | 4288 | 46662 | 46677 | AGTGTGACTCAGTTAA | kk-d8-kekekk | 47 | 1033 |
| 716787 | 4035 | 4050 | 46424 | 46439 | CCATTCAAAGTTCACA | kk-d9-kdkdk | 31 | 1034 |
| 716788 | 4273 | 4288 | 46662 | 46677 | AGTGTGACTCAGTTAA | kk-d9-kdkdk | 38 | 1035 |
| 716792 | 4035 | 4050 | 46424 | 46439 | CCATTCAAAGTTCACA | kkk-d8-kekek | 39 | 1036 |
| 716793 | 4273 | 4288 | 46662 | 46677 | AGTGTGACTCAGTTAA | kkk-d8-kekek | 29 | 1037 |
| 716797 | 4034 | 4049 | 46423 | 46438 | CATTCAAAGTTCACAT | k-d10-kekek | 43 | 1038 |
| 716798 | 4272 | 4287 | 46661 | 46676 | GTGTGACTCAGTTAAA | k-d10-kekek | 30 | 1039 |
| 716802 | 4034 | 4049 | 46423 | 46438 | CATTCAAAGTTCACAT | k-d9-kekeke | 24 | 1040 |
| 716803 | 4272 | 4287 | 46661 | 46676 | GTGTGACTCAGTTAAA | k-d9-kekeke | 25 | 1041 |
| 716807 | 4034 | 4049 | 46423 | 46438 | CATTCAAAGTTCACAT | kk-d8-kekekk | 96 | 1042 |
| 716808 | 4272 | 4287 | 46661 | 46676 | GTGTGACTCAGTTAAA | kk-d8-kekekk | 38 | 1043 |

### Example 7: Antisense Inhibition of Human K-Ras in A431 Cells by cEt Gapmers

Antisense oligonucleotides were designed targeting a K-Ras nucleic acid and were tested for their effects on K-Ras mRNA in vitro. The antisense oligonucleotides were tested in a series of experiments that had similar culture conditions. The results for each experiment are presented in separate tables shown below. Cultured A431cells at a density of 5,000 cells per well were treated with 1,000 nM antisense oligonucleotide by free uptake. After a treatment period of approximately 24 hours, RNA was isolated from the cells and K-Ras mRNA levels were measured by quantitative real-time PCR. Human primer probe set RTS3496_MGB was used to measure mRNA levels. K-Ras mRNA levels were adjusted according to total RNA content, as measured by RIBOGREEN®. Results are presented as percent inhibition of K-Ras, relative to untreated control cells. As used herein, a value of ‘0’ indicates that treatment with the antisense oligonucleotide did not inhibit mRNA levels.

The newly designed chimeric antisense oligonucleotides in the Tables below were designed as 3-10-3 cEt gapmers. The gapmers are 16 nucleosides in length, wherein the central gap segment comprises of ten 2′-deoxynucleosides and is flanked by wing segments on the 5′ direction and the 3′ direction comprising three nucleosides each. Each nucleoside in the 5′ wing segment and each nucleoside in the 3′ wing segment has a cEt sugar modification. The internucleoside linkages throughout each gapmer are phosphorothioate (P═S) linkages. All cytosine residues throughout each gapmer are 5-methylcytosines. “Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either SEQ ID NO: 1 or SEQ ID NO: 2. ‘N/A’ indicates that the antisense oligonucleotide does not target that particular gene sequence with 100% complementarity. In case the sequence alignment for a target gene in a particular table is not shown, it is understood that none of the oligonucleotides presented in that table align with 100% complementarity with that target gene.

TABLE 17
Inhibition of K-Ras mRNA by 3-10-3 cEt gapmers
targeting SEQ ID NO: 1 and 2

| | SEQ ID | SEQ ID | SEQ ID | SEQ ID | | | |
|---|---|---|---|---|---|---|---|
| | NO: 1 | NO: 1 | NO: 2 | NO: 2 | | | SEQ |
| ISIS | Start | Stop | Start | Stop | | % | ID |
| NO | Site | Site | Site | Site | Sequence | Inhibition | NO |
| 540806 | 2981 | 2996 | 45370 | 45385 | GCATGAAGATTTCTGG | 73 | 122 |
| 540806 | 2981 | 2996 | 45370 | 45385 | GCATGAAGATTTCTGG | 67 | 122 |
| 651626 | 2527 | 2542 | 44916 | 44931 | GAGAATGGATATTCAA | 26 | 909 |
| 651627 | 2533 | 2548 | 44922 | 44937 | TAAAACGAGAATGGAT | 11 | 1044 |
| 651633 | 2620 | 2635 | 45009 | 45024 | GGGAATTACAAGTATT | 38 | 842 |
| 651634 | 2634 | 2649 | 45023 | 45038 | AATCTTATGGTTAGGG | 63 | 316 |
| 651635 | 2654 | 2669 | 45043 | 45058 | AGATATCCACAGCAGC | 68 | 910 |
| 651636 | 2660 | 2675 | 45049 | 45064 | TCATGGAGATATCCAC | 35 | 1045 |
| 651644 | 2713 | 2728 | 45102 | 45117 | GCCCTGAGGAAATAAG | 11 | 1046 |
| 651651 | 2750 | 2765 | 45139 | 45154 | GTCAAATCCCTTTATG | 24 | 1047 |
| 651658 | 2813 | 2828 | 45202 | 45217 | TAATGGATTGGGCAGC | 41 | 1048 |
| 651663 | 2823 | 2838 | 45212 | 45227 | CTACTGTCGCTAATGG | 38 | 1049 |
| 696095 | 2458 | 2473 | 44847 | 44862 | GTAACATAGGTTAAAA | 18 | 1050 |
| 696096 | 2463 | 2478 | 44852 | 44867 | ATGGTGTAACATAGGT | 67 | 914 |
| 696097 | 2468 | 2483 | 44857 | 44872 | TGAAGATGGTGTAACA | 44 | 1051 |
| 696098 | 2470 | 2485 | 44859 | 44874 | ACTGAAGATGGTGTAA | 15 | 1052 |
| 696099 | 2474 | 2489 | 44863 | 44878 | TGGCACTGAAGATGGT | 46 | 1053 |
| 696100 | 2476 | 2491 | 44865 | 44880 | ACTGGCACTGAAGATG | 39 | 1054 |
| 696101 | 2480 | 2495 | 44869 | 44884 | CAAGACTGGCACTGAA | 32 | 1055 |
| 696102 | 2482 | 2497 | 44871 | 44886 | CCCAAGACTGGCACTG | 27 | 1056 |
| 696103 | 2488 | 2503 | 44877 | 44892 | ATTTTGCCCAAGACTG | 33 | 1057 |
| 696104 | 2492 | 2507 | 44881 | 44896 | CACAATTTTGCCCAAG | 36 | 1058 |
| 696105 | 2499 | 2514 | 44888 | 44903 | CCTCTTGCACAATTTT | 59 | 1059 |
| 696106 | 2504 | 2519 | 44893 | 44908 | CTTCACCTCTTGCACA | 28 | 1060 |
| 696107 | 2510 | 2525 | 44899 | 44914 | TATAAACTTCACCTCT | 17 | 1061 |
| 696108 | 2529 | 2544 | 44918 | 44933 | ACGAGAATGGATATTC | 58 | 915 |
| 696109 | 2535 | 2550 | 44924 | 44939 | CCTAAAACGAGAATGG | 10 | 1062 |
| 696110 | 2537 | 2552 | 44926 | 44941 | GTCCTAAAACGAGAAT | 22 | 1063 |
| 696111 | 2539 | 2554 | 44928 | 44943 | GAGTCCTAAAACGAGA | 39 | 1064 |
| 696112 | 2545 | 2560 | 44934 | 44949 | GAAGAAGAGTCCTAAA | 15 | 1065 |
| 696113 | 2549 | 2564 | 44938 | 44953 | TATGGAAGAAGAGTCC | 34 | 1066 |
| 696114 | 2553 | 2568 | 44942 | 44957 | CTAATATGGAAGAAGA | 10 | 1067 |
| 696115 | 2558 | 2573 | 44947 | 44962 | TGACACTAATATGGAA | 17 | 1068 |
| 696116 | 2563 | 2578 | 44952 | 44967 | CAAGATGACACTAATA | 18 | 916 |
| 696117 | 2565 | 2580 | 44954 | 44969 | GGCAAGATGACACTAA | 51 | 917 |
| 696118 | 2567 | 2582 | 44956 | 44971 | GAGGCAAGATGACACT | 18 | 918 |
| 696119 | 2585 | 2600 | 44974 | 44989 | GGGCATGTGGAAGGTA | 20 | 1069 |
| 696120 | 2609 | 2624 | 44998 | 45013 | GTATTAAAACTGCATC | 34 | 1070 |
| 696121 | 2635 | 2650 | 45024 | 45039 | AAATCTTATGGTTAGG | 40 | 844 |
| 696122 | 2636 | 2651 | 45025 | 45040 | TAAATCTTATGGTTAG | 13 | 1071 |
| 696123 | 2637 | 2652 | 45026 | 45041 | GTAAATCTTATGGTTA | 26 | 1072 |
| 696124 | 2638 | 2653 | 45027 | 45042 | AGTAAATCTTATGGTT | 28 | 1073 |
| 696125 | 2639 | 2654 | 45028 | 45043 | CAGTAAATCTTATGGT | 25 | 1074 |
| 696126 | 2640 | 2655 | 45029 | 45044 | GCAGTAAATCTTATGG | 50 | 1075 |
| 696127 | 2641 | 2656 | 45030 | 45045 | AGCAGTAAATCTTATG | 46 | 1076 |
| 696128 | 2642 | 2657 | 45031 | 45046 | CAGCAGTAAATCTTAT | 25 | 1077 |
| 696129 | 2645 | 2660 | 45034 | 45049 | CAGCAGCAGTAAATCT | 37 | 1078 |
| 696130 | 2647 | 2662 | 45036 | 45051 | CACAGCAGCAGTAAAT | 11 | 1079 |
| 696131 | 2649 | 2664 | 45038 | 45053 | TCCACAGCAGCAGTAA | 37 | 1080 |
| 696132 | 2656 | 2671 | 45045 | 45060 | GGAGATATCCACAGCA | 23 | 919 |
| 696133 | 2658 | 2673 | 45047 | 45062 | ATGGAGATATCCACAG | 17 | 1081 |
| 696134 | 2664 | 2679 | 45053 | 45068 | AACTTCATGGAGATAT | 38 | 1082 |
| 696135 | 2668 | 2683 | 45057 | 45072 | GGAAAACTTCATGGAG | 37 | 1083 |
| 696136 | 2671 | 2686 | 45060 | 45075 | GTGGGAAAACTTCATG | 12 | 1084 |
| 696137 | 2688 | 2703 | 45077 | 45092 | ATTTCTGATGTGACTC | 73 | 920 |
| 696138 | 2692 | 2707 | 45081 | 45096 | GGGCATTTCTGATGTG | 7 | 921 |
| 696139 | 2697 | 2712 | 45086 | 45101 | ATGTAGGGCATTTCTG | 24 | 922 |
| 696140 | 2701 | 2716 | 45090 | 45105 | TAAGATGTAGGGCATT | 30 | 1085 |
| 696141 | 2703 | 2718 | 45092 | 45107 | AATAAGATGTAGGGCA | 15 | 1086 |
| 696142 | 2705 | 2720 | 45094 | 45109 | GAAATAAGATGTAGGG | 26 | 1087 |
| 696143 | 2715 | 2730 | 45104 | 45119 | GAGCCCTGAGGAAATA | 19 | 1088 |
| 696144 | 2718 | 2733 | 45107 | 45122 | CTTGAGCCCTGAGGAA | 21 | 1089 |
| 696145 | 2727 | 2742 | 45116 | 45131 | TCAGATTCTCTTGAGC | 22 | 1090 |
| 696146 | 2728 | 2743 | 45117 | 45132 | GTCAGATTCTCTTGAG | 37 | 1091 |
| 696147 | 2729 | 2744 | 45118 | 45133 | TGTCAGATTCTCTTGA | 33 | 1092 |
| 696148 | 2732 | 2747 | 45121 | 45136 | ATCTGTCAGATTCTCT | 48 | 1093 |
| 696149 | 2745 | 2760 | 45134 | 45149 | ATCCCTTTATGGTATC | 14 | 1094 |
| 696150 | 2748 | 2763 | 45137 | 45152 | CAAATCCCTTTATGGT | 18 | 1095 |
| 696151 | 2759 | 2774 | 45148 | 45163 | AGTGATTAGGTCAAAT | 37 | 923 |
| 696152 | 2761 | 2776 | 45150 | 45165 | TTAGTGATTAGGTCAA | 74 | 924 |
| 696153 | 2766 | 2781 | 45155 | 45170 | GAAAATTAGTGATTAG | 34 | 1096 |
| 696154 | 2770 | 2785 | 45159 | 45174 | ACCTGAAAATTAGTGA | 24 | 1097 |
| 696155 | 2777 | 2792 | 45166 | 45181 | AGCCACCACCTGAAAA | 18 | 1098 |
| 696156 | 2787 | 2802 | 45176 | 45191 | TCAAAGCATCAGCCAC | 15 | 1099 |
| 696157 | 2801 | 2816 | 45190 | 45205 | CAGCAAAGAGATGTTC | 21 | 1100 |
| 696158 | 2808 | 2823 | 45197 | 45212 | GATTGGGCAGCAAAGA | 19 | 1101 |
| 696159 | 2811 | 2826 | 45200 | 45215 | ATGGATTGGGCAGCAA | 25 | 1102 |
| 696160 | 2825 | 2840 | 45214 | 45229 | TCCTACTGTCGCTAAT | 53 | 1103 |
| 696161 | 2827 | 2842 | 45216 | 45231 | AATCCTACTGTCGCTA | 37 | 1104 |

TABLE 18
Inhibition of K-Ras mRNA by 3-10-3 cEt
gapmers targeting SEQ ID NO: 2

| | SEQ ID | SEQ ID | | | |
|---|---|---|---|---|---|
| | NO: 2 | NO: 2 | | | SEQ |
| ISIS | Start | Stop | | % Inhi- | ID |
| NO | Site | Site | Sequence | bition | NO |
| 540806 | 45370 | 45385 | GCATGAAGATTTCTGG | 50 | 122 |
| 540806 | 45370 | 45385 | GCATGAAGATTTCTGG | 51 | 122 |
| 663688 | 9544 | 9559 | CCAGAGTCAAGTCTTC | 31 | 1105 |
| 696498 | 4399 | 4414 | ATTAGAGTTTGTGTAT | 11 | 1106 |
| 696499 | 4885 | 4900 | TTATAACAAGGTCTCA | 0 | 1107 |
| 696500 | 4949 | 4964 | CGGTAAATATTAATAA | 0 | 1108 |
| 696501 | 4958 | 4973 | TTTCCAATACGGTAAA | 9 | 1109 |
| 696502 | 4960 | 4975 | AATTTCCAATACGGTA | 28 | 1110 |
| 696503 | 5169 | 5184 | TAATAGATTGAATGCA | 5 | 1111 |
| 696504 | 5280 | 5295 | ACTAAGACTTTTCTGG | 5 | 1112 |
| 696505 | 5482 | 5497 | AAGTTAACAACCACTA | 5 | 1113 |
| 696506 | 5562 | 5577 | AATTGTATCACACACG | 28 | 1114 |
| 696507 | 5572 | 5587 | AGGCTAGAAAAATTGT | 5 | 1115 |
| 696508 | 5694 | 5709 | ATTGTAGATAAACACT | 13 | 1116 |
| 696509 | 5734 | 5749 | ATTATAGGATGAGTAG | 1 | 1117 |
| 696510 | 5754 | 5769 | GAGTAAATGCACAACT | 0 | 1118 |
| 696511 | 5904 | 5919 | GATTGTATAACAAACA | 5 | 1119 |
| 696512 | 5994 | 6009 | AGTGAATATATCTCAG | 20 | 1120 |
| 696513 | 6002 | 6017 | AAGATGCAAGTGAATA | 4 | 1121 |
| 696514 | 6729 | 6744 | AGAGAACTCCGAATTA | 0 | 1122 |
| 696515 | 6988 | 7003 | ATCAGATGAGAGTTGA | 2 | 1123 |
| 696516 | 7188 | 7203 | ACTCATGTAGAGACTT | 6 | 1124 |
| 696517 | 7200 | 7215 | TATCATGACTTCACTC | 16 | 1125 |
| 696518 | 7250 | 7265 | TAAATAGCCAGACTGC | 12 | 1126 |
| 696519 | 7292 | 7307 | TACATAGACAGTTCTT | 24 | 1127 |
| 696520 | 7301 | 7316 | CATAAATGCTACATAG | 7 | 1128 |
| 696521 | 7351 | 7366 | ATGAACTGTACTTCAT | 0 | 1129 |
| 696522 | 7471 | 7486 | ACTATCAAATACTCCA | 26 | 1130 |
| 696523 | 7503 | 7518 | ATATTAGAACATGTCA | 0 | 1131 |
| 696524 | 7545 | 7560 | ATTTTCAGCAGGCCTT | 23 | 1132 |
| 696525 | 7666 | 7681 | AACAAGATTTACCTCT | 0 | 1133 |
| 696526 | 7790 | 7805 | CAAGGTACATTTCAGA | 38 | 1134 |
| 696527 | 7935 | 7950 | CAGATAGGATACAAAT | 3 | 1135 |
| 696528 | 7960 | 7975 | CAATAAAAAGATTGTC | 0 | 1136 |
| 696529 | 8011 | 8026 | ACCTTTACATATGATG | 4 | 1137 |
| 696530 | 8034 | 8049 | TTTCACTAGTACAATT | 6 | 1138 |
| 696531 | 8179 | 8194 | AATAGTACCTTTATAT | 0 | 1139 |
| 696532 | 8412 | 8427 | CATCTGCTTGGGATGG | 11 | 1140 |
| 696533 | 8415 | 8430 | CCTCATCTGCTTGGGA | 16 | 1141 |
| 696534 | 8610 | 8625 | CCTAAGAAACAATCTA | 16 | 1142 |
| 696535 | 8751 | 8766 | ACTTTGACCTGTTCTA | 2 | 1143 |
| 696536 | 8789 | 8804 | TCTTCAAGACACTACA | 7 | 1144 |
| 696537 | 8800 | 8815 | CGCAAAGTGTCTCTTC | 10 | 1145 |
| 696538 | 8815 | 8830 | CAGAACTTGCCTCAGC | 26 | 1146 |
| 696539 | 8885 | 8900 | GATTAGTTATCTAATC | 0 | 1147 |
| 696540 | 8996 | 9011 | CTTAAAATTGGAAGCC | 30 | 1148 |
| 696541 | 9060 | 9075 | ATACAGAGACTATTGC | 34 | 1149 |
| 696542 | 9091 | 9106 | TACACATTGAATTAAC | 3 | 1150 |
| 696543 | 9140 | 9155 | ATTAAAATGGGTGCAC | 8 | 1151 |
| 696544 | 9325 | 9340 | CTGATTGGAAACAAAG | 13 | 1152 |
| 696545 | 9542 | 9557 | AGAGTCAAGTCTTCTG | 0 | 1153 |
| 696546 | 9555 | 9570 | ACCAATGCTTCCCAGA | 30 | 1154 |
| 696547 | 9627 | 9642 | AGATAATCTCAGATAC | 11 | 1155 |
| 696548 | 9736 | 9751 | CAACTATTTAACTACT | 0 | 1156 |
| 696549 | 9813 | 9828 | CAATGGCAGTGAAATC | 21 | 1157 |
| 696550 | 9824 | 9839 | ATCAAAACCTGCAATG | 3 | 1158 |
| 696551 | 9876 | 9891 | AGTCATATCCTTTCTA | 15 | 1159 |
| 696552 | 9889 | 9904 | TTCCAAAATGTGCAGT | 34 | 1160 |
| 696553 | 10022 | 10037 | AACAATAGCCACCCTC | 20 | 1161 |
| 696554 | 10141 | 10156 | CAAGAGTACAGTGCAA | 46 | 1162 |
| 696555 | 10179 | 10194 | TTTGAAAGATAGCTAA | 0 | 1163 |
| 696556 | 10213 | 10228 | CTGAATTAGTCTCCAT | 61 | 985 |
| 696557 | 10504 | 10519 | GATCTCTGAACTATAA | 0 | 1164 |
| 696558 | 10734 | 10749 | CCACAATAAAAGCATG | 15 | 1165 |
| 696559 | 10761 | 10776 | CATAATACTTGAACTG | 29 | 1166 |
| 696560 | 10791 | 10806 | TGAATAGGAAACTGTT | 0 | 1167 |
| 696561 | 10823 | 10838 | ACCAATCCAATGATTA | 29 | 1168 |
| 696562 | 10841 | 10856 | ACACTAAAGATGAAAC | 6 | 1169 |
| 696563 | 10867 | 10882 | GGTAAATAAATACTCT | 26 | 1170 |
| 696564 | 11016 | 11031 | TTACATAAGGCTTTTC | 16 | 1171 |
| 696565 | 11079 | 11094 | CAACCATCCCTCATTG | 8 | 1172 |
| 696566 | 11082 | 11097 | ACCCAACCATCCCTCA | 3 | 1173 |
| 696567 | 11694 | 11709 | ATACGAAATCAATCAT | 12 | 1174 |
| 696568 | 11982 | 11997 | AATTTGCCACTTCTGA | 19 | 1175 |
| 696569 | 12000 | 12015 | CAAAATGTGCACTTTC | 0 | 1176 |
| 696570 | 12091 | 12106 | TTCTTAATTTGACCTA | 20 | 1177 |
| 696571 | 12131 | 12146 | GACCAGTAAAGTTTTA | 27 | 1178 |
| 696572 | 12288 | 12303 | CTAGGATTAAGGAATT | 9 | 1179 |
| 696573 | 12369 | 12384 | AATCTGGTCTGTTTTG | 32 | 1180 |

TABLE 19
Inhibition of K-Ras mRNA by 3-10-3 cEt
gapmers targeting SEQ ID NO: 2

| | SEQ ID | SEQ ID | | | |
|---|---|---|---|---|---|
| | NO: 2 | NO: 2 | | | SEQ |
| ISIS | Start | Stop | | % Inhi- | ID |
| NO | Site | Site | Sequence | bition | NO |
| 540806 | 45370 | 45385 | GCATGAAGATTTCTGG | 69 | 122 |
| 540806 | 45370 | 45385 | GCATGAAGATTTCTGG | 73 | 122 |
| 696581 | 12559 | 12574 | GATTCAACCAATTATG | 29 | 1181 |
| 696582 | 12636 | 12651 | TAATTAGCATGATTGC | 14 | 1182 |
| 696583 | 12753 | 12768 | TATTAAGATCCCAATA | 0 | 1183 |
| 696584 | 12838 | 12853 | TAGTACAGCGAATAGC | 0 | 1184 |
| 696585 | 13223 | 13238 | AAGCAGTGACACTGCT | 0 | 1185 |
| 696586 | 13501 | 13516 | CTCAAGGGTGAAAAAT | 3 | 1186 |
| 696587 | 13593 | 13608 | ATTTTTATGCAGCCAG | 37 | 1187 |
| 696588 | 13672 | 13687 | TAAGATAGCTTCCTGT | 12 | 1188 |
| 696589 | 13684 | 13699 | CTAATTCATATATAAG | 0 | 1189 |
| 696590 | 13800 | 13815 | AGTAAGTGTCTTTTTA | 23 | 1190 |
| 696591 | 13944 | 13959 | CCATAAAGTCTGAGGG | 0 | 1191 |
| 696592 | 13993 | 14008 | GTCAAAGGACATGTAG | 13 | 1192 |
| 696593 | 14208 | 14223 | AGTATATCTAAATCTA | 0 | 1193 |
| 696594 | 14266 | 14281 | ACTCAACACAAGGTGC | 4 | 1194 |
| 696595 | 14514 | 14529 | TACCTCTCATATTATT | 4 | 1195 |
| 696596 | 14611 | 14626 | CCACAAGTGATCACTT | 0 | 1196 |
| 696597 | 14853 | 14868 | GACATTCACTAAAAGT | 0 | 1197 |
| 696598 | 15129 | 15144 | TTTATATACTACACGC | 37 | 1198 |
| 696599 | 15226 | 15241 | TAAAACTGCATACAGG | 4 | 1199 |
| 696600 | 15350 | 15365 | TAGACTTGGGAGTCTT | 0 | 1200 |
| 696601 | 15393 | 15408 | TACATACATGTCTGGT | 34 | 1201 |
| 696602 | 15716 | 15731 | AATTAGCAGTTTTTAG | 6 | 1202 |
| 696603 | 15840 | 15855 | GCAAAAACATAGACGA | 9 | 1203 |
| 696604 | 16077 | 16092 | CAAAGACAGAGCTACC | 0 | 1204 |
| 696605 | 16109 | 16124 | TACCAAAACCACTTGG | 0 | 1205 |
| 696606 | 16313 | 16328 | GTTAAAAATGGGTGGA | 11 | 1206 |
| 696607 | 16473 | 16488 | CAGCATTCCCTGAATC | 0 | 1207 |
| 696608 | 16495 | 16510 | TCTGATAAACCCCAAA | 9 | 1208 |
| 696609 | 16621 | 16636 | CAAAATGTTTTGGCCC | 0 | 1209 |
| 696610 | 16671 | 16686 | GGGAGATCAGATTCAT | 17 | 1210 |
| 696611 | 16679 | 16694 | AATAGGAAGGGAGATC | 14 | 1211 |
| 696612 | 16738 | 16753 | GTATATTAAGTAAGGA | 15 | 1212 |
| 696613 | 16784 | 16799 | GTGAAACTGGACAATC | 0 | 1213 |
| 696614 | 17106 | 17121 | ATTTTTCCAAGGACCG | 51 | 1214 |
| 696615 | 17204 | 17219 | CATGTTGAGGACACAG | 16 | 1215 |
| 696616 | 17383 | 17398 | GTGGAAGAGACATGAA | 15 | 1216 |
| 696617 | 17446 | 17461 | AATCTAGGTGTCACAT | 1 | 1217 |
| 696618 | 17600 | 17615 | CACTTTCCGTTTATAA | 1 | 1218 |
| 696619 | 17640 | 17655 | CGAAAGGTTATTTAAA | 4 | 1219 |
| 696620 | 17704 | 17719 | CACCTGTAGGAAAAGA | 5 | 1220 |
| 696621 | 17715 | 17730 | CTGCTTAATAACACCT | 20 | 1221 |
| 696622 | 17900 | 17915 | ACTGTCATAAGCATAT | 7 | 1222 |
| 696623 | 17902 | 17917 | ATACTGTCATAAGCAT | 23 | 1223 |
| 696624 | 17922 | 17937 | AGCCCTTACTTATATG | 0 | 1224 |
| 696625 | 18046 | 18061 | TACATTCCAAGTATAG | 0 | 1225 |
| 696626 | 18198 | 18213 | ACCAGAACATCAAGTT | 9 | 1226 |
| 696627 | 18203 | 18218 | CATTAACCAGAACATC | 7 | 1227 |
| 696628 | 18220 | 18235 | TCAAGATAAGATAACC | 7 | 1228 |
| 696629 | 18312 | 18327 | CTTCTTTTACACTGAC | 29 | 1229 |
| 696630 | 18523 | 18538 | TACTACTATTCTATAA | 0 | 1230 |
| 696631 | 18658 | 18673 | TAAGACTAGGGAAAAG | 30 | 1231 |
| 696632 | 19173 | 19188 | CTACCTAACAGTCTTG | 6 | 1232 |
| 696633 | 19192 | 19207 | ACTCACCACTACACAC | 0 | 1233 |
| 696634 | 19421 | 19436 | ATGAACGAAGGTAGGT | 24 | 1234 |
| 696635 | 19713 | 19728 | CACAATATAGTCTCCA | 38 | 1235 |
| 696636 | 19761 | 19776 | GGCAATCTGCAGCAAT | 9 | 1236 |
| 696637 | 19828 | 19843 | CTACATCCAACCACCT | 0 | 1237 |
| 696638 | 19926 | 19941 | TTAACATGGCATCCTA | 13 | 1238 |
| 696639 | 19934 | 19949 | AGAGATTCTTAACATG | 25 | 1239 |
| 696640 | 20250 | 20265 | TAACTTAAACTAACTC | 4 | 1240 |
| 696641 | 20285 | 20300 | AATTTGTAGCCTTAGG | 42 | 1241 |
| 696642 | 20289 | 20304 | TACTAATTTGTAGCCT | 9 | 1242 |
| 696643 | 20719 | 20734 | CTAAATAAGGTTTCAG | 7 | 1243 |
| 696644 | 20951 | 20966 | TATACACACGGCATTG | 0 | 1244 |
| 696645 | 21144 | 21159 | CTTAGAAGTGCAATTA | 18 | 1245 |
| 696646 | 21254 | 21269 | GTTTCAAAGTAATCTA | 0 | 1246 |
| 696647 | 21284 | 21299 | TATCGATAGCAAAGTT | 7 | 1247 |
| 696648 | 21398 | 21413 | TCATAAGATGCTTCCA | 10 | 1248 |
| 696649 | 21400 | 21415 | TTTCATAAGATGCTTC | 31 | 1249 |
| 696650 | 21437 | 21452 | AACCTGTAATGTGGGA | 31 | 1250 |
| 696651 | 21442 | 21457 | TAGTCAACCTGTAATG | 9 | 1251 |
| 696652 | 22061 | 22076 | ATTTGAGCATTCAGTT | 18 | 1252 |
| 696653 | 22728 | 22743 | AAAGATGTCTAAGTGC | 25 | 1253 |
| 696654 | 22748 | 22763 | TTACAGTATAAGGAGA | 36 | 1254 |
| 696655 | 22797 | 22812 | GAGAAAGAATGGTCAT | 0 | 1255 |
| 696656 | 23248 | 23263 | AAGAAGCAGGGCTAAC | 10 | 1256 |
| 696657 | 23428 | 23443 | ACTATAAGATTAAGTA | 13 | 1257 |

TABLE 20
Inhibition of K-Ras mRNA by 3-10-3 cEt
gapmers targeting SEQ ID NO: 2

| | SEQ ID | SEQ ID | | | |
|---|---|---|---|---|---|
| | NO: 2 | NO: 2 | | % | SEQ |
| ISIS | Start | Stop | | In- | ID |
| NO | Site | Site | Sequence | hibition | NO |
| 540806 | 45370 | 45385 | GCATGAAGATTTCTGG | 63 | 122 |
| 540806 | 45370 | 45385 | GCATGAAGATTTCTGG | 74 | 122 |
| 663670 | 37371 | 37386 | CTCTCTGCATTGTAAA | 31 | 1258 |
| 663729 | 37497 | 37512 | ACTTACCAGATTACAT | 3 | 1259 |
| 696733 | 34491 | 34506 | GAATTGGAAGCCAATA | 26 | 1260 |
| 696734 | 34493 | 34508 | GAGAATTGGAAGCCAA | 29 | 1261 |
| 696735 | 34496 | 34511 | AGAGAGAATTGGAAGC | 30 | 1262 |
| 696736 | 34502 | 34517 | CAATGCAGAGAGAATT | 1 | 1263 |
| 696737 | 34508 | 34523 | TTCCAGCAATGCAGAG | 1 | 1264 |
| 696738 | 34758 | 34773 | CCAGGTAAAAGCTCAT | 31 | 1265 |
| 696739 | 35416 | 35431 | ATTCTAAGAGCAGTCT | 16 | 1266 |
| 696740 | 35716 | 35731 | TAATTTTTGCATGCAG | 28 | 1267 |
| 696741 | 35718 | 35733 | CTTAATTTTTGCATGC | 22 | 1268 |
| 696742 | 35990 | 36005 | TAAAGCTGGTATATTT | 0 | 1269 |
| 696743 | 36111 | 36126 | AGAAAAGCATACCATC | 34 | 1270 |
| 696744 | 36181 | 36196 | TCCAATCTAGAAAATT | 9 | 1271 |
| 696745 | 36225 | 36240 | ACAATCATATATTGGC | 12 | 1272 |
| 696746 | 36329 | 36344 | TTAGAACAGTGTTCAA | 14 | 1273 |
| 696747 | 36668 | 36683 | ATCCTTACTACAAGTT | 15 | 1274 |
| 696748 | 36798 | 36813 | TACAAGTGAAGCTGAG | 30 | 1275 |
| 696749 | 37039 | 37054 | TTAAAGCCTAAACTGA | 2 | 1276 |
| 696750 | 37045 | 37060 | CTGGAATTAAAGCCTA | 0 | 1277 |
| 696751 | 37258 | 37273 | TTTAAACAGACATCAG | 8 | 1278 |
| 696752 | 37364 | 37379 | CATTGTAAAACACAAC | 1 | 1279 |
| 696753 | 37367 | 37382 | CTGCATTGTAAAACAC | 30 | 1280 |
| 696754 | 37373 | 37388 | CACTCTCTGCATTGTA | 12 | 1281 |
| 696755 | 37493 | 37508 | ACCAGATTACATTATA | 28 | 1282 |
| 696756 | 37495 | 37510 | TTACCAGATTACATTA | 8 | 1283 |
| 696757 | 37499 | 37514 | AAACTTACCAGATTAC | 6 | 1284 |
| 696758 | 37566 | 37581 | AAAGTGGTTGCCACCT | 14 | 1285 |
| 696759 | 37594 | 37609 | AGTTAGAATACTACAC | 7 | 1286 |
| 696760 | 37596 | 37611 | CAAGTTAGAATACTAC | 3 | 1287 |
| 696761 | 37715 | 37730 | ATGCCAAATATAGATT | 17 | 1288 |
| 696762 | 37880 | 37895 | ATATTACTGCTGTCTA | 26 | 1289 |
| 696763 | 37881 | 37896 | AATATTACTGCTGTCT | 11 | 1290 |
| 696764 | 38059 | 38074 | GAAAAGAGGGCGGTAG | 17 | 1291 |
| 696765 | 38181 | 38196 | TGGTAAACCAAATAGG | 35 | 1292 |
| 696766 | 38556 | 38571 | CTATAGCTAAAATGAC | 23 | 1293 |
| 696767 | 38587 | 38602 | CTGCAACACATGTGGA | 6 | 1294 |
| 696768 | 38623 | 38638 | AAAGAGCTGGAGTGGT | 15 | 1295 |
| 696769 | 38886 | 38901 | AAGCATATAATAGTTA | 6 | 1296 |
| 696770 | 38961 | 38976 | ATTCTGGCTAAGATTT | 0 | 1297 |
| 696771 | 39067 | 39082 | GTTTAGAAACGAAAAT | 10 | 1298 |
| 696772 | 39157 | 39172 | ACAAACAATATGCATC | 1 | 1299 |
| 696773 | 39196 | 39211 | CTGTAATTTTATTGCC | 18 | 1300 |
| 696774 | 39341 | 39356 | AGTAGATTAGTACACC | 32 | 1301 |
| 696775 | 39586 | 39601 | ACAATAGGAGGAGAAA | 16 | 1302 |
| 696776 | 39726 | 39741 | AGTCACTGTATAAAAC | 16 | 1303 |
| 696777 | 39750 | 39765 | CAAACAATTGTGACAT | 3 | 1304 |
| 696778 | 39820 | 39835 | AATTACCAAGTATACT | 21 | 1305 |
| 696779 | 40231 | 40246 | CTTTTTCAGGACTAAG | 8 | 1306 |
| 696780 | 40306 | 40321 | CAACCTACACAGAGCA | 9 | 1307 |
| 696781 | 40553 | 40568 | AAAGATTCTAGGCTTA | 11 | 1308 |
| 696782 | 40571 | 40586 | ACTTCCTAAGATTCTG | 5 | 1309 |
| 696783 | 40786 | 40801 | GTGATAGAATCTTAAA | 23 | 1310 |
| 696784 | 40945 | 40960 | AAGGTTTGATTACATA | 11 | 1311 |
| 696785 | 41190 | 41205 | TTTAAGAGAGGTAAAC | 0 | 1312 |
| 696786 | 41301 | 41316 | TACTAAGATTAACGAT | 23 | 1313 |
| 696787 | 41302 | 41317 | CTACTAAGATTAACGA | 7 | 1314 |
| 696788 | 41784 | 41799 | CCACTTTAGGAACAAT | 53 | 1315 |
| 696789 | 41810 | 41825 | CAACACATTAAGTTGT | 0 | 1316 |
| 696790 | 41812 | 41827 | CCCAACACATTAAGTT | 21 | 1317 |
| 696791 | 41844 | 41859 | CTAGACAGCAGAGGGA | 7 | 1318 |
| 696792 | 41994 | 42009 | GTCAATTCTTGTCATG | 34 | 1319 |
| 696793 | 42010 | 42025 | CAAGATCCATACACAA | 17 | 1320 |
| 696794 | 42086 | 42101 | ACCTAATAATCTACAG | 12 | 1321 |
| 696795 | 42094 | 42109 | ACTTTTCAACCTAATA | 0 | 1322 |
| 696796 | 42175 | 42190 | TAGTCATTGTGACCAC | 22 | 1323 |
| 696797 | 42312 | 42327 | TAGCTAAATCATTTGA | 20 | 1324 |
| 696798 | 42339 | 42354 | ACTAATACCTCAGATT | 30 | 1325 |
| 696799 | 42437 | 42452 | TGATATATATTAAGGG | 4 | 1326 |
| 696800 | 42662 | 42677 | ACTTAATTGTCCTTAT | 7 | 1327 |
| 696801 | 42664 | 42679 | ACACTTAATTGTCCTT | 28 | 1328 |
| 696802 | 42736 | 42751 | CTTAATTTGCTACTAT | 10 | 1329 |
| 696803 | 42764 | 42779 | ATAAGGTAACGACTTT | 18 | 1330 |
| 696804 | 42795 | 42810 | GACAAGGATAACCAAT | 18 | 1331 |
| 696805 | 42880 | 42895 | TATATTAGGACTTTTA | 5 | 1332 |
| 696806 | 42986 | 43001 | ATATATACGATGGCTT | 0 | 1333 |
| 696807 | 43412 | 43427 | CTGCATGCACCAAAAG | 15 | 1334 |

### Example 7: Antisense Inhibition of Human K-Ras in Hep3B Cells by cEt Gapmers

Antisense oligonucleotides were designed targeting a K-Ras nucleic acid and were tested for their effects on K-Ras mRNA in vitro. The antisense oligonucleotides were tested in a series of experiments that had similar culture conditions. The results for each experiment are presented in separate tables shown below. Cultured Hep3B cells at a density of 20,000 cells per well were transfected using electroporation with 2,000 nM antisense oligonucleotide. After a treatment period of approximately 24 hours, RNA was isolated from the cells and K-Ras mRNA levels were measured by quantitative real-time PCR. Human primer probe set RTS3496_MGB was used to measure mRNA levels. K-Ras mRNA levels were adjusted according to total RNA content, as measured by RIBOGREEN®. Results are presented as percent inhibition of K-Ras, relative to untreated control cells. As used herein, a value of ‘0’ indicates that treatment with the antisense oligonucleotide did not inhibit mRNA levels.

The newly designed chimeric antisense oligonucleotides in the Tables below were designed as 3-10-3 cEt gapmers. The gapmers are 16 nucleosides in length, wherein the central gap segment comprises of ten 2′-deoxynucleosides and is flanked by wing segments on the 5′ direction and the 3′ direction comprising three nucleosides each. Each nucleoside in the 5′ wing segment and each nucleoside in the 3′ wing segment has a cEt sugar modification. The internucleoside linkages throughout each gapmer are phosphorothioate (P═S) linkages. All cytosine residues throughout each gapmer are 5-methylcytosines. “Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either SEQ ID NO: 1 or SEQ ID NO: 2. Certain oligonucleotides are targeted to SEQ ID NO: 3. ‘N/A’ indicates that the antisense oligonucleotide does not target that particular gene sequence with 100% complementarity. In case the sequence alignment for a target gene in a particular table is not shown, it is understood that none of the oligonucleotides presented in that table align with 100% complementarity with that target gene.

TABLE 21
Inhibition of K-Ras mRNA by 3-10-3 cEt gapmers targeting
SEQ ID NO: 1 and 2

| | SEQ ID | SEQ ID | SEQ ID | SEQ ID | | | |
|---|---|---|---|---|---|---|---|
| | NO: 1 | NO: 1 | NO: 2 | NO: 2 | | | SEQ |
| ISIS | Start | Stop | Start | Stop | | % | ID |
| NO | Site | Site | Site | Site | Sequence | Inhibition | NO |
| 540806 | 2981 | 2996 | 45370 | 45385 | GCATGAAGATTTCTGG | 76 | 122 |
| 651672 | 2941 | 2956 | 45330 | 45345 | CTGTTACCAGGAGTAG | 75 | 1335 |
| 651675 | 2949 | 2964 | 45338 | 45353 | ATGTATTACTGTTACC | 72 | 1336 |
| 651677 | 2976 | 2991 | 45365 | 45380 | AAGATTTCTGGTTACT | 57 | 1337 |
| 651678 | 2978 | 2993 | 45367 | 45382 | TGAAGATTTCTGGTTA | 52 | 1338 |
| 651679 | 2980 | 2995 | 45369 | 45384 | CATGAAGATTTCTGGT | 51 | 1339 |
| 651681 | 2984 | 2999 | 45373 | 45388 | ATTGCATGAAGATTTC | 60 | 1340 |
| 651691 | 3379 | 3394 | 45768 | 45783 | ATTATGTCTCTTGTTT | 39 | 1341 |
| 651696 | 3481 | 3496 | 45870 | 45885 | CTTCTTTGCAAAACTA | 62 | 1342 |
| 651707 | 3622 | 3637 | 46011 | 46026 | ACAGTTATGCCAAATA | 67 | 1343 |
| 651708 | 3624 | 3639 | 46013 | 46028 | TCACAGTTATGCCAAA | 58 | 1344 |
| 651709 | 3626 | 3641 | 46015 | 46030 | AATCACAGTTATGCCA | 44 | 1345 |
| 651711 | 3630 | 3645 | 46019 | 46034 | AAAGAATCACAGTTAT | 17 | 1346 |
| 651712 | 3632 | 3647 | 46021 | 46036 | TAAAAGAATCACAGTT | 24 | 1347 |
| 651713 | 3642 | 3657 | 46031 | 46046 | GTAATTGTCCTAAAAG | 19 | 1348 |
| 651724 | 3786 | 3801 | 46175 | 46190 | TGTGAACTAGTTCAGG | 59 | 1349 |
| 651726 | 3800 | 3815 | 46189 | 46204 | GAAGTTTCCTTGTCTG | 68 | 1350 |
| 651732 | 3891 | 3906 | 46280 | 46295 | TACTGTGTAAGTCTTA | 60 | 1351 |
| 651733 | 3893 | 3908 | 46282 | 46297 | GGTACTGTGTAAGTCT | 76 | 1352 |
| 651734 | 3895 | 3910 | 46284 | 46299 | GAGGTACTGTGTAAGT | 58 | 1353 |
| 651736 | 3899 | 3914 | 46288 | 46303 | AAACGAGGTACTGTGT | 36 | 1354 |
| 651739 | 3939 | 3954 | 46328 | 46343 | CTGCAGTTCCTGAAGT | 17 | 1355 |
| 651741 | 3943 | 3958 | 46332 | 46347 | AGCACTGCAGTTCCTG | 72 | 1356 |
| 651742 | 3945 | 3960 | 46334 | 46349 | TAAGCACTGCAGTTCC | 65 | 1357 |
| 651743 | 3947 | 3962 | 46336 | 46351 | CATAAGCACTGCAGTT | 27 | 1358 |
| 652079 | 2925 | 2940 | 45314 | 45329 | TCCTAGTTATAGATTA | 44 | 1359 |
| 652080 | 2934 | 2949 | 45323 | 45338 | CAGGAGTAGTCCTAGT | 39 | 1360 |
| 652081 | 2962 | 2977 | 45351 | 45366 | CTAAAACAATGGAATG | 12 | 1361 |
| 652082 | 3004 | 3019 | 45393 | 45408 | CATGAATTAAAGTATT | 0 | 1362 |
| 652083 | 3013 | 3028 | 45402 | 45417 | AGTAAGCTTCATGAAT | 19 | 1363 |
| 652084 | 3038 | 3053 | 45427 | 45442 | CGAGACTCTGACACCA | 35 | 1364 |
| 652085 | 3271 | 3286 | 45660 | 45675 | GTTTATGAGGCCAAGG | 50 | 1365 |
| 652086 | 3280 | 3295 | 45669 | 45684 | GCAAAACAGGTTTATG | 59 | 1366 |
| 652087 | 3289 | 3304 | 45678 | 45693 | ATGAGTTCTGCAAAAC | 63 | 1367 |
| 652088 | 3325 | 3340 | 45714 | 45729 | CATCTGGTAGGCACTC | 62 | 1368 |
| 652089 | 3351 | 3366 | 45740 | 45755 | TACCCAGTGCCTTGTG | 15 | 1369 |
| 652090 | 3360 | 3375 | 45749 | 45764 | GATACCATATACCCAG | 64 | 1370 |
| 652091 | 3393 | 3408 | 45782 | 45797 | ACCTAAGGACCGGGAT | 30 | 1371 |
| 652092 | 3403 | 3418 | 45792 | 45807 | CACTAGCACTACCTAA | 8 | 1372 |
| 652093 | 3421 | 3436 | 45810 | 45825 | GTAAGATATTACAGAC | 56 | 1373 |
| 652094 | 3434 | 3449 | 45823 | 45838 | ACCAAAGGCCTTAGTA | 29 | 1374 |
| 652095 | 3469 | 3484 | 45858 | 45873 | ACTAAAATACGCATCG | 66 | 1375 |
| 652096 | 3490 | 3505 | 45879 | 45894 | ACCAAACCCCTTCTTT | 7 | 1376 |
| 652097 | 3500 | 3515 | 45889 | 45904 | TGGCACAGAGACCAAA | 8 | 1377 |
| 652098 | 3509 | 3524 | 45898 | 45913 | TTATAGAGCTGGCACA | 23 | 1378 |
| 652099 | 3518 | 3533 | 45907 | 45922 | GCAAAACAATTATAGA | 15 | 1379 |
| 652100 | 3538 | 3553 | 45927 | 45942 | AGAGTTTCAGTGGAAT | 74 | 1380 |
| 652101 | 3547 | 3562 | 45936 | 45951 | CTTGATCGAAGAGTTT | 73 | 1381 |
| 652102 | 3556 | 3571 | 45945 | 45960 | ATAAAGTAGCTTGATC | 31 | 1382 |
| 652103 | 3566 | 3581 | 45955 | 45970 | AGTGATTTACATAAAG | 38 | 1383 |
| 652104 | 3591 | 3606 | 45980 | 45995 | CAAGTTTATTCCTTTA | 57 | 1384 |
| 652105 | 3600 | 3615 | 45989 | 46004 | CAATATAATCAAGTTT | 0 | 1385 |
| 652106 | 3651 | 3666 | 46040 | 46055 | ATGTGTACAGTAATTG | 40 | 1386 |
| 652107 | 3661 | 3676 | 46050 | 46065 | ATACACCTTAATGTGT | 0 | 1387 |
| 652108 | 3678 | 3693 | 46067 | 46082 | CAATATGAATATCTGA | 17 | 1388 |
| 652109 | 3694 | 3709 | 46083 | 46098 | TATTACACATTTGGGT | 35 | 1389 |
| 652110 | 3703 | 3718 | 46092 | 46107 | AAACTGGAATATTACA | 16 | 1390 |
| 652111 | 3713 | 3728 | 46102 | 46117 | TATGCAGAGAAAACTG | 37 | 1391 |
| 652112 | 3722 | 3737 | 46111 | 46126 | TTAATTACTTATGCAG | 44 | 974 |
| 652113 | 3750 | 3765 | 46139 | 46154 | GATAAAACTATTAATT | 0 | 1392 |
| 652114 | 3759 | 3774 | 46148 | 46163 | TTGTACCCAGATAAAA | 21 | 1393 |
| 652115 | 3770 | 3785 | 46159 | 46174 | CACCTGTTTATTTGTA | 36 | 1394 |
| 652116 | 3809 | 3824 | 46198 | 46213 | TTTTACATAGAAGTTT | 24 | 1395 |
| 652117 | 3818 | 3833 | 46207 | 46222 | CATAGTGATTTTTACA | 43 | 1396 |
| 652118 | 3827 | 3842 | 46216 | 46231 | TTCAGAAATCATAGTG | 60 | 1397 |
| 652119 | 3839 | 3854 | 46228 | 46243 | TTCACATAGCAATTCA | 63 | 1398 |
| 652120 | 3848 | 3863 | 46237 | 46252 | ATCTGTAGTTTCACAT | 51 | 1399 |
| 652121 | 3857 | 3872 | 46246 | 46261 | GTTCCAAAGATCTGTA | 65 | 1400 |
| 652122 | 3876 | 3891 | 46265 | 46280 | AACACCCTACCTAAAC | 10 | 1401 |
| 652123 | 3931 | 3946 | 46320 | 46335 | CCTGAAGTATGGCCAT | 64 | 1402 |
| 652124 | 3959 | 3974 | 46348 | 46363 | TAAATATCCCCTCATA | 10 | 1403 |
| 652125 | 3968 | 3983 | 46357 | 46372 | CAAGAGGCCTAAATAT | 12 | 1404 |
| 652126 | 3977 | 3992 | 46366 | 46381 | TCAAAAATTCAAGAGG | 36 | 1405 |
| 652127 | 3986 | 4001 | 46375 | 46390 | CCATCTACATCAAAAA | 26 | 1406 |
| 652128 | 3995 | 4010 | 46384 | 46399 | AAAAAATGCCCATCTA | 21 | 1407 |
| 652129 | 4006 | 4021 | 46395 | 46410 | CCACTACCTTAAAAAA | 5 | 1408 |
| 652130 | 4018 | 4033 | 46407 | 46422 | AAAGGTAATTAACCAC | 0 | 1409 |
| 652131 | 4027 | 4042 | 46416 | 46431 | AGTTCACATAAAGGTA | 69 | 1410 |

TABLE 22
Inhibition of K-Ras mRNA by 3-10-3 cEt gapmers targeting
SEQ ID NO: 1 and 2

| | SEQ ID | SEQ ID | SEQ ID | SEQ ID | | | |
|---|---|---|---|---|---|---|---|
| | NO: 1 | NO: 1 | NO: 2 | NO: 2 | | | SEQ |
| ISIS | Start | Stop | Start | Stop | | % | ID |
| NO | Site | Site | Site | Site | Sequence | Inhibition | NO |
| 540806 | 2981 | 2996 | 45370 | 45385 | GCATGAAGATTTCTGG | 77 | 122 |
| 540832 | 4277 | 4292 | 46666 | 46681 | ATGCAGTGTGACTCAG | 56 | 135 |
| 540834 | 4279 | 4294 | 46668 | 46683 | CTATGCAGTGTGACTC | 50 | 136 |
| 540836 | 4338 | 4353 | 46727 | 46742 | CAAAATTGTGCAATGG | 52 | 137 |
| 540839 | 4343 | 4358 | 46732 | 46747 | TAGGACAAAATTGTGC | 42 | 64 |
| 540846 | 4579 | 4594 | 46968 | 46983 | AAGGTAACTGCTGGGT | 69 | 67 |
| 651757 | 4267 | 4282 | 46656 | 46671 | ACTCAGTTAAATAGAG | 2 | 1411 |
| 651759 | 4273 | 4288 | 46662 | 46677 | AGTGTGACTCAGTTAA | 64 | 970 |
| 651760 | 4274 | 4289 | 46663 | 46678 | CAGTGTGACTCAGTTA | 72 | 971 |
| 651762 | 4280 | 4295 | 46669 | 46684 | CCTATGCAGTGTGACT | 51 | 1412 |
| 651763 | 4281 | 4296 | 46670 | 46685 | TCCTATGCAGTGTGAC | 48 | 1413 |
| 651764 | 4283 | 4298 | 46672 | 46687 | ATTCCTATGCAGTGTG | 55 | 1414 |
| 651765 | 4287 | 4302 | 46676 | 46691 | CTAAATTCCTATGCAG | 30 | 1415 |
| 651768 | 4308 | 4323 | 46697 | 46712 | ATAACCTATAAAAGTT | 8 | 1416 |
| 651771 | 4340 | 4355 | 46729 | 46744 | GACAAAATTGTGCAAT | 27 | 1417 |
| 651772 | 4342 | 4357 | 46731 | 46746 | AGGACAAAATTGTGCA | 60 | 1418 |
| 651773 | 4344 | 4359 | 46733 | 46748 | TTAGGACAAAATTGTG | 28 | 1419 |
| 651774 | 4346 | 4361 | 46735 | 46750 | TATTAGGACAAAATTG | 0 | 1420 |
| 651775 | 4348 | 4363 | 46737 | 46752 | TATATTAGGACAAAAT | 0 | 1421 |
| 651780 | 4472 | 4487 | 46861 | 46876 | CCCTAAAAAAAGTTAT | 3 | 1422 |
| 651786 | 4574 | 4589 | 46963 | 46978 | AACTGCTGGGTTCTAA | 34 | 1423 |
| 651787 | 4576 | 4591 | 46965 | 46980 | GTAACTGCTGGGTTCT | 49 | 1424 |
| 651790 | 4582 | 4597 | 46971 | 46986 | TTTAAGGTAACTGCTG | 41 | 1425 |
| 651796 | 4626 | 4641 | 47015 | 47030 | TGCTATCCAGTATTAA | 46 | 1426 |
| 651800 | 4731 | 4746 | 47120 | 47135 | TCTTAATCTAGTTATG | 3 | 1427 |
| 651801 | 4761 | 4776 | 47150 | 47165 | GCACTTCAAACTATTA | 70 | 1428 |
| 651808 | 4893 | 4908 | 47282 | 47297 | ACTTTCGGATAAAACA | 19 | 1429 |
| 652132 | 4036 | 4051 | 46425 | 46440 | ACCATTCAAAGTTCAC | 73 | 975 |
| 652133 | 4046 | 4061 | 46435 | 46450 | CTTTTGTTAAACCATT | 42 | 1430 |
| 652134 | 4071 | 4086 | 46460 | 46475 | CTTTAAAATCTCTACA | 0 | 1431 |
| 652135 | 4080 | 4095 | 46469 | 46484 | ATTCTCCCCCTTTAAA | 15 | 1432 |
| 652136 | 4112 | 4127 | 46501 | 46516 | GCTGTAATAATTAGGT | 52 | 1433 |
| 652137 | 4121 | 4136 | 46510 | 46525 | GTCTTTAAGGCTGTAA | 41 | 1434 |
| 652138 | 4134 | 4149 | 46523 | 46538 | AACAAGGATTTTTGTC | 0 | 1435 |
| 652139 | 4143 | 4158 | 46532 | 46547 | AAAAACTTCAACAAGG | 34 | 1436 |
| 652140 | 4172 | 4187 | 46561 | 46576 | CTAAGTCTATGTAATT | 0 | 1437 |
| 652141 | 4181 | 4196 | 46570 | 46585 | TGTTAATGCCTAAGTC | 21 | 1438 |
| 652142 | 4190 | 4205 | 46579 | 46594 | CCACAAACATGTTAAT | 12 | 1439 |
| 652143 | 4200 | 4215 | 46589 | 46604 | CTATATTCTTCCACAA | 43 | 1440 |
| 652144 | 4225 | 4240 | 46614 | 46629 | ACTCAAATGATACAAT | 0 | 1441 |
| 652145 | 4249 | 4264 | 46638 | 46653 | TAGAATGCCTACTTGG | 30 | 1442 |
| 652146 | 4298 | 4313 | 46687 | 46702 | AAAGTTAGGTTCTAAA | 4 | 1443 |
| 652147 | 4317 | 4332 | 46706 | 46721 | ACAGTTTTGATAACCT | 57 | 1444 |
| 652148 | 4327 | 4342 | 46716 | 46731 | AATGGTGACAACAGTT | 58 | 1445 |
| 652149 | 4374 | 4389 | 46763 | 46778 | AACATGCCCCACAAAG | 20 | 1446 |
| 652150 | 4383 | 4398 | 46772 | 46787 | CTGTAACTTAACATGC | 28 | 1447 |
| 652151 | 4403 | 4418 | 46792 | 46807 | ATGAGATGAACTTGTG | 60 | 1448 |
| 652152 | 4412 | 4427 | 46801 | 46816 | GGAATACAAATGAGAT | 43 | 1449 |
| 652153 | 4461 | 4476 | 46850 | 46865 | GTTATATACTGTTTGA | 59 | 1450 |
| 652154 | 4496 | 4511 | 46885 | 46900 | GTTTTTGCTGTCTAAA | 53 | 1451 |
| 652155 | 4505 | 4520 | 46894 | 46909 | CTTCAGATAGTTTTTG | 39 | 1452 |
| 652156 | 4514 | 4529 | 46903 | 46918 | AATGGAAATCTTCAGA | 49 | 1453 |
| 652157 | 4524 | 4539 | 46913 | 46928 | CTTTTTGACAAATGGA | 71 | 976 |
| 652158 | 4537 | 4552 | 46926 | 46941 | CAAGAAATCATTACTT | 0 | 1454 |
| 652159 | 4551 | 4566 | 46940 | 46955 | TACTACACAATTATCA | 20 | 1455 |
| 652160 | 4561 | 4576 | 46950 | 46965 | TAAAAAACATTACTAC | 0 | 1456 |
| 652161 | 4606 | 4621 | 46995 | 47010 | GAAGTTACTAAATATA | 0 | 1457 |
| 652162 | 4615 | 4630 | 47004 | 47019 | ATTAACACAGAAGTTA | 22 | 1458 |
| 652163 | 4635 | 4650 | 47024 | 47039 | CAGAATTCATGCTATC | 64 | 1459 |
| 652164 | 4647 | 4662 | 47036 | 47051 | AGTTTCTCAATGCAGA | 32 | 1460 |
| 652165 | 4660 | 4675 | 47049 | 47064 | ATGACAGCTATTCAGT | 47 | 1461 |
| 652166 | 4670 | 4685 | 47059 | 47074 | GTTTCATTTTATGACA | 36 | 1462 |
| 652167 | 4681 | 4696 | 47070 | 47085 | TTAGAAAGAAAGTTTC | 0 | 1463 |
| 652168 | 4693 | 4708 | 47082 | 47097 | GAGTATCTTTCTTTAG | 28 | 1464 |
| 652169 | 4702 | 4717 | 47091 | 47106 | AACTCATGTGAGTATC | 54 | 1465 |
| 652170 | 4711 | 4726 | 47100 | 47115 | TTCTTCAAGAACTCAT | 44 | 1466 |
| 652171 | 4722 | 4737 | 47111 | 47126 | AGTTATGACTATTCTT | 31 | 1467 |
| 652172 | 4740 | 4755 | 47129 | 47144 | AAACACAGATCTTAAT | 0 | 1468 |
| 652173 | 4752 | 4767 | 47141 | 47156 | ACTATTAAACTAAAAC | 8 | 1469 |
| 652174 | 4770 | 4785 | 47159 | 47174 | CCCAAACAGGCACTTC | 62 | 1470 |
| 652175 | 4779 | 4794 | 47168 | 47183 | TATCATTATCCCAAAC | 4 | 1471 |
| 652176 | 4788 | 4803 | 47177 | 47192 | TAAATTACCTATCATT | 0 | 1472 |
| 652177 | 4800 | 4815 | 47189 | 47204 | CCTAAATTCATCTAAA | 0 | 1473 |
| 652178 | 4821 | 4836 | 47210 | 47225 | CTGCAGATAACTTTTT | 12 | 1474 |
| 652179 | 4831 | 4846 | 47220 | 47235 | CTCAACATATCTGCAG | 9 | 1475 |
| 652180 | 4877 | 4892 | 47266 | 47281 | CTGTAACCCAGTTAGC | 43 | 1476 |
| 652181 | 4902 | 4917 | 47291 | 47306 | GAATTGGAAACTTTCG | 29 | 1477 |
| 652182 | 4915 | 4930 | 47304 | 47319 | ACACAAGACAGTGGAA | 36 | 1478 |

TABLE 23
Inhibition of K-Ras mRNA by 3-10-3 cEt gapmers targeting
SEQ ID NO: 1 and 2

| | SEQ ID | SEQ ID | SEQ ID | SEQ ID | | | |
|---|---|---|---|---|---|---|---|
| | NO: 1 | NO: 1 | NO: 2 | NO: 2 | | | SEQ |
| ISIS | Start | Stop | Start | Stop | | % | ID |
| NO | Site | Site | Site | Site | Sequence | Inhibition | NO |
| 540806 | 2981 | 2996 | 45370 | 45385 | GCATGAAGATTTCTGG | 78 | 122 |
| 651824 | 5227 | 5242 | 47616 | 47631 | CAAATTTTAGATCACT | 5 | 1479 |
| 651827 | N/A | N/A | 37388 | 37403 | ATAAAAAGCATCCTCC | 27 | 1480 |
| 651831 | N/A | N/A | 37463 | 37478 | TTTCACACAGCCAGGA | 34 | 1481 |
| 652183 | 4971 | 4986 | 47360 | 47375 | TACTAGTAAGAAATTG | 9 | 1482 |
| 652184 | 4980 | 4995 | 47369 | 47384 | AAGAAATAGTACTAGT | 26 | 1483 |
| 652185 | 5013 | 5028 | 47402 | 47417 | GTTAAAATACATTCCA | 42 | 1484 |
| 652186 | 5028 | 5043 | 47417 | 47432 | CACTATACAAAAATAG | 4 | 1485 |
| 652187 | 5037 | 5052 | 47426 | 47441 | TTCAGTTTACACTATA | 45 | 1486 |
| 652188 | 5048 | 5063 | 47437 | 47452 | AATGTGCATGTTTCAG | 46 | 1487 |
| 652189 | 5061 | 5076 | 47450 | 47465 | GCACAATGTACAAAAT | 11 | 1488 |
| 652190 | 5076 | 5091 | 47465 | 47480 | GTCCCACAAAAGAAAG | 18 | 1489 |
| 652191 | 5097 | 5112 | 47486 | 47501 | CAACTGGATCACACTG | 27 | 1490 |
| 652192 | 5108 | 5123 | 47497 | 47512 | ATGATGGAAAACAACT | 19 | 1491 |
| 652193 | 5118 | 5133 | 47507 | 47522 | GCGCAACCAAATGATG | 12 | 1492 |
| 652194 | 5137 | 5152 | 47526 | 47541 | GACCAACATTCCTAGG | 24 | 1493 |
| 652195 | 5146 | 5161 | 47535 | 47550 | GTTTGATATGACCAAC | 33 | 1494 |
| 652196 | 5159 | 5174 | 47548 | 47563 | GGTCATTTTTAATGTT | 30 | 1495 |
| 652197 | 5168 | 5183 | 47557 | 47572 | TAAAAGAGTGGTCATT | 0 | 1496 |
| 652198 | 5200 | 5215 | 47589 | 47604 | ACTCCTATAAACATTT | 22 | 1497 |
| 652199 | 5209 | 5224 | 47598 | 47613 | ACAGCACATACTCCTA | 49 | 1498 |
| 652200 | 5218 | 5233 | 47607 | 47622 | GATCACTTCACAGCAC | 41 | 1499 |
| 652201 | 5249 | 5264 | 47638 | 47653 | ACAGTTCATGACAAAA | 40 | 1500 |
| 652202 | 5259 | 5274 | 47648 | 47663 | TAGGAGTAGTACAGTT | 17 | 1501 |
| 652203 | 5268 | 5283 | 47657 | 47672 | TACAATAATTAGGAGT | 4 | 1502 |
| 652206 | N/A | N/A | 37417 | 37432 | CTGTATTGTCGGATCT | 62 | 1503 |
| 652207 | N/A | N/A | 37430 | 37445 | GATTTTTTTCAATCTG | 36 | 1504 |
| 652208 | N/A | N/A | 37486 | 37501 | TACATTATAATGCATT | 21 | 1505 |
| 652209 | N/A | N/A | 2653 | 2668 | ATGCAGCAGGGAAGGC | 6 | 1506 |
| 652210 | N/A | N/A | 4249 | 4264 | TCCAAAGGAGTCTTAC | 47 | 1507 |
| 652211 | N/A | N/A | 7796 | 7811 | GAAACCCAAGGTACAT | 67 | 1508 |
| 652212 | N/A | N/A | 8388 | 8403 | CTCCATGACCTTCAAG | 49 | 1509 |
| 652213 | N/A | N/A | 9042 | 9057 | AGGCAGTCTACTTCAA | 44 | 1510 |
| 652214 | N/A | N/A | 9464 | 9479 | CCAAATAAAGGCTTAA | 0 | 1511 |
| 652215 | N/A | N/A | 10358 | 10373 | TACAAGTAAAGGTGAT | 27 | 1512 |
| 652216 | N/A | N/A | 10751 | 10766 | GAACTGAATTATAAGT | 31 | 1513 |
| 652217 | N/A | N/A | 11315 | 11330 | TATCAAGGTTTGGATC | 37 | 1514 |
| 652218 | N/A | N/A | 11502 | 11517 | TAAAATTGCTGTGTGT | 37 | 1515 |
| 652219 | N/A | N/A | 11687 | 11702 | ATCAATCATATAAGAC | 45 | 1516 |
| 652220 | N/A | N/A | 12032 | 12047 | TCACAACTATTCTACA | 15 | 1517 |
| 652221 | N/A | N/A | 12408 | 12423 | CTAGAGATACCTAAAA | 11 | 1518 |
| 652222 | N/A | N/A | 13439 | 13454 | AATCTATGTTACTTAG | 19 | 1519 |
| 652223 | N/A | N/A | 13991 | 14006 | CAAAGGACATGTAGTT | 35 | 1520 |
| 652224 | N/A | N/A | 14347 | 14362 | AGCCCAATGGTATAAG | 20 | 1521 |
| 652225 | N/A | N/A | 14965 | 14980 | ATCACAGGGAAGGATA | 6 | 1522 |
| 652226 | N/A | N/A | 15751 | 15766 | AATAATCAGAGTGGAC | 20 | 1523 |
| 652227 | N/A | N/A | 16942 | 16957 | ACAGGAGCTAAGGCAA | 13 | 1524 |
| 652228 | N/A | N/A | 17144 | 17159 | AACTTTTCCGGCATCA | 23 | 1525 |
| 652229 | N/A | N/A | 17450 | 17465 | TGAAAATCTAGGTGTC | 31 | 1526 |
| 652230 | N/A | N/A | 17739 | 17754 | AGTATTGTAAGGACTT | 41 | 1527 |
| 652231 | N/A | N/A | 17984 | 17999 | TAACTTTTACTAAAGG | 4 | 1528 |
| 652232 | N/A | N/A | 18104 | 18119 | ACTCAGGCAGTGACTC | 38 | 1529 |
| 652233 | N/A | N/A | 18935 | 18950 | ATGTAACAGTGTGCAA | 41 | 1530 |
| 652234 | N/A | N/A | 18964 | 18979 | GAATGTTCACGACAAA | 58 | 1531 |
| 652235 | N/A | N/A | 19258 | 19273 | AATTGTTTAAGTCTAT | 1 | 1532 |
| 652236 | N/A | N/A | 19785 | 19800 | GTCCATGATAACTATT | 37 | 1533 |
| 652237 | N/A | N/A | 21645 | 21660 | GTACAGATTGGCCAGG | 32 | 1534 |
| 652238 | N/A | N/A | 25871 | 25886 | ACTCCACTGCTCTAAT | 8 | 1535 |
| 652239 | N/A | N/A | 26391 | 26406 | ACTAGACTATACAGTA | 7 | 1536 |
| 652240 | N/A | N/A | 26720 | 26735 | CTAGAAAGATTTTGAT | 0 | 1537 |
| 652241 | N/A | N/A | 31136 | 31151 | AAGTTAGGGCATAAAA | 17 | 1538 |
| 652242 | N/A | N/A | 31818 | 31833 | TATTAAAGTTAGCCTG | 34 | 1539 |
| 652243 | N/A | N/A | 33116 | 33131 | GTTCAAAATATTGATC | 18 | 1540 |
| 652244 | N/A | N/A | 33201 | 33216 | AAAAACCACTACTTGG | 22 | 1541 |
| 652245 | N/A | N/A | 34689 | 34704 | AAGTTATAATGTCAAT | 6 | 1542 |
| 652246 | N/A | N/A | 35767 | 35782 | ACAGAGAATTGGCAAC | 48 | 1543 |
| 652247 | N/A | N/A | 35770 | 35785 | CACACAGAGAATTGGC | 71 | 1544 |
| 652248 | N/A | N/A | 35803 | 35818 | CACCAGTACCATTTGC | 36 | 1545 |
| 652249 | N/A | N/A | 36056 | 36071 | ATATATAGTGCAAATT | 15 | 1546 |
| 652250 | N/A | N/A | 36325 | 36340 | AACAGTGTTCAATCAT | 57 | 1547 |
| 652251 | N/A | N/A | 36875 | 36890 | TCTCAAAGGTGAGTCA | 41 | 1548 |
| 652252 | N/A | N/A | 37321 | 37336 | AGTAATTTACTGGGAA | 35 | 1549 |
| 652253 | N/A | N/A | 38872 | 38887 | TAAGAATAGTATTCTG | 2 | 1550 |
| 652254 | N/A | N/A | 41315 | 41330 | CTCCTTTACTGTACTA | 23 | 1551 |
| 652255 | N/A | N/A | 42293 | 42308 | GTCTTATAGTTTACCA | 55 | 1552 |
| 652256 | N/A | N/A | 42551 | 42566 | GTAAAATCCATTGGAT | 15 | 1553 |

TABLE 24
Inhibition of K-Ras mRNA by 3-10-3 cEt gapmers targeting
SEQ ID NO: 1 and 2

| | SEQ ID | SEQ ID | SEQ ID | | | | |
|---|---|---|---|---|---|---|---|
| | NO: 1 | NO: 1 | NO: 2 | SEQ ID | | | SEQ |
| ISIS | Start | Stop | Start | 2: Stop | | % | ID |
| NO | Site | Site | Site | Site | Sequence | Inhibition | NO |
| 540806 | 2981 | 2996 | 45370 | 45385 | GCATGAAGATTTCTGG | 74 | 122 |
| 540806 | 2981 | 2996 | 45370 | 45385 | GCATGAAGATTTCTGG | 85 | 122 |
| 651666 | 2855 | 2870 | 45244 | 45259 | GTTCTGTCTATTCATA | 43 | 912 |
| 651673 | 2943 | 2958 | 45332 | 45347 | TACTGTTACCAGGAGT | 44 | 1554 |
| 651674 | 2945 | 2960 | 45334 | 45349 | ATTACTGTTACCAGGA | 69 | 1555 |
| 651676 | 2965 | 2980 | 45354 | 45369 | TTACTAAAACAATGGA | 14 | 1556 |
| 651683 | 3008 | 3023 | 45397 | 45412 | GCTTCATGAATTAAAG | 23 | 1557 |
| 651692 | 3381 | 3396 | 45770 | 45785 | GGATTATGTCTCTTGT | 68 | 1558 |
| 651693 | 3400 | 3415 | 45789 | 45804 | TAGCACTACCTAAGGA | 2 | 1559 |
| 696162 | 2829 | 2844 | 45218 | 45233 | AAAATCCTACTGTCGC | 50 | 1560 |
| 696163 | 2835 | 2850 | 45224 | 45239 | GTTTGAAAAATCCTAC | 12 | 1561 |
| 696164 | 2838 | 2853 | 45227 | 45242 | CAGGTTTGAAAAATCC | 57 | 1562 |
| 696165 | 2843 | 2858 | 45232 | 45247 | CATACCAGGTTTGAAA | 37 | 1563 |
| 696166 | 2845 | 2860 | 45234 | 45249 | TTCATACCAGGTTTGA | 50 | 1564 |
| 696167 | 2850 | 2865 | 45239 | 45254 | GTCTATTCATACCAGG | 79 | 925 |
| 696168 | 2860 | 2875 | 45249 | 45264 | ATAGGGTTCTGTCTAT | 5 | 1565 |
| 696169 | 2866 | 2881 | 45255 | 45270 | CACTGGATAGGGTTCT | 51 | 1566 |
| 696170 | 2869 | 2884 | 45258 | 45273 | TTCCACTGGATAGGGT | 31 | 1567 |
| 696171 | 2871 | 2886 | 45260 | 45275 | CCTTCCACTGGATAGG | 3 | 1568 |
| 696172 | 2876 | 2891 | 45265 | 45280 | ATTCTCCTTCCACTGG | 42 | 1569 |
| 696173 | 2892 | 2907 | 45281 | 45296 | GCACTATCTTTATTAA | 19 | 1570 |
| 696174 | 2898 | 2913 | 45287 | 45302 | CTTTCAGCACTATCTT | 48 | 1571 |
| 696175 | 2900 | 2915 | 45289 | 45304 | TTCTTTCAGCACTATC | 51 | 1572 |
| 696176 | 2903 | 2918 | 45292 | 45307 | GAATTCTTTCAGCACT | 73 | 1573 |
| 696177 | 2909 | 2924 | 45298 | 45313 | CCTAAGGAATTCTTTC | 42 | 1574 |
| 696178 | 2911 | 2926 | 45300 | 45315 | TACCTAAGGAATTCTT | 33 | 1575 |
| 696179 | 2913 | 2928 | 45302 | 45317 | ATTACCTAAGGAATTC | 21 | 1576 |
| 696180 | 2917 | 2932 | 45306 | 45321 | ATAGATTACCTAAGGA | 37 | 1577 |
| 696181 | 2919 | 2934 | 45308 | 45323 | TTATAGATTACCTAAG | 14 | 1578 |
| 696182 | 2921 | 2936 | 45310 | 45325 | AGTTATAGATTACCTA | 10 | 1579 |
| 696183 | 2923 | 2938 | 45312 | 45327 | CTAGTTATAGATTACC | 34 | 1580 |
| 696184 | 2927 | 2942 | 45316 | 45331 | AGTCCTAGTTATAGAT | 35 | 1581 |
| 696185 | 2930 | 2945 | 45319 | 45334 | AGTAGTCCTAGTTATA | 44 | 1582 |
| 696186 | 2935 | 2950 | 45324 | 45339 | CCAGGAGTAGTCCTAG | 42 | 1583 |
| 696187 | 2936 | 2951 | 45325 | 45340 | ACCAGGAGTAGTCCTA | 62 | 1584 |
| 696188 | 2937 | 2952 | 45326 | 45341 | TACCAGGAGTAGTCCT | 45 | 1585 |
| 696189 | 2938 | 2953 | 45327 | 45342 | TTACCAGGAGTAGTCC | 46 | 1586 |
| 696190 | 2940 | 2955 | 45329 | 45344 | TGTTACCAGGAGTAGT | 35 | 1587 |
| 696191 | 2942 | 2957 | 45331 | 45346 | ACTGTTACCAGGAGTA | 29 | 1588 |
| 696192 | 2946 | 2961 | 45335 | 45350 | TATTACTGTTACCAGG | 68 | 1589 |
| 696193 | 2951 | 2966 | 45340 | 45355 | GAATGTATTACTGTTA | 56 | 1590 |
| 696194 | 2954 | 2969 | 45343 | 45358 | ATGGAATGTATTACTG | 52 | 1591 |
| 696195 | 2967 | 2982 | 45356 | 45371 | GGTTACTAAAACAATG | 27 | 1592 |
| 696196 | 2969 | 2984 | 45358 | 45373 | CTGGTTACTAAAACAA | 42 | 1593 |
| 696197 | 2973 | 2988 | 45362 | 45377 | ATTTCTGGTTACTAAA | 18 | 1594 |
| 696198 | 2983 | 2998 | 45372 | 45387 | TTGCATGAAGATTTCT | 59 | 1595 |
| 696199 | 2987 | 3002 | 45376 | 45391 | TTCATTGCATGAAGAT | 40 | 1596 |
| 696200 | 2989 | 3004 | 45378 | 45393 | TTTTCATTGCATGAAG | 39 | 1597 |
| 696201 | 3011 | 3026 | 45400 | 45415 | TAAGCTTCATGAATTA | 19 | 1598 |
| 696202 | 3273 | 3288 | 45662 | 45677 | AGGTTTATGAGGCCAA | 36 | 1599 |
| 696203 | 3276 | 3291 | 45665 | 45680 | AACAGGTTTATGAGGC | 50 | 1600 |
| 696204 | 3285 | 3300 | 45674 | 45689 | GTTCTGCAAAACAGGT | 64 | 1601 |
| 696205 | 3287 | 3302 | 45676 | 45691 | GAGTTCTGCAAAACAG | 57 | 1602 |
| 696206 | 3342 | 3357 | N/A | N/A | CCTTGTGCGGTGACTG | 11 | 1603 |
| 696207 | 3354 | 3369 | 45743 | 45758 | ATATACCCAGTGCCTT | 14 | 1604 |
| 696208 | 3356 | 3371 | 45745 | 45760 | CCATATACCCAGTGCC | 55 | 1605 |
| 696209 | 3358 | 3373 | 45747 | 45762 | TACCATATACCCAGTG | 47 | 1606 |
| 696210 | 3383 | 3398 | 45772 | 45787 | CGGGATTATGTCTCTT | 69 | 1607 |
| 696211 | 3390 | 3405 | 45779 | 45794 | TAAGGACCGGGATTAT | 19 | 1608 |
| 696212 | 3395 | 3410 | 45784 | 45799 | CTACCTAAGGACCGGG | 51 | 1609 |
| 696213 | 3397 | 3412 | 45786 | 45801 | CACTACCTAAGGACCG | 32 | 1610 |
| 696214 | 3405 | 3420 | 45794 | 45809 | CACACTAGCACTACCT | 41 | 1611 |
| 696215 | 3407 | 3422 | 45796 | 45811 | ACCACACTAGCACTAC | 48 | 1612 |
| 696216 | 3409 | 3424 | 45798 | 45813 | AGACCACACTAGCACT | 48 | 1613 |
| 696217 | 3411 | 3426 | 45800 | 45815 | ACAGACCACACTAGCA | 43 | 1614 |
| 696218 | 3413 | 3428 | 45802 | 45817 | TTACAGACCACACTAG | 16 | 1615 |
| 696219 | 3418 | 3433 | 45807 | 45822 | AGATATTACAGACCAC | 76 | 1616 |
| 696220 | 3423 | 3438 | 45812 | 45827 | TAGTAAGATATTACAG | 24 | 1617 |
| 696221 | 3425 | 3440 | 45814 | 45829 | CTTAGTAAGATATTAC | 5 | 1618 |
| 696222 | 3430 | 3445 | 45819 | 45834 | AAGGCCTTAGTAAGAT | 25 | 1619 |
| 696223 | 3432 | 3447 | 45821 | 45836 | CAAAGGCCTTAGTAAG | 18 | 1620 |
| 696224 | 3436 | 3451 | 45825 | 45840 | ATACCAAAGGCCTTAG | 48 | 1621 |
| 696225 | 3438 | 3453 | 45827 | 45842 | GTATACCAAAGGCCTT | 49 | 1622 |
| 696226 | 3471 | 3486 | 45860 | 45875 | AAACTAAAATACGCAT | 2 | 1623 |
| 696227 | 3473 | 3488 | 45862 | 45877 | CAAAACTAAAATACGC | 34 | 1624 |
| 696228 | 3486 | 3501 | 45875 | 45890 | AACCCCTTCTTTGCAA | 34 | 1625 |
| 696229 | 3492 | 3507 | 45881 | 45896 | AGACCAAACCCCTTCT | 45 | 1626 |
| 696230 | 3494 | 3509 | 45883 | 45898 | AGAGACCAAACCCCTT | 35 | 1627 |
| 696231 | 3496 | 3511 | 45885 | 45900 | ACAGAGACCAAACCCC | 28 | 1628 |

TABLE 25
Inhibition of K-Ras mRNA by 3-10-3 cEt gapmers targeting
SEQ ID NO: 1 and 2

| | SEQ ID | SEQ ID | SEQ ID | | | | |
|---|---|---|---|---|---|---|---|
| | NO: 1 | NO: 1 | NO: 2 | SEQ ID | | | SEQ |
| ISIS | Start | Stop | Start | 2: Stop | | % | ID |
| NO | Site | Site | Site | Site | Sequence | Inhibition | NO |
| 540806 | 2981 | 2996 | 45370 | 45385 | GCATGAAGATTTCTGG | 44 | 122 |
| 540806 | 2981 | 2996 | 45370 | 45385 | GCATGAAGATTTCTGG | 58 | 122 |
| 651703 | 3548 | 3563 | 45937 | 45952 | GCTTGATCGAAGAGTT | 62 | 1629 |
| 651704 | 3554 | 3569 | 45943 | 45958 | AAAGTAGCTTGATCGA | 69 | 1630 |
| 651705 | 3568 | 3583 | 45957 | 45972 | GAAGTGATTTACATAA | 40 | 1631 |
| 651706 | 3595 | 3610 | 45984 | 45999 | TAATCAAGTTTATTCC | 40 | 1632 |
| 651719 | 3700 | 3715 | 46089 | 46104 | CTGGAATATTACACAT | 48 | 1633 |
| 651720 | 3715 | 3730 | 46104 | 46119 | CTTATGCAGAGAAAAC | 26 | 968 |
| 651727 | 3806 | 3821 | 46195 | 46210 | TACATAGAAGTTTCCT | 38 | 1634 |
| 651728 | 3812 | 3827 | 46201 | 46216 | GATTTTTACATAGAAG | 18 | 1635 |
| 651730 | 3859 | 3874 | 46248 | 46263 | GTGTTCCAAAGATCTG | 59 | 1636 |
| 696232 | 3498 | 3513 | 45887 | 45902 | GCACAGAGACCAAACC | 31 | 1637 |
| 696233 | 3507 | 3522 | 45896 | 45911 | ATAGAGCTGGCACAGA | 33 | 1638 |
| 696234 | 3511 | 3526 | 45900 | 45915 | AATTATAGAGCTGGCA | 29 | 1639 |
| 696235 | 3513 | 3528 | 45902 | 45917 | ACAATTATAGAGCTGG | 53 | 1640 |
| 696236 | 3515 | 3530 | 45904 | 45919 | AAACAATTATAGAGCT | 8 | 1641 |
| 696237 | 3536 | 3551 | 45925 | 45940 | AGTTTCAGTGGAATCG | 66 | 1642 |
| 696238 | 3537 | 3552 | 45926 | 45941 | GAGTTTCAGTGGAATC | 56 | 1643 |
| 696239 | 3543 | 3558 | 45932 | 45947 | ATCGAAGAGTTTCAGT | 49 | 1644 |
| 696240 | 3544 | 3559 | 45933 | 45948 | GATCGAAGAGTTTCAG | 51 | 1645 |
| 696241 | 3549 | 3564 | 45938 | 45953 | AGCTTGATCGAAGAGT | 71 | 1646 |
| 696242 | 3550 | 3565 | 45939 | 45954 | TAGCTTGATCGAAGAG | 62 | 1647 |
| 696243 | 3551 | 3566 | 45940 | 45955 | GTAGCTTGATCGAAGA | 53 | 1648 |
| 696244 | 3552 | 3567 | 45941 | 45956 | AGTAGCTTGATCGAAG | 56 | 1649 |
| 696245 | 3553 | 3568 | 45942 | 45957 | AAGTAGCTTGATCGAA | 50 | 1650 |
| 696246 | 3558 | 3573 | 45947 | 45962 | ACATAAAGTAGCTTGA | 21 | 1651 |
| 696247 | 3560 | 3575 | 45949 | 45964 | TTACATAAAGTAGCTT | 37 | 1652 |
| 696248 | 3563 | 3578 | 45952 | 45967 | GATTTACATAAAGTAG | 11 | 1653 |
| 696249 | 3572 | 3587 | 45961 | 45976 | CAATGAAGTGATTTAC | 34 | 1654 |
| 696250 | 3593 | 3608 | 45982 | 45997 | ATCAAGTTTATTCCTT | 57 | 1655 |
| 696251 | 3594 | 3609 | 45983 | 45998 | AATCAAGTTTATTCCT | 46 | 1656 |
| 696252 | 3596 | 3611 | 45985 | 46000 | ATAATCAAGTTTATTC | 23 | 1657 |
| 696253 | 3636 | 3651 | 46025 | 46040 | GTCCTAAAAGAATCAC | 46 | 1658 |
| 696254 | 3639 | 3654 | 46028 | 46043 | ATTGTCCTAAAAGAAT | 41 | 1659 |
| 696255 | 3644 | 3659 | 46033 | 46048 | CAGTAATTGTCCTAAA | 35 | 1660 |
| 696256 | 3646 | 3661 | 46035 | 46050 | TACAGTAATTGTCCTA | 48 | 1661 |
| 696257 | 3649 | 3664 | 46038 | 46053 | GTGTACAGTAATTGTC | 43 | 1662 |
| 696258 | 3653 | 3668 | 46042 | 46057 | TAATGTGTACAGTAAT | 0 | 1663 |
| 696259 | 3655 | 3670 | 46044 | 46059 | CTTAATGTGTACAGTA | 46 | 1664 |
| 696260 | 3657 | 3672 | 46046 | 46061 | ACCTTAATGTGTACAG | 39 | 1665 |
| 696261 | 3659 | 3674 | 46048 | 46063 | ACACCTTAATGTGTAC | 15 | 1666 |
| 696262 | 3663 | 3678 | 46052 | 46067 | ACATACACCTTAATGT | 0 | 1667 |
| 696263 | 3665 | 3680 | 46054 | 46069 | TGACATACACCTTAAT | 37 | 1668 |
| 696264 | 3667 | 3682 | 46056 | 46071 | TCTGACATACACCTTA | 33 | 1669 |
| 696265 | 3669 | 3684 | 46058 | 46073 | TATCTGACATACACCT | 50 | 1670 |
| 696266 | 3671 | 3686 | 46060 | 46075 | AATATCTGACATACAC | 39 | 1671 |
| 696267 | 3680 | 3695 | 46069 | 46084 | GTCAATATGAATATCT | 53 | 1672 |
| 696268 | 3686 | 3701 | 46075 | 46090 | ATTTGGGTCAATATGA | 31 | 1673 |
| 696269 | 3690 | 3705 | 46079 | 46094 | ACACATTTGGGTCAAT | 37 | 1674 |
| 696270 | 3692 | 3707 | 46081 | 46096 | TTACACATTTGGGTCA | 47 | 1675 |
| 696271 | 3719 | 3734 | 46108 | 46123 | ATTACTTATGCAGAGA | 73 | 977 |
| 696272 | 3755 | 3770 | 46144 | 46159 | ACCCAGATAAAACTAT | 16 | 1676 |
| 696273 | 3757 | 3772 | 46146 | 46161 | GTACCCAGATAAAACT | 12 | 1677 |
| 696274 | 3761 | 3776 | 46150 | 46165 | ATTTGTACCCAGATAA | 29 | 1678 |
| 696275 | 3763 | 3778 | 46152 | 46167 | TTATTTGTACCCAGAT | 38 | 1679 |
| 696276 | 3765 | 3780 | 46154 | 46169 | GTTTATTTGTACCCAG | 67 | 1680 |
| 696277 | 3773 | 3788 | 46162 | 46177 | AGGCACCTGTTTATTT | 46 | 1681 |
| 696278 | 3777 | 3792 | 46166 | 46181 | GTTCAGGCACCTGTTT | 30 | 1682 |
| 696279 | 3782 | 3797 | 46171 | 46186 | AACTAGTTCAGGCACC | 39 | 1683 |
| 696280 | 3791 | 3806 | 46180 | 46195 | TTGTCTGTGAACTAGT | 54 | 1684 |
| 696281 | 3793 | 3808 | 46182 | 46197 | CCTTGTCTGTGAACTA | 63 | 1685 |
| 696282 | 3802 | 3817 | 46191 | 46206 | TAGAAGTTTCCTTGTC | 50 | 1686 |
| 696283 | 3804 | 3819 | 46193 | 46208 | CATAGAAGTTTCCTTG | 51 | 1687 |
| 696284 | 3825 | 3840 | 46214 | 46229 | CAGAAATCATAGTGAT | 37 | 1688 |
| 696285 | 3837 | 3852 | 46226 | 46241 | CACATAGCAATTCAGA | 50 | 1689 |
| 696286 | 3841 | 3856 | 46230 | 46245 | GTTTCACATAGCAATT | 47 | 769 |
| 696287 | 3844 | 3859 | 46233 | 46248 | GTAGTTTCACATAGCA | 79 | 770 |
| 696288 | 3846 | 3861 | 46235 | 46250 | CTGTAGTTTCACATAG | 51 | 771 |
| 696289 | 3850 | 3865 | 46239 | 46254 | AGATCTGTAGTTTCAC | 74 | 1690 |
| 696290 | 3852 | 3867 | 46241 | 46256 | AAAGATCTGTAGTTTC | 47 | 1691 |
| 696291 | 3854 | 3869 | 46243 | 46258 | CCAAAGATCTGTAGTT | 43 | 1692 |
| 696292 | 3861 | 3876 | 46250 | 46265 | CAGTGTTCCAAAGATC | 56 | 1693 |
| 696293 | 3873 | 3888 | 46262 | 46277 | ACCCTACCTAAACAGT | 22 | 1694 |
| 696294 | 3878 | 3893 | 46267 | 46282 | TTAACACCCTACCTAA | 18 | 1695 |
| 696295 | 3880 | 3895 | 46269 | 46284 | TCTTAACACCCTACCT | 20 | 1696 |
| 696296 | 3887 | 3902 | 46276 | 46291 | GTGTAAGTCTTAACAC | 19 | 1697 |
| 696297 | 3892 | 3907 | 46281 | 46296 | GTACTGTGTAAGTCTT | 59 | 1698 |
| 696298 | 3902 | 3917 | 46291 | 46306 | TAGAAACGAGGTACTG | 41 | 1699 |
| 696299 | 3905 | 3920 | 46294 | 46309 | GTGTAGAAACGAGGTA | 73 | 1700 |

TABLE 26
Inhibition of K-Ras mRNA by 3-10-3
cEt gapmers targeting SEQ ID NO: 1 and 2

| | SEQ ID | SEQ ID | SEQ ID | | | | |
|---|---|---|---|---|---|---|---|
| | NO: 1 | NO: 1 | NO: 2 | SEQ ID | | | SEQ |
| ISIS | Start | Stop | Start | 2: Stop | | % | ID |
| NO | Site | Site | Site | Site | Sequence | Inhibition | NO |
| 540806 | 2981 | 2996 | 45370 | 45385 | GCATGAAGATTTCTGG | 37 | 122 |
| 540806 | 2981 | 2996 | 45370 | 45385 | GCATGAAGATTTCTGG | 60 | 122 |
| 651745 | 3972 | 3987 | 46361 | 46376 | AATTCAAGAGGCCTAA | 1 | 1701 |
| 651749 | 4087 | 4102 | 46476 | 46491 | TTCTAGAATTCTCCCC | 50 | 1702 |
| 651750 | 4140 | 4155 | 46529 | 46544 | AACTTCAACAAGGATT | 30 | 1703 |
| 651755 | 4233 | 4248 | 46622 | 46637 | GAACATTCACTCAAAT | 8 | 1704 |
| 651756 | 4252 | 4267 | 46641 | 46656 | GCCTAGAATGCCTACT | 38 | 1705 |
| 651758 | 4271 | 4286 | 46660 | 46675 | TGTGACTCAGTTAAAT | 33 | 1706 |
| 651766 | 4296 | 4311 | 46685 | 46700 | AGTTAGGTTCTAAATT | 0 | 1707 |
| 651767 | 4302 | 4317 | 46691 | 46706 | TATAAAAGTTAGGTTC | 0 | 1708 |
| 663588 | 4185 | 4200 | 46574 | 46589 | AACATGTTAATGCCTA | 46 | 1709 |
| 696300 | 3910 | 3925 | 46299 | 46314 | TCTCTGTGTAGAAACG | 41 | 1710 |
| 696301 | 3935 | 3950 | 46324 | 46339 | AGTTCCTGAAGTATGG | 59 | 1711 |
| 696302 | 3944 | 3959 | 46333 | 46348 | AAGCACTGCAGTTCCT | 52 | 1712 |
| 696303 | 3949 | 3964 | 46338 | 46353 | CTCATAAGCACTGCAG | 54 | 1713 |
| 696304 | 3951 | 3966 | 46340 | 46355 | CCCTCATAAGCACTGC | 57 | 1714 |
| 696305 | 3956 | 3971 | 46345 | 46360 | ATATCCCCTCATAAGC | 21 | 1715 |
| 696306 | 3961 | 3976 | 46350 | 46365 | CCTAAATATCCCCTCA | 20 | 1716 |
| 696307 | 3963 | 3978 | 46352 | 46367 | GGCCTAAATATCCCCT | 29 | 1717 |
| 696308 | 3970 | 3985 | 46359 | 46374 | TTCAAGAGGCCTAAAT | 31 | 1718 |
| 696309 | 3988 | 4003 | 46377 | 46392 | GCCCATCTACATCAAA | 51 | 1719 |
| 696310 | 3992 | 4007 | 46381 | 46396 | AAATGCCCATCTACAT | 16 | 1720 |
| 696311 | 4009 | 4024 | 46398 | 46413 | TAACCACTACCTTAAA | 21 | 1721 |
| 696312 | 4011 | 4026 | 46400 | 46415 | ATTAACCACTACCTTA | 0 | 1722 |
| 696313 | 4015 | 4030 | 46404 | 46419 | GGTAATTAACCACTAC | 20 | 1723 |
| 696314 | 4020 | 4035 | 46409 | 46424 | ATAAAGGTAATTAACC | 0 | 1724 |
| 696315 | 4028 | 4043 | 46417 | 46432 | AAGTTCACATAAAGGT | 43 | 1725 |
| 696316 | 4029 | 4044 | 46418 | 46433 | AAAGTTCACATAAAGG | 37 | 978 |
| 696317 | 4035 | 4050 | 46424 | 46439 | CCATTCAAAGTTCACA | 71 | 979 |
| 696318 | 4037 | 4052 | 46426 | 46441 | AACCATTCAAAGTTCA | 67 | 980 |
| 696319 | 4038 | 4053 | 46427 | 46442 | AAACCATTCAAAGTTC | 26 | 1726 |
| 696320 | 4043 | 4058 | 46432 | 46447 | TTGTTAAACCATTCAA | 21 | 1727 |
| 696321 | 4075 | 4090 | 46464 | 46479 | CCCCCTTTAAAATCTC | 10 | 1728 |
| 696322 | 4084 | 4099 | 46473 | 46488 | TAGAATTCTCCCCCTT | 45 | 1729 |
| 696323 | 4109 | 4124 | 46498 | 46513 | GTAATAATTAGGTAAC | 18 | 1730 |
| 696324 | 4114 | 4129 | 46503 | 46518 | AGGCTGTAATAATTAG | 39 | 1731 |
| 696325 | 4116 | 4131 | 46505 | 46520 | TAAGGCTGTAATAATT | 6 | 1732 |
| 696326 | 4119 | 4134 | 46508 | 46523 | CTTTAAGGCTGTAATA | 15 | 1733 |
| 696327 | 4136 | 4151 | 46525 | 46540 | TCAACAAGGATTTTTG | 3 | 1734 |
| 696328 | 4138 | 4153 | 46527 | 46542 | CTTCAACAAGGATTTT | 40 | 1735 |
| 696329 | 4169 | 4184 | 46558 | 46573 | AGTCTATGTAATTTAG | 21 | 1736 |
| 696330 | 4174 | 4189 | 46563 | 46578 | GCCTAAGTCTATGTAA | 28 | 1737 |
| 696331 | 4176 | 4191 | 46565 | 46580 | ATGCCTAAGTCTATGT | 30 | 1738 |
| 696332 | 4178 | 4193 | 46567 | 46582 | TAATGCCTAAGTCTAT | 24 | 1739 |
| 696333 | 4183 | 4198 | 46572 | 46587 | CATGTTAATGCCTAAG | 52 | 1740 |
| 696334 | 4187 | 4202 | 46576 | 46591 | CAAACATGTTAATGCC | 42 | 1741 |
| 696335 | 4202 | 4217 | 46591 | 46606 | TGCTATATTCTTCCAC | 53 | 1742 |
| 696336 | 4227 | 4242 | 46616 | 46631 | TCACTCAAATGATACA | 58 | 1743 |
| 696337 | 4230 | 4245 | 46619 | 46634 | CATTCACTCAAATGAT | 27 | 1744 |
| 696338 | 4235 | 4250 | 46624 | 46639 | GGGAACATTCACTCAA | 47 | 1745 |
| 696339 | 4242 | 4257 | 46631 | 46646 | CCTACTTGGGAACATT | 34 | 1746 |
| 696340 | 4244 | 4259 | 46633 | 46648 | TGCCTACTTGGGAACA | 22 | 1747 |
| 696341 | 4254 | 4269 | 46643 | 46658 | GAGCCTAGAATGCCTA | 33 | 1748 |
| 696342 | 4257 | 4272 | 46646 | 46661 | ATAGAGCCTAGAATGC | 0 | 1749 |
| 696343 | 4259 | 4274 | 46648 | 46663 | AAATAGAGCCTAGAAT | 0 | 1750 |
| 696344 | 4263 | 4278 | 46652 | 46667 | AGTTAAATAGAGCCTA | 13 | 1751 |
| 696345 | 4268 | 4283 | 46657 | 46672 | GACTCAGTTAAATAGA | 33 | 1752 |
| 696346 | 4269 | 4284 | 46658 | 46673 | TGACTCAGTTAAATAG | 23 | 1753 |
| 696347 | 4272 | 4287 | 46661 | 46676 | GTGTGACTCAGTTAAA | 51 | 1754 |
| 696348 | 4289 | 4304 | 46678 | 46693 | TTCTAAATTCCTATGC | 4 | 1755 |
| 696349 | 4292 | 4307 | 46681 | 46696 | AGGTTCTAAATTCCTA | 3 | 1756 |
| 696350 | 4300 | 4315 | 46689 | 46704 | TAAAAGTTAGGTTCTA | 8 | 1757 |
| 696351 | 4310 | 4325 | 46699 | 46714 | TGATAACCTATAAAAG | 20 | 1758 |
| 696352 | 4314 | 4329 | 46703 | 46718 | GTTTTGATAACCTATA | 32 | 1759 |
| 696353 | 4319 | 4334 | 46708 | 46723 | CAACAGTTTTGATAAC | 36 | 1760 |
| 696354 | 4321 | 4336 | 46710 | 46725 | GACAACAGTTTTGATA | 38 | 1761 |
| 696355 | 4323 | 4338 | 46712 | 46727 | GTGACAACAGTTTTGA | 60 | 1762 |
| 696356 | 4329 | 4344 | 46718 | 46733 | GCAATGGTGACAACAG | 69 | 1763 |
| 696357 | 4331 | 4346 | 46720 | 46735 | GTGCAATGGTGACAAC | 22 | 845 |
| 696358 | 4334 | 4349 | 46723 | 46738 | ATTGTGCAATGGTGAC | 63 | 846 |
| 696359 | 4336 | 4351 | 46725 | 46740 | AAATTGTGCAATGGTG | 39 | 847 |
| 696360 | 4353 | 4368 | 46742 | 46757 | ATGTATATATTAGGAC | 20 | 1764 |
| 696361 | 4355 | 4370 | 46744 | 46759 | CTATGTATATATTAGG | 22 | 1765 |
| 696362 | 4367 | 4382 | 46756 | 46771 | CCCACAAAGTTTCTAT | 22 | 1766 |
| 696363 | 4369 | 4384 | 46758 | 46773 | GCCCCACAAAGTTTCT | 23 | 1767 |
| 696364 | 4376 | 4391 | 46765 | 46780 | TTAACATGCCCCACAA | 20 | 1768 |
| 696365 | 4378 | 4393 | 46767 | 46782 | ACTTAACATGCCCCAC | 47 | 1769 |
| 696366 | 4380 | 4395 | 46769 | 46784 | TAACTTAACATGCCCC | 45 | 1770 |
| 696367 | 4385 | 4400 | 46774 | 46789 | AACTGTAACTTAACAT | 0 | 1771 |

TABLE 27
Inhibition of K-Ras mRNA by 3-10-3
cEt gapmers targeting SEQ ID NO: 1 and 2

| | SEQ ID | SEQ ID | SEQ ID | SEQ ID | | | |
|---|---|---|---|---|---|---|---|
| | NO: 1 | NO: 1 | NO: 2 | NO: 2 | | | SEQ |
| ISIS | Start | Stop | Start | Stop | | % | ID |
| NO | Site | Site | Site | Site | Sequence | Inhibition | NO |
| 540806 | 2981 | 2996 | 45370 | 45385 | GCATGAAGATTTCTGG | 72 | 122 |
| 540806 | 2981 | 2996 | 45370 | 45385 | GCATGAAGATTTCTGG | 77 | 122 |
| 651778 | 4389 | 4404 | 46778 | 46793 | TGCAAACTGTAACTTA | 43 | 1772 |
| 651781 | 4502 | 4517 | 46891 | 46906 | CAGATAGTTTTTGCTG | 13 | 1773 |
| 651782 | 4508 | 4523 | 46897 | 46912 | AATCTTCAGATAGTTT | 48 | 1774 |
| 651784 | 4566 | 4581 | 46955 | 46970 | GGTTCTAAAAAACATT | 21 | 1775 |
| 651791 | 4584 | 4599 | 46973 | 46988 | GCTTTAAGGTAACTGC | 45 | 1776 |
| 651792 | 4591 | 4606 | 46980 | 46995 | AAATTCAGCTTTAAGG | 31 | 1777 |
| 651795 | 4620 | 4635 | 47009 | 47024 | CCAGTATTAACACAGA | 70 | 973 |
| 651798 | 4705 | 4720 | 47094 | 47109 | AAGAACTCATGTGAGT | 40 | 1778 |
| 651799 | 4719 | 4734 | 47108 | 47123 | TATGACTATTCTTCAA | 46 | 1779 |
| 651804 | 4792 | 4807 | 47181 | 47196 | CATCTAAATTACCTAT | 0 | 1780 |
| 651809 | 4899 | 4914 | 47288 | 47303 | TTGGAAACTTTCGGAT | 32 | 1781 |
| 663600 | 4637 | 4652 | 47026 | 47041 | TGCAGAATTCATGCTA | 15 | 1782 |
| 696368 | 4387 | 4402 | 46776 | 46791 | CAAACTGTAACTTAAC | 31 | 1783 |
| 696369 | 4406 | 4421 | 46795 | 46810 | CAAATGAGATGAACTT | 29 | 1784 |
| 696370 | 4408 | 4423 | 46797 | 46812 | TACAAATGAGATGAAC | 13 | 1785 |
| 696371 | 4458 | 4473 | 46847 | 46862 | ATATACTGTTTGAAGA | 14 | 1786 |
| 696372 | 4475 | 4490 | 46864 | 46879 | ATCCCCTAAAAAAAGT | 14 | 1787 |
| 696373 | 4498 | 4513 | 46887 | 46902 | TAGTTTTTGCTGTCTA | 29 | 1788 |
| 696374 | 4500 | 4515 | 46889 | 46904 | GATAGTTTTTGCTGTC | 37 | 1789 |
| 696375 | 4510 | 4525 | 46899 | 46914 | GAAATCTTCAGATAGT | 43 | 1790 |
| 696376 | 4512 | 4527 | 46901 | 46916 | TGGAAATCTTCAGATA | 32 | 1791 |
| 696377 | 4525 | 4540 | 46914 | 46929 | ACTTTTTGACAAATGG | 76 | 981 |
| 696378 | 4530 | 4545 | 46919 | 46934 | TCATTACTTTTTGACA | 0 | 982 |
| 696379 | 4544 | 4559 | 46933 | 46948 | CAATTATCAAGAAATC | 2 | 1792 |
| 696380 | 4549 | 4564 | 46938 | 46953 | CTACACAATTATCAAG | 8 | 1793 |
| 696381 | 4554 | 4569 | 46943 | 46958 | CATTACTACACAATTA | 3 | 1794 |
| 696382 | 4556 | 4571 | 46945 | 46960 | AACATTACTACACAAT | 16 | 1795 |
| 696383 | 4586 | 4601 | 46975 | 46990 | CAGCTTTAAGGTAACT | 51 | 1796 |
| 696384 | 4589 | 4604 | 46978 | 46993 | ATTCAGCTTTAAGGTA | 57 | 1797 |
| 696385 | 4617 | 4632 | 47006 | 47021 | GTATTAACACAGAAGT | 46 | 983 |
| 696386 | 4622 | 4637 | 47011 | 47026 | ATCCAGTATTAACACA | 43 | 984 |
| 696387 | 4628 | 4643 | 47017 | 47032 | CATGCTATCCAGTATT | 45 | 1798 |
| 696388 | 4631 | 4646 | 47020 | 47035 | ATTCATGCTATCCAGT | 52 | 1799 |
| 696389 | 4633 | 4648 | 47022 | 47037 | GAATTCATGCTATCCA | 57 | 1800 |
| 696390 | 4639 | 4654 | 47028 | 47043 | AATGCAGAATTCATGC | 43 | 1801 |
| 696391 | 4662 | 4677 | 47051 | 47066 | TTATGACAGCTATTCA | 48 | 1802 |
| 696392 | 4697 | 4712 | 47086 | 47101 | ATGTGAGTATCTTTCT | 46 | 1803 |
| 696393 | 4700 | 4715 | 47089 | 47104 | CTCATGTGAGTATCTT | 62 | 1804 |
| 696394 | 4707 | 4722 | 47096 | 47111 | TCAAGAACTCATGTGA | 33 | 1805 |
| 696395 | 4709 | 4724 | 47098 | 47113 | CTTCAAGAACTCATGT | 7 | 1806 |
| 696396 | 4713 | 4728 | 47102 | 47117 | TATTCTTCAAGAACTC | 50 | 1807 |
| 696397 | 4715 | 4730 | 47104 | 47119 | ACTATTCTTCAAGAAC | 39 | 1808 |
| 696398 | 4717 | 4732 | 47106 | 47121 | TGACTATTCTTCAAGA | 39 | 1809 |
| 696399 | 4724 | 4739 | 47113 | 47128 | CTAGTTATGACTATTC | 56 | 1810 |
| 696400 | 4726 | 4741 | 47115 | 47130 | ATCTAGTTATGACTAT | 19 | 1811 |
| 696401 | 4728 | 4743 | 47117 | 47132 | TAATCTAGTTATGACT | 9 | 1812 |
| 696402 | 4733 | 4748 | 47122 | 47137 | GATCTTAATCTAGTTA | 25 | 1813 |
| 696403 | 4735 | 4750 | 47124 | 47139 | CAGATCTTAATCTAGT | 51 | 1814 |
| 696404 | 4737 | 4752 | 47126 | 47141 | CACAGATCTTAATCTA | 39 | 1815 |
| 696405 | 4763 | 4778 | 47152 | 47167 | AGGCACTTCAAACTAT | 39 | 1816 |
| 696406 | 4766 | 4781 | 47155 | 47170 | AACAGGCACTTCAAAC | 20 | 1817 |
| 696407 | 4768 | 4783 | 47157 | 47172 | CAAACAGGCACTTCAA | 33 | 1818 |
| 696408 | 4772 | 4787 | 47161 | 47176 | ATCCCAAACAGGCACT | 45 | 1819 |
| 696409 | 4775 | 4790 | 47164 | 47179 | ATTATCCCAAACAGGC | 53 | 1820 |
| 696410 | 4782 | 4797 | 47171 | 47186 | ACCTATCATTATCCCA | 48 | 1821 |
| 696411 | 4785 | 4800 | 47174 | 47189 | ATTACCTATCATTATC | 24 | 1822 |
| 696412 | 4790 | 4805 | 47179 | 47194 | TCTAAATTACCTATCA | 16 | 1823 |
| 696413 | 4795 | 4810 | 47184 | 47199 | ATTCATCTAAATTACC | 4 | 1824 |
| 696414 | 4802 | 4817 | 47191 | 47206 | CCCCTAAATTCATCTA | 18 | 1825 |
| 696415 | 4824 | 4839 | 47213 | 47228 | TATCTGCAGATAACTT | 3 | 1826 |
| 696416 | 4826 | 4841 | 47215 | 47230 | CATATCTGCAGATAAC | 3 | 1827 |
| 696417 | 4828 | 4843 | 47217 | 47232 | AACATATCTGCAGATA | 0 | 1828 |
| 696418 | 4833 | 4848 | 47222 | 47237 | CCCTCAACATATCTGC | 20 | 1829 |
| 696419 | 4869 | 4884 | 47258 | 47273 | CAGTTAGCTCTGTGGG | 48 | 1830 |
| 696420 | 4879 | 4894 | 47268 | 47283 | CACTGTAACCCAGTTA | 37 | 1831 |
| 696421 | 4881 | 4896 | 47270 | 47285 | AACACTGTAACCCAGT | 58 | 1832 |
| 696422 | 4883 | 4898 | 47272 | 47287 | AAAACACTGTAACCCA | 44 | 1833 |
| 696423 | 4889 | 4904 | 47278 | 47293 | TCGGATAAAACACTGT | 46 | 1834 |
| 696424 | 4891 | 4906 | 47280 | 47295 | TTTCGGATAAAACACT | 14 | 1835 |
| 696425 | 4895 | 4910 | 47284 | 47299 | AAACTTTCGGATAAAA | 0 | 1836 |
| 696426 | 4897 | 4912 | 47286 | 47301 | GGAAACTTTCGGATAA | 56 | 1837 |
| 696427 | 4904 | 4919 | 47293 | 47308 | TGGAATTGGAAACTTT | 48 | 1838 |
| 696428 | 4906 | 4921 | 47295 | 47310 | AGTGGAATTGGAAACT | 14 | 1839 |
| 696429 | 4913 | 4928 | 47302 | 47317 | ACAAGACAGTGGAATT | 31 | 1840 |
| 696430 | 4917 | 4932 | 47306 | 47321 | AAACACAAGACAGTGG | 17 | 1841 |
| 696431 | 4959 | 4974 | 47348 | 47363 | ATTGGCACTCAAAGGA | 35 | 1842 |
| 696432 | 4961 | 4976 | 47350 | 47365 | AAATTGGCACTCAAAG | 30 | 1843 |

TABLE 28
Inhibition of K-Ras mRNA by 3-10-3 cEt gapmers targeting SEQ ID NO: 1, 2 and 3

| | SEQ ID | SEQ ID | SEQ ID | SEQ ID | SEQ ID | SEQ ID | | | |
|---|---|---|---|---|---|---|---|---|---|
| | NO: 1 | NO: 1 | NO: 2 | NO: 2 | NO: 3 | NO: 3 | | | SEQ |
| ISIS | Start | Stop | Start | Stop | Start | Stop | | % | ID |
| NO | Site | Site | Site | Site | Site | Site | Sequence | Inhibition | NO |
| 540806 | 2981 | 2996 | 45370 | 45385 | 3105 | 3120 | GCATGAAGATTTCTGG | 77 | 122 |
| 540806 | 2981 | 2996 | 45370 | 45385 | 3105 | 3120 | GCATGAAGATTTCTGG | 81 | 122 |
| 651813 | 5033 | 5048 | 47422 | 47437 | 5157 | 5172 | GTTTACACTATACAAA | 18 | 1844 |
| 651814 | 5039 | 5054 | 47428 | 47443 | 5163 | 5178 | GTTTCAGTTTACACTA | 43 | 1845 |
| 651815 | 5054 | 5069 | 47443 | 47458 | 5178 | 5193 | GTACAAAATGTGCATG | 26 | 1846 |
| 651816 | 5103 | 5118 | 47492 | 47507 | 5227 | 5242 | GGAAAACAACTGGATC | 40 | 1847 |
| 651818 | 5144 | 5159 | 47533 | 47548 | 5268 | 5283 | TTGATATGACCAACAT | 0 | 1848 |
| 651830 | N/A | N/A | 37421 | 37436 | 681 | 696 | CAATCTGTATTGTCGG | 67 | 1849 |
| 651837 | N/A | N/A | 3302 | 3317 | N/A | N/A | AGTCTATTTCAGGCGG | 76 | 1850 |
| 663623 | 5065 | 5080 | 47454 | 47469 | 5189 | 5204 | GAAAGCACAATGTACA | 24 | 1851 |
| 663626 | 5086 | 5101 | 47475 | 47490 | 5210 | 5225 | CACTGCATATGTCCCA | 54 | 1852 |
| 663627 | 5094 | 5109 | 47483 | 47498 | 5218 | 5233 | CTGGATCACACTGCAT | 49 | 1853 |
| 663630 | 5123 | 5138 | 47512 | 47527 | 5247 | 5262 | GGTCAGCGCAACCAAA | 57 | 1854 |
| 663635 | 5150 | 5165 | 47539 | 47554 | 5274 | 5289 | TAATGTTTGATATGAC | 0 | 1855 |
| 696433 | 4968 | 4983 | 47357 | 47372 | 5092 | 5107 | TAGTAAGAAATTGGCA | 32 | 1856 |
| 696434 | 4973 | 4988 | 47362 | 47377 | 5097 | 5112 | AGTACTAGTAAGAAAT | 10 | 1857 |
| 696435 | 4975 | 4990 | 47364 | 47379 | 5099 | 5114 | ATAGTACTAGTAAGAA | 19 | 1858 |
| 696436 | 4977 | 4992 | 47366 | 47381 | 5101 | 5116 | AAATAGTACTAGTAAG | 9 | 1859 |
| 696437 | 4984 | 4999 | 47373 | 47388 | 5108 | 5123 | CATTAAGAAATAGTAC | 12 | 1860 |
| 696438 | 5000 | 5015 | 47389 | 47404 | 5124 | 5139 | CCAGGTAAACATGTTA | 59 | 1861 |
| 696439 | 5002 | 5017 | 47391 | 47406 | 5126 | 5141 | TTCCAGGTAAACATGT | 38 | 1862 |
| 696440 | 5004 | 5019 | 47393 | 47408 | 5128 | 5143 | CATTCCAGGTAAACAT | 47 | 1863 |
| 696441 | 5035 | 5050 | 47424 | 47439 | 5159 | 5174 | CAGTTTACACTATACA | 46 | 1864 |
| 696442 | 5041 | 5056 | 47430 | 47445 | 5165 | 5180 | ATGTTTCAGTTTACAC | 34 | 1865 |
| 696443 | 5045 | 5060 | 47434 | 47449 | 5169 | 5184 | GTGCATGTTTCAGTTT | 44 | 1866 |
| 696444 | 5079 | 5094 | 47468 | 47483 | 5203 | 5218 | TATGTCCCACAAAAGA | 38 | 1867 |
| 696445 | 5082 | 5097 | 47471 | 47486 | 5206 | 5221 | GCATATGTCCCACAAA | 51 | 1868 |
| 696446 | 5084 | 5099 | 47473 | 47488 | 5208 | 5223 | CTGCATATGTCCCACA | 62 | 1869 |
| 696447 | 5099 | 5114 | 47488 | 47503 | 5223 | 5238 | AACAACTGGATCACAC | 27 | 1870 |
| 696448 | 5101 | 5116 | 47490 | 47505 | 5225 | 5240 | AAAACAACTGGATCAC | 23 | 1871 |
| 696449 | 5115 | 5130 | 47504 | 47519 | 5239 | 5254 | CAACCAAATGATGGAA | 54 | 1872 |
| 696450 | 5128 | 5143 | 47517 | 47532 | 5252 | 5267 | TCCTAGGTCAGCGCAA | 33 | 1873 |
| 696451 | 5130 | 5145 | 47519 | 47534 | 5254 | 5269 | ATTCCTAGGTCAGCGC | 71 | 1874 |
| 696452 | 5134 | 5149 | 47523 | 47538 | 5258 | 5273 | CAACATTCCTAGGTCA | 25 | 1875 |
| 696453 | 5140 | 5155 | 47529 | 47544 | 5264 | 5279 | TATGACCAACATTCCT | 29 | 1876 |
| 696454 | 5142 | 5157 | 47531 | 47546 | 5266 | 5281 | GATATGACCAACATTC | 27 | 1877 |
| 696455 | 5148 | 5163 | 47537 | 47552 | 5272 | 5287 | ATGTTTGATATGACCA | 35 | 1878 |
| 696456 | 5170 | 5185 | 47559 | 47574 | 5294 | 5309 | ATTAAAAGAGTGGTCA | 25 | 1879 |
| 696457 | 5172 | 5187 | 47561 | 47576 | 5296 | 5311 | CAATTAAAAGAGTGGT | 13 | 1880 |
| 696458 | 5206 | 5221 | 47595 | 47610 | 5330 | 5345 | GCACATACTCCTATAA | 29 | 1881 |
| 696459 | 5213 | 5228 | 47602 | 47617 | 5337 | 5352 | CTTCACAGCACATACT | 17 | 1882 |
| 696460 | 5215 | 5230 | 47604 | 47619 | 5339 | 5354 | CACTTCACAGCACATA | 43 | 1883 |
| 696461 | 5221 | 5236 | 47610 | 47625 | 5345 | 5360 | TTAGATCACTTCACAG | 32 | 1884 |
| 696462 | 5223 | 5238 | 47612 | 47627 | 5347 | 5362 | TTTTAGATCACTTCAC | 25 | 1885 |
| 696463 | 5251 | 5266 | 47640 | 47655 | 5375 | 5390 | GTACAGTTCATGACAA | 35 | 1886 |
| 696464 | 5254 | 5269 | 47643 | 47658 | 5378 | 5393 | GTAGTACAGTTCATGA | 33 | 1887 |
| 696465 | 5256 | 5271 | 47645 | 47660 | 5380 | 5395 | GAGTAGTACAGTTCAT | 34 | 1888 |
| 696466 | 5261 | 5276 | 47650 | 47665 | 5385 | 5400 | ATTAGGAGTAGTACAG | 12 | 1889 |
| 696467 | 5263 | 5278 | 47652 | 47667 | 5387 | 5402 | TAATTAGGAGTAGTAC | 12 | 1890 |
| 696468 | 5265 | 5280 | 47654 | 47669 | 5389 | 5404 | AATAATTAGGAGTAGT | 16 | 1891 |
| 696469 | 5271 | 5286 | 47660 | 47675 | 5395 | 5410 | CATTACAATAATTAGG | 0 | 1892 |
| 696470 | 5293 | 5308 | 47682 | 47697 | 5417 | 5432 | GTCACTGTAACTATTT | 7 | 1893 |
| 696473 | N/A | N/A | 37399 | 37414 | 659 | 674 | CTCACCAATGTATAAA | 17 | 1894 |
| 696474 | N/A | N/A | 37406 | 37421 | 666 | 681 | GATCTCCCTCACCAAT | 4 | 1895 |
| 696475 | N/A | N/A | 37413 | 37428 | 673 | 688 | ATTGTCGGATCTCCCT | 39 | 1896 |
| 696476 | N/A | N/A | 37419 | 37434 | 679 | 694 | ATCTGTATTGTCGGAT | 25 | 1897 |
| 696477 | N/A | N/A | 37423 | 37438 | 683 | 698 | TTCAATCTGTATTGTC | 48 | 1898 |
| 696478 | N/A | N/A | 37453 | 37468 | 713 | 728 | CCAGGAGTCTTTTCTT | 35 | 1899 |
| 696479 | N/A | N/A | 37458 | 37473 | 718 | 733 | CACAGCCAGGAGTCTT | 43 | 1900 |
| 696480 | N/A | N/A | 37465 | 37480 | 725 | 740 | ATTTTCACACAGCCAG | 58 | 1901 |
| 696481 | N/A | N/A | 37467 | 37482 | 727 | 742 | TAATTTTCACACAGCC | 52 | 1902 |
| 696482 | N/A | N/A | 37489 | 37504 | 749 | 764 | GATTACATTATAATGC | 15 | 1903 |
| 696483 | N/A | N/A | 37492 | 37507 | 752 | 767 | CCAGATTACATTATAA | 28 | 1904 |
| 696484 | N/A | N/A | N/A | N/A | 756 | 771 | ACACCCAGATTACATT | 12 | 1905 |
| 696485 | N/A | N/A | N/A | N/A | 761 | 776 | CATCAACACCCAGATT | 0 | 1906 |
| 696486 | N/A | N/A | N/A | N/A | 763 | 778 | ATCATCAACACCCAGA | 37 | 1907 |
| 696487 | N/A | N/A | 2233 | 2248 | N/A | N/A | CGGCAAAGAGGGTCGG | 0 | 1908 |
| 696488 | N/A | N/A | 2550 | 2565 | N/A | N/A | AACCTCCACCGCACCC | 2 | 1909 |
| 696489 | N/A | N/A | 2715 | 2730 | N/A | N/A | ACCACTATCCGTCCAG | 45 | 1910 |
| 696490 | N/A | N/A | 2817 | 2832 | N/A | N/A | CCAAACACAATAACCT | 32 | 1911 |
| 696491 | N/A | N/A | 3068 | 3083 | N/A | N/A | CAACTAGCAAGGAAAA | 17 | 1912 |
| 696492 | N/A | N/A | 3175 | 3190 | N/A | N/A | AGTATAAAAGAGACGA | 25 | 1913 |
| 696493 | N/A | N/A | 3951 | 3966 | N/A | N/A | GTTAATTCTGAGCTGA | 53 | 1914 |
| 696494 | N/A | N/A | 3991 | 4006 | N/A | N/A | CATTTTGGACCTCAGT | 33 | 1915 |
| 696495 | N/A | N/A | 3993 | 4008 | N/A | N/A | AGCATTTTGGACCTCA | 71 | 1916 |
| 696496 | N/A | N/A | 4065 | 4080 | N/A | N/A | ATGGCTACAGTCTCAA | 35 | 1917 |
| 696497 | N/A | N/A | 4079 | 4094 | N/A | N/A | CAAATATACTGTGGAT | 26 | 1918 |

TABLE 29
Inhibition of K-Ras mRNA by 3-10-3
cEt gapmers targeting SEQ ID NO: 1 and 2

| | SEQ ID | SEQ ID | | | |
|---|---|---|---|---|---|
| | NO: 2 | NO: 2 | | % | SEQ |
| ISIS | Start | Stop | | Inhibi- | ID |
| NO | Site | Site | Sequence | tion | NO |
| 540806 | 45370 | 45385 | GCATGAAGATTTCTGG | 69 | 122 |
| 540806 | 45370 | 45385 | GCATGAAGATTTCTGG | 77 | 122 |
| 663720 | 27165 | 27180 | TAATTTGTTCTCTGGG | 22 | 1919 |
| 696658 | 23436 | 23451 | GAACTGCAACTATAAG | 18 | 1920 |
| 696659 | 23439 | 23454 | AGAGAACTGCAACTAT | 22 | 1921 |
| 696660 | 23507 | 23522 | ATCTCTAAAGAGCAAT | 24 | 1922 |
| 696661 | 23579 | 23594 | CAATACTCAAGATTCT | 18 | 1923 |
| 696662 | 23688 | 23703 | CAACTCTATTATTCAA | 14 | 1924 |
| 696663 | 24168 | 24183 | CTTAAAATTAACTACC | 0 | 1925 |
| 696664 | 24292 | 24307 | CAGGTACAGAATTCTA | 31 | 1926 |
| 696665 | 24486 | 24501 | AACCTGTATATACATG | 20 | 1927 |
| 696666 | 24583 | 24598 | GAACCAGTTAAGTATC | 28 | 1928 |
| 696667 | 24605 | 24620 | GGATTTTTGGACGAGG | 45 | 1929 |
| 696668 | 24889 | 24904 | ATAGGTTGAGCATTAA | 34 | 1930 |
| 696669 | 24895 | 24910 | TTTCATATAGGTTGAG | 28 | 1931 |
| 696670 | 25198 | 25213 | AAATCTTTGTGCATTG | 16 | 1932 |
| 696671 | 25489 | 25504 | TTATTACAGTGCACCT | 5 | 1933 |
| 696672 | 25494 | 25509 | CTGGATTATTACAGTG | 1 | 1934 |
| 696673 | 25499 | 25514 | ACAGTCTGGATTATTA | 5 | 1935 |
| 696674 | 25501 | 25516 | ACACAGTCTGGATTAT | 0 | 1936 |
| 696675 | 25503 | 25518 | AAACACAGTCTGGATT | 12 | 1937 |
| 696676 | 25696 | 25711 | ACCTATAATGGTGAAT | 1 | 1938 |
| 696677 | 25698 | 25713 | CCACCTATAATGGTGA | 0 | 1939 |
| 696678 | 25701 | 25716 | AACCCACCTATAATGG | 8 | 1940 |
| 696679 | 25704 | 25719 | TTAAACCCACCTATAA | 10 | 1941 |
| 696680 | 25706 | 25721 | ATTTAAACCCACCTAT | 0 | 1942 |
| 696681 | 25855 | 25870 | CCCCCAAGAACTTCAT | 3 | 1943 |
| 696682 | 26058 | 26073 | GTTAAAGTGACACCAT | 40 | 1944 |
| 696683 | 26101 | 26116 | ATCCAAGCAATTCTAT | 5 | 1945 |
| 696684 | 26252 | 26267 | CCCTCAAAGAAATAGA | 11 | 1946 |
| 696685 | 26395 | 26410 | TATTACTAGACTATAC | 0 | 1947 |
| 696686 | 26396 | 26411 | CTATTACTAGACTATA | 0 | 1948 |
| 696687 | 26489 | 26504 | CCATTAGCTGGGTAAA | 31 | 1949 |
| 696688 | 26520 | 26535 | CAGAATTGGCTCAAAT | 13 | 1950 |
| 696689 | 26910 | 26925 | TTAATATGCAGGTAGA | 43 | 1951 |
| 696690 | 26921 | 26936 | AACCTAATAGGTTAAT | 4 | 1952 |
| 696691 | 26939 | 26954 | GAAGTATAGTAAAACT | 23 | 1953 |
| 696692 | 27497 | 27512 | AGCCAAAAGCAGTACC | 64 | 1954 |
| 696693 | 28073 | 28088 | TAGAAAGTATCCCTGT | 21 | 1955 |
| 696694 | 28150 | 28165 | GGTTATACTACCAAGG | 46 | 1956 |
| 696695 | 28205 | 28220 | ACAGGTTTGTATCCCT | 48 | 1957 |
| 696696 | 28230 | 28245 | AGTCATTAGTACAGTT | 44 | 1958 |
| 696697 | 28284 | 28299 | CCAAGTGTAGGTTTAG | 58 | 1959 |
| 696698 | 28347 | 28362 | AGTAAAGTAAGGTTAA | 24 | 1960 |
| 696699 | 28799 | 28814 | GTATAATGGTATAGCA | 50 | 1961 |
| 696700 | 28874 | 28889 | TAACACTGTAGTACGA | 10 | 1962 |
| 696701 | 29016 | 29031 | TATAGATGGATCAATT | 28 | 1963 |
| 696702 | 29038 | 29053 | AGCCCTAAACAAATTG | 36 | 1964 |
| 696703 | 29443 | 29458 | GTAAAGTGATATATGA | 22 | 1965 |
| 696704 | 30003 | 30018 | CTCTTTTTATGTCCTC | 56 | 1966 |
| 696705 | 30110 | 30125 | ATTAGTACTTCTGAGG | 41 | 1967 |
| 696706 | 30205 | 30220 | CCTAAAAATCTCTTAT | 0 | 1968 |
| 696707 | 30356 | 30371 | AAGTATTCTTTCATAC | 5 | 1969 |
| 696708 | 30418 | 30433 | TACATAATAACATCAG | 24 | 1970 |
| 696709 | 30590 | 30605 | CTTTAAAGTCTTCCAG | 43 | 1971 |
| 696710 | 30673 | 30688 | ATTTTCACCAGTAACT | 19 | 1972 |
| 696711 | 30706 | 30721 | TAACAAAATACTCTGC | 0 | 1973 |
| 696712 | 31090 | 31105 | GCACACTAATTTTGTT | 41 | 1974 |
| 696713 | 31124 | 31139 | AAAACAACTTGCCGAT | 52 | 1975 |
| 696714 | 31150 | 31165 | GATCAAGACCCCAAAA | 26 | 1976 |
| 696715 | 31361 | 31376 | AACGATTTTTGCATTT | 52 | 1977 |
| 696716 | 31759 | 31774 | ACTAAAGTTACCCAGA | 38 | 1978 |
| 696717 | 31816 | 31831 | TTAAAGTTAGCCTGTA | 29 | 1979 |
| 696718 | 32195 | 32210 | AAATACTAGAGACCAG | 0 | 1980 |
| 696719 | 32583 | 32598 | TATGTAACGCATTATA | 36 | 1981 |
| 696720 | 32735 | 32750 | GTCCAAAGGGACCAGG | 31 | 1982 |
| 696721 | 32833 | 32848 | AACCCTCCCACTTTTG | 17 | 1983 |
| 696722 | 33039 | 33054 | AAAGCATTCTTTAACG | 26 | 1984 |
| 696723 | 33293 | 33308 | ACAAGATGTATTCTAA | 24 | 1985 |
| 696724 | 33365 | 33380 | CAACACATCAAATACC | 22 | 1986 |
| 696725 | 33478 | 33493 | CCAAAGTATCATTCTA | 41 | 1987 |
| 696726 | 33514 | 33529 | GAAACAAAGCACTCCA | 44 | 1988 |
| 696727 | 33551 | 33566 | CTCAACTATTATCTGA | 23 | 1989 |
| 696728 | 33642 | 33657 | CTTTAAGAACAACTGA | 35 | 1990 |
| 696729 | 34076 | 34091 | TAGCACACAATAATTT | 14 | 1991 |
| 696730 | 34367 | 34382 | ATAAGAAACTTAGGTT | 8 | 1992 |
| 696731 | 34412 | 34427 | TAATTAACAGCACAGG | 64 | 1993 |
| 696732 | 34488 | 34503 | TTGGAAGCCAATAATT | 19 | 1994 |

### Example 8: Antisense Inhibition of Human K-Ras in HepG2 Cells by cEt Gapmers

Antisense oligonucleotides were designed targeting a K-Ras nucleic acid and were tested for their effects on K-Ras mRNA in vitro. Cultured HepG2 cells at a density of 20,000 cells per well were transfected using electroporation with 4,000 nM antisense oligonucleotide. After a treatment period of approximately 24 hours, RNA was isolated from the cells and K-Ras mRNA levels were measured by quantitative real-time PCR. Human primer probe set RTS132 was used to measure mRNA levels. K-Ras mRNA levels were adjusted according to total RNA content, as measured by RIBOGREEN®. Results are presented as percent inhibition of K-Ras, relative to untreated control cells. As used herein, a value of ‘0’ indicates that treatment with the antisense oligonucleotide did not inhibit mRNA levels.

The newly designed chimeric antisense oligonucleotides in the Tables below were designed as 3-10-3 cEt gapmers. The gapmers are 16 nucleosides in length, wherein the central gap segment comprises of ten 2′-deoxynucleosides and is flanked by wing segments on the 5′ direction and the 3′ direction comprising three nucleosides each. Each nucleoside in the 5′ wing segment and each nucleoside in the 3′ wing segment has a cEt sugar modification. The internucleoside linkages throughout each gapmer are phosphorothioate (P═S) linkages. All cytosine residues throughout each gapmer are 5-methylcytosines. “Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either SEQ ID NO: 1 or SEQ ID NO: 2. Certain antisense oligonucleotides target the target sequence with one mismatch. These antisense oligonucleotides are presented in the Table below with bold underlining on the mismatched nucleoside.

TABLE 30
Inhibition of K-Ras mRNA by 3-10-3 cEt gapmers targeting SEQ ID NO: 1 and 2

| | SEQ ID | SEQ ID | SEQ ID | SEQ ID | | | | |
|---|---|---|---|---|---|---|---|---|
| | NO: 1 | NO: 1 | NO: 2 | NO: 2 | | | | SEQ |
| ISIS | Start | Stop | Start | Stop | | | % | ID |
| NO | Site | Site | Site | Site | Mismatches | Sequence | Inhibition | NO |
| 540737 | 219 | 234 | 7585 | 7600 | 0 | TACGCCACCAGCTCCA | 68 | 1995 |
| 540738 | 220 | 235 | 7586 | 7601 | 0 | CTACGCCACCAGCTCC | 64 | 1996 |
| 540739 | 221 | 236 | 7587 | 7602 | 0 | CCTACGCCACCAGCTC | 86 | 1997 |
| 540740 | 222 | 237 | 7588 | 7603 | 0 | GCCTACGCCACCAGCT | 67 | 1998 |
| 540801 | 2818 | 2833 | 45207 | 45222 | 0 | GTCGCTAATGGATTGG | 85 | 46 |
| 540806 | 2981 | 2996 | 45370 | 45385 | 0 | GCATGAAGATTTCTGG | 52 | 122 |
| 540806 | 2981 | 2996 | 45370 | 45385 | 0 | GCATGAAGATTTCTGG | 85 | 122 |
| 540808 | 3376 | 3391 | 45765 | 45780 | 0 | ATGTCTCTTGTTTGGG | 85 | 123 |
| 651530 | 1313 | 1328 | 43702 | 43717 | 0 | TGACTAATAGCAGTGG | 90 | 239 |
| 651634 | 2634 | 2649 | 45023 | 45038 | 0 | AATCTTATGGTTAGGG | 85 | 316 |
| 651645 | 2733 | 2748 | 45122 | 45137 | 0 | TATCTGTCAGATTCTC | 87 | 321 |
| 651733 | 3893 | 3908 | 46282 | 46297 | 0 | GGTACTGTGTAAGTCT | 90 | 1352 |
| 651972 | 1213 | 1228 | 43602 | 43617 | 0 | TCATCAGGAAGCCCAT | 79 | 226 |
| 651987 | 1447 | 1462 | 43836 | 43851 | 0 | GCTATTAGGAGTCTTT | 89 | 272 |
| 651990 | 1493 | 1508 | 43882 | 43897 | 0 | GCTATAATAATCCCCA | 88 | 275 |
| 652004 | 1685 | 1700 | 44074 | 44089 | 0 | TTAATGTCACAAGCAG | 92 | 289 |
| 652019 | 1918 | 1933 | 44307 | 44322 | 0 | CTTGATTTGTCAGCAG | 92 | 304 |
| 652132 | 4036 | 4051 | 46425 | 46440 | 0 | ACCATTCAAAGTTCAC | 88 | 975 |
| 663454 | 225 | 240 | 7591 | 7606 | 0 | CTTGCCTACGCCACCA | 66 | 1999 |
| 667541 | 211 | 226 | 7577 | 7592 | 0 | CAGCTCCAACTACCAC | 87 | 2000 |
| 667542 | 212 | 227 | 7578 | 7593 | 0 | CCAGCTCCAACTACCA | 81 | 2001 |
| 667543 | 213 | 228 | 7579 | 7594 | 0 | ACCAGCTCCAACTACC | 76 | 2002 |
| 667544 | 214 | 229 | 7580 | 7595 | 0 | CACCAGCTCCAACTAC | 64 | 2003 |
| 667545 | 215 | 230 | 7581 | 7596 | 0 | CCACCAGCTCCAACTA | 70 | 2004 |
| 667546 | 216 | 231 | 7582 | 7597 | 0 | GCCACCAGCTCCAACT | 70 | 2005 |
| 667547 | 217 | 232 | 7583 | 7598 | 0 | CGCCACCAGCTCCAAC | 78 | 2006 |
| 667548 | 218 | 233 | 7584 | 7599 | 0 | ACGCCACCAGCTCCAA | 89 | 2007 |
| 667549 | 223 | 238 | 7589 | 7604 | 0 | TGCCTACGCCACCAGC | 78 | 2008 |
| 667550 | 224 | 239 | 7590 | 7605 | 0 | TTGCCTACGCCACCAG | 82 | 550 |
| 667551 | 226 | 241 | 7592 | 7607 | 0 | TCTTGCCTACGCCACC | 67 | 2009 |
| 667552 | 227 | 242 | 7593 | 7608 | 0 | CTCTTGCCTACGCCAC | 74 | 2010 |
| 667553 | 211 | 226 | 7577 | 7592 | 1 | AAGCTCCAACTACCAC | 81 | 2011 |
| 667554 | 212 | 227 | 7578 | 7593 | 1 | CAAGCTCCAACTACCA | 82 | 2012 |
| 667555 | 213 | 228 | 7579 | 7594 | 1 | ACAAGCTCCAACTACC | 41 | 2013 |
| 667556 | 214 | 229 | 7580 | 7595 | 1 | CACAAGCTCCAACTAC | 34 | 2014 |
| 667557 | 215 | 230 | 7581 | 7596 | 1 | CCACAAGCTCCAACTA | 49 | 2015 |
| 667558 | 216 | 231 | 7582 | 7597 | 1 | GCCACAAGCTCCAACT | 47 | 2016 |
| 667559 | 217 | 232 | 7583 | 7598 | 1 | CGCCACAAGCTCCAAC | 55 | 2017 |
| 667560 | 218 | 233 | 7584 | 7599 | 1 | ACGCCACAAGCTCCAA | 64 | 2018 |
| 667561 | 219 | 234 | 7585 | 7600 | 1 | TACGCCACAAGCTCCA | 72 | 2019 |
| 667562 | 220 | 235 | 7586 | 7601 | 1 | CTACGCCACAAGCTCC | 60 | 2020 |
| 667563 | 221 | 236 | 7587 | 7602 | 1 | CCTACGCCACAAGCTC | 56 | 2021 |
| 667564 | 222 | 237 | 7588 | 7603 | 1 | GCCTACGCCACAAGCT | 47 | 2022 |
| 667565 | 223 | 238 | 7589 | 7604 | 1 | TGCCTACGCCACAAGC | 47 | 2023 |
| 667566 | 224 | 239 | 7590 | 7605 | 1 | TTGCCTACGCCACAAG | 62 | 2024 |
| 667567 | 225 | 240 | 7591 | 7606 | 1 | CTTGCCTACGCCACAA | 62 | 2025 |
| 667568 | 226 | 241 | 7592 | 7607 | 1 | TCTTGCCTACGCCACA | 73 | 2026 |
| 667569 | 212 | 227 | 7578 | 7593 | 1 | TCAGCTCCAACTACCA | 79 | 2027 |
| 667570 | 213 | 228 | 7579 | 7594 | 1 | ATCAGCTCCAACTACC | 61 | 2028 |
| 667571 | 214 | 229 | 7580 | 7595 | 1 | CATCAGCTCCAACTAC | 28 | 2029 |
| 667572 | 215 | 230 | 7581 | 7596 | 1 | CCATCAGCTCCAACTA | 36 | 2030 |
| 667573 | 216 | 231 | 7582 | 7597 | 1 | GCCATCAGCTCCAACT | 6 | 2031 |
| 667574 | 217 | 232 | 7583 | 7598 | 1 | CGCCATCAGCTCCAAC | 16 | 2032 |
| 667575 | 218 | 233 | 7584 | 7599 | 1 | ACGCCATCAGCTCCAA | 57 | 2033 |
| 667576 | 219 | 234 | 7585 | 7600 | 1 | TACGCCATCAGCTCCA | 57 | 2034 |
| 667577 | 220 | 235 | 7586 | 7601 | 1 | CTACGCCATCAGCTCC | 58 | 2035 |
| 667578 | 221 | 236 | 7587 | 7602 | 1 | CCTACGCCATCAGCTC | 58 | 2036 |
| 667579 | 222 | 237 | 7588 | 7603 | 1 | GCCTACGCCATCAGCT | 0 | 2037 |
| 667580 | 223 | 238 | 7589 | 7604 | 1 | TGCCTACGCCATCAGC | 40 | 2038 |
| 667581 | 224 | 239 | 7590 | 7605 | 1 | TTGCCTACGCCATCAG | 58 | 2039 |
| 667582 | 225 | 240 | 7591 | 7606 | 1 | CTTGCCTACGCCATCA | 53 | 2040 |
| 667583 | 226 | 241 | 7592 | 7607 | 1 | TCTTGCCTACGCCATC | 58 | 2041 |
| 667584 | 227 | 242 | 7593 | 7608 | 1 | CTCTTGCCTACGCCAT | 73 | 2042 |
| 667585 | 212 | 227 | 7578 | 7593 | 1 | ACAGCTCCAACTACCA | 83 | 2043 |
| 667586 | 213 | 228 | 7579 | 7594 | 1 | AACAGCTCCAACTACC | 62 | 2044 |
| 667587 | 214 | 229 | 7580 | 7595 | 1 | CAACAGCTCCAACTAC | 28 | 2045 |
| 667588 | 215 | 230 | 7581 | 7596 | 1 | CCAACAGCTCCAACTA | 35 | 2046 |
| 667589 | 216 | 231 | 7582 | 7597 | 1 | GCCAACAGCTCCAACT | 26 | 2047 |
| 667590 | 217 | 232 | 7583 | 7598 | 1 | CGCCAACAGCTCCAAC | 37 | 2048 |
| 667591 | 218 | 233 | 7584 | 7599 | 1 | ACGCCAACAGCTCCAA | 83 | 2049 |
| 667592 | 219 | 234 | 7585 | 7600 | 1 | TACGCCAACAGCTCCA | 77 | 2050 |
| 667593 | 220 | 235 | 7586 | 7601 | 1 | CTACGCCAACAGCTCC | 70 | 2051 |
| 667594 | 221 | 236 | 7587 | 7602 | 1 | CCTACGCCAACAGCTC | 64 | 2052 |
| 667595 | 222 | 237 | 7588 | 7603 | 1 | GCCTACGCCAACAGCT | 29 | 2053 |
| 667596 | 223 | 238 | 7589 | 7604 | 1 | TGCCTACGCCAACAGC | 24 | 2054 |
| 667597 | 224 | 239 | 7590 | 7605 | 1 | TTGCCTACGCCAACAG | 51 | 2055 |
| 667598 | 225 | 240 | 7591 | 7606 | 1 | CTTGCCTACGCCAACA | 45 | 2056 |
| 667599 | 226 | 241 | 7592 | 7607 | 1 | TCTTGCCTACGCCAAC | 63 | 2057 |
| 667600 | 227 | 242 | 7593 | 7608 | 1 | CTCTTGCCTACGCCAA | 72 | 2058 |

### Example 9: Antisense Inhibition of Human K-Ras in A431 Cells

Antisense oligonucleotides were designed targeting a K-Ras nucleic acid and were tested for their effects on K-Ras mRNA in vitro. The antisense oligonucleotides were tested in a series of experiments that had similar culture conditions. The results for each experiment are presented in separate tables shown below. Cultured A431cells at a density of 5,000 cells per well were treated with 2,000 nM antisense oligonucleotide by free uptake. After a treatment period of approximately 24 hours, RNA was isolated from the cells and K-Ras mRNA levels were measured by quantitative real-time PCR. Human primer probe set RTS3496_MGB was used to measure mRNA levels. K-Ras mRNA levels were adjusted according to total RNA content, as measured by RIBOGREEN®. Results are presented as percent inhibition of K-Ras, relative to untreated control cells. As used herein, a value of ‘0’ indicates that treatment with the antisense oligonucleotide did not inhibit mRNA levels.

The newly designed chimeric antisense oligonucleotides in the Tables below were designed as 3-10-3 cEt gapmers or deoxy, MOE, and (S)-cEt gapmers. The 3-10-3 cEt gapmers are 16 nucleosides in length, wherein the central gap segment comprises of ten 2′-deoxynucleosides and is flanked by wing segments on the 5′ direction and the 3′ direction comprising three nucleosides each. The deoxy, MOE and (S)-cEt oligonucleotides are 16 nucleosides in length wherein the nucleoside have either a MOE sugar modification, an (S)-cEt sugar modification, or a deoxy modification. The ‘Chemistry’ column describes the sugar modifications of each oligonucleotide. ‘k’ indicates an (S)-cEt sugar modification; ‘d’ indicates deoxyribose; the number after ‘d’ indicates the number of deoxynucleosides; and ‘e’ indicates a MOE modification. The internucleoside linkages throughout each gapmer are phosphorothioate (P═S) linkages. All cytosine residues throughout each gapmer are 5-methylcytosines. “Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either SEQ ID NO: 1 or SEQ ID NO: 2. ‘N/A’ indicates that the antisense oligonucleotide does not target that particular gene sequence with 100% complementarity.

TABLE 31
Inhibition of K-Ras mRNA by gapmers targeting SEQ ID NO: 1 and 2

| | SEQ ID | SEQ ID | SEQ ID | SEQ ID | | | | |
|---|---|---|---|---|---|---|---|---|
| | NO: 1 | NO: 1 | NO: 2 | NO: 2 | | | | SEQ |
| ISIS | Start | Stop | Start | Stop | | | % | ID |
| NO | Site | Site | Site | Site | Sequence | Chemistry | Inhibition | NO |
| 651987 | 1447 | 1462 | 43836 | 43851 | GCTATTAGGAGTCTTT | kkk-d10-kkk | 49 | 272 |
| 651987 | 1447 | 1462 | 43836 | 43851 | GCTATTAGGAGTCTTT | kkk-d10-kkk | 62 | 272 |
| 695867 | 1130 | 1145 | 43519 | 43534 | TCCATTTATGTGACTA | kkk-d10-kkk | 46 | 506 |
| 695924 | 1441 | 1456 | 43830 | 43845 | AGGAGTCTTTATAGTA | kkk-d10-kkk | 65 | 420 |
| 695998 | 1790 | 1805 | 44179 | 44194 | ATGCTGTGAAACTCTC | kkk-d10-kkk | 52 | 658 |
| 696017 | 1917 | 1932 | 44306 | 44321 | TTGATTTGTCAGCAGG | kkk-d10-kkk | 66 | 677 |
| 696044 | 2115 | 2130 | 44504 | 44519 | GTGTTTATGCAATGTT | kkk-d10-kkk | 74 | 715 |
| 696091 | 2436 | 2451 | 44825 | 44840 | AGATTGTGCTGAGCTT | kkk-d10-kkk | 29 | 762 |
| 696096 | 2463 | 2478 | 44852 | 44867 | ATGGTGTAACATAGGT | kkk-d10-kkk | 47 | 914 |
| 696152 | 2761 | 2776 | 45150 | 45165 | TTAGTGATTAGGTCAA | kkk-d10-kkk | 51 | 924 |
| 716764 | 1917 | 1932 | 44306 | 44321 | TTGATTTGTCAGCAGG | kkk-d9-kkke | 30 | 890 |
| 716769 | 1917 | 1932 | 44306 | 44321 | TTGATTTGTCAGCAGG | kk-d10-keke | 45 | 892 |
| 716774 | 1917 | 1932 | 44306 | 44321 | TTGATTTGTCAGCAGG | kk-d9-kekek | 22 | 894 |
| 716779 | 1917 | 1932 | 44306 | 44321 | TTGATTTGTCAGCAGG | kk-d8-kekekk | 0 | 896 |
| 716789 | 1917 | 1932 | 44306 | 44321 | TTGATTTGTCAGCAGG | kkk-d8-kekek | 12 | 900 |
| 716804 | 1916 | 1931 | 44305 | 44320 | TGATTTGTCAGCAGGA | kk-d8-kekekk | 17 | 906 |
| 740162 | 1128 | 1143 | 43517 | 43532 | CATTTATGTGACTAGA | k-d10-kekek | 24 | 2059 |
| 740163 | 1439 | 1454 | 43828 | 43843 | GAGTCTTTATAGTAAT | k-d10-kekek | 11 | 2060 |
| 740164 | 1915 | 1930 | 44304 | 44319 | GATTTGTCAGCAGGAC | k-d10-kekek | 57 | 2061 |
| 740168 | 1130 | 1145 | 43519 | 43534 | TCCATTTATGTGACTA | k-d10-kekek | 38 | 2062 |
| 740169 | 1441 | 1456 | 43830 | 43845 | AGGAGTCTTTATAGTA | k-d10-kekek | 31 | 2063 |
| 740170 | 1917 | 1932 | 44306 | 44321 | TTGATTTGTCAGCAGG | k-d10-kekek | 17 | 2064 |
| 740174 | 1128 | 1143 | 43517 | 43532 | CATTTATGTGACTAGA | k-d9-kekeke | 24 | 2065 |
| 740175 | 1439 | 1454 | 43828 | 43843 | GAGTCTTTATAGTAAT | k-d9-kekeke | 21 | 2066 |
| 740176 | 1915 | 1930 | 44304 | 44319 | GATTTGTCAGCAGGAC | k-d9-kekeke | 32 | 2067 |
| 740180 | 1130 | 1145 | 43519 | 43534 | TCCATTTATGTGACTA | k-d9-kekeke | 15 | 2068 |
| 740181 | 1441 | 1456 | 43830 | 43845 | AGGAGTCTTTATAGTA | k-d9-kekeke | 24 | 2069 |
| 740182 | 1917 | 1932 | 44306 | 44321 | TTGATTTGTCAGCAGG | k-d9-kekeke | 22 | 2070 |
| 740186 | 1129 | 1144 | 43518 | 43533 | CCATTTATGTGACTAG | kk-d10-keke | 46 | 2071 |
| 740187 | 1440 | 1455 | 43829 | 43844 | GGAGTCTTTATAGTAA | kk-d10-keke | 55 | 2072 |
| 740188 | 1916 | 1931 | 44305 | 44320 | TGATTTGTCAGCAGGA | kk-d10-keke | 55 | 2073 |
| 740192 | 1130 | 1145 | 43519 | 43534 | TCCATTTATGTGACTA | kk-d10-keke | 22 | 2074 |
| 740193 | 1441 | 1456 | 43830 | 43845 | AGGAGTCTTTATAGTA | kk-d10-keke | 40 | 2075 |
| 740197 | 1129 | 1144 | 43518 | 43533 | CCATTTATGTGACTAG | kk-d8-kekekk | 0 | 2076 |
| 740198 | 1440 | 1455 | 43829 | 43844 | GGAGTCTTTATAGTAA | kk-d8-kekekk | 25 | 2077 |
| 740202 | 1130 | 1145 | 43519 | 43534 | TCCATTTATGTGACTA | kk-d8-kekekk | 29 | 2078 |
| 740203 | 1441 | 1456 | 43830 | 43845 | AGGAGTCTTTATAGTA | kk-d8-kekekk | 21 | 2079 |
| 740207 | 1129 | 1144 | 43518 | 43533 | CCATTTATGTGACTAG | kk-d9-kdkdk | 43 | 2080 |
| 740208 | 1440 | 1455 | 43829 | 43844 | GGAGTCTTTATAGTAA | kk-d9-kdkdk | 29 | 2081 |
| 740209 | 1916 | 1931 | 44305 | 44320 | TGATTTGTCAGCAGGA | kk-d9-kdkdk | 15 | 2082 |
| 740213 | 1129 | 1144 | 43518 | 43533 | CCATTTATGTGACTAG | kk-d9-kekek | 25 | 2083 |
| 740214 | 1440 | 1455 | 43829 | 43844 | GGAGTCTTTATAGTAA | kk-d9-kekek | 21 | 2084 |
| 740215 | 1916 | 1931 | 44305 | 44320 | TGATTTGTCAGCAGGA | kk-d9-kekek | 45 | 2085 |
| 740219 | 1130 | 1145 | 43519 | 43534 | TCCATTTATGTGACTA | kk-d9-kekek | 32 | 2086 |
| 740220 | 1441 | 1456 | 43830 | 43845 | AGGAGTCTTTATAGTA | kk-d9-kekek | 31 | 2087 |
| 740224 | 1128 | 1143 | 43517 | 43532 | CATTTATGTGACTAGA | kk-d8-kekekk | 20 | 2088 |
| 740225 | 1439 | 1454 | 43828 | 43843 | GAGTCTTTATAGTAAT | kk-d8-kekekk | 0 | 2089 |
| 740226 | 1915 | 1930 | 44304 | 44319 | GATTTGTCAGCAGGAC | kk-d8-kekekk | 0 | 2090 |
| 740230 | 1130 | 1145 | 43519 | 43534 | TCCATTTATGTGACTA | kkk-d8-kdkdk | 16 | 2091 |
| 740231 | 1441 | 1456 | 43830 | 43845 | AGGAGTCTTTATAGTA | kkk-d8-kdkdk | 30 | 2092 |
| 740232 | 1917 | 1932 | 44306 | 44321 | TTGATTTGTCAGCAGG | kkk-d8-kdkdk | 19 | 2093 |
| 740236 | 1130 | 1145 | 43519 | 43534 | TCCATTTATGTGACTA | kkk-d8-kekek | 0 | 2094 |
| 740237 | 1441 | 1456 | 43830 | 43845 | AGGAGTCTTTATAGTA | kkk-d8-kekek | 22 | 2095 |
| 740241 | 1129 | 1144 | 43518 | 43533 | CCATTTATGTGACTAG | kkk-d8-kekek | 36 | 2096 |
| 740242 | 1440 | 1455 | 43829 | 43844 | GGAGTCTTTATAGTAA | kkk-d8-kekek | 0 | 2097 |
| 740243 | 1916 | 1931 | 44305 | 44320 | TGATTTGTCAGCAGGA | kkk-d8-kekek | 41 | 2098 |
| 740247 | 1130 | 1145 | 43519 | 43534 | TCCATTTATGTGACTA | kkk-d9-keke | 8 | 2099 |
| 740248 | 1441 | 1456 | 43830 | 43845 | AGGAGTCTTTATAGTA | kkk-d9-keke | 31 | 2100 |
| 740249 | 1917 | 1932 | 44306 | 44321 | TTGATTTGTCAGCAGG | kkk-d9-keke | 40 | 2101 |
| 740253 | 1129 | 1144 | 43518 | 43533 | CCATTTATGTGACTAG | kkk-d9-kkke | 49 | 2102 |
| 740254 | 1440 | 1455 | 43829 | 43844 | GGAGTCTTTATAGTAA | kkk-d9-kkke | 41 | 2103 |
| 740255 | 1916 | 1931 | 44305 | 44320 | TGATTTGTCAGCAGGA | kkk-d9-kkke | 63 | 2104 |
| 740259 | 1130 | 1145 | 43519 | 43534 | TCCATTTATGTGACTA | kkk-d9-kkke | 23 | 2105 |
| 740260 | 1441 | 1456 | 43830 | 43845 | AGGAGTCTTTATAGTA | kkk-d9-kkke | 6 | 2106 |
| 740273 | 1788 | 1803 | 44177 | 44192 | GCTGTGAAACTCTCTA | k-d10-kekek | 41 | 2107 |
| 740275 | 1790 | 1805 | 44179 | 44194 | ATGCTGTGAAACTCTC | k-d10-kekek | 18 | 2108 |
| 740277 | 1788 | 1803 | 44177 | 44192 | GCTGTGAAACTCTCTA | k-d9-kekeke | 2 | 2109 |
| 740279 | 1790 | 1805 | 44179 | 44194 | ATGCTGTGAAACTCTC | k-d9-kekeke | 24 | 2110 |
| 740281 | 1789 | 1804 | 44178 | 44193 | TGCTGTGAAACTCTCT | kk-d10-keke | 30 | 2111 |
| 740283 | 1790 | 1805 | 44179 | 44194 | ATGCTGTGAAACTCTC | kk-d10-keke | 35 | 2112 |
| 740285 | 1788 | 1803 | 44177 | 44192 | GCTGTGAAACTCTCTA | kk-d8-kekekk | 16 | 2113 |
| 740287 | 1789 | 1804 | 44178 | 44193 | TGCTGTGAAACTCTCT | kk-d8-kekekk | 5 | 2114 |
| 740289 | 1790 | 1805 | 44179 | 44194 | ATGCTGTGAAACTCTC | kk-d8-kekekk | 25 | 2115 |
| 740291 | 1789 | 1804 | 44178 | 44193 | TGCTGTGAAACTCTCT | kk-d9-kdkdk | 26 | 2116 |
| 740293 | 1789 | 1804 | 44178 | 44193 | TGCTGTGAAACTCTCT | kk-d9-kekek | 0 | 2117 |
| 740295 | 1790 | 1805 | 44179 | 44194 | ATGCTGTGAAACTCTC | kk-d9-kekek | 16 | 2118 |
| 740297 | 1790 | 1805 | 44179 | 44194 | ATGCTGTGAAACTCTC | kkk-d9-keke | 2 | 2119 |
| 740299 | 1789 | 1804 | 44178 | 44193 | TGCTGTGAAACTCTCT | kkk-d9-kkke | 23 | 2120 |
| 740301 | 1790 | 1805 | 44179 | 44194 | ATGCTGTGAAACTCTC | kkk-d9-kkke | 37 | 2121 |

TABLE 32
Inhibition of K-Ras mRNA by gapmers targeting SEQ ID NO: 1 and 2

| | SEQ ID | SEQ ID | SEQ ID | SEQ ID | | | | |
|---|---|---|---|---|---|---|---|---|
| | NO: 1 | NO: 1 | NO: 2 | NO: 2 | | | | SEQ |
| ISIS | Start | Stop | Start | Stop | | | % | ID |
| NO | Site | Site | Site | Site | Sequence | Chemistry | Inhibition | NO |
| 651987 | 1447 | 1462 | 43836 | 43851 | GCTATTAGGAGTCTTT | kkk-d10-kkk | 55 | 272 |
| 651987 | 1447 | 1462 | 43836 | 43851 | GCTATTAGGAGTCTTT | kkk-d10-kkk | 65 | 272 |
| 695867 | 1130 | 1145 | 43519 | 43534 | TCCATTTATGTGACTA | kkk-d10-kkk | 52 | 506 |
| 695924 | 1441 | 1456 | 43830 | 43845 | AGGAGTCTTTATAGTA | kkk-d10-kkk | 57 | 420 |
| 695998 | 1790 | 1805 | 44179 | 44194 | ATGCTGTGAAACTCTC | kkk-d10-kkk | 39 | 658 |
| 696017 | 1917 | 1932 | 44306 | 44321 | TTGATTTGTCAGCAGG | kkk-d10-kkk | 74 | 677 |
| 696044 | 2115 | 2130 | 44504 | 44519 | GTGTTTATGCAATGTT | kkk-d10-kkk | 60 | 715 |
| 696091 | 2436 | 2451 | 44825 | 44840 | AGATTGTGCTGAGCTT | kkk-d10-kkk | 11 | 762 |
| 696096 | 2463 | 2478 | 44852 | 44867 | ATGGTGTAACATAGGT | kkk-d10-kkk | 52 | 914 |
| 696152 | 2761 | 2776 | 45150 | 45165 | TTAGTGATTAGGTCAA | kkk-d10-kkk | 52 | 924 |
| 740165 | 2113 | 2128 | 44502 | 44517 | GTTTATGCAATGTTAA | k-d10-kekek | 37 | 2122 |
| 740166 | 2434 | 2449 | 44823 | 44838 | ATTGTGCTGAGCTTGA | k-d10-kekek | 34 | 2123 |
| 740167 | 2461 | 2476 | 44850 | 44865 | GGTGTAACATAGGTTA | k-d10-kekek | 64 | 2124 |
| 740171 | 2115 | 2130 | 44504 | 44519 | GTGTTTATGCAATGTT | k-d10-kekek | 53 | 2125 |
| 740172 | 2436 | 2451 | 44825 | 44840 | AGATTGTGCTGAGCTT | k-d10-kekek | 7 | 2126 |
| 740173 | 2463 | 2478 | 44852 | 44867 | ATGGTGTAACATAGGT | k-d10-kekek | 47 | 2127 |
| 740177 | 2113 | 2128 | 44502 | 44517 | GTTTATGCAATGTTAA | k-d9-kekeke | 31 | 2128 |
| 740178 | 2434 | 2449 | 44823 | 44838 | ATTGTGCTGAGCTTGA | k-d9-kekeke | 18 | 2129 |
| 740179 | 2461 | 2476 | 44850 | 44865 | GGTGTAACATAGGTTA | k-d9-kekeke | 57 | 2130 |
| 740183 | 2115 | 2130 | 44504 | 44519 | GTGTTTATGCAATGTT | k-d9-kekeke | 41 | 2131 |
| 740184 | 2436 | 2451 | 44825 | 44840 | AGATTGTGCTGAGCTT | k-d9-kekeke | 0 | 2132 |
| 740185 | 2463 | 2478 | 44852 | 44867 | ATGGTGTAACATAGGT | k-d9-kekeke | 39 | 2133 |
| 740189 | 2114 | 2129 | 44503 | 44518 | TGTTTATGCAATGTTA | kk-d10-keke | 23 | 2134 |
| 740190 | 2435 | 2450 | 44824 | 44839 | GATTGTGCTGAGCTTG | kk-d10-keke | 19 | 2135 |
| 740191 | 2462 | 2477 | 44851 | 44866 | TGGTGTAACATAGGTT | kk-d10-keke | 67 | 2136 |
| 740194 | 2115 | 2130 | 44504 | 44519 | GTGTTTATGCAATGTT | kk-d10-keke | 73 | 2137 |
| 740195 | 2436 | 2451 | 44825 | 44840 | AGATTGTGCTGAGCTT | kk-d10-keke | 40 | 2138 |
| 740196 | 2463 | 2478 | 44852 | 44867 | ATGGTGTAACATAGGT | kk-d10-keke | 65 | 2139 |
| 740199 | 2114 | 2129 | 44503 | 44518 | TGTTTATGCAATGTTA | kk-d8-kekekk | 56 | 2140 |
| 740200 | 2435 | 2450 | 44824 | 44839 | GATTGTGCTGAGCTTG | kk-d8-kekekk | 14 | 2141 |
| 740201 | 2462 | 2477 | 44851 | 44866 | TGGTGTAACATAGGTT | kk-d8-kekekk | 55 | 2142 |
| 740204 | 2115 | 2130 | 44504 | 44519 | GTGTTTATGCAATGTT | kk-d8-kekekk | 9 | 2143 |
| 740205 | 2436 | 2451 | 44825 | 44840 | AGATTGTGCTGAGCTT | kk-d8-kekekk | 0 | 2144 |
| 740206 | 2463 | 2478 | 44852 | 44867 | ATGGTGTAACATAGGT | kk-d8-kekekk | 40 | 2145 |
| 740210 | 2114 | 2129 | 44503 | 44518 | TGTTTATGCAATGTTA | kk-d9-kdkdk | 46 | 2146 |
| 740211 | 2435 | 2450 | 44824 | 44839 | GATTGTGCTGAGCTTG | kk-d9-kdkdk | 57 | 2147 |
| 740212 | 2462 | 2477 | 44851 | 44866 | TGGTGTAACATAGGTT | kk-d9-kdkdk | 57 | 2148 |
| 740216 | 2114 | 2129 | 44503 | 44518 | TGTTTATGCAATGTTA | kk-d9-kekek | 56 | 2149 |
| 740217 | 2435 | 2450 | 44824 | 44839 | GATTGTGCTGAGCTTG | kk-d9-kekek | 45 | 2150 |
| 740218 | 2462 | 2477 | 44851 | 44866 | TGGTGTAACATAGGTT | kk-d9-kekek | 62 | 2151 |
| 740221 | 2115 | 2130 | 44504 | 44519 | GTGTTTATGCAATGTT | kk-d9-kekek | 65 | 2152 |
| 740222 | 2436 | 2451 | 44825 | 44840 | AGATTGTGCTGAGCTT | kk-d9-kekek | 38 | 2153 |
| 740223 | 2463 | 2478 | 44852 | 44867 | ATGGTGTAACATAGGT | kk-d9-kekek | 53 | 2154 |
| 740227 | 2113 | 2128 | 44502 | 44517 | GTTTATGCAATGTTAA | kk-d8-kekekk | 57 | 2155 |
| 740228 | 2434 | 2449 | 44823 | 44838 | ATTGTGCTGAGCTTGA | kk-d8-kekekk | 31 | 2156 |
| 740229 | 2461 | 2476 | 44850 | 44865 | GGTGTAACATAGGTTA | kk-d8-kekekk | 54 | 2157 |
| 740233 | 2115 | 2130 | 44504 | 44519 | GTGTTTATGCAATGTT | kkk-d8-kdkdk | 65 | 2158 |
| 740234 | 2436 | 2451 | 44825 | 44840 | AGATTGTGCTGAGCTT | kkk-d8-kdkdk | 42 | 2159 |
| 740235 | 2463 | 2478 | 44852 | 44867 | ATGGTGTAACATAGGT | kkk-d8-kdkdk | 36 | 2160 |
| 740238 | 2115 | 2130 | 44504 | 44519 | GTGTTTATGCAATGTT | kkk-d8-kekek | 66 | 2161 |
| 740239 | 2436 | 2451 | 44825 | 44840 | AGATTGTGCTGAGCTT | kkk-d8-kekek | 26 | 2162 |
| 740240 | 2463 | 2478 | 44852 | 44867 | ATGGTGTAACATAGGT | kkk-d8-kekek | 43 | 2163 |
| 740244 | 2114 | 2129 | 44503 | 44518 | TGTTTATGCAATGTTA | kkk-d8-kekek | 52 | 2164 |
| 740245 | 2435 | 2450 | 44824 | 44839 | GATTGTGCTGAGCTTG | kkk-d8-kekek | 37 | 2165 |
| 740246 | 2462 | 2477 | 44851 | 44866 | TGGTGTAACATAGGTT | kkk-d8-kekek | 67 | 2166 |
| 740250 | 2115 | 2130 | 44504 | 44519 | GTGTTTATGCAATGTT | kkk-d9-keke | 66 | 2167 |
| 740251 | 2436 | 2451 | 44825 | 44840 | AGATTGTGCTGAGCTT | kkk-d9-keke | 43 | 2168 |
| 740252 | 2463 | 2478 | 44852 | 44867 | ATGGTGTAACATAGGT | kkk-d9-keke | 43 | 2169 |
| 740256 | 2114 | 2129 | 44503 | 44518 | TGTTTATGCAATGTTA | kkk-d9-kkke | 72 | 2170 |
| 740257 | 2435 | 2450 | 44824 | 44839 | GATTGTGCTGAGCTTG | kkk-d9-kkke | 30 | 2171 |
| 740258 | 2462 | 2477 | 44851 | 44866 | TGGTGTAACATAGGTT | kkk-d9-kkke | 56 | 2172 |
| 740261 | 2115 | 2130 | 44504 | 44519 | GTGTTTATGCAATGTT | kkk-d9-kkke | 59 | 2173 |
| 740262 | 2436 | 2451 | 44825 | 44840 | AGATTGTGCTGAGCTT | kkk-d9-kkke | 42 | 2174 |
| 740263 | 2463 | 2478 | 44852 | 44867 | ATGGTGTAACATAGGT | kkk-d9-kkke | 47 | 2175 |
| 740274 | 2759 | 2774 | 45148 | 45163 | AGTGATTAGGTCAAAT | k-d10-kekek | 21 | 2176 |
| 740276 | 2761 | 2776 | 45150 | 45165 | TTAGTGATTAGGTCAA | k-d10-kekek | 12 | 2177 |
| 740278 | 2759 | 2774 | 45148 | 45163 | AGTGATTAGGTCAAAT | k-d9-kekeke | 12 | 2178 |
| 740280 | 2761 | 2776 | 45150 | 45165 | TTAGTGATTAGGTCAA | k-d9-kekeke | 0 | 2179 |
| 740282 | 2760 | 2775 | 45149 | 45164 | TAGTGATTAGGTCAAA | kk-d10-keke | 34 | 2180 |
| 740284 | 2761 | 2776 | 45150 | 45165 | TTAGTGATTAGGTCAA | kk-d10-keke | 22 | 2181 |
| 740286 | 2759 | 2774 | 45148 | 45163 | AGTGATTAGGTCAAAT | kk-d8-kekekk | 46 | 2182 |
| 740288 | 2760 | 2775 | 45149 | 45164 | TAGTGATTAGGTCAAA | kk-d8-kekekk | 42 | 2183 |
| 740290 | 2761 | 2776 | 45150 | 45165 | TTAGTGATTAGGTCAA | kk-d8-kekekk | 33 | 2184 |
| 740292 | 2760 | 2775 | 45149 | 45164 | TAGTGATTAGGTCAAA | kk-d9-kdkdk | 25 | 2185 |
| 740294 | 2760 | 2775 | 45149 | 45164 | TAGTGATTAGGTCAAA | kk-d9-kekek | 51 | 2186 |
| 740296 | 2761 | 2776 | 45150 | 45165 | TTAGTGATTAGGTCAA | kk-d9-kekek | 48 | 2187 |
| 740298 | 2761 | 2776 | 45150 | 45165 | TTAGTGATTAGGTCAA | kkk-d9-keke | 32 | 2188 |
| 740300 | 2760 | 2775 | 45149 | 45164 | TAGTGATTAGGTCAAA | kkk-d9-kkke | 43 | 2189 |
| 740302 | 2761 | 2776 | 45150 | 45165 | TTAGTGATTAGGTCAA | kkk-d9-kkke | 53 | 2190 |

### Example 10: Dose-Dependent Inhibition of Human K-Ras mRNA Expression in A431 Cells

Antisense oligonucleotides described in the studies above were tested at various doses in A431 cells. Isis No. 549148 (3-10-3 cEt gapmer, GGCTACTACGCCGTCA, designated herein as SEQ ID NO: 2191) or ISIS 141923 (5-10-5 MOE gapmer, CCTTCCCTGAAGGTTCCTCC, designated herein as SEQ ID NO: 2192), control oligonucleotides that do not target K-Ras, were included in each experiment as negative controls.

### Study 1

Cells were plated at a density of 5,000 cells per well. Cells were incubated with concentrations of antisense oligonucleotide specified in the tables below. Each table represents a separate experiment. After approximately 72 hours, RNA was isolated from the cells and K-Ras mRNA levels were measured by quantitative real-time PCR. Human K-Ras primer probe set RTS3496_MGB, described above, was used to measure mRNA levels. K-Ras mRNA levels were normalized to beta-actin mRNA levels or RIBOGREEN©. Results are presented as percent inhibition of K-Ras, relative to untreated control cells. As used herein, a value of ‘0’ indicates that treatment with the antisense oligonucleotide did not inhibit mRNA levels.

For some antisense oligonucleotides, the half maximal inhibitory concentration (IC₅₀) is also presented. As illustrated in the tables below, oligonucleotides were successfully taken up by the cells and K-Ras mRNA levels were significantly reduced in a dose-dependent manner in antisense oligonucleotide treated cells.

TABLE 33
Dose-dependent inhibition of human K-Ras mRNA expression
by free-uptake of ISIS oligonucleotides

| ISIS. | Inhibition (%) | | | | | IC₅₀ |
|---|---|---|---|---|---|---|
| No | 12.3 nM | 37.0 nM | 111 nM | 333 nM | 1000 nM | (μM) |
| 549148 | 10 | 10 | 8 | 9 | 18 | >1000 |
| 651530 | 24 | 64 | 84 | 92 | 93 | 24 |
| 651555 | 23 | 58 | 73 | 86 | 88 | 30 |
| 651587 | 31 | 68 | 84 | 89 | 91 | 22 |
| 651987 | 51 | 79 | 86 | 90 | 93 | 12 |
| 695785 | 25 | 58 | 75 | 86 | 88 | 30 |
| 695823 | 24 | 46 | 68 | 82 | 89 | 74 |
| 695980 | 44 | 74 | 86 | 92 | 95 | 18 |
| 695995 | 29 | 61 | 81 | 91 | 93 | 24 |

TABLE 34
Dose-dependent inhibition of human K-Ras mRNA expression
by free-uptake of ISIS oligonucleotides

| ISIS | Inhibition (%) | | | | |
|---|---|---|---|---|---|
| No. | 49.4 nM | 148.1 nM | 444.4 nM | 1333 nM | 4000 nM |
| 540806 | 0 | 1 | 54 | 73 | 80 |
| 651530 | 5 | 27 | 62 | 82 | 90 |
| 651634 | 0 | 11 | 59 | 78 | 86 |
| 651645 | 0 | 11 | 26 | 65 | 79 |
| 651733 | 0 | 0 | 25 | 44 | 56 |
| 651760 | 0 | 10 | 61 | 79 | 87 |
| 651972 | 1 | 6 | 38 | 60 | 80 |
| 651987 | 2 | 25 | 71 | 86 | 89 |
| 651990 | 0 | 18 | 46 | 65 | 79 |
| 652004 | 0 | 9 | 55 | 82 | 90 |
| 652019 | 0 | 14 | 53 | 75 | 85 |
| 652028 | 0 | 0 | 31 | 73 | 81 |
| 652034 | 0 | 0 | 35 | 61 | 78 |
| 652100 | 0 | 18 | 47 | 69 | 80 |
| 652132 | 0 | 23 | 61 | 78 | 88 |

TABLE 35
Dose-dependent inhibition of human K-Ras mRNA expression
by free-uptake of ISIS oligonucleotides

| Isis | Inhibition (%) | | | | |
|---|---|---|---|---|---|
| No. | 62 nM | 185 nM | 556 nM | 1667 nM | 5000 nM |
| 141923 | 0 | 0 | 4 | 0 | 22 |
| 651588 | 8 | 13 | 22 | 60 | 75 |
| 651987 | 10 | 33 | 50 | 77 | 83 |
| 651990 | 4 | 1 | 24 | 55 | 68 |
| 652028 | 17 | 5 | 39 | 59 | 72 |
| 716583 | 9 | 34 | 46 | 45 | 70 |
| 716587 | 18 | 18 | 45 | 61 | 74 |
| 716588 | 7 | 24 | 46 | 67 | 77 |
| 716600 | 22 | 30 | 56 | 79 | 83 |
| 716608 | 22 | 36 | 53 | 64 | 75 |
| 716612 | 20 | 28 | 45 | 66 | 75 |
| 716625 | 5 | 28 | 38 | 61 | 82 |
| 716628 | 2 | 19 | 58 | 72 | 81 |
| 716655 | 13 | 21 | 40 | 53 | 79 |
| 716656 | 11 | 27 | 46 | 65 | 83 |
| 716769 | 8 | 25 | 48 | 62 | 83 |

TABLE 36
Dose-dependent inhibition of human K-Ras mRNA expression
by free-uptake of ISIS oligonucleotides

| Isis | Inhibition (%) | | | | IC₅₀ |
|---|---|---|---|---|---|
| No. | 148 nM | 444 nM | 1333 nM | 4000 nM | (μM) |
| 540806 | 15 | 14 | 55 | 62 | 1.7 |
| 651587 | 6 | 32 | 63 | 76 | 1.0 |
| 651990 | 5 | 23 | 34 | 59 | 2.7 |
| 652004 | 3 | 29 | 62 | 73 | 1.1 |
| 663455 | 0 | 23 | 38 | 47 | 3.7 |
| 695815 | 28 | 26 | 40 | 58 | 2.7 |
| 695909 | 0 | 18 | 32 | 50 | 4.0 |
| 695940 | 0 | 11 | 32 | 55 | 3.2 |
| 695958 | 0 | 8 | 33 | 36 | 8.8 |
| 695976 | 3 | 26 | 57 | 73 | 1.2 |
| 695977 | 14 | 19 | 44 | 62 | 2.1 |
| 695980 | 17 | 40 | 60 | 78 | 0.8 |
| 695981 | 1 | 28 | 51 | 65 | 1.6 |
| 695995 | 13 | 22 | 52 | 64 | 1.6 |
| 696105 | 7 | 22 | 45 | 58 | 2.3 |
| 696108 | 3 | 40 | 47 | 65 | 1.5 |
| 696117 | 2 | 17 | 41 | 59 | 2.4 |
| 696160 | 10 | 7 | 15 | 33 | >4 |
| 696176 | 2 | 0 | 0 | 10 | >4 |
| 696289 | 3 | 0 | 0 | 2 | >4 |

TABLE 37
Dose-dependent inhibition of human K-Ras mRNA expression
by free-uptake of ISIS oligonucleotides

| ISIS | Inhibition (%) | | | | |
|---|---|---|---|---|---|
| No. | 19.5 nM | 78.1 nM | 312.5 nM | 1250 nM | 5000 nM |
| 141923 | 10 | 15 | 12 | 0 | 14 |
| 651530 | 59 | 86 | 93 | 95 | 96 |
| 651555 | 49 | 83 | 93 | 96 | 96 |
| 651587 | 53 | 85 | 93 | 95 | 96 |
| 651987 | 73 | 91 | 94 | 95 | 95 |
| 695785 | 40 | 82 | 94 | 96 | 97 |
| 695980 | 52 | 85 | 95 | 96 | 97 |
| 695995 | 53 | 82 | 93 | 95 | 96 |

TABLE 38
Dose-dependent inhibition of human K-Ras mRNA expression
by free-uptake of ISIS oligonucleotides

| | Inhibition (%) | | | | | |
|---|---|---|---|---|---|---|
| ISIS | 19.5 | 78.1 | 312.5 | 1250 | 5000 | IC₅₀ |
| No. | nM | nM | nM | nM | nM | (μM) |
| 141923 | 0 | 7 | 3 | 0 | 2 | >5 |
| 651530 | 24 | 55 | 82 | 90 | 93 | 0.07 |
| 651555 | 13 | 62 | 80 | 91 | 93 | 0.09 |
| 651587 | 17 | 55 | 76 | 88 | 93 | 0.10 |
| 651987 | 39 | 72 | 87 | 91 | 93 | 0.02 |
| 695785 | 20 | 40 | 71 | 84 | 90 | 0.13 |
| 695980 | 31 | 67 | 86 | 93 | 96 | 0.04 |
| 695995 | 18 | 54 | 78 | 8 | 92 | 0.09 |

TABLE 39
Dose-dependent inhibition of human K-Ras mRNA expression
by free-uptake of ISIS oligonucleotides

| ISIS | Inhibition (%) | | | | | IC₅₀ |
|---|---|---|---|---|---|---|
| No. | 12.3 nM | 37.0 nM | 111 nM | 333 nM | 1000 nM | (μM) |
| 549148 | 0 | 0 | 0 | 0 | 0 | >1000 |
| 651987 | 19 | 41 | 60 | 80 | 92 | 60 |
| 696018 | 23 | 66 | 73 | 80 | 83 | 30 |
| 716744 | 39 | 62 | 76 | 83 | 88 | 20 |
| 716749 | 28 | 51 | 68 | 75 | 81 | 35 |
| 716754 | 27 | 55 | 60 | 70 | 76 | 40 |
| 746273 | 23 | 52 | 67 | 77 | 81 | 35 |
| 746274 | 29 | 49 | 65 | 70 | 74 | 35 |
| 746275 | 62 | 84 | 91 | 95 | 95 | 9 |
| 746276 | 49 | 70 | 81 | 87 | 88 | 12 |
| 746277 | 7 | 26 | 38 | 46 | 57 | 450 |
| 746278 | 22 | 36 | 46 | 50 | 64 | 200 |
| 746279 | 37 | 57 | 74 | 77 | 89 | 25 |
| 746280 | 47 | 61 | 74 | 84 | 88 | 15 |
| 746281 | 0 | 36 | 50 | 69 | 73 | 100 |
| 746282 | 20 | 35 | 50 | 70 | 70 | 100 |
| 746283 | 6 | 24 | 30 | 54 | 60 | 300 |
| 746284 | 21 | 47 | 60 | 72 | 78 | 45 |
| 746285 | 37 | 60 | 72 | 79 | 81 | 25 |
| 746286 | 48 | 72 | 84 | 90 | 92 | 12 |
| 746287 | 32 | 62 | 71 | 80 | 82 | 25 |

TABLE 40
Dose-dependent inhibition of human K-Ras mRNA expression
by free-uptake of ISIS oligonucleotides

| Isis | Inhibition (%) | | | | IC₅₀ |
|---|---|---|---|---|---|
| No. | 148 nM | 444 nM | 1333 nM | 4000 nM | (μM) |
| 141923 | 0 | 0 | 0 | 0 | NA |
| 651499 | 22 | 49 | 63 | 73 | 0.7 |
| 651530 | 44 | 63 | 78 | 89 | 0.2 |
| 651541 | 31 | 61 | 71 | 80 | 0.4 |
| 651555 | 30 | 59 | 75 | 85 | 0.4 |
| 651603 | 36 | 59 | 70 | 80 | 0.3 |
| 651634 | 22 | 54 | 69 | 80 | 0.5 |
| 651635 | 31 | 52 | 65 | 74 | 0.5 |
| 651704 | 26 | 35 | 43 | 56 | 2.3 |
| 651795 | 22 | 47 | 59 | 74 | 0.7 |
| 651837 | 3 | 15 | 27 | 38 | 5 |
| 651987 | 42 | 70 | 85 | 88 | 0.2 |
| 695785 | 35 | 63 | 75 | 79 | 0.3 |
| 695809 | 32 | 52 | 71 | 80 | 0.4 |
| 695823 | 19 | 41 | 63 | 73 | 0.8 |
| 695847 | 25 | 55 | 77 | 87 | 0.4 |
| 695852 | 20 | 33 | 49 | 65 | 1.4 |
| 695867 | 59 | 79 | 86 | 92 | 0.04 |
| 695883 | 17 | 18 | 43 | 57 | 2.7 |
| 695885 | 21 | 55 | 69 | 84 | 0.5 |
| 695912 | 45 | 69 | 72 | 82 | 0.1 |
| 695917 | 32 | 55 | 73 | 76 | 0.4 |
| 695924 | 44 | 65 | 73 | 83 | 0.2 |
| 695930 | 31 | 54 | 75 | 82 | 0.4 |
| 695998 | 37 | 54 | 74 | 80 | 0.3 |
| 696012 | 31 | 69 | 77 | 89 | 0.3 |
| 696013 | 33 | 64 | 73 | 83 | 0.3 |
| 696017 | 55 | 65 | 85 | 90 | 0.1 |
| 696018 | 37 | 58 | 73 | 80 | 0.3 |
| 696026 | 34 | 68 | 61 | 76 | 0.3 |
| 696043 | 28 | 43 | 69 | 81 | 0.6 |
| 696044 | 55 | 76 | 85 | 90 | 0.1 |
| 696090 | 43 | 64 | 78 | 83 | 0.2 |
| 696091 | 24 | 38 | 44 | 66 | 1.4 |
| 696096 | 23 | 62 | 74 | 82 | 0.4 |
| 696137 | 42 | 66 | 74 | 83 | 0.2 |
| 696152 | 27 | 61 | 73 | 81 | 0.4 |
| 696167 | 0 | 55 | 67 | 77 | 0.8 |
| 696176 | 0 | 0 | 11 | 21 | >4 |
| 696219 | 7 | 37 | 51 | 63 | 1.5 |
| 696241 | 14 | 18 | 15 | 32 | 5 |
| 696271 | 34 | 53 | 69 | 76 | 0.4 |
| 696276 | 26 | 39 | 52 | 63 | 1.2 |
| 696287 | 29 | 40 | 57 | 72 | 0.8 |
| 696289 | 2 | 13 | 7 | 7 | >4 |
| 696299 | 29 | 30 | 50 | 62 | 1.5 |
| 696317 | 15 | 49 | 66 | 75 | 0.7 |
| 696318 | 14 | 38 | 47 | 60 | 1.7 |
| 696355 | 16 | 38 | 53 | 65 | 1.2 |
| 696356 | 29 | 33 | 55 | 64 | 1.2 |
| 696358 | 28 | 40 | 60 | 69 | 0.8 |
| 696377 | 15 | 42 | 59 | 75 | 0.9 |
| 696495 | 18 | 48 | 52 | 71 | 0.9 |
| 696554 | 3 | 28 | 47 | 60 | 1.9 |
| 696556 | 25 | 46 | 62 | 73 | 0.7 |

TABLE 41
Dose-dependent inhibition of human K-Ras mRNA expression
by free-uptake of ISIS oligonucleotides

| Isis | Inhibition (%) | | | | IC₅₀ |
|---|---|---|---|---|---|
| No. | 185 nM | 556 nM | 1667 nM | 5000 nM | (μM) |
| 141923 | 0 | 0 | 0 | 0 | >10,000 |
| 651987 | 16 | 47 | 67 | 77 | 1.3 |
| 695867 | 29 | 59 | 65 | 78 | N/A |
| 695924 | 14 | 42 | 58 | 77 | 1.5 |
| 695998 | 26 | 42 | 47 | 72 | N/A |
| 696017 | 41 | 60 | 77 | 87 | NA |
| 696044 | 31 | 52 | 77 | 89 | 0.9 |
| 696096 | 17 | 36 | 59 | 75 | N/A |
| 696152 | 32 | 56 | 68 | 82 | N/A |
| 740164 | 16 | 46 | 62 | 78 | N/A |
| 740167 | 15 | 39 | 62 | 80 | 1.0 |
| 740179 | 1 | 35 | 56 | 71 | 1.5 |
| 740187 | 27 | 45 | 57 | 82 | N/A |
| 740188 | 31 | 48 | 67 | 8 | N/A |
| 740191 | 28 | 62 | 74 | 87 | N/A |
| 740194 | 44 | 71 | 83 | 92 | N/A |
| 740196 | 22 | 39 | 60 | 69 | 1.1 |
| 740199 | 22 | 47 | 68 | 76 | 0.8 |
| 740201 | 11 | 33 | 58 | 68 | 1.4 |
| 740211 | 0 | 17 | 48 | 58 | 2.7 |
| 740212 | 13 | 33 | 63 | 75 | 1.2 |
| 740216 | 50 | 46 | 69 | 82 | 0.3 |
| 740218 | 21 | 45 | 66 | 83 | 0.8 |
| 740221 | 22 | 54 | 77 | 83 | 0.6 |
| 740227 | 27 | 46 | 45 | 79 | 1 |
| 740229 | 0 | 2 | 40 | 58 | 3.5 |
| 740233 | 27 | 47 | 73 | 81 | 0.7 |
| 740238 | 33 | 55 | 69 | 78 | NA |
| 740246 | 31 | 53 | 79 | 82 | NA |
| 740250 | 30 | 55 | 80 | 85 | 0.5 |
| 740253 | 28 | 47 | 53 | 66 | NA |
| 740255 | 26 | 52 | 70 | 80 | NA |
| 740256 | 43 | 57 | 78 | 90 | NA |
| 740258 | 21 | 46 | 69 | 80 | 0.8 |
| 740261 | 19 | 63 | 79 | 88 | 0.5 |

TABLE 42
Dose-dependent inhibition of human K-Ras mRNA expression
by free-uptake of ISIS oligonucleotides

| Isis | Inhibition (%) | | | | | IC₅₀ |
|---|---|---|---|---|---|---|
| No. | 62 nM | 185 nM | 556 nM | 1667 nM | 5000 nM | (μM) |
| 141923 | 2 | 0 | 0 | 0 | 0 | >5 |
| 651588 | 12 | 0 | 21 | 47 | 69 | 2.2 |
| 651634 | 11 | 2 | 2 | 35 | 52 | >5 |
| 651653 | 3 | 2 | 37 | 47 | 71 | 1.4 |
| 651987 | 0 | 26 | 44 | 58 | 77 | 1.0 |
| 651634 | 11 | 2 | 2 | 35 | 52 | >5 |
| 651653 | 3 | 25 | 37 | 47 | 71 | 1.4 |
| 652028 | 3 | 3 | 19 | 43 | 60 | 3.2 |
| 716583 | 0 | 6 | 5 | 31 | 65 | 4.2 |
| 716587 | 0 | 9 | 17 | 43 | 64 | 2.3 |
| 716588 | 0 | 13 | 35 | 52 | 71 | 1.5 |
| 716600 | 0 | 14 | 35 | 53 | 72 | 1.4 |
| 716608 | 0 | 0 | 27 | 45 | 70 | 2 |
| 716612 | 16 | 3 | 34 | 39 | 69 | 2.2 |
| 716625 | 0 | 3 | 29 | 40 | 64 | 2.5 |
| 716628 | 0 | 0 | 36 | 58 | 74 | 1.3 |
| 716655 | 0 | 0 | 27 | 53 | 68 | 2 |
| 716656 | 0 | 9 | 21 | 46 | 67 | 2 |
| 716673 | 3 | 7 | 29 | 47 | 67 | 2 |
| 716674 | 1 | 0 | 35 | 61 | 78 | 1.2 |
| 716675 | 8 | 8 | 30 | 56 | 68 | 1.6 |
| 716683 | 19 | 0 | 23 | 43 | 60 | 2.7 |
| 716716 | 0 | 0 | 26 | 48 | 68 | 2 |
| 716728 | 1 | 28 | 24 | 46 | 71 | 1.7 |
| 716758 | 10 | 0 | 36 | 58 | 67 | 1.6 |
| 716763 | 0 | 10 | 24 | 45 | 66 | 2 |
| 716772 | 0 | 19 | 32 | 50 | 67 | 1.7 |
| 716782 | 0 | 0 | 19 | 31 | 39 | >5 |
| 716807 | 3 | 5 | 7 | 9 | 16 | >5 |

### Study 2

A431 cells were plated at a density of 10,000 cells per well. Cells were incubated with concentrations of antisense oligonucleotide specified in the tables below. Each table represents a separate experiment. After approximately 48 hours, RNA was isolated from the cells and K-Ras mRNA levels were measured by quantitative real-time PCR. Human K-Ras primer probe set RTS3496_MGB, described above, was used to measure mRNA levels. K-Ras mRNA levels were normalized to RIBOGREEN©. Results are presented as percent inhibition of K-Ras, relative to untreated control cells. A negative value for percent inhibition indicates that the K-Ras mRNA level was higher than in untreated cells.

For some antisense oligonucleotides, the half maximal inhibitory concentration (IC₅₀) is also presented. As illustrated in the tables below, oligonucleotides were successfully taken up by the cells and K-Ras mRNA levels were significantly reduced in a dose-dependent manner in antisense oligonucleotide treated cells.

TABLE 43
Dose-dependent inhibition of human K-Ras mRNA expression
by free-uptake of ISIS oligonucleotides

| Isis | Inhibition (%) | | | | |
|---|---|---|---|---|---|
| No. | 7.81 nM | 31.25 nM | 125.0 nM | 500.0 nM | 2000 nM |
| 141923 | 0 | 0 | 0 | 0 | 0 |
| 651987 | 30 | 39 | 68 | 87 | 93 |
| 696018 | 24 | 41 | 64 | 87 | 93 |
| 696044 | 29 | 54 | 83 | 94 | 97 |
| 716600 | 20 | 45 | 70 | 89 | 94 |
| 716655 | 2 | 20 | 49 | 79 | 92 |
| 740233 | 29 | 46 | 70 | 86 | 93 |
| 746275 | 25 | 47 | 65 | 85 | 91 |

TABLE 44
Dose-dependent inhibition of human K-Ras mRNA expression
by free-uptake of ISIS oligonucleotides

| | Inhibition (%) | | | | | |
|---|---|---|---|---|---|---|
| Isis | 7.81 | 31.25 | 125.0 | 500.0 | 2000 | IC₅₀ |
| No. | nM | nM | nM | nM | nM | (μM) |
| 141923 | 2 | 1 | 0 | 3 | 4 | >2 |
| 651987 | 18 | 30 | 67 | 87 | 93 | 0.06 |
| 696018 | 9 | 26 | 57 | 80 | 90 | 0.11 |
| 696044 | 15 | 44 | 83 | 92 | 95 | 0.04 |
| 716600 | 13 | 36 | 67 | 88 | 94 | 0.06 |
| 716655 | 10 | 23 | 53 | 82 | 92 | 0.11 |
| 740233 | 7 | 32 | 58 | 80 | 89 | 0.09 |
| 746275 | 6 | 24 | 61 | 84 | 92 | 0.09 |

### Study 3

Hep3B cells were plated at a density of 20,000 cells per well. Cells were transfected using electroporation with increasing concentrations of antisense oligonucleotide, as shown below. After a treatment period of approximately 24 hours, RNA was isolated from the cells and human K-Ras mRNA levels were measured by quantitative real-time PCR. Human K-Ras primer probe set RTS3496_MGB, described above, was used to measure mRNA levels. K-Ras mRNA levels were normalized to Ribogreen. Results are presented as percent inhibition of K-Ras, relative to untreated control cells.

The half maximal inhibitory concentration (IC₅₀) is also presented. As illustrated in the table below, K-Ras mRNA levels were significantly reduced in a dose-dependent manner in antisense oligonucleotide treated cells.

TABLE 45
Dose-dependent inhibition of human K-Ras mRNA expression
by electroporation of ISIS oligonucleotides

| | Inhibition (%) | | | | | |
|---|---|---|---|---|---|---|
| ISIS | 61.7 | 185.2 | 555.6 | 1666.7 | 5000.0 | IC₅₀ |
| No. | nM | nM | nM | nM | nM | (μM) |
| 540806 | 14 | 36 | 67 | 75 | 90 | 0.4 |
| 651530 | 25 | 39 | 66 | 82 | 92 | 0.3 |
| 651551 | 38 | 65 | 80 | 87 | 93 | 0.1 |
| 651586 | 28 | 51 | 73 | 86 | 91 | 0.2 |
| 651595 | 15 | 40 | 65 | 80 | 37 | 0.3 |
| 651634 | 14 | 36 | 57 | 74 | 90 | 0.4 |
| 651645 | 9 | 43 | 67 | 83 | 84 | 0.4 |
| 651646 | 44 | 65 | 79 | 90 | 93 | 0.1 |
| 651647 | 12 | 40 | 61 | 83 | 89 | 0.4 |
| 651672 | 2 | 15 | 47 | 69 | 83 | 0.8 |
| 651733 | 16 | 42 | 66 | 79 | 86 | 0.3 |
| 651741 | 14 | 34 | 57 | 82 | 88 | 0.4 |
| 651760 | 8 | 37 | 56 | 74 | 89 | 0.5 |
| 651923 | 6 | 27 | 54 | 84 | 95 | 0.5 |
| 651951 | 13 | 30 | 55 | 76 | 89 | 0.5 |
| 651953 | 19 | 40 | 64 | 83 | 89 | 0.3 |
| 651959 | 9 | 31 | 66 | 79 | 85 | 0.4 |
| 651972 | 25 | 33 | 58 | 82 | 86 | 0.4 |
| 651987 | 35 | 46 | 66 | 82 | 90 | 0.2 |
| 651990 | 18 | 36 | 64 | 84 | 91 | 0.3 |
| 652002 | 13 | 20 | 53 | 77 | 85 | 0.6 |
| 652004 | 18 | 48 | 67 | 85 | 91 | 0.3 |
| 652019 | 21 | 37 | 68 | 84 | 89 | 0.3 |
| 652028 | 17 | 41 | 58 | 77 | 89 | 0.4 |
| 652034 | 11 | 36 | 63 | 79 | 87 | 0.4 |
| 652050 | 21 | 34 | 63 | 79 | 87 | 0.4 |
| 652100 | 2 | 32 | 58 | 72 | 88 | 0.5 |
| 652101 | 16 | 25 | 52 | 72 | 86 | 0.6 |
| 652132 | 19 | 42 | 72 | 76 | 83 | 0.3 |
| 652157 | 0 | 22 | 54 | 77 | 88 | 0.6 |

### Example 11: Dose-Dependent Inhibition of Antisense Oligonucleotides Targeting K-Ras in Cynomolgus Monkey Primary Hepatocytes

At the time this study was undertaken, the cynomolgus monkey genomic sequence was not available in the National Center for Biotechnology Information (NCBI) database; therefore, cross-reactivity with the cynomolgus monkey gene sequence could not be confirmed. Instead, the sequences of the ISIS antisense oligonucleotides used in the cynomolgus monkeys were compared to a rhesus monkey genomic DNA sequence for complementarity. It is expected that ISIS oligonucleotides with complementarity to the rhesus monkey sequence are fully cross-reactive with the cynomolgus monkey sequence as well. The human antisense oligonucleotides tested had at most one mismatch with the rhesus genomic sequence (the complement of GENBANK Accession NC_007868.1 truncated from nucleotide 25479955 to 25525362, designated herein as SEQ ID NO: 2194). In the table below, the number of mismatches of the oligonucleotides with respect to the rhesus genomic sequence is indicated as “# MM.”

TABLE 46

| | | | | SEQ |
|---|---|---|---|---|
| | | | # | ID |
| Isis No. | Sequence | Chemistry | MM | NO. |
| 141923 | CCTTCCCTGAAGGTTCCTCC | 5-10-5 MOE | | 2192 |
| 651987 | GCTATTAGGAGTCTTT | kkk-10-kkk | 0 | 272 |
| 696018 | CTCTTGATTTGTCAGC | kkk-10-kkk | 0 | 678 |
| 696044 | GTGTTTATGCAATGTT | kkk-10-kkk | 1 | 715 |
| 716600 | CCATTTATGTGACTAG | kkk-10-kkk | 1 | 790 |
| 716655 | TGTTTATGCAATGTTA | kkk-10-kkk | 1 | 854 |
| 740233 | GTGTTTATGCAATGTT | kkk-8-kdkdk | 1 | 2158 |
| 746275 | TCTTGATTTGTCAGCA | kk-10-keke | 0 | 804 |

Antisense oligonucleotides described above were tested at various doses in cynomolgus monkey hepatocytes for ability to reduce K-Ras expression. Cryopreserved cynomolgus monkey primary hepatocytes were plated at a density of 35,000 cells per well and transfected using electroporation with various concentrations of antisense oligonucleotide, as specified in the Tables below. After a treatment period of approximately 24 hours, the cells were washed and lysed, and RNA was isolated. Monkey K-Ras mRNA levels were measured by quantitative real-time PCR, using primer probe set RTS3496_MGB, as described above. K-Ras mRNA target levels were adjusted according to total RNA content, as measured by RIBOGREEN®. In the tables below, results are presented as percent inhibition of K-Ras, relative to untreated control cells. As used herein, a value of ‘0’ indicates that treatment with the antisense oligonucleotide did not inhibit mRNA levels.

TABLE 47
Dose dependent inhibition of monkey K-Ras mRNA expression
by electroporation of ISIS oligonucleotides into
in primary cynomolgus monkey hepatocytes

| | Inhibition (%) | | | | | |
|---|---|---|---|---|---|---|
| ISIS | 19.5 | 78.1 | 312.5 | 1250 | 5000 | IC₅₀ |
| No. | nM | nM | nM | nM | nM | (μM) |
| 141923 | 0 | 4 | 0 | 0 | 0 | >5 |
| 651530 | 37 | 64 | 80 | 83 | 84 | 0.03 |
| 651555 | 26 | 63 | 68 | 90 | 94 | 0.07 |
| 651587 | 48 | 69 | 75 | 85 | 78 | 0.01 |
| 651987 | 50 | 75 | 81 | 88 | 87 | 0.01 |
| 695785 | 14 | 40 | 72 | 81 | 83 | 0.2 |
| 695980 | 0 | 0 | 17 | 28 | 50 | 6 |
| 695995 | 33 | 46 | 70 | 74 | 90 | 0.1 |

TABLE 48
Dose dependent inhibition of monkey K-Ras mRNA expression
by electroporation of ISIS oligonucleotides into
in primary cynomolgus monkey hepatocytes

| | Inhibition (%) | | | | | |
|---|---|---|---|---|---|---|
| Isis | 7.81 | 31.25 | 125.0 | 500.0 | 2000 | IC₅₀ |
| No. | nM | nM | nM | nM | nM | (μM) |
| 141923 | 2 | 0 | 2 | 0 | 0 | >2 |
| 651987 | 0 | 20 | 64 | 83 | 91 | 0.09 |
| 696018 | 11 | 26 | 57 | 83 | 91 | 0.10 |
| 696044 | 0 | 1 | 0 | 0 | 0 | >2 |
| 716600 | 2 | 19 | 52 | 79 | 91 | 0.14 |
| 716655 | 0 | 0 | 0 | 0 | 4 | >2 |
| 740233 | 4 | 0 | 0 | 0 | 0 | >2 |
| 746275 | 8 | 22 | 51 | 78 | 88 | 0.13 |

### Example 12: Tolerability of Antisense Oligonucleotides Targeting Human K-Ras mRNA in Lean BALB/c Mice

### Treatment

Six-to-seven week old male BALB/c mice (Jackson Laboratory, Bar Harbor, Me.) were injected subcutaneously two times a week for four weeks (for a total of 8 treatments) at 100 mg/kg/week with the antisense oligonucleotides or with saline. Each treatment group consisted of 4 animals. The mice were sacrificed 72 hours following the final administration.

### Plasma Chemistry Markers

To evaluate the effect of antisense oligonucleotides on liver and kidney function, plasma levels of ALT transaminase, albumin, blood urea nitrogen (BUN) and total bilirubin were measured using an automated clinical chemistry analyzer (Hitachi Olympus AU400e, Melville, N.Y.). The results are presented in the Table below. Antisense oligonucleotides causing changes in the levels of any of the liver or kidney function markers outside the expected range for antisense oligonucleotides were excluded from further studies.

TABLE 49
Plasma chemistry markers in male BALB/c mice

| ISIS | ALT | Albumin | BUN | T. Bilirubin |
|---|---|---|---|---|
| No. | (U/L) | (g/dL) | (mg/dL) | (mg/dL) |
| PBS | 65 | 2.8 | 25 | 0.17 |
| 549148 | 57 | 2.8 | 26 | 0.27 |
| 651530 | 143 | 2.7 | 26 | 0.22 |
| 651551 | 5927 | 3.3 | 26 | 4.62 |
| 651634 | 56 | 2.7 | 29 | 0.21 |
| 651645 | 4944 | 3.1 | 27 | 0.25 |
| 651646 | 1449 | 1.8 | 25 | 0.27 |
| 651647 | 2061 | 2.7 | 23 | 0.25 |
| 651672 | 4070 | 2.9 | 31 | 0.48 |
| 651923 | 4157 | 3.1 | 21 | 0.36 |
| 651972 | 2105 | 3.3 | 29 | 0.22 |
| 651987 | 71 | 2.7 | 29 | 0.16 |
| 651990 | 32 | 2.5 | 30 | 0.18 |
| 652002 | 106 | 3.2 | 29 | 0.20 |
| 652004 | 145 | 3.0 | 31 | 0.19 |
| 652019 | 5205 | 3.6 | 24 | 0.41 |
| 652028 | 3712 | 3.8 | 17 | 4.43 |
| 652034 | 1167 | 3.6 | 27 | 0.32 |
| 652101 | 48 | 3.1 | 23 | 0.22 |
| 652132 | 1399 | 3.3 | 28 | 0.22 |
| 652157 | 68 | 2.9 | 24 | 0.23 |

### Body and Organ Weights

Body weights of BALB/c mice were measured at days 1 and 27, and the average body weight for each group is presented in the Table below. Liver, spleen and kidney weights were measured at the end of the study, and are presented in the Table below. Antisense oligonucleotides that caused any changes in organ weights outside the expected range for antisense oligonucleotides were excluded from further studies.

TABLE 50
Body and organ weights (in grams)

| | | | | |
|---|---|---|---|---|
| ISIS | body (g) | kidney | liver | spleen |

| No. | Day 1 | Day 27 | (g) | (g) | (g) |
|---|---|---|---|---|---|
| PBS | 24 | 25 | 0.4 | 1.7 | 0.09 |
| 549148 | 24 | 27 | 0.5 | 1.5 | 0.10 |
| 651530 | 23 | 27 | 0.5 | 1.7 | 0.13 |
| 651551 | 24 | 23 | 0.4 | 1.7 | 0.09 |
| 651634 | 24 | 27 | 0.3 | 1.7 | 0.12 |
| 651645 | 24 | 27 | 0.4 | 1.4 | 0.14 |
| 651646 | 24 | 21 | 0.4 | 1.4 | 0.18 |
| 651647 | 24 | 25 | 0.4 | 1.5 | 0.18 |
| 651672 | 23 | 20 | 0.4 | 1.4 | 0.07 |
| 651733 | 24 | N/A | N/A | N/A | N/A |
| 651760 | 23 | N/A | N/A | N/A | N/A |
| 651923 | 23 | 25 | 0.3 | 1.9 | 0.15 |
| 651972 | 24 | 25 | 0.4 | 1.5 | 0.11 |
| 651987 | 24 | 27 | 0.4 | 1.9 | 0.11 |
| 651990 | 24 | 28 | 0.4 | 2.2 | 0.12 |
| 652002 | 24 | 29 | 0.4 | 1.8 | 0.13 |
| 652004 | 23 | 27 | 0.4 | 1.5 | 0.10 |
| 652019 | 24 | 24 | 0.4 | 1.8 | 0.14 |
| 652028 | 23 | 18 | 0.3 | 1.0 | 0.05 |
| 652034 | 23 | 25 | 0.4 | 1.4 | 0.11 |
| 652100 | 25 | N/A | N/A | N/A | N/A |
| 652101 | 23 | 28 | 0.5 | 1.4 | 0.10 |
| 652132 | 25 | 27 | 0.4 | 1.8 | 0.09 |
| 652157 | 23 | 28 | 0.5 | 1.7 | 0.13 |

### Example 13: Pharmacodynamics and Toxicological Profile of Antisense Oligonucleotides Targeting K-Ras in an A431 Epidermoid Carcinoma Xenograft Model

Female, 6-8 week old NCr nude mice (Taconic Biosciences, Hudson, N.Y.) were inoculated with human epidermoid carcinoma A431 cells and treated with an antisense oligonucleotide described in the tables above or with PBS. Effects of the oligonucleotides on K-Ras mRNA expression in the tumor and tolerability in the mice were evaluated.

### Treatment

The mice each were inoculated with 5×10⁶ A431 cells in 50% Matrigel (BD Bioscience) for tumor development. Antisense oligonucleotide treatment started at day 10-14 after tumor inoculation when the mean tumor size reached approximately 200 mm³. The mice were subcutaneously injected with 50 mg/kg three times per week (150 mg/kg/week) for three weeks, for a total of nine doses, with an antisense oligonucleotide or PBS. The body weights of the mice were measured once per week. Three weeks after the start of treatment, the mice were sacrificed, K-Ras mRNA levels in the tumor, spleen weights, and body weights were measured.

### Study 1

### RNA Analysis

RNA was extracted from tumor tissue for real-time PCR analysis and measurement of human K-Ras mRNA levels using primer probe set RTS3496_MGB, described herein above. Results are presented as average percent inhibition of K-Ras for each treatment group, relative to PBS control, normalized to glyceraldehyde-3-phosphate dehydrogenase or beta-actin mRNA levels. As shown in the Tables below, treatment with Isis antisense oligonucleotides resulted in reduction of human K-Ras mRNA in comparison to the PBS control.

TABLE 51
Antisense mediated inhibition of human K-Ras
mRNA expression in A431 xenograft model

| ISIS | Inhibition |
|---|---|
| No. | (%) |
| 651499 | 39 |
| 651530 | 55 |
| 651541 | 32 |
| 651555 | 51 |
| 651587 | 51 |
| 651603 | 41 |
| 651634 | 37 |
| 651795 | 46 |
| 651987 | 50 |
| 651990 | 46 |
| 652004 | 47 |
| 695815 | 24 |
| 695823 | 54 |
| 695847 | 50 |
| 695867 | 68 |
| 695912 | 34 |
| 695930 | 45 |
| 695976 | 42 |
| 695980 | 56 |
| 695981 | 35 |
| 695995 | 47 |
| 696026 | 35 |
| 696317 | 40 |
| 696816 | 31 |

### Body Weight Measurements

Body weights were measured throughout the treatment period. The data is presented in the Table below as the average for each treatment group at various time points. Spleen weights were measured at the end of the study and are presented in the Table below.

TABLE 52
Body and spleen weight measurements in A431 xenograft model

| Isis | Body weight (g) | | | | Spleen (g) |
|---|---|---|---|---|---|
| No. | Day 1 | Day 6 | Day 12 | Day 19 | Day 20 |
| PBS | 21.5 | 23.0 | 23.4 | 22.9 | 0.09 |
| 481464 | 21.4 | 22.4 | 22.0 | 22.4 | 0.11 |
| 549148 | 21.8 | 23.0 | 22.1 | 23.0 | 0.10 |
| 560131 | 19.9 | 20.4 | 20.2 | 19.9 | 0.10 |
| 651499 | 20.8 | 21.9 | 22.2 | 22.5 | 0.13 |
| 651530 | 21.1 | 22.5 | 23.1 | 22.1 | 0.10 |
| 651541 | 20.5 | 21.4 | 21.4 | 21.0 | 0.09 |
| 651555 | 21.5 | 22.2 | 22.4 | 22.1 | 0.09 |
| 651587 | 21.5 | 22.6 | 22.6 | 20.8 | 0.11 |
| 651603 | 22.7 | 24.7 | 24.8 | 22.0 | 0.36 |
| 651634 | 20.6 | 21.9 | 21.9 | 21.9 | 0.09 |
| 651635 | 20.3 | 21.7 | 17.9 | N/A | N/A |
| 651795 | 21.4 | 22.8 | 23.3 | 22.8 | 0.09 |
| 651987 | 22.8 | 24.2 | 24.1 | 24.1 | 0.09 |
| 651990 | 22.3 | 23.3 | 23.3 | 23.0 | 0.12 |
| 652004 | 21.0 | 22.3 | 23.3 | 22.8 | 0.16 |
| 695815 | 22.7 | 23.4 | 23.4 | 21.4 | 0.09 |
| 695823 | 21.4 | 22.6 | 22.5 | 21.9 | 0.09 |
| 695847 | 21.5 | 22.1 | 22.1 | 20.4 | 0.09 |
| 695867 | 21.1 | 22.6 | 22.2 | 19.6 | 0.06 |
| 695912 | 21.4 | 22.3 | 22.4 | 22.5 | 0.09 |
| 695930 | 21.7 | 22.2 | 22.8 | 22.5 | 0.09 |
| 695976 | 20.5 | 22.1 | 22.1 | 21.6 | 0.11 |
| 695980 | 21.1 | 22.5 | 22.1 | 21.2 | 0.07 |
| 695981 | 19.9 | 21.6 | 22.1 | 21.7 | 0.08 |
| 695995 | 20.7 | 21.8 | 22.0 | 21.6 | 0.10 |
| 696026 | 21.7 | 22.8 | 23.4 | 22.8 | 0.09 |
| 696317 | 21.5 | 23.1 | 23.0 | 22.9 | 0.13 |
| 696816 | 21.6 | 22.3 | 22.3 | 22.1 | 0.12 |

### Plasma Chemistry Markers

To evaluate the effect of antisense oligonucleotides on liver and kidney function, plasma levels of transaminases, total bilirubin and blood urea nitrogen (BUN) were measured using an automated clinical chemistry analyzer (Hitachi Olympus AU400e, Melville, N.Y.). The results are presented in the Table below.

TABLE 53
Plasma chemistry markers in A431 xenograft model

| ISIS | ALT | AST | Albumin | T. Bilirubin | BUN |
|---|---|---|---|---|---|
| No. | (U/L) | (U/L) | g/dL | (mg/dL) | (mg/dL) |
| PBS | 37.0 | 117.5 | 3.1 | 0.17 | 22 |
| 481464 | 74.0 | 158.8 | 2.8 | 0.13 | 21 |
| 549148 | 48.3 | 107.7 | 3.2 | 0.16 | 20 |
| 560131 | 88.3 | 198.0 | 3.0 | 0.13 | 31 |
| 651499 | 69.8 | 142.0 | 2.8 | 0.17 | 22 |
| 651530 | 163.0 | 261.8 | 3.2 | 0.15 | 20 |
| 651541 | 37.3 | 67.3 | 2.7 | 0.14 | 22 |
| 651555 | 53.3 | 111.5 | 3.2 | 0.17 | 24 |
| 651587 | 151.8 | 239.3 | 3.3 | 0.13 | 16 |
| 651603 | 585.3 | 766.0 | 2.5 | 0.18 | 29 |
| 651634 | 54.3 | 134.8 | 3.1 | 0.15 | 20 |
| 651795 | 76.0 | 139.8 | 3.0 | 0.13 | 24 |
| 651987 | 51.3 | 115.5 | 3.0 | 0.13 | 18 |
| 651990 | 33.8 | 76.5 | 2.5 | 0.13 | 20 |
| 652004 | 96.3 | 154.0 | 3.0 | 0.12 | 21 |
| 695815 | 40.3 | 95.8 | 2.9 | 0.13 | 22 |
| 695823 | 27.7 | 116.7 | 2.9 | 0.15 | 22 |
| 695847 | 326.3 | 396.0 | 2.9 | 0.16 | 20 |
| 695867 | 722.8 | 1200.5 | 3.1 | 0.94 | 19 |
| 695912 | 28.8 | 80.5 | 3.1 | 0.16 | 23 |
| 695930 | 50.5 | 131.8 | 3.0 | 0.20 | 22 |
| 695976 | 101.8 | 281.0 | 2.7 | 0.17 | 20 |
| 695980 | 42.5 | 104.0 | 3.1 | 0.19 | 25 |
| 695981 | 53.8 | 132.3 | 2.8 | 0.12 | 21 |
| 695995 | 37.8 | 122.5 | 2.9 | 0.13 | 22 |
| 696026 | 56.5 | 101.5 | 3.1 | 0.19 | 23 |
| 696317 | 46.8 | 133.5 | 2.7 | 0.10 | 23 |
| 696816 | 44.0 | 83.3 | 2.7 | 0.13 | 24 |

### Study 2

### RNA Analysis

RNA was extracted from tumor tissue for real-time PCR analysis and measurement of human K-Ras mRNA levels using primer probe set RTS3496_MGB, described herein above. Results are presented as average percent inhibition of K-Ras for each treatment group, relative to PBS control. As shown in the Table below, treatment with Isis antisense oligonucleotides resulted in reduction of human K-Ras mRNA in comparison to the PBS control.

TABLE 54
Antisense mediated inhibition of human K-Ras
mRNA expression in A431 xenograft model

| ISIS | Inhibition |
|---|---|
| No. | (%) |
| 481464 | 33 |
| 549148 | 28 |
| 651987 | 64 |
| 695785 | 54 |
| 695809 | 28 |
| 695917 | 45 |
| 695924 | 87 |
| 695977 | 36 |
| 695998 | 55 |
| 696012 | 24 |
| 696013 | 54 |
| 696017 | 70 |
| 696018 | 71 |
| 696043 | 41 |
| 696044 | 72 |
| 696091 | 79 |
| 696096 | 75 |
| 696108 | 53 |
| 696117 | 40 |
| 696137 | 45 |
| 696152 | 58 |
| 696167 | 53 |
| 696271 | 50 |
| 696287 | 47 |
| 696358 | 46 |
| 696377 | 37 |
| 696556 | 41 |

### Body Weight Measurements

Body weights were measured throughout the treatment period. The data is presented in the Table below as the average for each treatment group at various time points. Spleen weights were measured at the end of the study (Day 21) and are presented in the Table below.

TABLE 55
Body and spleen weight measurements in A431 xenograft model

| | | |
|---|---|---|
| Isis | Body weight (g) | Spleen (g) |

| No. | Day 8 | Day 19 | Day 21 |
|---|---|---|---|
| PBS | 23.2 | 23.5 | 0.09 |
| 481464 | 20.9 | 21.2 | 0.09 |
| 549148 | 22.8 | 22.0 | 0.10 |
| 651987 | 20.8 | 20.5 | 0.09 |
| 695785 | 21.7 | 21.5 | 0.09 |
| 695809 | 22.0 | 21.5 | 0.08 |
| 695917 | 21.7 | 21.6 | 0.10 |
| 695924 | 22.4 | 18.8 | 0.05 |
| 695977 | 22.3 | 22.9 | 0.10 |
| 695998 | 22.9 | 23.2 | 0.10 |
| 696012 | 23.0 | 22.5 | 0.11 |
| 696013 | 21.6 | 22.7 | 0.14 |
| 696017 | 24.4 | 23.1 | 0.10 |
| 696018 | 23.0 | 23.1 | 0.09 |
| 696043 | 22.5 | 22.2 | 0.13 |
| 696044 | 21.6 | 20.5 | 0.09 |
| 696090 | 22.4 | N/A | N/A |
| 696091 | 22.1 | 20.8 | 0.08 |
| 696096 | 21.7 | 19.8 | 0.07 |
| 696108 | 22.5 | 22.8 | 0.11 |
| 696117 | 22.6 | 22.6 | 0.10 |
| 696137 | 23.0 | 21.9 | 0.10 |
| 696152 | 22.2 | 22.3 | 0.12 |
| 696167 | 22.7 | 22.5 | 0.12 |
| 696271 | 21.4 | 21.5 | 0.09 |
| 696287 | 24.0 | 24.4 | 0.15 |
| 696358 | 22.2 | 21.2 | 0.08 |
| 696377 | 23.2 | 23.6 | 0.10 |
| 696556 | 23.3 | 22.9 | 0.10 |

### Plasma Chemistry Markers

To evaluate the effect of antisense oligonucleotides on liver and kidney function, plasma levels of transaminases, total bilirubin and blood urea nitrogen (BUN) were measured using an automated clinical chemistry analyzer (Hitachi Olympus AU400e, Melville, N.Y.). The results are presented in the Table below.

TABLE 56
Plasma chemistry markers in A431 xenograft model

| ISIS | ALT | AST | Albumin | T. Bilirubin | BUN |
|---|---|---|---|---|---|
| No. | (U/L) | (U/L) | g/dL | (mg/dL) | (mg/dL) |
| PBS | 35.5 | 73.6 | 3.0 | 0.13 | 20 |
| 481464 | 42.3 | 91.0 | 2.9 | 0.10 | 21 |
| 549148 | 33.3 | 81.3 | 2.9 | 0.13 | 14 |
| 651987 | 43.5 | 101.0 | 2.8 | 0.12 | 25 |
| 695785 | 56.5 | 146.5 | 2.8 | 0.17 | 25 |
| 695809 | 264.8 | 312.0 | 2.7 | 0.18 | 17 |
| 695917 | 270.0 | 389.3 | 2.8 | 0.16 | 19 |
| 695924 | 540.0 | 5421.0 | 3.4 | 2.20 | 48 |
| 695977 | 32.0 | 70.0 | 2.9 | 0.15 | 22 |
| 695998 | 3197.3 | 3515.8 | 3.0 | 2.96 | 17 |
| 696012 | 41.0 | 135.3 | 2.8 | 0.15 | 21 |
| 696013 | 687.0 | 1511.0 | 2.4 | 0.17 | 20 |
| 696017 | 4189.3 | 3429.0 | 2.6 | 0.84 | 18 |
| 696018 | 126.8 | 197.8 | 3.2 | 0.18 | 19 |
| 696043 | 57.0 | 146.0 | 2.8 | 0.15 | 21 |
| 696044 | 734.5 | 871.3 | 3.0 | 0.24 | 16 |
| 696091 | 2168.0 | 6776.0 | 2.5 | 7.61 | 35 |
| 696096 | 4400.3 | 2436.0 | 2.1 | 2.35 | 18 |
| 696108 | 45.8 | 105.0 | 3.0 | 0.13 | 19 |
| 696117 | 1699.3 | 1426.3 | 3.2 | 0.22 | 18 |
| 696137 | 5308.3 | 6041.3 | 2.9 | 0.52 | 24 |
| 696152 | 1818.3 | 1798.0 | 2.9 | 0.30 | 22 |
| 696167 | 271.0 | 312.5 | 3.0 | 0.17 | 17 |
| 696271 | 41.3 | 84.8 | 2.9 | 0.16 | 20 |
| 696287 | 92.0 | 183.3 | 2.8 | 0.13 | 17 |
| 696358 | 2284.3 | 1820.0 | 3.0 | 0.27 | 18 |
| 696377 | 34.8 | 106.5 | 3.0 | 0.12 | 18 |
| 696556 | 36.0 | 87.0 | 3.0 | 0.15 | 17 |

### Study 3

### RNA Analysis

RNA was extracted from tumor tissue for real-time PCR analysis and measurement of human K-Ras mRNA levels using primer probe set RTS3496_MGB, described herein above. Results are presented as average percent inhibition of K-Ras for each treatment group, relative to PBS control. As shown in the Table below, treatment with Isis antisense oligonucleotides resulted in reduction of human K-Ras mRNA in comparison to the PBS control.

TABLE 57
Antisense mediated inhibition of human K-Ras
mRNA expression in A431 xenograft model

| ISIS | Inhibition |
|---|---|
| No. | (%) |
| 651588 | 69 |
| 651653 | 41 |
| 651987 | 51 |
| 716587 | 40 |
| 716588 | 37 |
| 716600 | 55 |
| 716608 | 43 |
| 716612 | 39 |
| 716628 | 48 |
| 716655 | 49 |
| 716656 | 64 |
| 716673 | 54 |
| 716683 | 50 |
| 716716 | 51 |
| 716758 | 74 |
| 716769 | 52 |
| 716772 | 47 |

### Body Weight Measurements

Body weights were measured throughout the treatment period. The data is presented in the Table below as the average for each treatment group at various time points. Organ weights were measured at the end of the study (Day 23) and are presented in the Table below.

TABLE 58
Body and organ weight measurements in A431 xenograft model

| Isis | Body weight (g) | | | | Organ weights (g) | | |
|---|---|---|---|---|---|---|---|
| No. | Day −1 | Day 7 | Day 14 | Day 21 | kidney | liver | spleen |
| PBS | 20.5 | 21.0 | 21.5 | 21.9 | 0.30 | 1.06 | 0.09 |
| 651588 | 19.9 | 20.9 | 20.3 | 18.0 | 0.29 | 1.41 | 0.07 |
| 651653 | 20.1 | 21.1 | 20.5 | 20.4 | 0.32 | 1.63 | 0.13 |
| 651987 | 21.2 | 21.6 | 21.7 | 22.2 | 0.33 | 1.42 | 0.12 |
| 716587 | 21.7 | 23.2 | 21.4 | 22.4 | 0.36 | 2.38 | 0.15 |
| 716588 | 20.4 | 21.7 | 21.7 | 22.1 | 0.33 | 1.74 | 0.09 |
| 716600 | 20.3 | 21.3 | 22.0 | 22.7 | 0.34 | N/A | 0.13 |
| 716608 | 21.0 | 19.9 | 17.3 | 20.9 | 0.39 | 2.07 | 0.11 |
| 716612 | 21.7 | 22.1 | 22.4 | 23.5 | 0.34 | 2.78 | 0.14 |
| 716625 | 20.1 | 20.3 | 17.2 | N/A | N/A | N/A | N/A |
| 716628 | 21.0 | 21.7 | 20.8 | 20.1 | 0.36 | 1.55 | 0.12 |
| 716655 | 19.6 | 20.6 | 20.9 | 21.5 | 0.33 | 1.47 | 0.13 |
| 716656 | 20.5 | 20.9 | 19.9 | 18.5 | 0.29 | 1.69 | 0.07 |
| 716673 | 19.2 | 20.5 | 19.0 | 18.9 | 0.32 | 2.47 | 0.08 |
| 716674 | 20.3 | 21.0 | N/A | N/A | N/A | N/A | N/A |
| 716675 | 21.4 | 20.0 | N/A | N/A | N/A | N/A | N/A |
| 716683 | 21.2 | 21.0 | 19.2 | 19.9 | 0.35 | 2.58 | 0.09 |
| 716716 | 19.7 | 20.5 | 20.8 | 21.4 | 0.34 | 1.43 | 0.11 |
| 716728 | 21.7 | 18.4 | N/A | N/A | N/A | N/A | N/A |
| 716758 | 20.6 | 20.3 | 17.6 | 16.2 | 0.30 | 1.29 | 0.05 |
| 716763 | 21.8 | N/A | N/A | N/A | N/A | N/A | N/A |
| 716769 | 19.6 | 20.6 | 20.8 | 20.7 | 0.38 | 1.54 | 0.12 |
| 716772 | 19.9 | 20.9 | 21.3 | 22.1 | 0.29 | 1.16 | 0.10 |

### Plasma Chemistry Markers

To evaluate the effect of antisense oligonucleotides on liver and kidney function, plasma levels of transaminases, total bilirubin and blood urea nitrogen (BUN) were measured using an automated clinical chemistry analyzer (Hitachi Olympus AU400e, Melville, N.Y.). The results are presented in the Table below.

TABLE 59
Plasma chemistry markers in A431 xenograft model

| ISIS | ALT | AST | T. Bilirubin | BUN |
|---|---|---|---|---|
| No. | (U/L) | (U/L) | (mg/dL) | (mg/dL) |
| PBS | 31.4 | 109.8 | 0.17 | 20 |
| 651588 | 4064.3 | 3039.5 | 0.21 | 23 |
| 651653 | 1900.5 | 1571.3 | 0.20 | 21 |
| 651987 | 88.5 | 150.8 | 0.11 | 20 |
| 716587 | 640.0 | 401.5 | 0.22 | 18 |
| 716588 | 1418.8 | 1067.8 | 0.17 | 20 |
| 716600 | 217.3 | 316.0 | 0.11 | 19 |
| 716608 | 950.0 | 1622.0 | 0.26 | 22 |
| 716612 | 281.0 | 267.5 | 0.10 | 19 |
| 716628 | 2662.7 | 3046.0 | 0.18 | 18 |
| 716655 | 125.0 | 208.8 | 0.11 | 23 |
| 716656 | 3306.3 | 2432.3 | 2.43 | 24 |
| 716673 | 3574.3 | 2518.5 | 1.01 | 24 |
| 716683 | 2644.5 | 3775.0 | 0.22 | 28 |

### Study 4

### RNA Analysis

RNA was extracted from tumor tissue for real-time PCR analysis and measurement of human K-Ras mRNA levels using primer probe set RTS3496_MGB, described herein above. Results are presented as average percent inhibition of K-Ras for each treatment group, relative to PBS control. As shown in the Tables below, treatment with Isis antisense oligonucleotides resulted in reduction of human K-Ras mRNA in comparison to the PBS control.

TABLE 60
Antisense mediated inhibition of human K-Ras
mRNA expression in A431 xenograft model

| ISIS | Inhibition |
|---|---|
| No. | (%) |
| 481464 | 0 |
| 651987 | 45 |
| 696816 | 14 |
| 740167 | 40 |
| 740171 | 31 |
| 740173 | 50 |
| 740179 | 50 |
| 740187 | 39 |
| 740188 | 62 |
| 740191 | 53 |
| 740194 | 56 |
| 740196 | 62 |
| 740199 | 57 |
| 740201 | 51 |
| 740212 | 41 |
| 740216 | 42 |
| 740218 | 48 |
| 740221 | 59 |
| 740223 | 57 |
| 740227 | 42 |
| 740233 | 54 |
| 740238 | 38 |
| 740244 | 22 |
| 740246 | 47 |
| 740250 | 50 |
| 740255 | 47 |
| 740256 | 67 |
| 740261 | 0 |
| 740294 | 30 |

### Body Weight Measurements

Body weights were measured throughout the treatment period. The data is presented in the Table below as the average for each treatment group at various time points. Organ weights were measured at the end of the study and are presented in the Table below.

TABLE 61
Body weight measurements in A431 xenograft model

| Isis | Body weight (g) | | | | | Organ weights (g) | | |
|---|---|---|---|---|---|---|---|---|
| No. | Day 3 | Day 8 | Day 15 | Day 22 | Day 24 | kidney | liver | spleen |
| PBS | 22.4 | 23.0 | 23.3 | 23.6 | 23.3 | 0.33 | 1.27 | 0.08 |
| 481464 | 19.9 | 20.5 | 20.8 | 21.9 | 22.1 | 0.30 | 1.32 | 0.11 |
| 651987 | 22.8 | 23.4 | 22.9 | 23.7 | 24.1 | 0.35 | 1.59 | 0.13 |
| 696816 | 21.1 | 21.7 | 21.6 | 22.7 | 23.0 | 0.33 | 1.33 | 0.11 |
| 740167 | 20.3 | 20.7 | 20.4 | 20.9 | 20.5 | 0.30 | 1.26 | 0.09 |
| 740171 | 22.1 | 22.4 | 23.1 | 24.2 | 24.5 | 0.36 | 1.51 | 0.37 |
| 740173 | 21.0 | 21.6 | 21.5 | 21.0 | 20.9 | 0.31 | 2.38 | 0.14 |
| 740179 | 21.4 | 20.9 | 22.2 | 22.8 | 22.7 | 0.36 | 1.41 | 0.12 |
| 740187 | 23.0 | 23.2 | 23.4 | 24.2 | 24.4 | 0.34 | 1.53 | 0.11 |
| 740188 | 22.6 | 21.7 | 19.6 | 19.4 | 19.7 | 0.35 | 3.23 | 0.03 |
| 740191 | 23.9 | 24.7 | 25.2 | 24.6 | 24.7 | 0.37 | 1.51 | 0.13 |
| 740194 | 23.5 | 24.3 | 23.4 | 21.7 | 21.8 | 0.40 | 1.61 | 0.16 |
| 740196 | 21.2 | 21.7 | 18.0 | 21.9 | 23.8 | 0.37 | 2.66 | 0.09 |
| 740199 | 21.5 | 22.3 | 21.8 | 21.5 | 21.8 | 0.39 | 2.40 | 0.12 |
| 740201 | 22.7 | 22.9 | 23.1 | 23.7 | 23.5 | 0.36 | 1.70 | 0.13 |
| 740212 | 21.9 | 22.4 | 22.2 | 22.5 | 22.3 | 0.33 | 1.44 | 0.14 |
| 740216 | 22.1 | 22.7 | 22.4 | 22.3 | 22.5 | 0.41 | 1.89 | 0.16 |
| 740218 | 20.8 | 21.4 | 21.3 | 21.0 | 21.7 | 0.33 | 2.72 | 0.13 |
| 740221 | 23.0 | 23.9 | 22.1 | 22.7 | 23.4 | 0.41 | 3.43 | 0.09 |
| 740223 | 20.6 | 21.1 | 21.1 | 21.2 | 21.2 | 0.36 | 1.28 | 0.09 |
| 740227 | 20.3 | 21.2 | 21.0 | 21.6 | 21.4 | 0.34 | 2.24 | 0.14 |
| 740233 | 21.8 | 22.4 | 22.5 | 22.8 | 22.9 | 0.36 | 1.37 | 0.10 |
| 740238 | 23.8 | 24.5 | 24.5 | 25.0 | 25.1 | 0.38 | 1.61 | 0.13 |
| 740244 | 21.3 | 22.8 | 22.7 | 22.9 | 23.2 | 0.38 | 1.65 | 0.12 |
| 740246 | 20.9 | 21.5 | 21.5 | 21.9 | 21.7 | 0.37 | 3.48 | 0.13 |
| 740250 | 23.1 | 23.2 | 19.8 | 20.5 | 21.5 | 0.39 | 2.69 | 0.10 |
| 740255 | 21.9 | 22.6 | 22.4 | 22.7 | 22.7 | 0.33 | 2.18 | 0.09 |
| 740256 | 21.1 | 21.3 | 19.5 | 21.2 | 21.5 | 0.37 | 2.81 | 0.09 |
| 740258 | 21.8 | 21.2 | 17.3 | N/A | N/A | N/A | N/A | N/A |
| 740261 | 21.3 | 21.9 | 21.0 | 22.3 | 22.8 | 0.33 | 1.42 | 0.12 |
| 740294 | 20.8 | 21.5 | 21.9 | 22.0 | 21.8 | 0.36 | 1.75 | 0.11 |
| 740302 | 22.8 | 22.9 | N/A | N/A | N/A | N/A | N/A | N/A |

### Plasma Chemistry Markers

To evaluate the effect of antisense oligonucleotides on liver and kidney function, plasma levels of transaminases, total bilirubin and blood urea nitrogen (BUN) were measured using an automated clinical chemistry analyzer (Hitachi Olympus AU400e, Melville, N.Y.). The results are presented in the Table below.

TABLE 62
Plasma chemistry markers in A431 xenograft model

| ISIS | ALT | AST | Albumin | T. Bilirubin | BUN |
|---|---|---|---|---|---|
| No. | (U/L) | (U/L) | g/dL | (mg/dL) | (mg/dL) |
| PBS | 45.0 | 85.3 | 3.2 | 0.14 | 25 |
| 481464 | 47.0 | 138.8 | 2.8 | 0.14 | 21 |
| 651987 | 68.3 | 239.3 | 2.9 | 0.18 | 18 |
| 696816 | 55.5 | 139.5 | 2.5 | 0.14 | 31 |
| 740167 | 66.0 | 167.0 | 2.8 | 0.16 | 18 |
| 740171 | 59.5 | 182.3 | 3.0 | 0.18 | 18 |
| 740173 | 1375.8 | 964.8 | 2.8 | 5.05 | 19 |
| 740179 | 105.5 | 262.3 | 3.1 | 0.21 | 17 |
| 740187 | 129.3 | 164.5 | 2.9 | 0.14 | 22 |
| 740188 | 3098.0 | 3275.0 | 2.3 | 0.24 | 50 |
| 740191 | 69.8 | 161.8 | 2.9 | 0.14 | 18 |
| 740194 | 2729.8 | 3065.5 | 2.5 | 0.68 | 20 |
| 740196 | 2553.0 | 1607.3 | 3.2 | 0.17 | 24 |
| 740199 | 3552.8 | 3428.5 | 3.4 | 0.28 | 19 |
| 740201 | 190.5 | 367.0 | 2.9 | 0.19 | 19 |
| 740212 | 59.8 | 184.3 | 3.2 | 0.19 | 22 |
| 740216 | 2297.0 | 2663.5 | 2.9 | 0.95 | 20 |
| 740218 | 3984.8 | 2519.8 | 2.1 | 2.77 | 21 |
| 740221 | 4076.0 | 2571.5 | 2.4 | 0.33 | 22 |
| 740223 | 142.3 | 355.5 | 3.2 | 0.19 | 18 |
| 740227 | 3511.5 | 3993.0 | 2.4 | 0.62 | 16 |
| 740233 | 67.3 | 162.5 | 2.8 | 0.16 | 21 |
| 740238 | 165.0 | 362.0 | 2.8 | 0.16 | 20 |
| 740244 | 118.3 | 276.0 | 3.1 | 0.18 | 20 |
| 740246 | 3198.8 | 1579.8 | 2.2 | 0.42 | 22 |
| 740250 | 1710.5 | 1364.3 | 2.8 | 0.16 | 25 |
| 740255 | 1579.5 | 827.3 | 2.8 | 0.34 | 21 |
| 740256 | 3725.5 | 2068.5 | 2.8 | 0.21 | 27 |
| 740261 | 84.3 | 238.8 | 3.0 | 0.20 | 22 |
| 740294 | 1596.3 | 1357.0 | 3.0 | 0.31 | 22 |

### Study 5

### RNA Analysis

RNA was extracted from tumor tissue for real-time PCR analysis and measurement of human K-Ras mRNA levels using primer probe set RTS3496_MGB, described herein above. Results are presented as average percent inhibition of K-Ras for each treatment group, relative to PBS control, normalized to glyceraldehyde-3-phosphate dehydrogenase or beta-actin mRNA levels. As shown in the Tables below, treatment with Isis antisense oligonucleotides resulted in reduction of human K-Ras mRNA in comparison to the PBS control.

TABLE 63
Antisense mediated inhibition of human K-Ras
mRNA expression in A431 xenograft model

| ISIS No. | Inhibition (%) |
|---|---|
| 651555 | 48 |
| 651987 | 36 |
| 695823 | 26 |
| 695980 | 35 |
| 696018 | 47 |
| 716744 | 25 |
| 716749 | 0 |
| 716754 | 26 |
| 746273 | 9 |
| 746275 | 51 |
| 746276 | 26 |
| 746279 | 31 |
| 746280 | 43 |
| 746285 | 24 |
| 746286 | 51 |
| 746287 | 45 |

### Body Weight Measurements

Body weights were measured throughout the treatment period. The data is presented in the Table below as the average for each treatment group at various time points. At the end of the study (day 23), organ weights were measured and are presented in the Table below.

TABLE 64
Body and organ weight measurements in A431 xenograft model

| Isis | Body weight (g) | | | | Organ weights (g) | | |
|---|---|---|---|---|---|---|---|
| No. | Day 1 | Day 8 | Day 15 | Day 23 | kidney | liver | spleen |
| PBS | 19.2 | 19.8 | 20.2 | 20.6 | 0.34 | 1.11 | 0.10 |
| 651555 | 18.0 | 17.8 | 18.4 | 18.9 | 0.33 | 1.26 | 0.08 |
| 651987 | 19.1 | 19.4 | 20.2 | 21.4 | 0.30 | 1.34 | 0.14 |
| 695823 | 18.8 | 19.6 | 19.4 | 19.8 | 0.26 | 1.17 | 0.09 |
| 695980 | 20.1 | 20.2 | 20.7 | 21.6 | 0.37 | 1.32 | 0.09 |
| 696018 | 19.0 | 20.3 | 19.7 | 19.8 | 0.31 | 1.32 | 0.08 |
| 716744 | 18.3 | 18.3 | 18.8 | 19.8 | 0.28 | 1.17 | 0.09 |
| 716749 | 19.8 | 20.9 | 20.6 | 21.3 | 0.31 | 1.27 | 0.09 |
| 716754 | 19.4 | 19.2 | 19.5 | 20.0 | 0.28 | 1.11 | 0.09 |
| 746273 | 18.8 | 19.3 | 19.4 | 20.1 | 0.28 | 1.04 | 0.08 |
| 746275 | 18.1 | 18.3 | 18.7 | 19.7 | 0.29 | 1.12 | 0.07 |
| 746276 | 19.9 | 20.9 | 21.1 | 21.7 | 0.33 | 1.31 | 0.09 |
| 746279 | 18.7 | 19.2 | 19.9 | 20.6 | 0.30 | 1.22 | 0.11 |
| 746280 | 18.5 | 19.3 | 19.5 | 20.3 | 0.29 | 1.19 | 0.10 |
| 746285 | 20.2 | 20.5 | 20.8 | 21.3 | 0.33 | 1.33 | 0.08 |
| 746286 | 19.8 | 19.9 | 19.9 | 19.7 | 0.31 | 1.41 | 0.06 |
| 746287 | 19.2 | 19.6 | 19.1 | 19.4 | 0.30 | 1.51 | 0.07 |

### Plasma Chemistry Markers

To evaluate the effect of antisense oligonucleotides on liver and kidney function, plasma levels of transaminases, total bilirubin and blood urea nitrogen (BUN) were measured using an automated clinical chemistry analyzer (Hitachi Olympus AU400e, Melville, N.Y.). The results are presented in the Table below.

TABLE 65
Plasma chemistry markers in A431 xenograft model

| | ALT | AST | T. Bilirubin | BUN |
|---|---|---|---|---|
| ISIS No. | (U/L) | (U/L) | (mg/dL) | (mg/dL) |
| PBS | 41.8 | 125.0 | 0.12 | 21 |
| 651555 | 65.3 | 207.3 | 0.16 | 23 |
| 651987 | 47.5 | 185.3 | 0.17 | 23 |
| 695823 | 35.0 | 106.8 | 0.15 | 21 |
| 695980 | 36.5 | 115.3 | 0.11 | 18 |
| 696018 | 381.5 | 634.8 | 0.16 | 21 |
| 716744 | 37.3 | 103.5 | .09 | 22 |
| 716749 | 43.0 | 141.0 | .12 | 22 |
| 716754 | 82.8 | 232.3 | .12 | 23 |
| 746273 | 38.8 | 133.3 | .13 | 22 |
| 746275 | 79.5 | 275.5 | 0.18 | 24 |
| 746276 | 95.8 | 340.3 | .19 | 18 |
| 746279 | 85.8 | 276.5 | .17 | 22 |
| 746280 | 84.8 | 281.0 | .18 | 24 |
| 746285 | 336.0 | 696.8 | .20 | 21 |
| 746286 | 1345.8 | 1871.5 | .21 | 20 |
| 746287 | 1454.0 | 2072.5 | .31 | 22 |

TABLE 66
Plasma chemistry markers in A431 xenograft model

| | ALT | AST | T. Bilirubin | BUN |
|---|---|---|---|---|
| ISIS No. | (U/L) | (U/L) | (mg/dL) | (mg/dL) |
| PBS | 35.5 | 73.6 | 0.13 | 20 |
| 651987 | 43.5 | 101.0 | 0.12 | 25 |
| 695785 | 56.5 | 146.5 | 0.17 | 25 |
| 696018 | 126.8 | 197.8 | 0.18 | 19 |
| 696044 | 734.5 | 871.3 | 0.24 | 16 |

### Example 14: Tolerability of Antisense Oligonucleotides Targeting Human K-Ras mRNA in Sprague-Dawley Rats

The antisense oligonucleotides described in the studies above were also tested for in vivo tolerability in Sprague-Dawley rats.

Groups of four Sprague-Dawley rats were injected subcutaneously once per week for 6 weeks, for a total of 7 treatments, with 50 mg/kg of an antisense oligonucleotide. A control group of rats was injected subcutaneously once per week for 6 weeks with PBS. Two days after the last dose rats were euthanized and organs and plasma were harvested for further analysis. Body weights were measured throughout the study.

To evaluate the effect of the antisense oligonucleotides on hepatic function, plasma concentrations of transaminases (ALT, AST), Albumin (Alb) and total bilirubin (T. Bil.) were measured using an automated clinical chemistry analyzer (Hitachi Olympus AU400e, Melville, N.Y.).

To evaluate the effect of the antisense oligonucleotides on kidney function, plasma concentrations of blood urea nitrogen (BUN) and creatinine (Cre) were measured using an automated clinical chemistry analyzer (Hitachi Olympus AU400e, Melville, N.Y.). Albumin (Alb) was also measured. Total urine protein (Micro Total Protein (MTP)) and urine creatinine levels as well as the ratio of total urine protein to creatinine (MTP/CREA) were also determined.

Liver, spleen, and kidney weights were measured at the end of the study.

The results are presented in the Tables below and show that many antisense oligonucleotides targeting human K-Ras were well tolerated in Sprague Dawley rats.

TABLE 67
Body and organ weights

| Isis | Body weight (g) on indicated study day | | | | | | | Liver | Spleen | Kidney |
|---|---|---|---|---|---|---|---|---|---|---|
| No. | 1 | 8 | 15 | 22 | 29 | 36 | 43 | (g) | (g) | (g) |
| PBS | 348.8 | 371.0 | 407.0 | 425.8 | 443.3 | 463.0 | 478.0 | 16.9 | 0.84 | 3.6 |
| 651530 | 351.3 | 377.8 | 408.5 | 432.0 | 449.5 | 461.5 | 466.0 | 17.1 | 1.26 | 3.5 |
| 651555 | 369.8 | 385.5 | 414.3 | 424.3 | 433.3 | 439.8 | 443.3 | 17.7 | 2.50 | 3.6 |
| 651587 | 357.0 | 375.3 | 412.0 | 429.3 | 437.0 | 442.5 | 450.5 | 16.8 | 1.40 | 3.7 |
| 651987 | 350.8 | 375.3 | 403.0 | 415.5 | 425.8 | 434.5 | 439.3 | 19.2 | 1.74 | 3.5 |
| 695785 | 351.5 | 364.3 | 384.5 | 389.5 | 393.0 | 395.3 | 409.5 | 17.8 | 2.51 | 3.9 |
| 695823 | 352.8 | 372.5 | 398.5 | 414.3 | 423.8 | 434.0 | 440.8 | 16.7 | 1.23 | 3.4 |
| 695980 | 363.5 | 379.8 | 407.8 | 416.8 | 419.5 | 422.8 | 429.3 | 17.6 | 3.06 | 3.8 |
| 695995 | 332.3 | 349.5 | 371.8 | 372.5 | 370.0 | 368.8 | 374.3 | 13.4 | 1.29 | 2.9 |

TABLE 68
Plasma and urine clinical chemistry

| | plasma | | | | | | urine | | |
|---|---|---|---|---|---|---|---|---|---|
| | ALT | AST | Alb | BUN | Cre | T. Bil. | Cre | MTP | MTP/ |
| Isis No. | (U/L) | (U/L) | (g/dL) | (mg/dL) | (mg/dL) | (mg/dL) | (mg/dL) | (mg/dL) | CREA |
| PBS | 74.3 | 83.3 | 3.3 | 21.6 | 0.32 | 0.14 | 118.5 | 117.8 | 1.0 |
| 549148 | 56.5 | 89.8 | 3.4 | 22.5 | 0.42 | 0.13 | 96.8 | 366.3 | 3.8 |
| 651530 | 90.8 | 96.0 | 3.4 | 20.6 | 0.38 | 0.14 | 104.8 | 415.8 | 4.0 |
| 651555 | 95.3 | 116.0 | 3.0 | 27.8 | 0.40 | 0.13 | 84.5 | 547.3 | 6.5 |
| 651587 | 88.8 | 102.8 | 3.1 | 40.6 | 0.66 | 0.15 | 66.5 | 675.0 | 10.2 |
| 651987 | 62.8 | 65.8 | 2.1 | 46.5 | 0.58 | 0.19 | 92.8 | 569.8 | 6.1 |
| 695785 | 97.0 | 87.3 | 3.0 | 24.8 | 0.38 | 0.13 | 63.0 | 585.3 | 9.3 |
| 695823 | 73.5 | 87.8 | 3.9 | 27.1 | 0.47 | 0.16 | 89.3 | 421.3 | 4.7 |
| 695980 | 69.0 | 109.8 | 3.3 | 27.1 | 0.50 | 0.13 | 71.0 | 488.8 | 6.9 |
| 695995 | 240.3 | 203.5 | 4.0 | 29.3 | 0.53 | 0.25 | 61.0 | 244.8 | 4.0 |

### Example 15: Tolerability of Antisense Oligonucleotides Targeting Human K-Ras mRNA in Sprague-Dawley Rats

The antisense oligonucleotides described in the studies above were also tested for in vivo tolerability in Sprague-Dawley rats.

Groups of four Sprague-Dawley rats were injected subcutaneously once per week for 6 weeks, for a total of 7 treatments, with 50 mg/kg of an antisense oligonucleotide. A control group of rats was injected subcutaneously once per week for 6 weeks with PBS. Two days after the last dose rats were euthanized and organs and plasma were harvested for further analysis. Body weights were measured throughout the study.

To evaluate the effect of the antisense oligonucleotides on hepatic function, plasma concentrations of transaminases (ALT, AST), Albumin (Alb) and total bilirubin (T. Bil.) were measured using an automated clinical chemistry analyzer (Hitachi Olympus AU400e, Melville, N.Y.).

To evaluate the effect of the antisense oligonucleotides on kidney function, plasma concentrations of blood urea nitrogen (BUN) and creatinine (Cre) were measured using an automated clinical chemistry analyzer (Hitachi Olympus AU400e, Melville, N.Y.). Albumin (Alb) was also measured. Total urine protein (Micro Total Protein (MTP)) and urine creatinine levels as well as the ratio of total urine protein to creatinine (MTP/CREA) were also determined.

Liver, spleen, and kidney weights were measured at the end of the study.

The results are presented in the Tables below and show that many antisense oligonucleotides targeting human K-Ras were well tolerated in Sprague Dawley rats.

TABLE 69
Body and organ weights

| | Body weight (g) on indicated study day | | | | | | | | | |
|---|---|---|---|---|---|---|---|---|---|---|
| Isis No. | 1 | 8 | 15 | 22 | 29 | 36 | 43 | Liver (g) | Spleen (g) | Kidney (g) |
| PBS | 252.3 | 321.5 | 377.8 | 417.8 | 456.8 | 453.8 | 492.8 | 20.5 | 0.91 | 3.42 |
| 696018 | 243.3 | 298.8 | 329.5 | 344.5 | 348.8 | 340.8 | 333.3 | 13.5 | 2.03 | 3.38 |
| 696044 | 245.5 | 299.0 | 336.5 | 350.5 | 345.0 | 351.5 | 348.5 | 15.8 | 2.57 | 3.52 |
| 716600 | 240.0 | 287.5 | 316.8 | 333.5 | 347.0 | 351.5 | 360.3 | 17.2 | 1.87 | 3.28 |
| 716655 | 247.8 | 305.0 | 341.8 | 360.3 | 370.5 | 374.3 | 363.5 | 16.6 | 3.23 | 4.86 |
| 740233 | 247.0 | 305.3 | 347.3 | 375.3 | 393.0 | 389.0 | 383.5 | 16.9 | 2.13 | 3.82 |
| 746275 | 242.8 | 305.5 | 350.8 | 372.0 | 392.5 | 401.8 | 405.0 | 17.5 | 2.51 | 3.85 |

TABLE 70
Plasma and urine clinical chemistry

| | plasma | | | | | | urine | | |
|---|---|---|---|---|---|---|---|---|---|
| Isis | ALT | AST | Alb | BUN | Cre | T. Bil. | Cre | MTP | MTP/ |
| No. | (U/L) | (U/L) | (g/dL) | (mg/dL) | (mg/dL) | (mg/dL) | (mg/dL) | (mg/dL) | CREA |
| PBS | 42.0 | 59.5 | 3.3 | 17.1 | 0.32 | 0.14 | 60.8 | 69.3 | 1.14 |
| 696018 | 47.5 | 84.8 | 2.9 | 20.5 | 0.41 | 0.19 | 32.0 | 113.3 | 3.54 |
| 696044 | 49.5 | 104.8 | 2.2 | 20.7 | 0.34 | 0.12 | 42.0 | 543.5 | 12.94 |
| 716600 | 38.3 | 71.8 | 2.5 | 17.2 | 0.32 | 0.10 | 60.3 | 455.3 | 7.56 |
| 716655 | 48.3 | 62.8 | 1.8 | 60.9 | 0.66 | 0.08 | 67.5 | 618.5 | 9.16 |
| 740233 | 53.3 | 126.3 | 2.8 | 19.3 | 0.33 | 0.13 | 52.5 | 421.0 | 8.02 |
| 746275 | 212.8 | 222.0 | 2.9 | 18.6 | 0.37 | 0.17 | 50.5 | 330.8 | 6.55 |

### Example 16: Comparative Evaluation of Potency for Gen1.0 and Gen2.5 Human K-RAS Antisense Oligonucleotides

Antisense oligonucleotides described above and Isis No. 6957 were tested at various doses in A431 cells. Isis No. 6957, described in U.S. Pat. No. 6,784,290, consists of 2′-deoxynucleosides linked via phosphorothioate internucleoside linkages, and the sequence is CAGTGCCTGCGCCGCGCTCG (SEQ ID NO: 2193). Isis No. 549148, which does not target K-Ras, was included as a negative control. A431 cells were plated at a density of 10,000 cells per well and incubated with concentrations of antisense oligonucleotide specified in Table 24 below. After 24 hours, RNA was isolated from the cells and K-Ras mRNA levels were measured by quantitative real-time PCR. RTS3496_MGB primer probe set was used to measure K-Ras mRNA levels. K-Ras mRNA levels were normalized to beta-actin mRNA levels. Results are presented as percent inhibition of K-Ras mRNA, relative to untreated control cells.

As illustrated in the Table below, the new antisense oligonucleotides were much more potent than Isis No. 6957, which exhibited minimal inhibition of K-Ras.

TABLE 71
Dose-dependent inhibition of human K-Ras mRNA expression
by free-uptake of ISIS oligonucleotides

| | Inhibition (%) | | | | | |
|---|---|---|---|---|---|---|
| | | | | | 3333 | |
| ISIS No. | 41.2 nM | 123 nM | 370 nM | 1111 nM | nM | 10000 nM |
| 6957 | 0 | 1 | 2 | 0 | 7 | 17 |
| 549148 | 6 | 4 | 1 | 2 | 3 | 1 |
| 651530 | 14 | 26 | 54 | 69 | 90 | 92 |
| 651555 | 15 | 32 | 57 | 63 | 77 | 84 |
| 651587 | 17 | 22 | 69 | 78 | 80 | 87 |
| 651987 | 27 | 40 | 70 | 79 | 88 | 91 |
| 695785 | 13 | 39 | 53 | 73 | 85 | 87 |
| 695823 | 18 | 31 | 43 | 67 | 77 | 81 |
| 695980 | 19 | 37 | 65 | 76 | 84 | 88 |
| 695995 | 18 | 32 | 45 | 74 | 84 | 90 |

### Example 17: Pharmacodynamics and Toxicological Profile of Human K-Ras Antisense Oligonucleotides in COLO205 Adenocarcinoma Xenograft Model

Female, 6-8 week old NCr nude mice (Taconic Biosciences, Hudson, N.Y.) were inoculated with human colorectal adenocarcinoma COLO205 cells and treated with antisense oligonucleotides or with PBS. K-Ras expression and tolerability of the oligonucleotides in the mice were evaluated.

### Treatment

For tumor development, the mice were each inoculated in the right lateral fat pad with 3×10⁶ COLO205 cells in 50% Matrigel (BD Bioscience). Antisense oligonucleotide treatment started around day 10 after tumor inoculation when the mean tumor size reached approximately 200 mm³. The mice were subcutaneously injected with 30 or 50 mg/kg/week three times per week for three weeks, for a total of nine doses at 150 or 250 mg/kg/week, with antisense oligonucleotide or PBS. RNA was extracted from tumor tissue for real-time PCR analysis. The mice were euthanized 24 hours after the last dose, and organs and plasma were harvested for further analysis. Body weights were measured throughout the study. Liver, spleen, and kidney weights were measured at the end of the study. The results are presented in the Tables below, demonstrating that many antisense oligonucleotides targeting human K-Ras resulted in reduction of K-Ras mRNA levels, and were well tolerated.

### RNA Analysis

RNA was extracted from tumor tissue for real-time PCR analysis and measurement of human K-Ras mRNA levels using primer probe set RTS3496_MGB, described herein above. Results are presented as average percent inhibition of K-Ras for each treatment group, relative to PBS control, normalized to glyceraldehyde-3-phosphate dehydrogenase mRNA levels. As shown in the tables below, treatment with Isis antisense oligonucleotides resulted in reduction of human K-Ras mRNA in comparison to the PBS control.

TABLE 72
Inhibition of human K-Ras mRNA
expression in COLO205 xenograft model

| ISIS No. | mg/kg/week | Inhibition (%) |
|---|---|---|
| PBS | N/A | 0 |
| 651555 | 250 | 47 |
| | 150 | 23 |
| 651987 | 250 | 36 |
| | 150 | 30 |
| 695980 | 250 | 41 |
| | 150 | 28 |
| 696044 | 250 | 43 |
| | 150 | 45 |
| 716600 | 250 | 49 |
| | 150 | 43 |
| 716655 | 250 | 36 |
| | 150 | 36 |
| 716772 | 250 | 33 |
| | 150 | 27 |
| 740179 | 250 | 24 |
| | 150 | 28 |
| 740256 | 150 | 46 |

### Body Weight Measurements

Body weights were measured throughout the treatment period. The data is presented in the tables below as the average for each treatment group at various time points. At the end of the study, organ weights were measured and are presented in the table below.

TABLE 73
Body and organ weight measurements

| | | Body weight (g) | | | | | | |
|---|---|---|---|---|---|---|---|---|
| ISIS | mg/kg/ | | | day | day | Organ weight (g) | | |
| No. | week | day 1 | day 9 | 16 | 23 | kidney | liver | spleen |
| PBS | N/A | 21.9 | 21.5 | 19.7 | 21.5 | 0.3 | 1.1 | 0.10 |
| 651555 | 250 | 20.4 | 19.0 | 18.2 | 17.6 | 0.3 | 1.0 | 0.05 |
| | 150 | 18.5 | 21.9 | 19.9 | 21.3 | 0.2 | 1.2 | 0.09 |
| 651987 | 250 | 20.7 | 20.3 | 18.0 | 16.7 | 0.3 | 1.1 | 0.05 |
| | 150 | 20.6 | 18.1 | 16.0 | 16.4 | 0.2 | 1.0 | 0.06 |
| 695980 | 250 | 20.6 | 19.7 | 17.9 | 19.4 | 0.4 | 1.2 | 0.14 |
| | 150 | 19.5 | 19.8 | 20.3 | 20.9 | 0.3 | 1.1 | 0.06 |
| 696044 | 250 | 21.1 | 20.7 | 17.0 | 18.0 | 0.3 | 1.7 | 0.14 |
| | 150 | 21.1 | 20.7 | 19.3 | 19.1 | 0.3 | 1.6 | 0.09 |
| 716600 | 250 | 19.9 | 19.7 | 18.5 | 18.2 | 0.3 | 1.5 | 0.11 |
| | 150 | 20.3 | 20.2 | 19.7 | 19.2 | 0.3 | 1.3 | 0.07 |
| 716655 | 250 | 22.8 | 19.1 | 18.3 | 16.7 | 0.3 | 1.4 | 0.05 |
| | 150 | 23.8 | 19.1 | 17.5 | 17.1 | 0.3 | 1.4 | 0.03 |
| 716772 | 250 | 21.5 | 20.0 | 17.6 | 19.7 | 0.3 | 1.3 | 0.09 |
| | 150 | 20.7 | 18.5 | 17.3 | 17.1 | 0.2 | 1.1 | 0.06 |
| 740179 | 250 | 21.0 | 22.3 | 20.4 | 21.6 | 0.3 | 1.8 | 0.18 |
| | 150 | 20.9 | 17.4 | 17.9 | 18.0 | 0.3 | 1.2 | 0.09 |
| 740256 | 150 | 21.2 | 21.2 | 18.9 | 19.9 | 0.3 | 1.4 | 0.09 |

### Plasma Chemistry Markers

Using an automated clinical chemistry analyzer (Hitachi Olympus AU400e, Melville, N.Y.), plasma concentrations of transaminases (ALT, AST) and total bilirubin (T. Bil.) were measured to evaluate the effect of the antisense oligonucleotides on hepatic function, and plasma concentrations of blood urea nitrogen (BUN) were measured to evaluate the effect of the antisense oligonucleotides on kidney function. Albumin (Alb) was also measured. The results are presented in the Tables below and show that many antisense oligonucleotides targeting human K-Ras mRNA were well tolerated in the COLO205 adenocarcinoma xenograft model.

TABLE 74
Plasma clinical chemistry

| ISIS | mg/kg/ | ALT | AST | Alb | BUN | T. bil |
|---|---|---|---|---|---|---|
| No. | week | (U/L) | (U/L) | (g/dL) | (mg/dL) | (mg/dL) |
| PBS | N/A | 29.5 | 127.0 | 3.7 | 16.7 | 0.14 |
| 651555 | 250 | 218.3 | 548.0 | 3.7 | 23.6 | 0.16 |
| | 150 | 47.8 | 145.3 | 3.6 | 18.5 | 0.15 |
| 651987 | 250 | 371.8 | 620.8 | 3.8 | 23.1 | 0.19 |
| | 150 | 232.8 | 493.0 | 3.9 | 20.9 | 0.20 |
| 695980 | 250 | 64.3 | 248.3 | 3.4 | 22.4 | 0.11 |
| | 150 | 37.0 | 146.3 | 3.9 | 20.1 | 0.15 |
| 696044 | 250 | 1176.5 | 1267.0 | 3.7 | 20.2 | 0.85 |
| | 150 | 96.8 | 298.8 | 4.4 | 20.8 | 0.24 |
| 716600 | 250 | 647.3 | 1128.0 | 3.5 | 22.9 | 0.38 |
| | 150 | 49.3 | 294.0 | 4.1 | 19.7 | 0.18 |
| 716655 | 250 | 367.5 | 923.0 | 3.9 | 29.2 | 0.94 |
| | 150 | 86.5 | 398.0 | 4.4 | 27.8 | 0.17 |
| 716772 | 250 | 65.0 | 285.0 | 4.0 | 29.3 | 0.13 |
| | 150 | 50.8 | 301.5 | 4.0 | 28.7 | 0.13 |
| 740179 | 250 | 396.5 | 400.0 | 3.6 | 20.5 | 0.15 |
| | 150 | 95.7 | 255.7 | 4.3 | 20.6 | 0.16 |
| 740256 | 150 | 225.3 | 488.5 | 3.6 | 22.0 | 0.32 |

### Example 18: Effect of Human K-Ras Antisense Oligonucleotides on Proliferation of H460 Cells (3D Assays)

An in vitro three-dimensional (3D) model was used to assess the effects of human K-Ras antisense oligonucleotides on mutant K-Ras cancer tumor cell growth. Human mutant K-Ras non-small-cell lung cancer cells (NCI-H460) were grown as spheroids on Thermo Scientific™ Nunclon™ Sphera™ ultra-low attachment microwell plates. Cancer spheroids simulate the 3D structures of tumor growth, allowing the study of tumor progression and efficacy of antisense oligonucleotides in vitro.

### Treatment

NCI-H460 cells were plated at a density of 1000 cells per well and incubated with various doses of antisense oligonucleotide or with PBS for a period of eight days. K-Ras mRNA expression and effects of the oligonucleotides on spheroid volume were evaluated and are presented in the tables below.

### RNA Analysis

At day six, RNA was isolated from the cells for real-time PCR analysis and human K-Ras mRNA levels were measured using primer probe set RTS3496_MGB, described herein above. Results are presented as average percent inhibition of K-Ras for each treatment group, relative to PBS control, normalized to beta-actin mRNA levels. Treatment with Isis antisense oligonucleotides resulted in reduction of human K-Ras mRNA in comparison to the PBS control. The half maximal inhibitory concentration (IC₅₀) of each oligonucleotide is also presented.

TABLE 75
Dose-dependent inhibition of human K-Ras mRNA
expression by antisense oligonucleotides

| | Inhibition (%) | | | | | IC₅₀ |
|---|---|---|---|---|---|---|
| ISIS No. | 12.3 nM | 37.0 nM | 111 nM | 333 nM | 1000 nM | (μM) |
| 651530 | 21.9 | 53.3 | 71.8 | 81.6 | 93.1 | 0.33 |
| 651555 | 19.9 | 44.2 | 75.9 | 84.3 | 94.5 | 0.43 |
| 651587 | 36.2 | 60.1 | 79.1 | 86.4 | 93.7 | 0.23 |
| 651987 | 32.9 | 69.9 | 81.8 | 85.7 | 93.3 | 0.2 |
| 695785 | 3.6 | 33.3 | 63.1 | 75.9 | 77.6 | 0.65 |
| 695823 | 20.4 | 37.3 | 56.2 | 79.3 | 84.3 | 0.75 |
| 695980 | 29.0 | 65.3 | 82.9 | 83.4 | 94.0 | 0.23 |
| 695995 | 20.8 | 49.7 | 69.0 | 81.4 | 86.5 | 0.35 |
| 696018 | 19.4 | 55.4 | 76.6 | 81.5 | 91.4 | 0.3 |
| 696044 | 43.7 | 76.8 | 86.8 | 94.2 | 97.6 | 0.15 |
| 716600 | 20.4 | 52.4 | 79.9 | 87.5 | 95.6 | 0.33 |
| 716655 | 10.8 | 41.0 | 73.4 | 82.7 | 92.5 | 0.7 |
| 716772 | 20.1 | 54.7 | 74.6 | 79.0 | 87.1 | 0.3 |
| 740179 | 17.2 | 52.2 | 79.0 | 84.7 | 93.6 | 0.33 |
| 740191 | 33.0 | 64.3 | 80.6 | 90.0 | 95.0 | 0.23 |
| 740223 | 12.9 | 52.7 | 75.3 | 83.4 | 92.7 | 0.38 |
| 740256 | 24.9 | 65.6 | 80.1 | 88.2 | 94.6 | 0.3 |
| 746275 | 16.5 | 67.6 | 79.6 | 87.8 | 94.5 | 0.3 |

### Spheroid Volume Analysis

At day eight, H460 spheroids were photographed and their relative volume was measurement using ImageJ. Results are presented as average percent reduction in spheroid volume for each treatment group, relative to PBS control. The half maximal growth inhibitory concentration (GI %) of each oligonucleotide is also presented.

TABLE 76
Relative spheroid volume at 8 days relative to untreated NCI-H460 cells

| ISIS No. | GI₅₀ (μM) |
|---|---|
| 651530 | 1.7 |
| 651555 | 2.0 |
| 651587 | 1.1 |
| 651987 | 1.1 |
| 695785 | 5.0 |
| 695823 | 6.0 |
| 695980 | 0.7 |
| 695995 | 2.5 |
| 696018 | 0.8 |
| 696044 | 0.8 |
| 716600 | 1.3 |
| 716655 | 2.2 |
| 716772 | 4.0 |
| 740179 | 1.1 |
| 740191 | 1.2 |
| 740223 | 1.5 |
| 740256 | 0.8 |
| 746275 | 0.9 |

### Example 19: H358 Xenograft Study of Tumor Volume

A K-Ras mutant mouse xenograft model for non-small cell lung cancer (NSCLC) was generated and used to study the efficacy of lead antisense oligonucleotides ISIS Nos. 651987 and 746275, as compared to untreated mice and to mice treated with ISIS No. 549148 as a negative control. The mice each were inoculated with NCI-H358 human NSCLC cells for tumor development.

### Treatment

Thirty-two female, athymic nude mice (CrTac:NCr-Foxn1^{nu}; Taconic Biosciences, Inc., Hudson, N.Y.), 6-8 weeks old with starting weights of 19-21 g, were divided into four groups, eight subjects per treatment group with exception of the control group treated with ISIS No. 549148, which contained five subjects. The mice were inoculated with 5×10⁶ NCI-H358 cells in 50% Matrigel (BD Bioscience) into the mammary fat pad. Antisense oligonucleotide treatment started at day 10-14 after tumor inoculation when the mean tumor size reached approximately 200 mm³. The mice were subcutaneously injected with antisense oligonucleotide at 50 mg/kg, five times per week (250 mg/kg/week) for 4.5 weeks (for a total of 22 doses), or with PBS as untreated control. Effects of KRAS antisense oligonucleotides on tumor K-Ras mRNA expression and tumor growth as well as tolerability of KRAS oligonucleotides in mice were evaluated. The body weights of the mice were measured once per week. At the end of the study (day 33), the mice were sacrificed, organs and tumor harvested, and K-Ras mRNA levels in the tumor were measured.

### RNA Analysis

RNA was extracted from tumor tissue for real-time PCR analysis and measurement of human K-Ras mRNA levels using primer probe set RTS3496_MGB, described herein above. Results are presented the Table below as average percent inhibition of K-Ras for each treatment group, relative to PBS control, normalized to beta-actin mRNA levels.

TABLE 77
Percent inhibition of human K-Ras mRNA expression
relative to control in H358 xenograft model

| ISIS No. | Inhibition (%) |
|---|---|
| 549148 | 0 |
| 651987 | 36 |
| 746275 | 56 |

### Body Weight Measurements

Body weights were measured throughout the treatment period. At the end of the study (day 33), organs were weighed and the data is presented in the Table below as the average for each treatment group at various time points.

TABLE 78
Body and organ weight measurements in H358 xenograft model

| | Body weight (g) | | | | | Organ weights (g) | | |
|---|---|---|---|---|---|---|---|---|
| Isis No. | Day 6 | Day 15 | Day 20 | Day 26 | Day 32 | kidney | liver | spleen |
| PBS | 22.6 | 23.2 | 23.4 | 23.0 | 23.2 | 0.32 | 1.29 | 0.14 |
| 549148 | 20.6 | 21.3 | 21.8 | 22.0 | 22.6 | 0.34 | 1.83 | 0.18 |
| 651987 | 23.1 | 22.5 | 21.7 | 21.2 | 21.0 | 0.34 | 1.54 | 0.10 |
| 746275 | 22.0 | 22.3 | 22.6 | 22.6 | 22.9 | 0.34 | 1.62 | 0.15 |

### Plasma Chemistry Markers

To evaluate the effect of antisense oligonucleotides on liver and kidney function, plasma levels of transaminases, total bilirubin and blood urea nitrogen (BUN) were measured using an automated clinical chemistry analyzer (Hitachi Olympus AU400e, Melville, N.Y.). The results are presented in the Table below.

TABLE 79
Plasma chemistry markers in H358 xenograft model

| | ALT | AST | Albumin | BUN | T. Bilirubin |
|---|---|---|---|---|---|
| ISIS No. | (U/L) | (U/L) | g/dL | (mg/dL) | (mg/dL) |
| PBS | 27.5 | 74.5 | 2.7 | 19.1 | 0.14 |
| 549148 | 68.6 | 102.2 | 2.8 | 19.0 | 0.11 |
| 651987 | 235.6 | 347.3 | 2.5 | 21.6 | 0.14 |
| 746275 | 258.6 | 403.8 | 2.4 | 23.5 | 0.12 |

### Tumor Volume

To evaluate the effect of antisense oligonucleotides on tumor volume, tumor sites were measured at various time points. The results are presented in the Table below.

TABLE 80
Relative tumor volume, % from day one in H358 xenograft model

| Isis No. | Day 1 | Day 5 | Day 14 | Day 19 | Day 25 | Day 31 |
|---|---|---|---|---|---|---|
| PBS | 100 | 186 | 337 | 378 | 1138 | 1908 |
| 549148 | 100 | 170 | 322 | 353 | 968 | 1109 |
| 651987 | 100 | 141 | 184 | 154 | 381 | 387 |
| 746275 | 100 | 142 | 162 | 148 | 265 | 250 |

Two lead antisense oligonucleotides, ISIS 651987 and ISIS 746275, inhibited tumor growth over the course of the study.

### Example 20: Effect of a KRAS ASO on the Proliferation of KRAS Mutant and KRAS Wild Type Tumour Cells In Vitro (3D)

The effect of 651987 on KRAS mRNA levels and proliferation in 3D was assessed in vitro in a panel of lung, colon and pancreatic cancer cell lines expressing mutant or wild type KRAS. The correlation between down regulation of KRAS mRNA (IC₅₀) and inhibition of growth in soft agar (IC₅₀) is shown in the Table below. The observations from this study show that 651987 down-regulates mutant and wild type KRAS isoforms and has selective phenotypic effects on KRAS mutant cells in vitro.

### RNA Analysis

For analysis of effect on KRAS mRNA expression cells were plated into 96-well plates and treated with dose responses of KRAS ASO for a minimum of 48 hours. For analysis of mRNA expression cell lysates prepared using FastLane Cell Probe kit (Qiagen) were used in real-time one-step RT-PCR reactions performed on a ABI 7900HT instrument (Applied Biosystems, Thermo Fisher Scientific) or a Lightcycler 480 instrument (Roche). Gene expression values were calculated using the using the comparative Ct (−ΔΔCt) method as previously described in User Bulletin #2 ABI PRISM 7700 Sequence Detection System 10/2001, using GAPDH or 18S rRNA CT values for normalisation. ABI FAM MGB Assay Probes for human KRAS (Hs00364284_g1), human GAPDH (4333764F), eukaryotic 18S rRNA (4333760F) were from Thermo Fisher Scientific.

### 3D Colony Assays

Colony assays were performed in 96 well plates. Cells (500-2000 cells per well) were seeded in 75 μl of 0.3% agar onto a 50 μl 1% agar layer in 10% RPMI-1640 growth media. The agar layers were then covered with 50 μl of media containing treatment taking into account the entire volume of agar and media. Colonies were grown for 7 to 24 days depending upon the cell line and colony formation assessed by scanning on a GelCount scanner (Oxford Optronix, Abingdon, UK) and counting colonies of a specified diameter. PC9 cells were obtained from Akiko Hiraide, Preclinical Sciences R&D, AZ, Japan. All other cells were obtained from ATCC.

TABLE 81
Details of the cell lines used in this study including results of STR
finger print testing, KRAS and other key mutations. Correlation
between IC₅₀ (μM) of KRAS mRNA down-regulation
and inhibition of colony formation by 651987 in KRAS wild type
and mutant cell lines.

| | | STR | | | KRAS | Inhibition |
|---|---|---|---|---|---|---|
| | | finger | | Other | mRNA | of colony |
| | | print | KRAS | mutations/amplifications/ | knockdown | formation |
| Cell Line | Tissue Type | tested | Mutation | deletions | (IC50 μM) | (IC50 μM) |
| A549 | Lung | Pass | G12S | CDKN2A, STK11 | 1.1810 | 1.0065 |
| NCI-H358 | Lung | Pass | G12C | KRAS amp PIK3CA, | 0.5520 | 0.3313 |
| | | | | STK11 | | |
| NCI-H460 | Lung | Pass | Q61H | CDKN2A, TP53, STK11 | 0.3240 | 0.3936 |
| NCI-H2122 | Lung | Pass | G12C | CDKN2A, TP53, STK11 | 0.3740 | 0.4580 |
| SW900 | Lung | Pass | G12V | KRAS amp, TP53, NF1 | 0.7470 | 0.3248 |
| SW480 | Colon | Pass | G12V | KRAS amp, TP53 | 0.4530 | 1.5303 |
| PANC1 | Pancreas | Pass | G12D | CDKN2A, TP53 | 0.2200 | 0.2573 |
| PC9 | Lung | Pass | WT | CDKN2A, EGFR | 0.0710 | 5.9597 |
| NCI-H1437 | Lung | Pass | WT | TP53, MEK | 0.1010 | 6.7957 |
| NCI-H1299 | Lung | Pass | WT | NRAS | 0.7130 | >10.0 |
| NCI-H1793 | Lung | Pass | WT | CDKN2A, TP53 | 0.0410 | >10.0 |
| COLO201 | Colon | Pass | WT | BRAF, TP53 | 0.2270 | >10.0 |

### Example 21: Tolerability of Antisense Oligonucleotides Targeting K-Ras in Cynomolgus Monkeys

Eight antisense oligonucleotides were compared for their relative efficacy, tolerability, pharmacokinetic and pharmacodynamic profiles in a repeated-dose study of male cynomolgus monkeys following six weeks of treatment by subcutaneous administration. These antisense oligonucleotides used in the study are described in the table below.

TABLE 82

| | | | SEQ |
|---|---|---|---|
| | | | ID |
| Isis No. | Sequence | Chemistry | NO. |
| 651530 | TGACTAATAGCAGTGG | kkk-10-kkk | 239 |
| 651555 | TTTAATGTCACAAGCA | kkk-10-kkk | 615 |
| 651587 | GATTTGTCAGCAGGAC | kkk-10-kkk | 621 |
| 651987 | GCTATTAGGAGTCTTT | kkk-10-kkk | 272 |
| 695785 | AATGGTGAATATCTTC | kkk-10-kkk | 569 |
| 695823 | AGGTAAAAGCTAACAG | kkk-10-kkk | 607 |
| 695980 | ATCTTTTAATGTCACA | kkk-10-kkk | 640 |
| 695995 | TCTCTATGAAAGCTCA | kkk-10-kkk | 655 |

### Treatment

Prior to the study, the monkeys were kept in quarantine during which the animals were observed daily for general health. The monkeys were two to three years old and weighed two to three kg. Observations were recorded for all animals once daily during the acclimation and pre-treatment period, twice daily (before and after dosing on the day of dosing, in the morning and afternoon on non-dosing day) during the treatment period, and prior to the necropsy.

All study animals were weighed once prior to group assignment during the acclimation period and once weekly during the treatment period. Body weights were taken prior to the necropsy on the day of scheduled sacrifice. Blood samples were collected from the cephalic or femoral vein for evaluation of hematology, coagulation, and clinical chemistry. Fresh urine samples were collected from all available animals for urinalysis/urine chemistry parameters Animals were fasted overnight prior to blood collection for clinical chemistry and urine collection.

At the end of the study, the monkeys were sacrificed, necropsied and organs removed. The protocols described in the Example were approved by the Institutional Animal Care and Use Committee (IACUC).

Thirty-six male cynomolgus monkeys were divided into nine groups of four monkeys each, with one group treated with 0.9% saline as a negative control. The eight antisense oligonucleotides were subcutaneously administered 40 mg/kg every other day for the first week (Days 1, 3, 5 and 7) for a total of four loading doses, and once a week thereafter (days 14, 21, 28, 35, and 42 or 43) for 6 weeks. Several clinical endpoints were measured over the course of the study. Tail bleeds were conducted at 1 week prior to the first subcutaneous administration, then again at days 9, 16, 30, 44, 58, 72, and 86.

### Body and Organ Weights

Body weight was assessed weekly. Body weights at some of these time points and organ weights (at day 44) are presented in the Table below. No remarkable effects of the antisense oligonucleotides on body weight were observed.

TABLE 83
Body and organ weights of cynomolgus monkeys treated with antisense oligonucleotides

| | | | | | Organ (g) | | | | | | | |
|---|---|---|---|---|---|---|---|---|---|---|---|---|
| | | | | | | | | | mesenteric | | | |
| | Body (g) | | | | | | | mandibular | lymph | | | |
| Isis No. | day 1 | day 14 | day 28 | day 44 | heart | kidney | liver | lymph node | node | spleen | testes | thymus |
| Saline | 2632 | 2653 | 2737 | 2788 | 11.1 | 12.5 | 54.4 | 0.5 | 1.5 | 3.2 | 1.1 | 4.1 |
| 651530 | 2503 | 2563 | 2646 | 2695 | 10.2 | 14.0 | 59.3 | 0.6 | 1.5 | 3.0 | 1.3 | 2.3 |
| 651555 | 2572 | 2628 | 2718 | 2735 | 10.6 | 15.0 | 65.0 | 0.5 | 2.3 | 4.8 | 1.5 | 3.6 |
| 651587 | 2326 | 2354 | 2409 | 2393 | 9.3 | 13.7 | 53.4 | 0.6 | 1.4 | 3.3 | 1.2 | 1.7 |
| 651987 | 2579 | 2615 | 2715 | 2833 | 10.5 | 15.4 | 70.5 | 0.7 | 2.0 | 5.1 | 1.1 | 2.5 |
| 695785 | 2652 | 2730 | 2756 | 2753 | 11.1 | 16.4 | 68.5 | 0.6 | 2.5 | 3.5 | 0.9 | 2.5 |
| 695823 | 2741 | 2819 | 2883 | 2930 | 10.0 | 13.5 | 62.3 | 0.5 | 1.7 | 3.3 | 1.5 | 3.0 |
| 695980 | 2856 | 2947 | 2990 | 3008 | 11.0 | 14.5 | 72.4 | 0.6 | 1.9 | 4.7 | 1.3 | 3.2 |
| 695995 | 2874 | 2991 | 3074 | 3283 | 11.8 | 13.9 | 66.4 | 0.6 | 2.3 | 3.4 | 2.2 | 2.5 |

### RNA Analysis

At the end of the study, RNA was extracted from monkey livers and kidneys for real-time PCR analysis of measurement of mRNA expression of K-Ras. As above, primer probe set RTS3496_MGB was used, and the results for each group were averaged and presented as percent inhibition of mRNA, relative to the PBS control, normalized with rhesus cyclophilin A. The results of two trials were averaged and are presented in the Table below.

TABLE 84
Percent inhibition of K-Ras mRNA in the cynomolgus
monkey liver relative to the PBS control

| ISIS No. | % inhibition | SEQ ID NO |
|---|---|---|
| 651530 | 73 | 239 |
| 651555 | 81 | 615 |
| 651587 | 78 | 621 |
| 651987 | 84 | 272 |
| 695785 | 88 | 569 |
| 695823 | 71 | 607 |
| 695980 | 45 | 640 |
| 695995 | 71 | 655 |

TABLE 85
Inhibition of K-Ras mRNA relative to the PBS control
in various monkey tissues after treatment with ISIS 651987

| | Oligo ID |
|---|---|
| | 651987 |
| Tissue | % Inhibition |
| Liver | 84 |
| Kidney | 69 |
| Lung | 23 |
| Duodenum | 53 |
| Pancreas | 21 |
| Heart | 32 |

### Hematology

To evaluate any effect of ISIS oligonucleotides in cynomolgus monkeys on hematologic parameters, blood samples of approximately 1.3 mL of blood were collected on day 44 from each of the study animals in tubes containing K₂-EDTA. Samples were analyzed for red blood cell (RBC) count, white blood cells (WBC) count, basophil count (BAS), as well as for platelet count (PLT) and mean platelet volume (MPV) using an ADVIA120 hematology analyzer (Bayer, USA). The data is presented in the Table below.

TABLE 86
Hematology

| | BAS | PLT | MPV | RBC | WBC |
|---|---|---|---|---|---|
| ISIS No. | 10³/uL | 10³/uL | fL | 10⁶/uL | 10³/uL |
| Saline | 0.02 | 346 | 8.4 | 5 | 12 |
| 651530 | 0.02 | 385 | 8.3 | 5 | 11 |
| 651555 | 0.03 | 340 | 8.8 | 6 | 12 |
| 651587 | 0.03 | 450 | 7.3 | 6 | 12 |
| 651987 | 0.02 | 362 | 8.1 | 6 | 9 |
| 695785 | 0.03 | 339 | 8.5 | 6 | 11 |
| 695823 | 0.03 | 305 | 8.3 | 6 | 9 |
| 695980 | 0.02 | 301 | 8.4 | 5 | 7 |
| 695995 | 0.03 | 288 | 11.2 | 5 | 12 |

The data indicate the oligonucleotides did not cause any significant changes in hematologic parameters outside the expected range for antisense oligonucleotides at this dose. These antisense oligonucleotides were well tolerated in terms of hematologic parameters in the monkeys.

### Liver and Kidney Function

To evaluate the effect of these antisense oligonucleotides on liver and kidney function, samples of blood, plasma, serum and urine were collected from all study groups on day 44. The blood samples were collected via femoral venipuncture, 48 hrs post-dosing. The monkeys were fasted overnight prior to blood collection. Approximately 1.5 mL of blood was collected from each animal into tubes without anticoagulant for serum separation. Levels of the various markers were measured using an automated clinical chemistry analyzer (Hitachi Olympus AU400e, Melville, N.Y.). Total urine protein and urine creatinine levels were measured, and the ratio of total urine protein to creatinine (P/C Ratio) was determined.

To evaluate the effect of the antisense oligonucleotides on hepatic function, plasma concentrations of transaminases (ALT, AST), Albumin (Alb) and total bilirubin (“T. Bil”) were measured. To evaluate the effect of the antisense oligonucleotides on kidney function, plasma concentrations of blood urea nitrogen (BUN) and creatinine (Cre) were measured. Urine levels of albumin (Alb), creatinine (Cre) and total urine protein (Micro Total Protein (MTP)) were measured, and the ratio of total urine protein to creatinine (P/C ratio) was determined.

To evaluate any inflammatory effect of the ISIS oligonucleotides in cynomolgus monkeys, C-reactive protein (CRP), which is synthesized in the liver and which serves as a marker of inflammation, was measured on day 42. For this, blood samples were taken from fasted monkeys, the tubes were kept at room temperature for a minimum of 90 min., and centrifuged at 3,000 rpm for 10 min at room temperature to obtain serum. The results are presented in the Tables below and indicate that most of the antisense oligonucleotides targeting human K-Ras were well tolerated in cynomolgus monkeys.

TABLE 87
Serum and urine clinical chemistry

| | Serum (day 44) | | | | | | | | Urine (day 44) | | | |
|---|---|---|---|---|---|---|---|---|---|---|---|---|
| ISIS | C3 | ALT | AST | Alb | BUN | CRP | Cre | T.bil | Alb | Cre | MTP | P/C |
| No. | mg/dL | U/L | U/L | g/dL | mg/dL | mg/L | mg/dL | mg/dL | g/dL | mg/dL | mg/dL | ratio |
| Saline | 103.3 | 43.3 | 60.0 | 4.0 | 25.1 | 0.27 | 0.78 | 0.24 | 1.0 | 21.5 | 0.00 | 0.00 |
| 651530 | 78.9 | 42.9 | 75.6 | 4.1 | 25.1 | 0.14 | 0.79 | 0.25 | 2.1 | 32.5 | 0.23 | 0.01 |
| 651555 | 101.8 | 83.1 | 92.7 | 4.0 | 24.6 | 0.21 | 0.73 | 0.19 | 11.8 | 34.0 | 1.75 | 0.06 |
| 651587 | 93.9 | 93.7 | 94.3 | 4.0 | 22.4 | 0.13 | 0.81 | 0.22 | 8.4 | 76.2 | 5.10 | 0.07 |
| 651987 | 83.1 | 67.0 | 77.9 | 3.9 | 27.6 | 0.23 | 0.81 | 0.21 | 4.8 | 59.3 | 0.96 | 0.02 |
| 695785 | 100.4 | 72.2 | 107.1 | 4.1 | 24.6 | 0.11 | 0.81 | 0.20 | 6.6 | 42.9 | 0.51 | 0.01 |
| 695823 | 97.1 | 68.5 | 67.9 | 4.2 | 23.9 | 0.14 | 0.85 | 0.23 | 4.2 | 33.6 | 0.12 | 0.00 |
| 695980 | 85.9 | 60.2 | 63.8 | 4.0 | 27.2 | 0.19 | 0.84 | 0.26 | 4.4 | 37.4 | 0.17 | 0.01 |
| 695995 | 93.4 | 47.8 | 82.5 | 4.1 | 28.2 | 0.28 | 0.90 | 0.25 | 3.5 | 44.2 | 0.09 | 0.00 |

### Complement C3 Levels

C3 levels were measured on several days during the study period prior to dosing and on Day 42 (pre- and post-dosing). When compared on Day 42 pre-dose to concurrent control (saline) and baseline (Day −14 pre-dose), a decreasing trend was noted in all antisense oligonucleotide-treated groups except animals treated with ISIS No. 651555. The lowest C3 level (82% of Day 42 pre-dose baseline value) was shown in animals treated with ISIS No. 651987 on Day 42 compared to pre-dose. The results of the complement C3 analysis are shown in the Table below.

TABLE 88
C3 Analysis on Day 42 as compared to
baseline and control groups (mg/dL)

| | | | | % decrease |
|---|---|---|---|---|
| | | | | compared to |
| | | | % decrease compared | control group |
| ISIS | Day −14 | Day 42 | to baseline | (pre-dose |
| No. | (pre-dose) | (pre-dose) | (pre-dose on Day −14) | on Day 42) |
| Saline | 110 | 103 | −7 | 0 |
| 651530 | 94 | 81 | −14 | −21 |
| 651555 | 108 | 110 | +2 | +7 |
| 651587 | 105 | 93 | −12 | −9 |
| 651987 | 109 | 89 | −18 | −13 |
| 695785 | 112 | 101 | −10 | −2 |
| 695823 | 105 | 98 | −7 | −4 |
| 695980 | 91 | 86 | −6 | −16 |
| 695995 | 108 | 95 | −12 | −7 |

Decreased C3 levels (approximately decreased by 6 to 18% compared to baseline control) were observed in all oligonucleotide-treated groups except animals treated with ISIS No. 651555. The lowest C3 level was shown in animals treated with ISIS No. 651987.

TABLE 89
Concentrations of ISIS antisense oligonucleotide
in liver and lung in cynomolgus monkeys

| | | |
|---|---|---|
| Study | Concentration | Liver/lung |

| ISIS No. | day | Liver (μg/g) | Lung (μg/g) | ratio |
|---|---|---|---|---|
| 695785 | 44 | 658 | 69 | 9.6 |
| 695823 | 45 | 339 | 29 | 11.7 |
| 695995 | 45 | 556 | 38 | 14.5 |

TABLE 90
K-Ras concentrations in liver and kidney cortex in
cynomolgus monkeys following 6 weeks of dosing

| | | | Kidney/liver |
|---|---|---|---|
| ISIS No. | Liver | Kidney | ratio |
| 651530 | 416 | 1012 | 2.4 |
| 651555 | 467 | 1127 | 2.4 |
| 651587 | 318 | 1070 | 3.4 |
| 651987 | 310 | 832 | 2.7 |
| 695785 | 587 | 1719 | 2.9 |
| 695823 | 519 | 2748 | 5.3 |
| 695980 | 601 | 2807 | 4.7 |
| 695995 | 464 | 1325 | 2.9 |

In conclusion, subcutaneous injection of eight antisense oligonucleotides targeting K-Ras mRNA for 6 weeks was well tolerated with no overt toxicity. No treatment-related changes in mortality, body weight, coagulation and urinalysis/urine chemistry were observed in this study.

### Example 22: Tolerability of Antisense Oligonucleotides Targeting K-Ras in Cynomolgus Monkeys

Six antisense oligonucleotides were compared for their relative efficacy, tolerability, pharmacokinetic and pharmacodynamic profiles in a repeated-dose study of male cynomolgus monkeys following six weeks of treatment by subcutaneous administration. These antisense oligonucleotides used in the study are described in the table below.

TABLE 91

| | | | SEQ |
|---|---|---|---|
| | | | ID |
| Isis No. | Sequence | Chemistry | NO. |
| 696018 | CTCTTGATTTGTCAGC | kkk-10-kkk | 678 |
| 696044 | GTGTTTATGCAATGTT | kkk-10-kkk | 715 |
| 716600 | CCATTTATGTGACTAG | kkk-10-kkk | 790 |
| 716655 | TGTTTATGCAATGTTA | kkk-10-kkk | 854 |
| 740233 | GTGTTTATGCAATGTT | kkk-8-kdkdk | 2158 |
| 746275 | TCTTGATTTGTCAGCA | kk-10-keke | 804 |

### Treatment

Prior to the study, the monkeys were kept in quarantine during which the animals were observed daily for general health. The monkeys were two to three years old and weighed two to three kg. Observations were recorded for all animals once daily during the acclimation and pre-treatment period, twice daily (before and after dosing on the day of dosing, in the morning and afternoon on non-dosing day) during the treatment period, and prior to the necropsy.

All study animals were weighed once prior to group assignment during the acclimation period and once weekly during the treatment period. Body weights were taken prior to the necropsy on the day of scheduled sacrifice. Blood samples were collected from the cephalic or femoral vein for evaluation of hematology, coagulation, and clinical chemistry. Fresh urine samples were collected from all available animals for urinalysis/urine chemistry parameters Animals were fasted overnight prior to blood collection for clinical chemistry and urine collection.

At the end of the study, the monkeys were sacrificed, necropsied and organs removed. The protocols described in the Example were approved by the Institutional Animal Care and Use Committee (IACUC).

Twenty-eight male cynomolgus monkeys were divided into seven groups of four monkeys each, with one group treated with 0.9% saline as a negative control. The six antisense oligonucleotides were subcutaneously administered 40 mg/kg every other day for the first week (Days 1, 3, 5 and 7) for a total of four loading doses, and once a week thereafter (days 14, 21, 28, 35, and 4) for 6 weeks. Several clinical endpoints were measured over the course of the study. Tail bleeds were conducted at 2 weeks and 1 week prior to the first subcutaneous administration, then again at days 16, 30, and 44. Serum was tested at 2 weeks prior to the first subcutaneous administration and at day 42 and urine was collected at 1 week prior to study start and at day 44.

### Body and Organ Weights

Body weight was assessed weekly. Body weights at some of these time points and organ weights (at day 44) are presented in the Table below. No remarkable effects of the antisense oligonucleotides on body weight were observed.

TABLE 92
Body weights of cynomolgus monkeys treated with antisense oligonucleotides

| | Body (g) | | | | | | | |
|---|---|---|---|---|---|---|---|---|
| Isis No. | day 1 | day 7 | day 14 | day 21 | day 28 | day 35 | day 42 | day 44 |
| PBS | 2512.5 | 2630.8 | 2536.0 | 2542.5 | 2606.3 | 2565.8 | 2557.8 | 2,614.5 |
| 696018 | 2478.0 | 2596.8 | 2537.3 | 2527.5 | 2586.0 | 2539.3 | 2534.0 | 2,596.8 |
| 696044 | 2507.0 | 2624.5 | 2574.5 | 2607.0 | 2656.0 | 2610.3 | 2589.5 | 2,630.0 |
| 716600 | 2458.5 | 2537.5 | 2496.0 | 2503.8 | 2560.8 | 2526.5 | 2528.5 | 2,543.8 |
| 716655 | 2454.3 | 2506.8 | 2470.8 | 2508.8 | 2569.5 | 2520.8 | 2551.8 | 2,599.8 |
| 740233 | 2522.5 | 2554.3 | 2501.0 | 2545.5 | 2591.5 | 2540.5 | 2541.8 | 2,601.0 |
| 746275 | 2365.5 | 2411.0 | 2374.5 | 2408.5 | 2469.3 | 2419.5 | 2410.8 | 2,421.8 |

TABLE 93
Organ weights of cynomolgus monkeys treated with antisense oligonucleotides

| | Organ (g) | | | | | | | |
|---|---|---|---|---|---|---|---|---|
| | | | | mandibular | mesenteric | | | |
| | | | | lymph | lymph | | | |
| Isis No. | heart | kidney | liver | node | node | spleen | testes | thymus |
| PBS | 8.89 | 14.10 | 58.39 | 0.29 | 1.03 | 2.62 | 0.80 | 2.20 |
| 696018 | 10.06 | 13.72 | 67.02 | 0.51 | 2.67 | 4.03 | 1.20 | 0.68 |
| 696044 | 9.25 | 17.02 | 77.61 | 0.57 | 2.82 | 6.23 | 1.46 | 1.24 |
| 716600 | 8.96 | 13.34 | 69.54 | 0.43 | 2.43 | 3.20 | 1.29 | 2.38 |
| 716655 | 8.67 | 14.85 | 69.76 | 0.48 | 2.05 | 3.45 | 1.32 | 1.04 |
| 740233 | 10.05 | 15.86 | 79.39 | 0.79 | 3.13 | 6.23 | 1.07 | 1.33 |
| 746275 | 8.40 | 12.96 | 65.13 | 0.87 | 4.10 | 5.14 | 0.94 | 1.12 |

### Hematology

To evaluate any effect of ISIS oligonucleotides in cynomolgus monkeys on hematologic parameters, blood samples of approximately 1.3 mL of blood were collected on day 44 from each of the study animals in tubes containing K₂-EDTA. Samples were analyzed for red blood cell (RBC) count, white blood cells (WBC) count, basophil count (BAS), as well as for platelet count (PLT) and mean platelet volume (MPV) using an ADVIA120 hematology analyzer (Bayer, USA). The data is presented in the Table below.

TABLE 94
Hematology

| | BAS | PLT | MPV | RBC | WBC |
|---|---|---|---|---|---|
| ISIS No. | 10³/uL | 10³/uL | fL | 10⁶/uL | 10³/uL |
| PBS | .043 | 379.3 | 8.00 | 5.51 | 14.04 |
| 696018 | .055 | 352.0 | 8.78 | 6.16 | 13.12 |
| 696044 | .048 | 293.0 | 8.75 | 5.23 | 10.23 |
| 716600 | .038 | 454.0 | 7.73 | 5.94 | 14.73 |
| 716655 | .030 | 408.0 | 6.88 | 6.04 | 11.47 |
| 740233 | .035 | 352.3 | 7.40 | 5.59 | 7.99 |
| 746275 | .043 | 352.8 | 7.13 | 5.53 | 9.24 |

The data indicate the oligonucleotides did not cause any significant changes in hematologic parameters outside the expected range for antisense oligonucleotides at this dose. These antisense oligonucleotides were well tolerated in terms of hematologic parameters in the monkeys.

### Liver and Kidney Function

To evaluate the effect of these antisense oligonucleotides on liver and kidney function, samples of blood, plasma, serum and urine were collected from all study groups on day 44. The blood samples were collected via femoral venipuncture, 48 hrs post-dosing. The monkeys were fasted overnight prior to blood collection. Approximately 1.5 mL of blood was collected from each animal into tubes without anticoagulant for serum separation. Levels of the various markers were measured using an automated clinical chemistry analyzer (Hitachi Olympus AU400e, Melville, N.Y.). Total urine protein and urine creatinine levels were measured, and the ratio of total urine protein to creatinine (P/C Ratio) was determined.

To evaluate the effect of the antisense oligonucleotides on hepatic function, plasma concentrations of transaminases (ALT, AST), Albumin (Alb) and total bilirubin (“T. Bil”) were measured. To evaluate the effect of the antisense oligonucleotides on kidney function, plasma concentrations of blood urea nitrogen (BUN) and creatinine (Cre) were measured. Urine levels of albumin (Alb), creatinine (Cre) and total urine protein (Micro Total Protein (MTP)) were measured, and the ratio of total urine protein to creatinine (P/C ratio) was determined.

To evaluate any inflammatory effect of the ISIS oligonucleotides in cynomolgus monkeys, C-reactive protein (CRP), which is synthesized in the liver and which serves as a marker of inflammation, was measured on day 42. For this, blood samples were taken from fasted monkeys, the tubes were kept at room temperature for a minimum of 90 min., and centrifuged at 3,000 rpm for 10 min at room temperature to obtain serum. The results are presented in the Tables below and indicate that most of the antisense oligonucleotides targeting human K-Ras were well tolerated in cynomolgus monkeys.

TABLE 95
Serum and urine clinical chemistry

| | Serum (day 44) | | | | | | | | Urine (day 42) | | |
|---|---|---|---|---|---|---|---|---|---|---|---|
| ISIS | C3 | ALT | AST | Alb | BUN | CRP | Cre | T.bil | Alb | Cre | P/C |
| No. | mg/dL | U/L | U/L | g/dL | mg/dL | mg/L | mg/dL | mg/dL | g/dL | mg/dL | ratio |
| PBS | 125.5 | 45.5 | 74.0 | 4.3 | 27.1 | .158 | .718 | .206 | .150 | 35.31 | .000 |
| 696018 | 114.5 | 79.2 | 95.1 | 3.9 | 34.1 | .278 | .988 | .157 | 3.000 | 42.10 | .175 |
| 696044 | 105.1 | 39.2 | 96.9 | 3.7 | 29.1 | .435 | .795 | .235 | 1.075 | 36.83 | .120 |
| 716600 | 92.0 | 72.0 | 130.7 | 3.9 | 33.9 | .190 | .903 | .192 | .450 | 39.47 | .018 |
| 716655 | 89.5 | 63.5 | 76.5 | 3.7 | 27.0 | .153 | .753 | .189 | .225 | 36.03 | .003 |
| 740233 | 90.6 | 58.9 | 101.8 | 3.7 | 24.2 | .283 | .668 | .190 | .125 | 34.68 | .000 |
| 746275 | 85.4 | 46.2 | 82.9 | 3.5 | 25.3 | .428 | .795 | .184 | 1.600 | 46.52 | .073 |

### RNA Analysis

At the end of the study, RNA was extracted from various monkey tissues for real-time PCR analysis of measurement of mRNA expression of K-Ras for the animals treated with ISIS 746275. As above, primer probe set RTS3496_MGB was used, and the results for each group were averaged and presented as percent inhibition of mRNA, relative to the PBS control, normalized with rhesus cyclophilin A.

TABLE 96
Inhibition of K-Ras mRNA relative to the PBS control in
various monkey tissues after treatment with ISIS 746275

| Tissue | % Inhibition |
|---|---|
| Liver | 69 |
| Kidney | 51 |
| Lung | 27 |
| Duodenum | 39 |
| Pancreas | 0 |
| Heart | 28 |

What is claimed:

1. A compound comprising a modified oligonucleotide consisting of 8 to 80 linked nucleosides and having a nucleobase sequence comprising at least 8, 9, 10, 11, or 12 contiguous nucleobases of any of the nucleobase sequences of SEQ ID NOs: 13-2190.

2. A compound comprising a modified oligonucleotide consisting of 8 to 80 linked nucleosides and having a nucleobase sequence comprising the nucleobase sequence of any one of SEQ ID NOs: 13-2190.

3. A compound comprising a modified oligonucleotide consisting of the nucleobase sequence of any one of SEQ ID NOs: 13-2190.

4. A compound comprising a modified oligonucleotide consisting of 8 to 80 linked nucleosides complementary within nucleobases 463-478, 877-892, 1129-1144, 1313-1328, 1447-1462, 1686-1701, 1690-1705, 1778-1793, 1915-1930, 1919-1934, 1920-1935, 2114-2129, 2115-2130, 2461-2476, 2462-2477, 2463-2478, 4035-4050 of SEQ ID NO: 1, wherein said modified oligonucleotide is at least 85%, 90%, 95%, or 100% complementary to SEQ ID NO: 1.

5. A compound comprising a modified oligonucleotide consisting of 8 to 80 linked nucleosides having a nucleobase sequence comprising at least 8, 9, 10, 11, or 12 contiguous nucleobases of any of the nucleobase sequences of any one of SEQ ID NOs: 272, 804, 239, 569, 607, 615, 621, 640, 655, 678, 715, 790, 854, 1028, 2130, 2136, 2142, 2154, and 2158.

6. A compound comprising a modified oligonucleotide consisting of 16 to 80 linked nucleosides having a nucleobase sequence comprising the nucleobase sequences of any one of SEQ ID NOs: 272, 804, 239, 569, 607, 615, 621, 640, 655, 678, 715, 790, 854, 1028, 2130, 2136, 2142, 2154, and 2158.

7. A compound comprising a modified oligonucleotide consisting of 16 linked nucleosides having a nucleobase sequence consisting of any one of SEQ ID NOs: 272, 804, 239, 569, 607, 615, 621, 640, 655, 678, 715, 790, 854, 1028, 2130, 2136, 2142, 2154, and 2158.

8. The compound of any one of claims 1-7, wherein the modified oligonucleotide comprises:
a gap segment consisting of linked deoxynucleosides;
a 5′ wing segment consisting of linked nucleosides; and
a 3′ wing segment consisting of linked nucleosides;
wherein the gap segment is positioned between the 5′ wing segment and the 3′ wing segment and wherein each nucleoside of each wing segment comprises a modified sugar.

9. A compound comprising a modified oligonucleotide consisting of 16 to 80 linked nucleosides having a nucleobase sequence comprising the nucleobase sequence of any one of SEQ ID NOs: 272, 239, 569, 607, 615, 621, 640, 655, 678, 715, 790, and 854, wherein the modified oligonucleotide comprises:
a gap segment consisting of ten linked deoxynucleosides;
a 5′ wing segment consisting of three linked nucleosides; and
a 3′ wing segment consisting of three linked nucleosides;
wherein the gap segment is positioned between the 5′ wing segment and the 3′ wing segment, wherein each nucleoside of each wing segment comprises a constrained ethyl (cEt) nucleoside; wherein each internucleoside linkage is a phosphorothioate linkage and wherein each cytosine is a 5-methylcytosine.

10. A compound comprising a modified oligonucleotide consisting of 16 to 80 linked nucleosides having a nucleobase sequence comprising the nucleobase sequence of SEQ ID NO: 2130, wherein the modified oligonucleotide comprises:
a gap segment consisting of nine linked deoxynucleosides;
a 5′ wing segment consisting of one linked nucleoside; and
a 3′ wing segment consisting of six linked nucleosides;
wherein the gap segment is positioned between the 5′ wing segment and the 3′ wing segment; wherein the 5′ wing segment comprises a cEt nucleoside; wherein the 3′ wing segment comprises a cEt nucleoside, a 2′-O-methoxyethyl nucleoside, a cEt nucleoside, a 2′-O-methoxyethyl nucleoside, a cEt nucleoside, and 2′-O-methoxyethyl nucleoside in the 5′ to 3′ direction; wherein each internucleoside linkage is a phosphorothioate linkage; and wherein each cytosine is a 5-methylcytosine.

11. A compound comprising a modified oligonucleotide consisting of 16 to 80 linked nucleosides having a nucleobase sequence comprising the nucleobase sequence of any one of SEQ ID NOs: 804, 1028, and 2136, wherein the modified oligonucleotide comprises:
a gap segment consisting of ten linked deoxynucleosides;
a 5′ wing segment consisting of two linked nucleosides; and
a 3′ wing segment consisting of four linked nucleosides;
wherein the gap segment is positioned between the 5′ wing segment and the 3′ wing segment; wherein the 5′ wing segment comprises a cEt nucleoside and a cEt nucleoside in the 5′ to 3′ direction; wherein the 3′ wing segment comprises a cEt nucleoside, a 2′-O-methoxyethyl nucleoside, a cEt nucleoside, and a 2′-O-methoxyethyl nucleoside in the 5′ to 3′ direction; wherein each internucleoside linkage is a phosphorothioate linkage; and wherein each cytosine is a 5-methylcytosine.

12. A compound comprising a modified oligonucleotide consisting of 16 to 80 linked nucleosides having a nucleobase sequence comprising the nucleobase sequence of SEQ ID NO: 2142, wherein the modified oligonucleotide comprises:
a gap segment consisting of eight linked deoxynucleosides;
a 5′ wing segment consisting of two linked nucleosides; and
a 3′ wing segment consisting of six linked nucleosides;
wherein the gap segment is positioned between the 5′ wing segment and the 3′ wing segment; wherein the 5′ wing segment comprises a cEt nucleoside and a cEt nucleoside in the 5′ to 3′ direction; wherein the 3′ wing segment comprises a cEt nucleoside, a 2′-O-methoxyethyl nucleoside, a cEt nucleoside, a 2′-O-methoxyethyl nucleoside, a cEt nucleoside, and a cEt nucleoside in the 5′ to 3′ direction; wherein each internucleoside linkage is a phosphorothioate linkage; and wherein each cytosine is a 5-methylcytosine.

13. A compound comprising a modified oligonucleotide consisting of 16 to 80 linked nucleosides having a nucleobase sequence comprising the nucleobase sequence of SEQ ID NO: 2154, wherein the modified oligonucleotide comprises:
a gap segment consisting of nine linked deoxynucleosides;
a 5′ wing segment consisting of two linked nucleosides; and
a 3′ wing segment consisting of five linked nucleosides;
wherein the gap segment is positioned between the 5′ wing segment and the 3′ wing segment; wherein the 5′ wing segment comprises a cEt nucleoside and a cEt nucleoside in the 5′ to 3′ direction; wherein the 3′ wing segment comprises a cEt nucleoside, a 2′-O-methoxyethyl nucleoside, a cEt nucleoside, a 2′-O-methoxyethyl nucleoside, and a cEt nucleoside in the 5′ to 3′ direction; wherein each internucleoside linkage is a phosphorothioate linkage; and wherein each cytosine is a 5-methylcytosine.

14. A compound comprising a modified oligonucleotide consisting of 16 to 80 linked nucleosides having a nucleobase sequence comprising the nucleobase sequence of SEQ ID NO: 2158, wherein the modified oligonucleotide comprises:
a gap segment consisting of eight linked deoxynucleosides;
a 5′ wing segment consisting of three linked nucleosides; and
a 3′ wing segment consisting of five linked nucleosides;
wherein the gap segment is positioned between the 5′ wing segment and the 3′ wing segment; wherein the 5′ wing segment comprises a cEt nucleoside, a cEt nucleoside, and a cEt nucleoside in the 5′ to 3′ direction; wherein the 3′ wing segment comprises a cEt nucleoside, a deoxynucleoside, a cEt nucleoside, a deoxynucleoside, and a cEt nucleoside in the 5′ to 3′ direction; wherein each internucleoside linkage is a phosphorothioate linkage; and wherein each cytosine is a 5-methylcytosine.

15. The compound of any one of claims 1-14, wherein the oligonucleotide is at least 80%, 85%, 90%, 95% or 100% complementary to SEQ ID NO: 1 or 2.

16. The compound of any one of claims 1-15, wherein the modified oligonucleotide comprises at least one modified internucleoside linkage, at least one modified sugar, or at least one modified nucleobase.

17. The compound of claim 16, wherein the modified internucleoside linkage is a phosphorothioate internucleoside linkage.

18. The compound of claim 16 or 17, wherein the modified sugar is a bicyclic sugar.

19. The compound of claim 18, wherein the bicyclic sugar is selected from the group consisting of: 4′-(CH₂)—O-2′ (LNA); 4′-(CH₂)₂-O-2′ (ENA); and 4′-CH(CH₃)—O-2′ (cEt).

20. The compound of any one of claims 16-19, wherein the modified sugar is 2′-O-methoxyethyl.

21. The compound of any one of claims 16-20, wherein the modified nucleobase is a 5-methylcytosine.

22. The compound of any one of claims 1-21, wherein the modified oligonucleotide comprises:
a gap segment consisting of linked deoxynucleosides;
a 5′ wing segment consisting of linked nucleosides; and
a 3′ wing segment consisting of linked nucleosides;
wherein the gap segment is positioned immediately adjacent to and between the 5′ wing segment and the 3′ wing segment and wherein each nucleoside of each wing segment comprises a modified sugar.

23. The compound of any one of claims 1-22, wherein the compound is single-stranded.

24. The compound of any one of claims 1-23, wherein the compound is double-stranded.

25. The compound of any one of claims 1-24, wherein the compound comprises ribonucleotides.

26. The compound of claim 25, wherein the compound comprises a double-stranded RNA oligonucleotide, wherein one strand of the double-stranded RNA oligonucleotide is the modified oligonucleotide.

27. The compound of any one of claims 1-24, wherein the compound comprises deoxyribonucleotides.

28. The compound of any one of claims 1-27, wherein the modified oligonucleotide consists of 10 to 30, 12 to 30, 15 to 30, 16 to 30, or 16 linked nucleosides.

29. The compound of any one of claims 1-28, wherein the compound comprises a conjugate and the modified oligonucleotide.

30. The compound of any one of claims 1-28, wherein the compound consists of a conjugate and the modified oligonucleotide.

31. The compound of any one of claims 1-28, wherein the compound consists of the modified oligonucleotide.

32. A compound consisting of a pharmaceutically acceptable salt of any of the compounds of claims 1-31.

33. The compound of claim 32, wherein the pharmaceutically acceptable salt is a sodium salt.

34. The compound of claim 32, wherein the pharmaceutically acceptable salt is a potassium salt.

35. A compound comprising ISIS 651987, or a pharmaceutically acceptable salt thereof, having the formula:

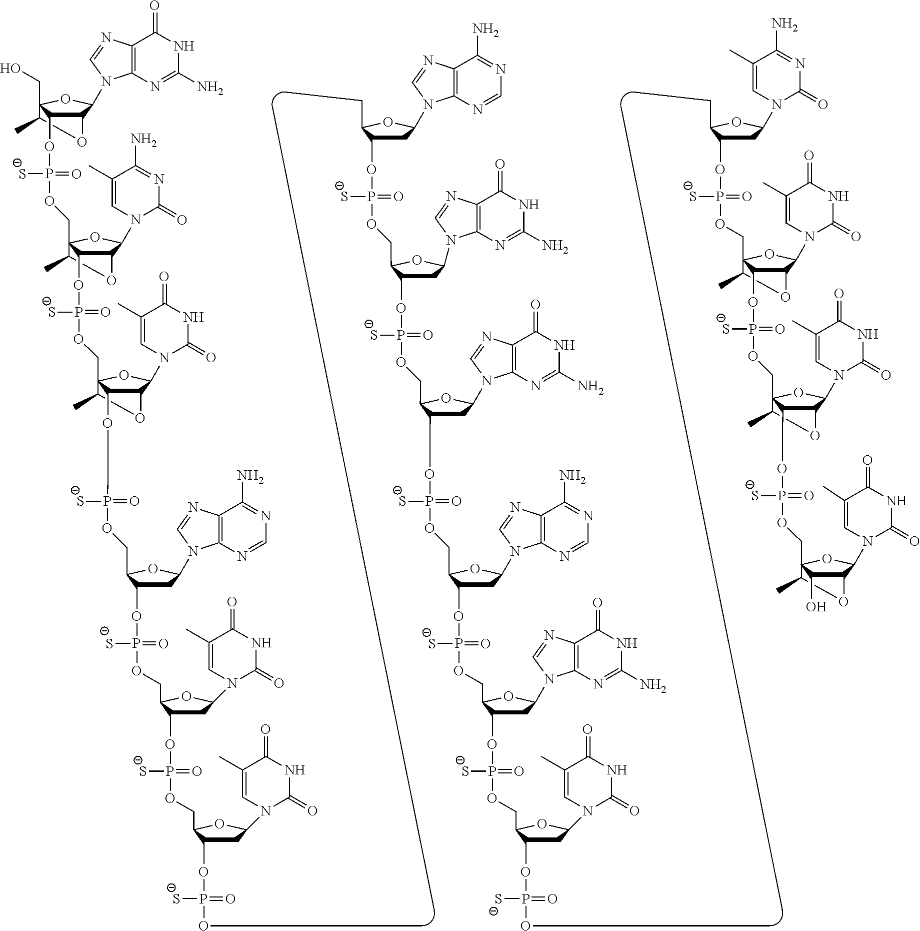

36. A compound consisting of ISIS 651987, or a pharmaceutically acceptable salt thereof, having the formula:

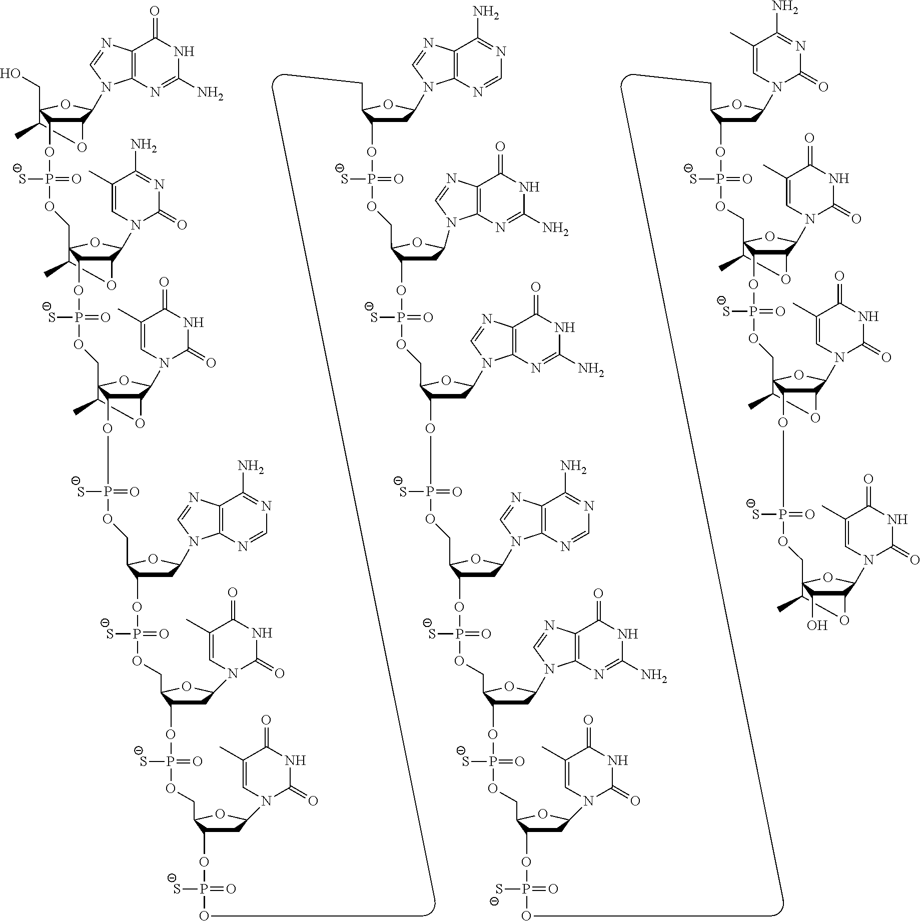

37. The compound of claim 35 or 36, wherein the pharmaceutically acceptable salt is a sodium salt.

38. A composition comprising the compound of any one of claims 1-37 and a pharmaceutically acceptable carrier.

39. A method of treating, preventing, or ameliorating cancer in an individual comprising administering to the individual the compound of any one of claims 1-37 or composition of claim 38, thereby treating, preventing, or ameliorating cancer in the individual.

40. The method of claim 39, wherein the cancer is lung cancer, non-small cell lung carcinoma (NSCLC), small-cell lung carcinoma (SCLC)), gastrointestinal cancer, large intestinal cancer, small intestinal cancer, colon cancer, colorectal cancer, bladder cancer, liver cancer, stomach cancer, esophageal cancer, pancreatic cancer, biliary tract cancer, breast cancer, ovarian cancer, endometrial cancer, cervical cancer, prostate cancer, hematopoetic cancer, brain cancer, glioblastoma, malignant peripheral nerve sheath tumor (MPNST), neurofibromatosis type 1 (NF1) mutant MPNST, neurofibroma, leukemia, myeloid leukemia, or lymphoma.

41. The method of claim 39 or 40, wherein administering the compound reduces the number of cancer cells in the individual, reduces the size of a tumor in the individual, reduces or inhibits growth or proliferation of a tumor in the individual, prevents metastasis or reduces the extent of metastasis in the individual, or extends survival of the individual.

42. A method of inhibiting expression of KRAS in a cell comprising contacting the cell with the compound of any one of claims 1-37 or composition of claim 38, thereby inhibiting expression of KRAS in the cell.

43. Use of the compound of any one of claims 1-37 or composition of claim 38 for treating, preventing, or ameliorating cancer in an individual.

44. Use of the compound of any one of claims 1-37 or composition of claim 38 for the manufacture of a medicament for treating cancer.

45. The use of claim 43 or 44, wherein the cancer is lung cancer, non-small cell lung carcinoma (NSCLC), small-cell lung carcinoma (SCLC)), gastrointestinal cancer, large intestinal cancer, small intestinal cancer, colon cancer, colorectal cancer, bladder cancer, liver cancer, stomach cancer, esophageal cancer, pancreatic cancer, biliary tract cancer, breast cancer, ovarian cancer, endometrial cancer, cervical cancer, prostate cancer, hematopoetic cancer, brain cancer, glioblastoma, malignant peripheral nerve sheath tumor (MPNST), neurofibromatosis type 1 (NF1) mutant MPNST, neurofibroma, leukemia, myeloid leukemia, or lymphoma.
